(19) United States
(12) Patent Application Publication
(10) Pub. No.: US 2024/0383904 A1
(43) Pub. Date: Nov. 21, 2024

(54) HETEROCYCLIC COMPOUNDS

(71) Applicant: Hoffmann-La Roche Inc., Little Falls, NJ (US)

(72) Inventors: Machoud AMOUSSA, Berlin (DE); Joerg BENZ, Rheinfelden (DE); Julie Elisabeth Francoise BLAISING, Basel (CH); Maude GIROUD, Basel (CH); Uwe GRETHER, Efringen-Kirchen (DE); Carsten KROLL, Basel (CH); Bernd KUHN, Reinach BL (CH); Rainer E. MARTIN, Basel (CH); Fionn Susannah O’HARA, Basel (CH); Bernd PUELLMANN, Aesch (CH); Martin RITTER, Mumpf (CH); Didier ROMBACH, Mulhouse (FR); Philipp Claudio SCHMID, Birsfelden (CH); Matthias Beat WITTWER, Riehen (CH)

(73) Assignee: Hoffmann-La Roche Inc., Little Falls, NJ (US)

(21) Appl. No.: 18/632,636

(22) Filed: Apr. 11, 2024

Related U.S. Application Data
(63) Continuation of application No. PCT/EP2022/078329, filed on Oct. 12, 2022.

(30) Foreign Application Priority Data
Oct. 13, 2021 (EP) 21202310.5
May 18, 2022 (EP) 22174059.0

(51) Int. Cl. C07D 487/04; A61K 9/20; A61K 9/48; A61K 31/407; A61K 31/4155; A61K 31/4178; A61K 31/4188; A61K 31/4192; A61K 31/4196; A61K 31/422; A61K 31/424; A61K 31/427; A61K 31/433; A61K 31/437; A61K 31/438; A61K 31/4439; A61K 31/497; A61K 31/501; A61K 31/506; A61K 31/537; A61K 31/5386; A61K 31/675; C07D 471/10; C07D 498/10; C07D 519/00; C07F 9/6561

(52) U.S. Cl. CPC C07D 487/04; A61K 9/2054; A61K 9/2059; A61K 9/4858; A61K 9/4866; A61K 31/407; A61K 31/4155; A61K 31/4178; A61K 31/4188; A61K 31/4192; A61K 31/4196; A61K 31/422; A61K 31/424; A61K 31/427; A61K 31/433; A61K 31/437; A61K 31/438; A61K 31/4439; A61K 31/497; A61K 31/501; A61K 31/506; A61K 31/537; A61K 31/5386; A61K 31/675; C07D 471/10; C07D 498/10; C07D 519/00; C07F 9/6561

(57) ABSTRACT

The invention provides new MAGL inhibitors having the general formula (I)

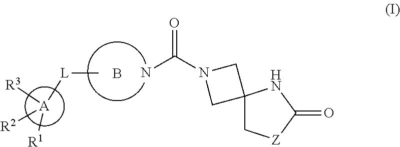

wherein the variables are as described herein, compositions including the compounds, processes of manufacturing the compounds and methods of using the compounds.

### CROSS REFERENCE TO RELATED APPLICATIONS

This application is a continuation of International Application No. PCT/EP2022/078329, filed Oct. 12, 2022, which claims priority to EP application Ser. No. 22/174,059.0, filed May 18, 2022 and EP application Ser. No. 21/202,310.5, filed Oct. 13, 2021, the disclosures of each of which are incorporated herein by reference in its entirety.

### FIELD OF THE INVENTION

The present invention relates to organic compounds useful for therapy or prophylaxis in a mammal, and in particular to monoacylglycerol lipase (MAGL) inhibitors that are useful for the treatment or prophylaxis of diseases or conditions that are associated with MAGL, e.g., neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, inflammatory bowel symptoms, gut motility, visceral pain, fibromyalgia, endometriosis, abdominal pain, abdominal pain associated with irritable bowel syndrome, asthma, COPD, and/or visceral pain.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28.). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809.). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798.). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation. e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2). F2 (PGF2), and thromboxane B2 (TXB2), are strongly decreased. Phospholipase A2 (PLA2) enzymes have been viewed as the principal source of AA, but cPLA2-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis. Alzheimer's disease. Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a). IL-1b. IL-6, and tumor necrosis factor-a (TNF-a) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403.). Importantly, genetic and/or pharmacological disruption of MAGL activity also blocks LPS-induced activation of microglial cells in the brain (Nomura, D. K., et al., Science 2011, 334, 809.).

In addition, genetic and/or pharmacological disruption of MAGL activity was shown to be protective in several animal models of neurodegeneration including, but not restricted to. Alzheimer's disease. Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37.). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809.), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424.) and on mental disorders, such as depression in chronic stress models (Zhong P. et al., Neuropsychopharmacology 2014, 39, 1763.).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163.). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421.). Inhibition of MAGL was also shown to promote remyelination and functional recovery in a mouse model of progressive multiple sclerosis (Feliu A. et al., Journal of Neuroscience 2017, 37 (35), 8385.).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140 (1), 49-61; Nomura D K et al., Chem. Biol. 2011, 18 (7), 846-856, Jinlong Yin et al, Nature Communications 2020, 11, 2978).

The endocannabinoid system is also involved in many gastrointestinal physiological and physiopathological actions (Marquez, Suarez et al. 2009). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright, Rooney et al. 2005), (Duncan, Davison et al. 2005). Activation of CB1 produces anti-emetic, anti-motility, and anti-inflammatory effect, and help to modulate pain (Perisetti, Rimu et al. 2020). CB2 receptors are expressed in immune cells such as plasma cells and macrophages, in the lamina propria of the GI tract (Wright, Rooney et al. 2005), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez, Suarez et al. 2009) and 2-AG levels are increased in plasma of IBD patients (Grill, Hogenauer et al. 2019). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez, Suarez et al. 2009). Furthermore, MAGL inhibition improves gut wall integrity and intestinal permeability via a CB1 driven MoA (Wang, Zhang et al. 2020).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides compounds of formula (I)

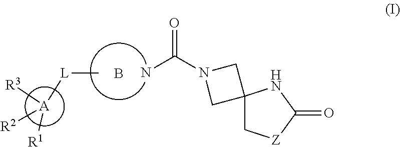

wherein the variables are as defined herein.

In a further aspect, the present invention provides processes for manufacturing the compounds of formula (I) described herein.

In a further aspect, the present invention provides a compound of formula (I) as described herein, or a pharmaceutically acceptable salt thereof, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use in the treatment or prophylaxis of a disease or condition associated with MAGL.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl(isopropyl), n-butyl, iso-butyl, sec-butyl, tert-butyl, and 2,2-dimethylpropyl. Particularly preferred, yet non-limiting examples of alkyl are methyl, tert-butyl, and 2,2-dimethylpropyl.

The term “thiadiazolyl” includes thiadiazolyl, 1,2,4-thiadiazolyl, and 1,3,4-thiadiazolyl.

The term “triazolyl” includes 2H-triazolyl and 1H-1,2,4-triazolyl, and 4H-1,2,4-triazolyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁₋₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl. A particularly preferred, yet non-limiting example of cycloalkyl is cyclopropyl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “cyano” refers to a-CN (nitrile) group.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl, difluoromethyl, 1,1-difluoroethyl, 2,2-difluoroethyl, and 2,2,2-trifluoroethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are trifluoromethoxy, difluoromethoxy, 2,2,2-trifluoro-1,1-dimethyl-ethoxy, (1,1,1-trifluoropropan-2-yl)oxy, and 2,2,2-trifluoroethoxy.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury.”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect, the present invention provides a compound of Formula (I)

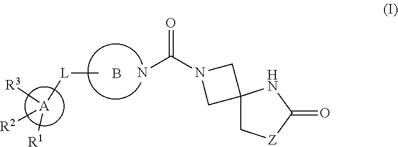

or a pharmaceutically acceptable salt thereof, wherein:
X¹, X², X³, and X⁴ are each independently selected from CH and N;
W is selected from O and NH;
Z is selected from O, CH₂, CH₂CH₂, NH, CH₂O, and OCH₂;
L is selected from a covalent bond, CH₂, NR₄, NHCH₂, CH₂NH, NHCO, CONH, CH₂O, OCH₂, O, NHSO₂, SO₂, SO₂NH,

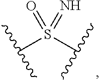 [SMILES: *S(*)(=N)=O]

and CH₂NHSO₂; A is selected from pyridyl, phenyl, azetidinyl, cyclopropyl, triazolyl, thiazolyl, isothiazolyl, thiadiazolyl, imidazolyl, pyrimidinyl, pyrazinyl, pyridazinyl, pyrazolyl, oxazolyl, isoxazolyl, [1,2,4]triazolo[1,5-a]pyridyl, 1H-pyrazolo[4,3-b]pyridyl, 1,2-dihydropyridyl, and bicyclo[1.1.1]pentanyl;
B is selected from:

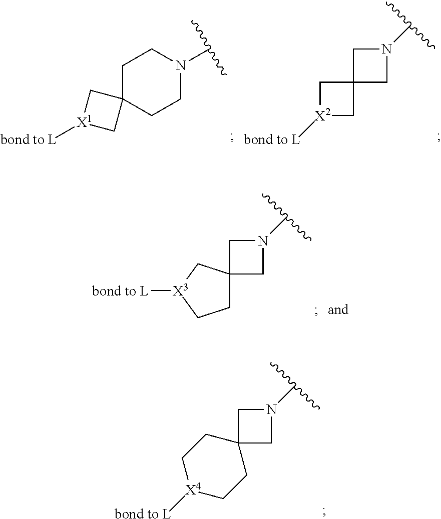

R¹ is selected from hydrogen, halogen, cyano, C₁-C₆-alkyl, C₁-C₆-alkyl-NHC(O)—, carbamoyl, C₁-C₆-alkoxy, halo-C₁-C₆-alkyl, halo-C₁-C₆-alkoxy, C₃-C₁₀-cycloalkyl, (C₁-C₆-alkyl)₂PO—, and a group

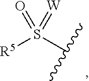

wherein said C₃-C₁₀-cycloalkyl is optionally substituted with one halo-C₁-C₆-alkyl substituent;
R² and R³ are independently selected from hydrogen, halogen, cyano, oxo, C₁-C₆-alkyl, halo-C₁-C₆-alkyl, and halo-C₁-C₆-alkoxy;
R⁴ is selected from hydrogen and C₁-C₆-alkyl; and
R⁵ is selected from C₃-C₁₀-cycloalkyl, C₁-C₆-alkyl and halo-C₁-C₆-alkyl.

In one embodiment, the present invention provides a compound of Formula (I) as described herein, or a pharmaceutically acceptable salt thereof, wherein:
X¹, X², X³, and X⁴ are each independently selected from CH and N;
W is selected from O and NH;
Z is selected from O, CH₂, CH₂CH₂, NH, CH₂O, and OCH₂;
L is selected from a covalent bond, CH₂, NR₄, NHCH₂, CH₂NH, NHCO, CONH, CH₂O, OCH₂, O, NHSO₂, SO₂, SO₂NH,

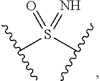 [SMILES: *S(*)(=N)=O]

and CH₂NHSO₂; A is selected from pyridyl, phenyl, azetidinyl, cyclopropyl, triazolyl, thiazolyl, thiadiazolyl, imidazolyl, pyrimidinyl, pyrazinyl, pyridazinyl, pyrazolyl, oxazolyl, [1,2,4]triazolo[1,5-a]pyridyl, 1H-pyrazolo[4,3-b]pyridyl, 1,2-dihydropyridyl, and bicyclo[1.1.1]pentanyl;
B is selected from:

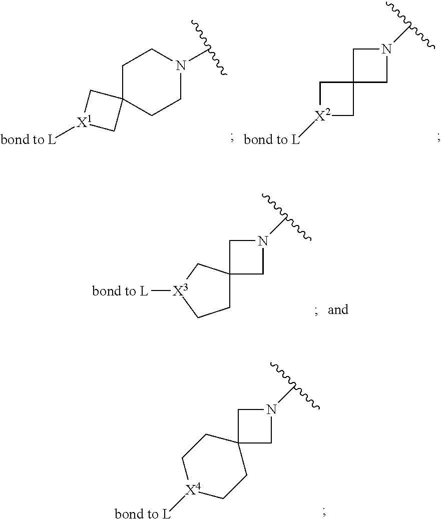

R¹ is selected from hydrogen, halogen, cyano, C₁-C₆-alkyl, C₁-C₆-alkyl-NHC(O)—, carbamoyl, C₁-C₆-alkoxy, halo-C₁-C₆-alkyl, halo-C₁-C₆-alkoxy, C₃-C₁₀-cycloalkyl, and a group

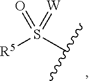

wherein said C₃-C₁₀-cycloalkyl is optionally substituted with one halo-C₁-C₆-alkyl substituent;
R² and R³ are independently selected from hydrogen, halogen, cyano, oxo, C₁-C₆-alkyl, halo-C₁-C₆-alkyl, and halo-C₁-C₆-alkoxy;
R⁴ is selected from hydrogen and C₁-C₆-alkyl; and
R⁵ is selected from C₃-C₁₀-cycloalkyl, C₁-C₆-alkyl and halo-C₁-C₆-alkyl.

In one embodiment, the present invention provides a compound of formula (I) as described herein, or a pharmaceutically acceptable salt thereof, wherein:
X is selected from CH and N;
W is selected from O and NH;
Z is selected from O, CH₂, NH, CH₂O, and OCH₂;
L is selected from a covalent bond, CH₂, NR₄, NHCH₂, CH₂NH, NHCO, CONH, CH₂O, OCH₂, O, NHSO₂, and SO₂NH;
A is selected from pyridyl, phenyl, azetidinyl, cyclopropyl, triazolyl, thiadiazolyl, imidazolyl, pyrimidinyl, pyrazinyl, pyridazinyl, pyrazolyl, and oxazolyl;
B is selected from:

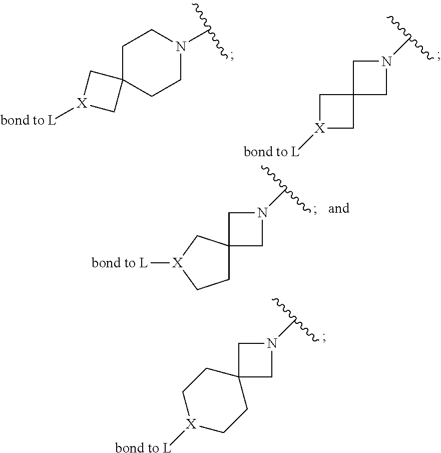

R¹ is selected from halogen, halo-C₁-C₆-alkyl, halo-C₁-C₆-alkoxy, C₃-C₁₀-cycloalkyl, and a group

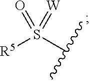

R² and R³ are independently selected from hydrogen, halogen, cyano, and halo-C₁-C₆-alkyl;
R⁴ is selected from hydrogen and C₁-C₆-alkyl; and
R⁵ is selected from C₁-C₆-alkyl and halo-C₁-C₆-alkyl.

In one embodiment, the present invention provides a compound of formula (I) as described herein, or a pharmaceutically acceptable salt thereof, wherein X is N.

In a preferred embodiment, the present invention provides a compound of formula (I) as described herein, or a pharmaceutically acceptable salt thereof, wherein X is CH.

In a preferred embodiment, the present invention provides a compound of formula (I) as described herein, or a pharmaceutically acceptable salt thereof, wherein B is selected from

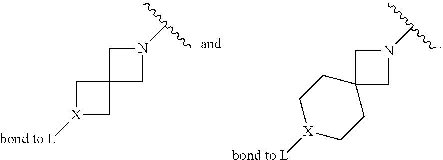

In a particularly preferred embodiment, the present invention provides a compound of formula (I) as described herein, or a pharmaceutically acceptable salt thereof, wherein B is

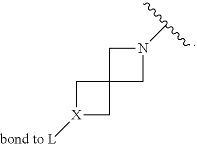

In a preferred embodiment, the present invention provides a compound of formula (I) as described herein, or a pharmaceutically acceptable salt thereof, wherein:
X is CH; and
B is selected from:

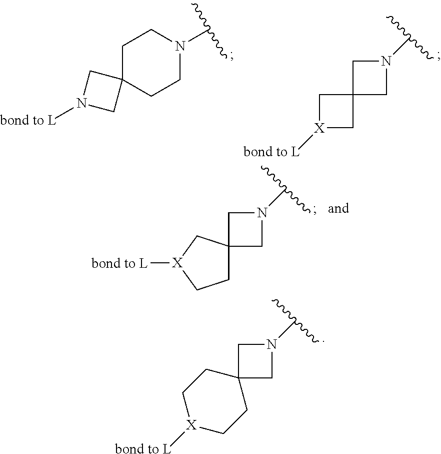

In a preferred embodiment, the present invention provides a compound of formula (I) as described herein, or a pharmaceutically acceptable salt thereof, wherein:
X is CH; and
B is selected from

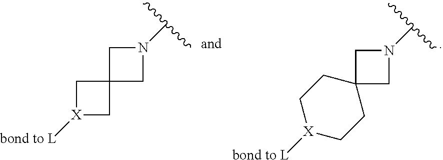

In a particularly preferred embodiment, the present invention provides a compound of formula (I) as described herein, or a pharmaceutically acceptable salt thereof, wherein:
X is CH; and

B is

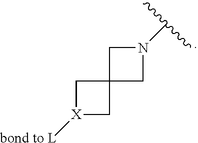

In a preferred embodiment, the present invention provides a compound of formula (I) as described herein, or a pharmaceutically acceptable salt thereof, wherein:
B is selected from:

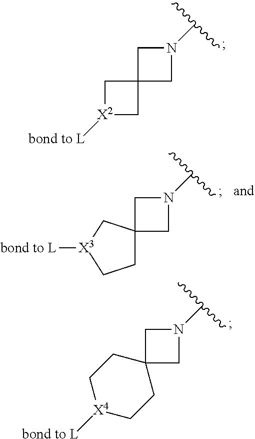

wherein
X² and X³ are both CH; and
X⁴ is selected from CH and N.

In a particularly preferred embodiment, the present invention provides a compound of formula (I) as described herein, or a pharmaceutically acceptable salt thereof, wherein:
B is

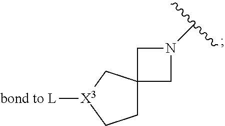

and
X³ is CH.

In a preferred embodiment, the present invention provides a compound of formula (I) as described herein, or a pharmaceutically acceptable salt thereof, wherein:
B is

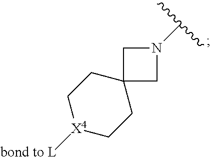

and
X⁴ is selected from CH and N.

In a preferred embodiment, the present invention provides a compound of formula (I) as described herein, or a pharmaceutically acceptable salt thereof, wherein Z is selected from O, CH₂, and NH.

In a preferred embodiment, the present invention provides a compound of formula (I) as described herein, or a pharmaceutically acceptable salt thereof, wherein Z is selected from O, CH₂, CH₂CH₂, CH₂O, and NH.

In a preferred embodiment, the present invention provides a compound of formula (I) as described herein, or a pharmaceutically acceptable salt thereof, wherein Z is CH₂.

In a preferred embodiment, the present invention provides a compound of formula (I) as described herein, or a pharmaceutically acceptable salt thereof, wherein Z is CH₂CH₂.

In a preferred embodiment, the present invention provides a compound of formula (I) as described herein, or a pharmaceutically acceptable salt thereof, wherein Z is CH₂O.

In a preferred embodiment, the present invention provides a compound of formula (I) as described herein, or a pharmaceutically acceptable salt thereof, wherein Z is O.

In a preferred embodiment, the present invention provides a compound of formula (I) as described herein, or a pharmaceutically acceptable salt thereof, wherein Z is NH.

In a particularly preferred embodiment, the present invention provides a compound of formula (I) as described herein, or a pharmaceutically acceptable salt thereof, wherein:

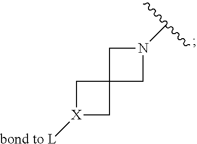

B is
X is CH; and
Z is selected from O, CH₂, and NH.

In a particularly preferred embodiment, the present invention provides a compound of formula (I) as described herein, or a pharmaceutically acceptable salt thereof, wherein:

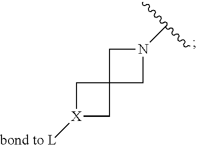

B is
X is CH; and
Z is CH₂.

In one embodiment, the present invention provides a compound of formula (I) as described herein, or a pharmaceutically acceptable salt thereof, wherein:
L is selected from a covalent bond, CH₂, NR₄, NHCH₂, CH₂NH, NHCO, CH₂O, O, and SO₂NH; and
R⁴ is selected from hydrogen and C₁-C₆-alkyl.

In one embodiment, the present invention provides a compound of formula (I) as described herein, or a pharmaceutically acceptable salt thereof, wherein:
L is selected from CH₂, NR₄, NHCH₂, CH₂NH, NHCO, CH₂O, O, and SO₂NH; and
R⁴ is selected from hydrogen and C₁-C₆-alkyl.

In one embodiment, the present invention provides a compound of formula (I) as described herein, or a pharmaceutically acceptable salt thereof, wherein L is a covalent bond.

In a preferred embodiment, the present invention provides a compound of formula (I) as described herein, or a pharmaceutically acceptable salt thereof, wherein:
L is selected from CH₂, NR₄, and O; and
R⁴ is hydrogen.

In a particularly preferred embodiment, the present invention provides a compound of formula (I) as described herein, or a pharmaceutically acceptable salt thereof, wherein L is CH₂.

In a preferred embodiment, the present invention provides a compound of formula (I) as described herein, or a pharmaceutically acceptable salt thereof, wherein:
L is selected from a covalent bond, CH₂, NR₄, NHCH₂, CH₂NH, NHCO, CH₂O, O, SO₂NH, NHSO₂, SO₂,

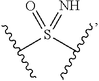 [SMILES: *S(*)(=N)=O]

and CH₂NHSO₂; and
R⁴ is selected from hydrogen and C₁-C₆-alkyl.

In a particularly preferred embodiment, the present invention provides a compound of formula (I) as described herein, or a pharmaceutically acceptable salt thereof, wherein:
L is selected from CH₂, SO₂, NR₄, and O; and
R⁴ is hydrogen.

In one embodiment, the present invention provides a compound of formula (I) as described herein, or a pharmaceutically acceptable salt thereof, wherein:
W is selected from O and NH;
A is selected from pyridyl, phenyl, azetidinyl, cyclopropyl, triazolyl, thiadiazolyl, imidazolyl, pyrimidinyl, pyrazinyl, pyridazinyl, pyrazolyl, and oxazolyl;
R¹ is selected from halogen, halo-C₁-C₆-alkyl, halo-C₁-C₆-alkoxy, C₃-C₁₀-cycloalkyl, and a group

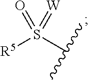

R² is selected from hydrogen, halogen, cyano, and halo-C₁-C₆-alkyl;
R³ is hydrogen; and
R⁵ is selected from C₁-C₆-alkyl and halo-C₁-C₆-alkyl.

In a preferred embodiment, the present invention provides a compound of formula (I) as described herein, or a pharmaceutically acceptable salt thereof, wherein:
W is selected from O and NH;
A is selected from pyridyl, phenyl, pyrimidinyl, and pyridazinyl;
R¹ is selected from halo-C₁-C₆-alkyl and a group

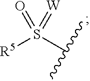

R² is selected from hydrogen and cyano;
R³ is hydrogen; and
R⁵ is selected from C₁-C₆-alkyl and halo-C₁-C₆-alkyl.

In a particularly preferred embodiment, the present invention provides a compound of formula (I) as described herein, or a pharmaceutically acceptable salt thereof, wherein:
W is selected from O and NH;
A is selected from pyridyl, phenyl, pyrimidinyl, and pyridazinyl;
R¹ is selected from CF₃ and a group

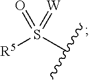

R² is selected from hydrogen and cyano;
R³ is hydrogen; and
R⁵ is selected from methyl and CF₃.

In a preferred embodiment, the present invention provides a compound of formula (I) as described herein, or a pharmaceutically acceptable salt thereof, wherein:
A is C₆-C₁₄-aryl;
R¹ is halo-C₁-C₆-alkyl;
R² is cyano; and
R³ is hydrogen.

In a preferred embodiment, the present invention provides a compound of formula (I) as described herein, or a pharmaceutically acceptable salt thereof, wherein:
A is phenyl;
R¹ is halo-C₁-C₆-alkyl;
R² is cyano; and
R³ is hydrogen.

In a particularly preferred embodiment, the present invention provides a compound of formula (I) as described herein, or a pharmaceutically acceptable salt thereof, wherein:
A is phenyl;
R¹ is CF₃;
R² is cyano; and
R³ is hydrogen.

In one embodiment, the present invention provides a compound of formula (I) as described herein, or a pharmaceutically acceptable salt thereof, wherein:
W is selected from O and NH;
A is selected from pyridyl, phenyl, azetidinyl, cyclopropyl, triazolyl, thiazolyl, thiadiazolyl, imidazolyl, pyrimidinyl, pyrazinyl, pyridazinyl, pyrazolyl, oxazolyl, [1,2,4]triazolo[1,5-a]pyridyl, 1H-pyrazolo[4,3-b]pyridyl, 1,2-dihydropyridyl, and bicyclo[1.1.1]pentanyl;
R¹ is selected from hydrogen, cyano, C₁-C₆-alkyl, C₁-C₆-alkyl-NHC(O)—, carbamoyl, halogen, halo-C₁-C₆-alkyl, halo-C₁-C₆-alkoxy, C₃-C₁₀-cycloalkyl, and a group

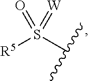

wherein said C₃-C₁₀-cycloalkyl is optionally substituted with one halo-C₁-C₆-alkyl substituent;
R² is selected from hydrogen, halogen, cyano, oxo, C₁-C₆-alkyl, halo-C₁-C₆-alkyl, and halo-C₁-C₆-alkoxy;
R³ is selected from hydrogen and C₁-C₆-alkyl; and
R⁵ is selected from C₃-C₁₀-cycloalkyl, C₁-C₆-alkyl and halo-C₁-C₆-alkyl.

In a preferred embodiment, the present invention provides a compound of formula (I) as described herein, or a pharmaceutically acceptable salt thereof, wherein:
W is selected from O and NH;
A is selected from pyridyl, phenyl, triazolyl, pyrazolyl, pyrimidinyl, and pyridazinyl;
R¹ is selected from halo-C₁-C₆-alkyl, halo-C₁-C₆-alkoxy, and a group

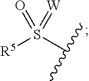

R² is selected from hydrogen, halogen, C₁-C₆-alkyl, and cyano;
R³ is hydrogen; and
R⁵ is selected from C₁-C₆-alkyl and halo-C₁-C₆-alkyl.

In a particularly preferred embodiment, the present invention provides a compound of formula (I) as described herein, or a pharmaceutically acceptable salt thereof, wherein:
W is selected from O and NH;
A is selected from pyridyl, phenyl, pyrimidinyl, triazolyl, pyrazolyl, and pyridazinyl;
R¹ is selected from CF₃, CF₃O and a group

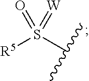

R² is selected from hydrogen, fluoro, methyl, and cyano;
R³ is hydrogen; and
R⁵ is selected from methyl and CF₃.

In one embodiment, the present invention provides a compound of formula (I) as described herein, or a pharmaceutically acceptable salt thereof, wherein:
X is selected from CH and N;
W is selected from O and NH;
Z is selected from O, CH₂, NH, CH₂O, and OCH₂;
L is selected from a covalent bond, CH₂, NR₄, NHCH₂, CH₂NH, NHCO, CH₂O, O, and SO₂NH;
A is selected from pyridyl, phenyl, azetidinyl, cyclopropyl, triazolyl, thiadiazolyl, imidazolyl, pyrimidinyl, pyrazinyl, pyridazinyl, pyrazolyl, and oxazolyl;
B is selected from:

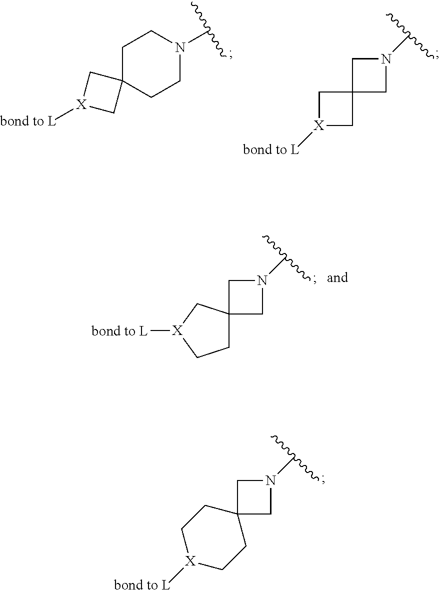

R¹ is selected from halogen, halo-C₁-C₆-alkyl, halo-C₁-C₆-alkoxy, C₃-C₁₀-cycloalkyl, and a group

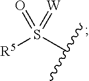

R² is selected from hydrogen, halogen, cyano, and halo-C₁-C₆-alkyl;
R³ is hydrogen;
R⁴ is selected from hydrogen and C₁-C₆-alkyl; and
R⁵ is selected from C₁-C₆-alkyl and halo-C₁-C₆-alkyl.

In a preferred embodiment, the present invention provides a compound of formula (I) as described herein, or a pharmaceutically acceptable salt thereof, wherein:
X is CH;
W is selected from O and NH;
Z is selected from O, CH₂, and NH;
L is selected from CH₂, NR₄, and O;
A is selected from pyridyl, phenyl, pyrimidinyl, and pyridazinyl;
B is selected from:

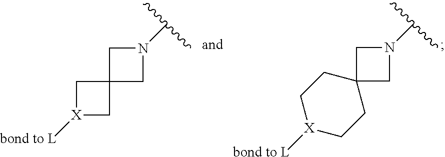

R¹ is selected from halo-C₁-C₆-alkyl and a group

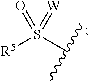

R² is selected from hydrogen and cyano;
R³ is hydrogen;
R⁴ is hydrogen; and
R⁵ is selected from C₁-C₆-alkyl and halo-C₁-C₆-alkyl.

In a particularly preferred embodiment, the present invention provides a compound of formula (I) as described herein, or a pharmaceutically acceptable salt thereof, wherein:
X is CH;
W is selected from O and NH;
Z is selected from O, CH₂, and NH;
L is selected from CH₂, NR₄, and O;
A is selected from pyridyl, phenyl, pyrimidinyl, and pyridazinyl;
B is selected from:

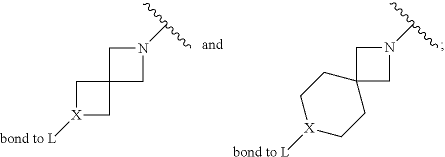

R¹ is selected from CF₃ and a group

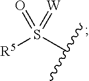

R² is selected from hydrogen and cyano;
R³ is hydrogen;
R⁴ is hydrogen; and
R⁵ is selected from methyl and CF₃.

In one embodiment, the present invention provides a compound of formula (I) as described herein, or a pharmaceutically acceptable salt thereof, wherein:
X is CH;
Z is CH₂;
L is CH₂;
A is C₆-C₁₄-aryl;
B is

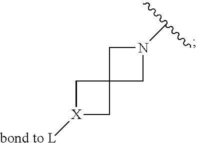

R¹ is halo-C₁-C₆-alkyl;
R² is cyano; and
R³ is hydrogen.

In a preferred embodiment, the present invention provides a compound of formula (I) as described herein, or a pharmaceutically acceptable salt thereof, wherein:
X is CH;
Z is CH₂;
L is CH₂;
A is phenyl;
B is

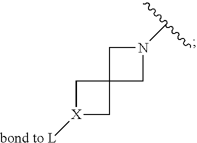

R¹ is halo-C₁-C₆-alkyl;
R² is cyano; and
R³ is hydrogen.

In a particularly preferred embodiment, the present invention provides a compound of formula (I) as described herein, or a pharmaceutically acceptable salt thereof, wherein:
X is CH;
Z is CH₂;
L is CH₂;
A is phenyl;
B is

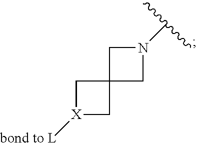

R¹ is CF₃;
R² is cyano; and
R³ is hydrogen.

In one embodiment, the present invention provides a compound of formula (I) as described herein, or a pharmaceutically acceptable salt thereof, wherein:
X¹, X², X³, and X⁴ are each independently selected from CH and N;
W is selected from O and NH;
Z is selected from O, CH₂, CH₂CH₂, NH, CH₂O, and OCH₂;
L is selected from a covalent bond, CH₂, NR₄, NHCH₂, CH₂NH, NHCO, CH₂O, O, SO₂NH, NHSO₂, SO₂,

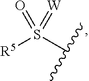

and CH₂NHSO₂;
A is selected from pyridyl, phenyl, azetidinyl, cyclopropyl, triazolyl, thiazolyl, isothiazolyl, thiadiazolyl, imidazolyl, pyrimidinyl, pyrazinyl, pyridazinyl, pyrazolyl, oxazolyl, isoxazolyl, [1,2,4]triazolo[1,5-a]pyridyl, 1H-pyrazolo[4,3-b]pyridyl, 1,2-dihydropyridyl, and bicyclo[1.1.1]pentanyl;
B is selected from:

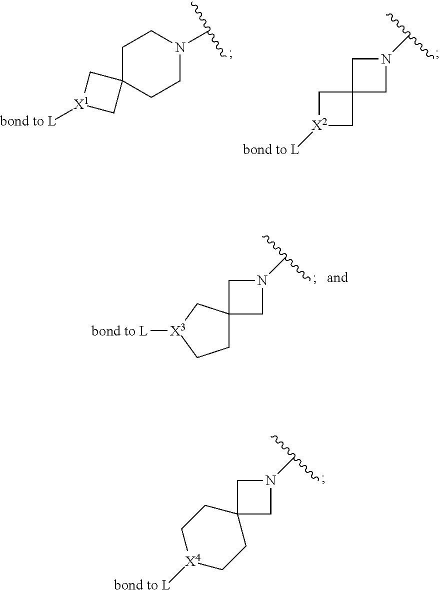

R¹ is selected from hydrogen, halogen, cyano, C₁-C₆-alkyl, C₁-C₆-alkyl-NHC(O)—, carbamoyl, halogen, halo-C₁-C₆-alkyl, halo-C₁-C₆-alkoxy, C₃-C₁₀-cycloalkyl, (C₁-C₆-alkyl)₂PO—, and a group

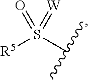

wherein said C₃-C₁₀-cycloalkyl is optionally substituted with one halo-C₁-C₆-alkyl substituent;
R² is selected from hydrogen, halogen, cyano, oxo, C₁-C₆-alkyl, halo-C₁-C₆-alkyl, and halo-C₁-C₆-alkoxy;
R³ is selected from hydrogen and C₁-C₆-alkyl;
R⁴ is selected from hydrogen and C₁-C₆-alkyl; and
R⁵ is selected from C₃-C₁₀-cycloalkyl, C₁-C₆-alkyl and halo-C₁-C₆-alkyl.

In one embodiment, the present invention provides a compound of formula (I) as described herein, or a pharmaceutically acceptable salt thereof, wherein:
X¹, X², X³, and X⁴ are each independently selected from CH and N;
W is selected from O and NH;
Z is selected from O, CH₂, CH₂CH₂, NH, CH₂O, and OCH₂;
L is selected from a covalent bond, CH₂, NR₄, NHCH₂, CH₂NH, NHCO, CH₂O, O, SO₂NH, NHSO₂, SO₂,

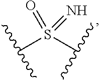 [SMILES: *S(*)(=N)=O]

and CH₂NHSO₂;
A is selected from pyridyl, phenyl, azetidinyl, cyclopropyl, triazolyl, thiazolyl, thiadiazolyl, imidazolyl, pyrimidinyl, pyrazinyl, pyridazinyl, pyrazolyl, oxazolyl, [1,2,4]triazolo[1,5-a]pyridyl, 1H-pyrazolo[4,3-b]pyridyl, 1,2-dihydropyridyl, and bicyclo[1.1.1]pentanyl;
B is selected from:

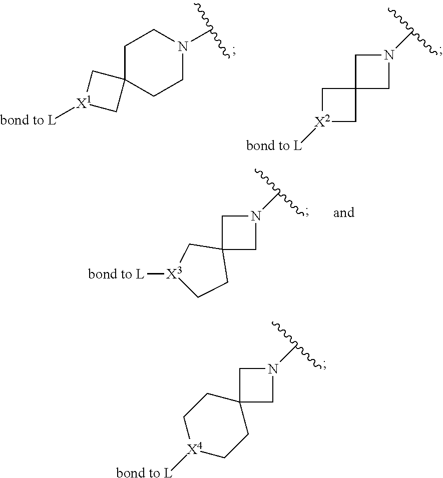

R¹ is selected from hydrogen, cyano, C₁-C₆-alkyl, C₁-C₆-alkyl-NHC(O)—, carbamoyl, halogen, halo-C₁-C₆-alkyl, halo-C₁-C₆-alkoxy, C₃-C₁₀-cycloalkyl, and a group

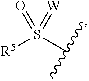

wherein said C₃-C₁₀-cycloalkyl is optionally substituted with one halo-C₁-C₆-alkyl substituent;
R² is selected from hydrogen, halogen, cyano, oxo, C₁-C₆-alkyl, halo-C₁-C₆-alkyl, and halo-C₁-C₆-alkoxy;
R³ is selected from hydrogen and C₁-C₆-alkyl;
R⁴ is selected from hydrogen and C₁-C₆-alkyl; and
R⁵ is selected from C₃-C₁₀-cycloalkyl, C₁-C₆-alkyl and halo-C₁-C₆-alkyl.

In a preferred embodiment, the present invention provides a compound of formula (I) as described herein, or a pharmaceutically acceptable salt thereof, wherein:
X² and X³ are both CH;
X⁴ is selected from CH and N;
W is selected from O and NH;
Z is selected from O, CH₂CH₂, CH₂O, CH₂, and NH;
L is selected from CH₂, SO₂, NR₄, and O;
A is selected from pyridyl, phenyl, triazolyl, pyrazolyl, pyrimidinyl, and pyridazinyl;
B is selected from:

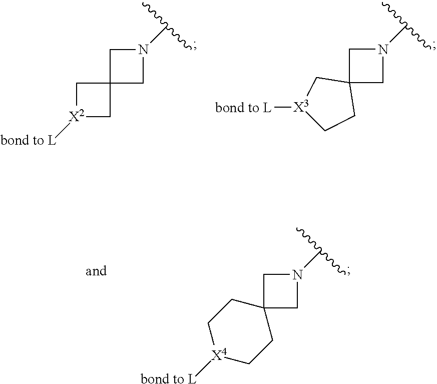

R¹ is selected from halo-C₁-C₆-alkyl, halo-C₁-C₆-alkoxy, and a group

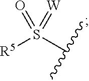

R² is selected from hydrogen, halogen, C₁-C₆-alkyl, and cyano;
R³ is hydrogen;
R⁴ is hydrogen; and
R⁵ is selected from C₁-C₆-alkyl and halo-C₁-C₆-alkyl.

In a particularly preferred embodiment, the present invention provides a compound of formula (I) as described herein, or a pharmaceutically acceptable salt thereof, wherein:
X² and X³ are both CH;
X⁴ is selected from CH and N;
W is selected from O, CH₂CH₂, CH₂O, and NH;
Z is selected from O, CH₂, and NH;
L is selected from CH₂, SO₂, NR₄, and O;
A is selected from pyridyl, phenyl, triazolyl, pyrazolyl, pyrimidinyl, and pyridazinyl;
B is selected from:

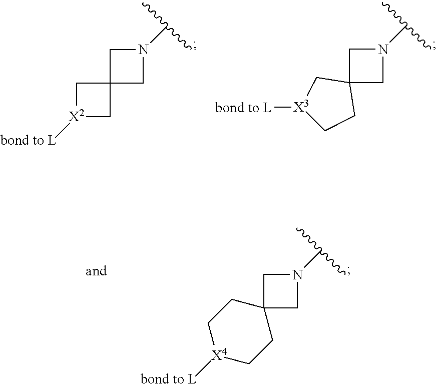

R¹ is selected from CF₃, CF₃O, and a group

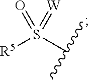

R² is selected from hydrogen, fluoro, methyl, and cyano;
R³ is hydrogen;
R⁴ is hydrogen; and
R⁵ is selected from methyl and CF₃.

In one embodiment, the present invention provides a compound of formula (I) as described herein, or a pharmaceutically acceptable salt thereof, selected from:
2-[6-(4-mesylbenzyl)-2-azaspiro[3.3]heptane-2-carbonyl]-7-oxa-2,5-diazaspiro[3.4]octan-6-one;
2-[7-[[5-(trifluoromethyl)-2-pyridyl]amino]-2-azaspiro[3.5]nonane-2-carbonyl]-7-oxa-2,5-diazaspiro[3.4]octan-6-one;
2-[6-[[4-(trifluoromethylsulfonimidoyl)benzyl]-2-azaspiro[3.3]heptane-2-carbonyl]-2,5-diazaspiro[3.4]octan-6-one;
2-[6-[[3-(trifluoromethylsulfonimidoyl)benzyl]-2-azaspiro[3.3]heptane-2-carbonyl]-2,5-diazaspiro[3.4]octan-6-one;
2-[6-[[6-(trifluoromethyl)-3-pyridyl]methyl]-2-azaspiro[3.3]heptane-2-carbonyl]-7-oxa-2,5-diazaspiro[3.4]octan-6-one;
2-[6-(4-fluoro-2-mesyl-benzyl)-2-azaspiro[3.3]heptane-2-carbonyl]-7-oxa-2,5-diazaspiro[3.4]octan-6-one;
2-[7-[[6-(trifluoromethyl)pyridazin-3-yl]oxy-2-azaspiro[3.5]nonane-2-carbonyl]-7-oxa-2,5-diazaspiro[3.4]octan-6-one;
5-[[2-(6-keto-2,5-diazaspiro[3.4]octane-2-carbonyl)-2-azaspiro[3.3]heptan-6-yl]methyl]-2-(trifluoromethyl)benzonitrile;
2-[7-[methyl-[5-(trifluoromethyl) pyrazin-2-yl]amino]-2-azaspiro[3.5]nonane-2-carbonyl]-7-oxa-2,5-diazaspiro[3.4]octan-6-one;
5-[[2-(6-keto-2,5,7-triazaspiro[3.4]octane-2-carbonyl)-2-azaspiro[3.3]heptan-6-yl]methyl]-2-(trifluoromethyl)benzonitrile;
2-[7-[[5-(trifluoromethyl)-2-pyridyl]methyl]-2-azaspiro[3.5]nonane-2-carbonyl]-2,5,7-triazaspiro[3.4]octan-6-one;
2-[7-[[2-(trifluoromethyl)pyrimidin-4-yl]oxy-2-azaspiro[3.5]nonane-2-carbonyl]-7-oxa-2,5-diazaspiro[3.4]octan-6-one;
5-[[2-(6-keto-7-oxa-2,5-diazaspiro[3.4]octane-2-carbonyl)-2-azaspiro[3.3]heptan-6-yl]methyl]-2-(trifluoromethyl)benzonitrile;
2-[7-[(5-chloro-2-pyridyl)methyl]-2-azaspiro[3.5]nonane-2-carbonyl]-7-oxa-2,5-diazaspiro[3.4]octan-6-one;
2-[7-[[5-(trifluoromethyl) pyrazin-2-yl]amino]-2-azaspiro[3.5]nonane-2-carbonyl]-7-oxa-2,5-diazaspiro[3.4]octan-6-one;
2-[7-[[5-(trifluoromethyl) pyrazin-2-yl]oxy-2-azaspiro[3.5]nonane-2-carbonyl]-7-oxa-2,5-diazaspiro[3.4]octan-6-one;
2-[6-[[5-(trifluoromethyl)pyrimidin-2-yl]methyl]-2-azaspiro[3.3]heptane-2-carbonyl]-2,5-diazaspiro[3.4]octan-6-one;
2-[7-[[5-(trifluoromethyl)-2-pyridyl]methyl]-2-azaspiro[3.5]nonane-2-carbonyl]-2,5-diazaspiro[3.4]octan-6-one;
2-[6-[[6-(trifluoromethyl)-3-pyridyl]methyl]-2-azaspiro[3.3]heptane-2-carbonyl]-2,5-diazaspiro[3.4]octan-6-one;
2-[7-[[5-(trifluoromethyl)-2-pyridyl]methyl]-2-azaspiro[3.5]nonane-2-carbonyl]-7-oxa-2,5-diazaspiro[3.4]octan-6-one;
2-[6-[[4-(trifluoromethyl) pyrazol-1-yl]methyl]-2-azaspiro[3.3]heptane-2-carbonyl]-7-oxa-2,5-diazaspiro[3.4]octan-6-one;
2-[6-[[5-(trifluoromethyl) pyrazin-2-yl]methyl]-2-azaspiro[3.3]heptane-2-carbonyl]-2,5-diazaspiro[3.4]octan-6-one;
2-[7-(4-fluoro-2-mesyl-phenoxy)-2-azaspiro[3.5]nonane-2-carbonyl]-7-oxa-2,5-diazaspiro[3.4]octan-6-one;
2-[(6S)-6-[5-(trifluoromethyl) pyrazin-2-yl]oxy-2-azaspiro[3.4]octane-2-carbonyl]-7-oxa-2,5-diazaspiro[3.4]octan-6-one;
2-[6-[(5-chloro-2-pyridyl)methyl]-2-azaspiro[3.3]heptane-2-carbonyl]-7-oxa-2,5-diazaspiro[3.4]octan-6-one;
2-[6-(3-triflylbenzyl)-2-azaspiro[3.3]heptane-2-carbonyl]-7-oxa-2,5-diazaspiro[3.4]octan-6-one;
2-[6-[[5-(trifluoromethyl) pyrazin-2-yl]methyl]-2-azaspiro[3.3]heptane-2-carbonyl]-7-oxa-2,5-diazaspiro[3.4]octan-6-one;
2-[7-[[6-(trifluoromethyl)-3-pyridyl]methyl]-2-azaspiro[3.5]nonane-2-carbonyl]-7-oxa-2,5-diazaspiro[3.4]octan-6-one;
2-[7-[[5-(trifluoromethyl) pyrazin-2-yl]methyl]-2-azaspiro[3.5]nonane-2-carbonyl]-2,5-diazaspiro[3.4]octan-6-one;
2-[6-[(3,5-difluoro-2-pyridyl)methyl]-2-azaspiro[3.4]octane-2-carbonyl]-7-oxa-2,5-diazaspiro[3.4]octan-6-one;
2-[6-(4-triflylphenyl)-2-azaspiro[3.3]heptane-2-carbonyl]-7-oxa-2,5-diazaspiro[3.4]octan-6-one; 2-[(6R)-6-[5-(trifluoromethyl) pyrazin-2-yl]oxy-2-azaspiro[3.4]octane-2-carbonyl]-7-oxa-2,5-diazaspiro[3.4]octan-6-one;
2-[7-[[6-(difluoromethoxy)-3-pyridyl]methyl]-2-azaspiro[3.5]nonane-2-carbonyl]-7-oxa-2,5-diazaspiro[3.4]octan-6-one;
2-[6-[[5-(trifluoromethoxy)-2-pyridyl]methyl]-2-azaspiro[3.3]heptane-2-carbonyl]-2,5-diazaspiro[3.4]octan-6-one;
2-[6-[[3-fluoro-5-(trifluoromethyl)-2-pyridyl]methyl]-2-azaspiro[3.3]heptane-2-carbonyl]-2,5-diazaspiro[3.4]octan-6-one;
2-[7-[[1-(trifluoromethyl)cyclopropyl]methoxy]-2-azaspiro[3.5]nonane-2-carbonyl]-7-oxa-2,5-diazaspiro[3.4]octan-6-one;
2-[7-(2,5-dichlorophenoxy)-2-azaspiro[3.5]nonane-2-carbonyl]-7-oxa-2,5-diazaspiro[3.4]octan-6-one;
2-[6-[[4-fluoro-2-(trifluoromethyl)benzyl]-2-azaspiro[3.3]heptane-2-carbonyl]-7-oxa-2,5-diazaspiro[3.4]octan-6-one;
2-[6-(2,4-difluorobenzyl)-2-azaspiro[3.3]heptane-2-carbonyl]-7-oxa-2,5-diazaspiro[3.4]octan-6-one;
2-[6-[[2-fluoro-4-(trifluoromethyl)benzyl]-2-azaspiro[3.3]heptane-2-carbonyl]-7-oxa-2,5-diazaspiro[3.4]octan-6-one;
2-[6-[[2-fluoro-4-(trifluoromethyl)benzyl]-2-azaspiro[3.3]heptane-2-carbonyl]-2,5,7-triazaspiro[3.4]octan-6-one;
2-[6-[[2-fluoro-4-(trifluoromethyl)phenyl]-2-azaspiro[3.3]heptane-2-carbonyl]-7-oxa-2,5-diazaspiro[3.4]octan-6-one;
2-[6-[[5-(trifluoromethoxy)-2-pyridyl]methyl]-2-azaspiro[3.3]heptane-2-carbonyl]-7-oxa-2,5-diazaspiro[3.4]octan-6-one;
2-[6-[[2-mesyl-4-(trifluoromethyl)benzyl]-2-azaspiro[3.3]heptane-2-carbonyl]-7-oxa-2,5-diazaspiro[3.4]octan-6-one;
2-[6-[[5-(trifluoromethyl)-2-pyridyl]methyl]-2-azaspiro[3.4]octane-2-carbonyl]-7-oxa-2,5-diazaspiro[3.4]octan-6-one;
2-[7-[(3,5-difluoro-2-pyridyl)methyl]-2-azaspiro[3.5]nonane-2-carbonyl]-7-oxa-2,5-diazaspiro[3.4]octan-6-one;
2-[6-[[4-mesyl-3-(trifluoromethyl)benzyl]-2-azaspiro[3.4]octane-2-carbonyl]-7-oxa-2,5-diazaspiro[3.4]octan-6-one;
2-[6-[[3-fluoro-5-(trifluoromethyl)-2-pyridyl]methyl]-2-azaspiro[3.3]heptane-2-carbonyl]-7-oxa-2,5-diazaspiro[3.4]octan-6-one;
2-[6-[[6-(trifluoromethyl)-3-pyridyl]methyl]-2-azaspiro[3.4]octane-2-carbonyl]-7-oxa-2,5-diazaspiro[3.4]octan-6-one;
2-[(6S)-6-[[5-(trifluoromethyl) pyrazin-2-yl]methyl]-2-azaspiro[3.4]octane-2-carbonyl]-7-oxa-2,5-diazaspiro[3.4]octan-6-one;
2-[6-[[4-mesyl-3-(trifluoromethyl)benzyl]-2-azaspiro[3.3]heptane-2-carbonyl]-7-oxa-2,5-diazaspiro[3.4]octan-6-one;
2-[6-[[3-mesyl-4-(trifluoromethyl)benzyl]-2-azaspiro[3.3]heptane-2-carbonyl]-7-oxa-2,5-diazaspiro[3.4]octan-6-one;
2-[6-(3-fluoro-5-mesyl-benzyl)-2-azaspiro[3.3]heptane-2-carbonyl]-7-oxa-2,5-diazaspiro[3.4]octan-6-one;
2-[7-[[5-(trifluoromethyl) pyrazin-2-yl]methyl]-2-azaspiro[3.5]nonane-2-carbonyl]-7-oxa-2,5-diazaspiro[3.4]octan-6-one;
2-[6-[(5-fluoro-2-pyridyl)methyl]-2-azaspiro[3.3]heptane-2-carbonyl]-7-oxa-2,5-diazaspiro[3.4]octan-6-one;
2-[(6R)-6-[[5-(trifluoromethyl) pyrazin-2-yl]methyl]-2-azaspiro[3.4]octane-2-carbonyl]-7-oxa-2,5-diazaspiro[3.4]octan-6-one;
N-[2-(6-keto-7-oxa-2,5-diazaspiro[3.4]octane-2-carbonyl)-2-azaspiro[3.5]nonan-7-yl]-3-(trifluoromethoxy)benzenesulfonamide;
2-[6-[[2-(trifluoromethyl)pyrimidin-5-yl]methyl]-2-azaspiro[3.3]heptane-2-carbonyl]-7-oxa-2,5-diazaspiro[3.4]octan-6-one;
2-[6-[[[1-(trifluoromethyl)cyclopropyl]amino]methyl]-2-azaspiro[3.3]heptane-2-carbonyl]-7-oxa-2,5-diazaspiro[3.4]octan-6-one;
N-[2-(6-keto-7-oxa-2,5-diazaspiro[3.4]octane-2-carbonyl)-2-azaspiro[3.3]heptan-6-yl]-3-(trifluoromethyl)benzenesulfonamide;
2-[6-(3-mesylbenzyl)-2-azaspiro[3.3]heptane-2-carbonyl]-7-oxa-2,5-diazaspiro[3.4]octan-6-one;
2-[6-(4-mesylbenzyl)-2-azaspiro[3.4]octane-2-carbonyl]-7-oxa-2,5-diazaspiro[3.4]octan-6-one;
2-[6-(4-fluorophenyl)-2-azaspiro[3.3]heptane-2-carbonyl]-7-oxa-2,5-diazaspiro[3.4]octan-6-one;
2-[6-[[4-fluoro-2-(trifluoromethyl)benzyl]-2-azaspiro[3.3]heptane-2-carbonyl]-2,5,7-triazaspiro[3.4]octan-6-one;
2-[6-[[5-(trifluoromethyl) pyrazin-2-yl]-2-azaspiro[3.3]heptane-2-carbonyl]-7-oxa-2,5-diazaspiro[3.4]octan-6-one;
2-[6-[[1-(trifluoromethyl)cyclopropyl]methoxy]-2-azaspiro[3.3]heptane-2-carbonyl]-7-oxa-2,5-diazaspiro[3.4]octan-6-one;
2-[6-[[2-mesyl-4-(trifluoromethyl)benzyl]-2-azaspiro[3.3]heptane-2-carbonyl]-2,5-diazaspiro[3.4]octan-6-one;
2-[6-[[6-(trifluoromethyl)pyridazin-3-yl]methyl]-2-azaspiro[3.3]heptane-2-carbonyl]-7-oxa-2,5-diazaspiro[3.4]octan-6-one;
N-[2-(6-keto-2,5-diazaspiro[3.4]octane-2-carbonyl)-2-azaspiro[3.5]nonan-7-yl]-3-(trifluoromethoxy)benzenesulfonamide;
2-[6-[[1-(trifluoromethyl)cyclopropyl]amino]-2-azaspiro[3.3]heptane-2-carbonyl]-7-oxa-2,5-diazaspiro[3.4]octan-6-one;
2-[6-[(5-fluoro-3-pyridyl)methyl]-2-azaspiro[3.3]heptane-2-carbonyl]-7-oxa-2,5-diazaspiro[3.4]octan-6-one;
2-[6-[[2-mesyl-4-(trifluoromethyl)phenyl]-2-azaspiro[3.3]heptane-2-carbonyl]-7-oxa-2,5-diazaspiro[3.4]octan-6-one;
2-[6-[[2-(trifluoromethyl)pyrimidin-5-yl]-2-azaspiro[3.3]heptane-2-carbonyl]-7-oxa-2,5-diazaspiro[3.4]octan-6-one;
2-[6-(2,4-difluorobenzyl)-2-azaspiro[3.3]heptane-2-carbonyl]-2,5,7-triazaspiro[3.4]octan-6-one;
2-[6-[[4-mesyl-3-(trifluoromethyl)benzyl]-2-azaspiro[3.3]heptane-2-carbonyl]-2,5-diazaspiro[3.4]octan-6-one;
2-[(6S)-6-[[4-mesyl-3-(trifluoromethyl) phenoxy]-2-azaspiro[3.4]octane-2-carbonyl]-7-oxa-2,5-diazaspiro[3.4]octan-6-one;
2-[6-[[1-(trifluoromethyl)cyclopropyl]methylamino]-2-azaspiro[3.3]heptane-2-carbonyl]-7-oxa-2,5-diazaspiro[3.4]octan-6-one;
2-[7-[(5-chloro-2-pyridyl)methyl]-2-azaspiro[3.5]nonane-2-carbonyl]-2,5-diazaspiro[3.4]octan-6-one;
2-[6-[[3-mesyl-4-(trifluoromethyl)benzyl]-2-azaspiro[3.3]heptane-2-carbonyl]-2,5-diazaspiro[3.4]octan-6-one;
2-[6-[[5-(trifluoromethyl) pyrazin-2-yl]oxy-2-azaspiro[3.4]octane-2-carbonyl]-7-oxa-2,5-diazaspiro[3.4]octan-6-one;
2-[6-[[5-(trifluoromethyl) pyrazin-2-yl]oxy-2-azaspiro[3.3]heptane-2-carbonyl]-7-oxa-2,5-diazaspiro[3.4]octan-6-one;
2-[6-[6-(trifluoromethyl)-3-pyridyl]-2-azaspiro[3.3]heptane-2-carbonyl]-7-oxa-2,5-diazaspiro[3.4]octan-6-one;
2-[7-[[6-(difluoromethoxy)-3-pyridyl]methyl]-2-azaspiro[3.5]nonane-2-carbonyl]-2,5-diazaspiro[3.4]octan-6-one;
2-[7-(5-chloro-2-mesyl-phenoxy)-2-azaspiro[3.5]nonane-2-carbonyl]-7-oxa-2,5-diazaspiro[3.4]octan-6-one;
2-[7-[[5-(trifluoromethyl)pyrimidin-2-yl]oxy-2-azaspiro[3.5]nonane-2-carbonyl]-7-oxa-2,5-diazaspiro[3.4]octan-6-one;
2-[6-(4-mesylphenyl)-2-azaspiro[3.3]heptane-2-carbonyl]-7-oxa-2,5-diazaspiro[3.4]octan-6-one;
2-[6-(4-fluoro-2-mesyl-benzyl)-2-azaspiro[3.3]heptane-2-carbonyl]-2,5-diazaspiro[3.4]octan-6-one;
2-[7-[(3,5-difluoro-2-pyridyl)methyl]-2-azaspiro[3.5]nonane-2-carbonyl]-2,5-diazaspiro[3.4]octan-6-one;
2-[6-[[3-(trifluoromethyl) azetidin-1-yl]-2-azaspiro[3.3]heptane-2-carbonyl]-7-oxa-2,5-diazaspiro[3.4]octan-6-one;
2-[6-[[3-(2,2,2-trifluoroethyl) azetidin-1-yl]-2-azaspiro[3.3]heptane-2-carbonyl]-7-oxa-2,5-diazaspiro[3.4]octan-6-one;
2-[6-[6-(trifluoromethyl)pyridazin-3-yl]oxy-2-azaspiro[3.3]heptane-2-carbonyl]-7-oxa-2,5-diazaspiro[3.4]octan-6-one;
2-[6-[[4-(trifluoromethyl) pyrazol-1-yl]methyl]-2-azaspiro[3.3]heptane-2-carbonyl]-2,5-diazaspiro[3.4]octan-6-one;
N-[2-(6-keto-2,5-diazaspiro[3.4]octane-2-carbonyl)-2-azaspiro[3.3]heptan-6-yl]-3-(trifluoromethyl)benzenesulfonamide;
2-[6-(3-cyclopropyl-1,2,4-triazol-1-yl)-2-azaspiro[3.3]heptane-2-carbonyl]-7-oxa-2,5-diazaspiro[3.4]octan-6-one;
2-[6-[[3-(trifluoromethyl)-1,2,4-thiadiazol-5-yl]methyl]-2-azaspiro[3.3]heptane-2-carbonyl]-2,5-diazaspiro[3.4]octan-6-one;
2-[6-(4-mesylbenzyl)-2-azaspiro[3.3]heptane-2-carbonyl]-2,5-diazaspiro[3.4]octan-6-one;
2-[6-[[4-(trifluoromethyl) oxazol-2-yl]methyl]-2-azaspiro[3.3]heptane-2-carbonyl]-2,5-diazaspiro[3.4]octan-6-one;
2-[6-(3-mesylbenzyl)-2-azaspiro[3.3]heptane-2-carbonyl]-2,5-diazaspiro[3.4]octan-6-one;
2-[6-(5-fluoro-3-pyridyl)-2-azaspiro[3.3]heptane-2-carbonyl]-7-oxa-2,5-diazaspiro[3.4]octan-6-one;
2-[6-[[6-(trifluoromethyl)pyridazin-3-yl]amino]-2-azaspiro[3.3]heptane-2-carbonyl]-7-oxa-2,5-diazaspiro[3.4]octan-6-one;
2-[6-(3-fluoro-5-mesyl-benzyl)-2-azaspiro[3.3]heptane-2-carbonyl]-2,5-diazaspiro[3.4]octan-6-one;
2-[6-[[3-(trifluoromethyl)-1,2,4-triazol-1-yl]methyl]-2-azaspiro[3.3]heptane-2-carbonyl]-2,5-diazaspiro[3.4]octan-6-one;
2-[7-[[1-(trifluoromethyl)cyclopropyl]methoxy]-2-azaspiro[3.5]nonane-2-carbonyl]-2,5,7-triazaspiro[3.4]octan-6-one;
2-[6-[[2-(trifluoromethyl)pyrimidin-5-yl]methyl]-2-azaspiro[3.3]heptane-2-carbonyl]-2,5-diazaspiro[3.4]octan-6-one;
2-[6-[[6-(trifluoromethyl)pyridazin-3-yl]methyl]-2-azaspiro[3.3]heptane-2-carbonyl]-2,5-diazaspiro[3.4]octan-6-one;
2-[6-[[4-(trifluoromethyl) imidazol-1-yl]methyl]-2-azaspiro[3.3]heptane-2-carbonyl]-2,5-diazaspiro[3.4]octan-6-one;
2-[7-[[6-(difluoromethoxy)-3-pyridyl]methyl]-2-azaspiro[3.5]nonane-2-carbonyl]-2,5,7-triazaspiro[3.4]octan-6-one;
2-[6-[[6-(trifluoromethyl)-3-pyridyl]methyl]-2-azaspiro[3.3]heptane-2-carbonyl]-2,5,7-triazaspiro[3.4]octan-6-one;
2-[7-[[5-(trifluoromethyl) pyrazin-2-yl]amino]-2-azaspiro[3.5]nonane-2-carbonyl]-2,5-diazaspiro[3.4]octan-6-one;
2-[(6 #R!)-6-[(4-methylsulfonylphenyl)methyl]-2-azaspiro[3.4]octane-2-carbonyl]-8-oxa-2,5-diazaspiro[3.5]nonan-6-one;
2-[(6 #S!)-6-[(4-methylsulfonylphenyl)methyl]-2-azaspiro[3.4]octane-2-carbonyl]-8-oxa-2,5-diazaspiro[3.5]nonan-6-one;
2-(6-keto-7-oxa-2,5-diazaspiro[3.4]octane-2-carbonyl)-N-[1-(trifluoromethyl)cyclopropyl]-2-azaspiro[3.3]heptane-6-carboxamide;
2-[6-(4-mesylbenzyl)-2-azaspiro[3.4]octane-2-carbonyl]-8-oxa-2,5-diazaspiro[3.5]nonan-6-one;
2-[7-[[1-(trifluoromethyl)cyclopropyl]methoxy]-2-azaspiro[3.5]nonane-2-carbonyl]-7-oxa-2,5-diazaspiro[3.5]nonan-6-one;
2-[6-(3-cyclopropyl-1,2,4-triazol-1-yl)-2-azaspiro[3.3]heptane-2-carbonyl]-2,5-diazaspiro[3.4]octan-6-one;
2-[6-[6-(trifluoromethyl)pyridazin-3-yl]oxy-2-azaspiro[3.3]heptane-2-carbonyl]-2,5-diazaspiro[3.4]octan-6-one;
2-[6-[[5-(trifluoromethyl) pyrazin-2-yl]methyl]-2-azaspiro[3.3]heptane-2-carbonyl]-7-oxa-2,5-diazaspiro[3.5]nonan-6-one;
N-[2-(6-keto-7-oxa-2,5-diazaspiro[3.5]nonane-2-carbonyl)-2-azaspiro[3.3]heptan-6-yl]-3-(trifluoromethyl)benzenesulfonamide;
2-[7-[[5-(trifluoromethyl)-2-pyridyl]methyl]-2-azaspiro[3.5]nonane-2-carbonyl]-8-oxa-2,5-diazaspiro[3.5]nonan-6-one;
2-[7-[[2-fluoro-4-(trifluoromethyl)benzyl]-2,7-diazaspiro[3.5]nonane-2-carbonyl]-2,5-diazaspiro[3.4]octan-6-one;
2-[7-[[6-(difluoromethoxy)-3-pyridyl]methyl]-2-azaspiro[3.5]nonane-2-carbonyl]-8-oxa-2,5-diazaspiro[3.5]nonan-6-one;
2-[2-[2-fluoro-4-(trifluoromethyl)benzyl]-2,7-diazaspiro[3.5]nonane-7-carbonyl]-2,5-diazaspiro[3.4]octan-6-one;
2-[2-[2-fluoro-4-(trifluoromethyl)benzyl]-2,7-diazaspiro[3.5]nonane-7-carbonyl]-7-oxa-2,5-diazaspiro[3.5]nonan-6-one;
2-[7-(3-triflylbenzyl)-2,7-diazaspiro[3.5]nonane-2-carbonyl]-8-oxa-2,5-diazaspiro[3.5]nonan-6-one;
2-[7-[[2-fluoro-4-(trifluoromethyl)benzyl]-2,7-diazaspiro[3.5]nonane-2-carbonyl]-7-oxa-2,5-diazaspiro[3.4]octan-6-one;
2-[7-[[1-(trifluoromethyl)cyclopropyl]methoxy]-2-azaspiro[3.5]nonane-2-carbonyl]-8-oxa-2,5-diazaspiro[3.5]nonan-6-one;
2-[2-[2-fluoro-4-(trifluoromethyl)benzyl]-2,7-diazaspiro[3.5]nonane-7-carbonyl]-7-oxa-2,5-diazaspiro[3.4]octan-6-one;
2-[2-[2-fluoro-4-(trifluoromethyl)benzyl]-2,7-diazaspiro[3.5]nonane-7-carbonyl]-7-oxa-2,5-diazaspiro[3.4]octan-6-one;
2-[7-(4-triflylbenzyl)-2,7-diazaspiro[3.5]nonane-2-carbonyl]-8-oxa-2,5-diazaspiro[3.5]nonan-6-one;
2-[7-(3-triflylbenzyl)-2,7-diazaspiro[3.5]nonane-2-carbonyl]-2,5-diazaspiro[3.4]octan-6-one;
2-[7-(4-triflylbenzyl)-2,7-diazaspiro[3.5]nonane-2-carbonyl]-2,5-diazaspiro[3.4]octan-6-one;
2-[6-[[[1-(trifluoromethyl)cyclopropyl]amino]methyl]-2-azaspiro[3.3]heptane-2-carbonyl]-8-oxa-2,5-diazaspiro[3.5]nonan-6-one;
2-[6-(2,4-difluorobenzyl)-2-azaspiro[3.3]heptane-2-carbonyl]-8-oxa-2,5-diazaspiro[3.5]nonan-6-one;
2-[2-[2-fluoro-4-(trifluoromethyl)benzyl]-2,7-diazaspiro[3.5]nonane-7-carbonyl]-2,5,7-triazaspiro[3.4]octan-6-one;
2-[6-[[5-(trifluoromethyl) pyrazin-2-yl]methyl]-2-azaspiro[3.3]heptane-2-carbonyl]-8-oxa-2,5-diazaspiro[3.5]nonan-6-one;
2-[7-(3-triflylbenzyl)-2,7-diazaspiro[3.5]nonane-2-carbonyl]-7-oxa-2,5-diazaspiro[3.4]octan-6-one;
2-[6-[[1-(trifluoromethyl)cyclopropyl]methoxy]-2-azaspiro[3.3]heptane-2-carbonyl]-8-oxa-2,5-diazaspiro[3.5]nonan-6-one;
2-[7-[[2-fluoro-4-(trifluoromethyl)phenyl]sulfonyl-2,7-diazaspiro[3.5]nonane-2-carbonyl]-8-oxa-2,5-diazaspiro[3.5]nonan-6-one;
2-[6-[[3-fluoro-5-(trifluoromethyl)benzyl]-2-azaspiro[3.3]heptane-2-carbonyl]-8-oxa-2,5-diazaspiro[3.5]nonan-6-one;
2-[6-[[4-fluoro-2-(trifluoromethyl)benzyl]-2-azaspiro[3.3]heptane-2-carbonyl]-8-oxa-2,5-diazaspiro[3.5]nonan-6-one;
2-[7-(4-triflylbenzyl)-2,7-diazaspiro[3.5]nonane-2-carbonyl]-7-oxa-2,5-diazaspiro[3.4]octan-6-one;
2-[7-[[2-fluoro-4-(trifluoromethyl)phenyl]sulfonyl-2,7-diazaspiro[3.5]nonane-2-carbonyl]-7-oxa-2,5-diazaspiro[3.5]nonan-6-one:
2-[6-[[2-fluoro-4-(trifluoromethyl)benzyl]-2-azaspiro[3.3]heptane-2-carbonyl]-8-oxa-2,5-diazaspiro[3.5]nonan-6-one;
2-[2-[3-(trifluoromethoxy)phenyl]sulfonyl-2,6-diazaspiro[3.3]heptane-6-carbonyl]-8-oxa-2,5-diazaspiro[3.5]nonan-6-one;
2-[7-[[5-(trifluoromethyl)-2-pyridyl]methyl]-2,7-diazaspiro[3.5]nonane-2-carbonyl]-2,5-diazaspiro[3.4]octan-6-one;
2-[6-(4-fluoro-2-mesyl-benzyl)-2-azaspiro[3.3]heptane-2-carbonyl]-8-oxa-2,5-diazaspiro[3.5]nonan-6-one;
2-[7-[[6-(trifluoromethyl)-3-pyridyl]methyl]-2,7-diazaspiro[3.5]nonane-2-carbonyl]-2,5-diazaspiro[3.4]octan-6-one;
2-[7-[[5-(trifluoromethyl)-2-pyridyl]methyl]-2,7-diazaspiro[3.5]nonane-2-carbonyl]-7-oxa-2,5-diazaspiro[3.4]octan-6-one;
2-[6-(4-triflylbenzyl)-2,6-diazaspiro[3.3]heptane-2-carbonyl]-7-oxa-2,5-diazaspiro[3.4]octan-6-one;
2-[7-[[5-(trifluoromethyl)-2-pyridyl]methyl]-2,7-diazaspiro[3.4]octane-2-carbonyl]-2,5-diazaspiro[3.4]octan-6-one;
2-[7-[[6-(trifluoromethyl)-3-pyridyl]methyl]-2,7-diazaspiro[3.4]octane-2-carbonyl]-2,5-diazaspiro[3.4]octan-6-one;
2-[7-[[6-(trifluoromethyl)-3-pyridyl]methyl]-2,7-diazaspiro[3.5]nonane-2-carbonyl]-7-oxa-2,5-diazaspiro[3.4]octan-6-one;
2-[6-(3,5-difluorobenzyl)-2,6-diazaspiro[3.3]heptane-2-carbonyl]-7-oxa-2,5-diazaspiro[3.4]octan-6-one;
2-[7-[[5-(trifluoromethyl)-2-pyridyl]methyl]-2,7-diazaspiro[3.4]octane-2-carbonyl]-7-oxa-2,5-diazaspiro[3.4]octan-6-one;
2-[7-[[5-(trifluoromethyl) pyrazin-2-yl]methyl]-2,7-diazaspiro[3.4]octane-2-carbonyl]-2,5-diazaspiro[3.4]octan-6-one;
2-[7-[[6-(trifluoromethyl)-3-pyridyl]methyl]-2,7-diazaspiro[3.4]octane-2-carbonyl]-7-oxa-2,5-diazaspiro[3.4]octan-6-one;
2-[7-[[5-(trifluoromethyl) pyrazin-2-yl]methyl]-2,7-diazaspiro[3.4]octane-2-carbonyl]-7-oxa-2,5-diazaspiro[3.4]octan-6-one;
2-[7-[[6-(trifluoromethyl)pyridazin-3-yl]methyl]-2,7-diazaspiro[3.4]octane-2-carbonyl]-7-oxa-2,5-diazaspiro[3.4]octan-6-one;
2-[6-[[6-(trifluoromethyl)pyridazin-3-yl]methyl]-2,6-diazaspiro[3.3]heptane-2-carbonyl]-7-oxa-2,5-diazaspiro[3.4]octan-6-one;
2-[7-(4-mesylbenzyl)-2,7-diazaspiro[3.5]nonane-2-carbonyl]-2,5-diazaspiro[3.4]octan-6-one;
2-[6-[[2-(trifluoromethyl)pyrimidin-5-yl]methyl]-2,6-diazaspiro[3.3]heptane-2-carbonyl]-7-oxa-2,5-diazaspiro[3.4]octan-6-one;
2-[6-[(3,5-difluoro-2-pyridyl)methyl]-2,6-diazaspiro[3.3]heptane-2-carbonyl]-7-oxa-2,5-diazaspiro[3.4]octan-6-one;
2-[7-(3-mesylbenzyl)-2,7-diazaspiro[3.5]nonane-2-carbonyl]-2,5-diazaspiro[3.4]octan-6-one;
2-[6-(4-mesylbenzyl)-2,6-diazaspiro[3.3]heptane-2-carbonyl]-7-oxa-2,5-diazaspiro[3.4]octan-6-one;
2-[6-[[1-methyl-5-(trifluoromethyl) pyrazol-4-yl]methyl]-2-azaspiro[3.3]heptane-2-carbonyl]-2,5-diazaspiro[3.4]octan-6-one;
2-[6-[[1-methyl-5-(trifluoromethyl) pyrazol-3-yl]methyl]-2-azaspiro[3.3]heptane-2-carbonyl]-2,5-diazaspiro[3.4]octan-6-one;
2-[6-[[2-methyl-4-(trifluoromethyl) pyrazol-3-yl]methyl]-2-azaspiro[3.3]heptane-2-carbonyl]-2,5-diazaspiro[3.4]octan-6-one;
2-[6-[[5-(trifluoromethyl) pyrazol-1-yl]methyl]-2-azaspiro[3.3]heptane-2-carbonyl]-2,5-diazaspiro[3.4]octan-6-one;
2-[6-[[1-methyl-3-(trifluoromethyl) pyrazol-4-yl]methyl]-2-azaspiro[3.3]heptane-2-carbonyl]-2,5-diazaspiro[3.4]octan-6-one;
2-[6-[[5-(trifluoromethyl)-2-pyridyl]methyl]-2-azaspiro[3.3]heptane-2-carbonyl]-8-oxa-2,5-diazaspiro[3.5]nonan-6-one;
2-[6-[[3-fluoro-5-(trifluoromethyl)-2-pyridyl]methyl]-2-azaspiro[3.3]heptane-2-carbonyl]-8-oxa-2,5-diazaspiro[3.5]nonan-6-one;
2-[6-[(5-chloro-3-pyridyl)methyl]-2-azaspiro[3.3]heptane-2-carbonyl]-8-oxa-2,5-diazaspiro[3.5]nonan-6-one;
2-[6-[[5-(trifluoromethyl)-3-pyridyl]methyl]-2-azaspiro[3.3]heptane-2-carbonyl]-8-oxa-2,5-diazaspiro[3.5]nonan-6-one;
2-[6-[[6-(trifluoromethyl)-3-pyridyl]methyl]-2-azaspiro[3.3]heptane-2-carbonyl]-8-oxa-2,5-diazaspiro[3.5]nonan-6-one;
2-[6-[(5-chloro-3-fluoro-2-pyridyl)methyl]-2-azaspiro[3.3]heptane-2-carbonyl]-2,5-diazaspiro[3.4]octan-6-one;
2-[6-[(3-chloro-5-fluoro-2-pyridyl)methyl]-2-azaspiro[3.3]heptane-2-carbonyl]-8-oxa-2,5-diazaspiro[3.5]nonan-6-one;
2-[6-[[5-(trifluoromethoxy)-2-pyridyl]methyl]-2-azaspiro[3.3]heptane-2-carbonyl]-8-oxa-2,5-diazaspiro[3.5]nonan-6-one;
2-[6-[[4-(trifluoromethyl)-2-pyridyl]methyl]-2-azaspiro[3.3]heptane-2-carbonyl]-8-oxa-2,5-diazaspiro[3.5]nonan-6-one;
2-[6-[[6-(trifluoromethoxy)-3-pyridyl]methyl]-2-azaspiro[3.3]heptane-2-carbonyl]-2,5-diazaspiro[3.4]octan-6-one;
2-[6-[(5-fluoro-3-pyridyl)methyl]-2-azaspiro[3.3]heptane-2-carbonyl]-8-oxa-2,5-diazaspiro[3.5]nonan-6-one;
2-[6-[[4-methylsulfonyl-3-(trifluoromethyl)phenyl]methyl]-2-azaspiro[3.3]heptane-2-carbonyl]-8-oxa-2,5-diazaspiro[3.5]nonan-6-one;
2-[6-[[3-(trifluoromethylsulfonyl)phenyl]methyl]-2-azaspiro[3.3]heptane-2-carbonyl]-8-oxa-2,5-diazaspiro[3.5]nonan-6-one;
2-[6-[[4-methylsulfonyl-3-(trifluoromethyl)phenyl]methyl]-2-azaspiro[3.3]heptane-2-carbonyl]-2,5-diazaspiro[3.5]nonan-6-one;
2-[6-[[2-methylsulfonyl-4-(trifluoromethyl)phenyl]methyl]-2-azaspiro[3.3]heptane-2-carbonyl]-8-oxa-2,5-diazaspiro[3.5]nonan-6-one;
2-[6-[[2-methylsulfonyl-4-(trifluoromethyl)phenyl]methyl]-2-azaspiro[3.3]heptane-2-carbonyl]-2,5-diazaspiro[3.5]nonan-6-one;
2-[6-[[3-(trifluoromethylsulfonyl)phenyl]methyl]-2-azaspiro[3.3]heptane-2-carbonyl]-2,5-diazaspiro[3.5]nonan-6-one;
2-[6-[(2-methylsulfonylphenyl)methyl]-2,6-diazaspiro[3.3]heptane-2-carbonyl]-7-oxa-2,5-diazaspiro[3.4]octan-6-one;
2-[7-[[2-fluoro-4-(trifluoromethyl)phenyl]sulfonyl-2,7-diazaspiro[3.5]nonane-2-carbonyl]-7-oxa-2,5-diazaspiro[3.4]octan-6-one;
2-[2-[2-(trifluoromethoxy)phenyl]sulfonyl-2,6-diazaspiro[3.3]heptane-6-carbonyl]-7-oxa-2,5-diazaspiro[3.4]octan-6-one;
2-[2-[4-(trifluoromethoxy)phenyl]sulfonyl-2,6-diazaspiro[3.3]heptane-6-carbonyl]-7-oxa-2,5-diazaspiro[3.4]octan-6-one;
2-[2-[3-(trifluoromethoxy)phenyl]sulfonyl-2,6-diazaspiro[3.3]heptane-6-carbonyl]-7-oxa-2,5-diazaspiro[3.4]octan-6-one;
N-(1-methylcyclopropyl)-6-(6-oxo-7-oxa-2,5-diazaspiro[3.4]octane-2-carbonyl)-2,6-diazaspiro[3.3]heptane-2-sulfonamide;
2-[7-[[2-fluoro-4-(trifluoromethyl)phenyl]sulfonyl-2,7-diazaspiro[3.5]nonane-2-carbonyl]-2,5-diazaspiro[3.4]octan-6-one;
2-[2-[4-fluoro-2-(trifluoromethyl)phenyl]sulfonyl-2,6-diazaspiro[3.3]heptane-6-carbonyl]-7-oxa-2,5-diazaspiro[3.4]octan-6-one;
2-[2-[[5-(trifluoromethyl)-3-pyridyl]sulfonyl]-2,6-diazaspiro[3.3]heptane-6-carbonyl]-7-oxa-2,5-diazaspiro[3.4]octan-6-one;
6-(6-oxo-7-oxa-2,5-diazaspiro[3.4]octane-2-carbonyl)-N-[[1-(trifluoromethyl)cyclopropyl]methyl]-2,6-diazaspiro[3.3]heptane-2-sulfonamide;
2-[2-[3-(trifluoromethoxy)phenyl]sulfonyl-2,6-diazaspiro[3.3]heptane-6-carbonyl]-2,5-diazaspiro[3.4]octan-6-one;
2-[2-[4-(trifluoromethoxy)phenyl]sulfonyl-2,6-diazaspiro[3.3]heptane-6-carbonyl]-2,5-diazaspiro[3.4]octan-6-one;
2-[2-[2-(trifluoromethoxy)phenyl]sulfonyl-2,6-diazaspiro[3.3]heptane-6-carbonyl]-2,5-diazaspiro[3.4]octan-6-one;
N-methyl-2-[[2-(6-oxo-7-oxa-2,5-diazaspiro[3.4]octane-2-carbonyl)-2,6-diazaspiro[3.3]heptan-6-yl]methyl]benzamide;
2-[6-[[5-(trifluoromethyl)-2-pyridyl]-2,6-diazaspiro[3.3]heptane-2-carbonyl]-7-oxa-2,5-diazaspiro[3.4]octan-6-one;
2-[6-[[3-fluoro-5-(trifluoromethyl)-2-pyridyl]-2,6-diazaspiro[3.3]heptane-2-carbonyl]-7-oxa-2,5-diazaspiro[3.4]octan-6-one;
2-[7-[[3-(trifluoromethyl)-1-bicyclo[1.1.1]pentanyl]sulfonyl]-2,7-diazaspiro[3.5]nonane-2-carbonyl]-7-oxa-2,5-diazaspiro[3.4]octan-6-one;
2-[7-[[3-(trifluoromethyl)-1-bicyclo[1.1.1]pentanyl]sulfonyl]-2,7-diazaspiro[3.5]nonane-2-carbonyl]-2,5-diazaspiro[3.4]octan-6-one;
2-[6-[[5-(trifluoromethyl) pyrazin-2-yl]-2,6-diazaspiro[3.3]heptane-2-carbonyl]-7-oxa-2,5-diazaspiro[3.4]octan-6-one;
2-[6-[[5-(trifluoromethyl)pyrimidin-2-yl]-2,6-diazaspiro[3.3]heptane-2-carbonyl]-7-oxa-2,5-diazaspiro[3.4]octan-6-one;
N-[2-(6-oxo-8-oxa-2,5-diazaspiro[3.5]nonane-2-carbonyl)-2-azaspiro[3.3]heptan-6-yl]-3-(trifluoromethyl)benzenesulfonamide;
5-[[(6S)-2-(6-oxo-7-oxa-2,5-diazaspiro[3.4]octane-2-carbonyl)-2-azaspiro[3.4]octan-6-yl]oxy]-2-(trifluoromethyl)pyridine-4-carbonitrile;
5-[[2-(6-oxo-7-oxa-2,5-diazaspiro[3.4]octane-2-carbonyl)-2-azaspiro[3.4]octan-6-yl]methyl]-2-(trifluoromethyl)pyridine-4-carbonitrile;
5-[[2-(6-oxo-7-oxa-2,5-diazaspiro[3.4]octane-2-carbonyl)-2-azaspiro[3.3]heptan-6-yl]methyl]-2-(trifluoromethyl)pyridine-4-carbonitrile;
5-[[(6S)-2-(6-oxo-7-oxa-2,5-diazaspiro[3.4]octane-2-carbonyl)-2-azaspiro[3.4]octan-6-yl]methyl]-2-(trifluoromethyl)pyridine-4-carbonitrile;
5-[[(6R)-2-(6-oxo-7-oxa-2,5-diazaspiro[3.4]octane-2-carbonyl)-2-azaspiro[3.4]octan-6-yl]methyl]-2-(trifluoromethyl)pyridine-4-carbonitrile;
5-[[(6S)-2-(6-oxo-8-oxa-2,5-diazaspiro[3.5]nonane-2-carbonyl)-2-azaspiro[3.4]octan-6-yl]oxy]-2-(trifluoromethyl)pyridine-4-carbonitrile;
2-[6-[(4-cyclopropylsulfonylphenyl)methyl]-2-azaspiro[3.3]heptane-2-carbonyl]-2,5-diazaspiro[3.4]octan-6-one;
2-[6-[[1-(trifluoromethyl) pyrazol-4-yl]methyl]-2-azaspiro[3.3]heptane-2-carbonyl]-2,5-diazaspiro[3.4]octan-6-one;
3-[[2-(6-oxo-2,5-diazaspiro[3.4]octane-2-carbonyl)-2-azaspiro[3.3]heptan-6-yl]methyl]-5-(trifluoromethyl)benzonitrile;
2-[7-[[4-(trifluoromethylsulfonyl)phenyl]methyl]-2,7-diazaspiro[3.4]octane-2-carbonyl]-2,5-diazaspiro[3.4]octan-6-one;
4-[[2-(6-oxo-2,5-diazaspiro[3.4]octane-2-carbonyl)-2-azaspiro[3.3]heptan-6-yl]methyl]-2-(trifluoromethyl)benzonitrile;
2-[6-[[3-(trifluoromethyl)-1H-pyrazol-5-yl]methyl]-2-azaspiro[3.3]heptane-2-carbonyl]-2,5-diazaspiro[3.4]octan-6-one;
2-[(6R)-6-[[4-(trifluoromethylsulfonyl)phenyl]methyl]-2-azaspiro[3.4]octane-2-carbonyl]-2,5-diazaspiro[3.4]octan-6-one;
2-[(6S)-6-[[4-(trifluoromethylsulfonyl)phenyl]methyl]-2-azaspiro[3.4]octane-2-carbonyl]-2,5-diazaspiro[3.4]octan-6-one;
2-[(6R)-6-[[3-(trifluoromethylsulfonyl)phenyl]methyl]-2-azaspiro[3.4]octane-2-carbonyl]-2,5-diazaspiro[3.4]octan-6-one;
2-[(6S)-6-(3-triflylbenzyl)-2-azaspiro[3.4]octane-2-carbonyl]-2,5-diazaspiro[3.4]octan-6-one; 2-[6-[[3-(trifluoromethyl) pyrazol-1-yl]methyl]-2-azaspiro[3.3]heptane-2-carbonyl]-2,5-diazaspiro[3.4]octan-6-one;
2-[7-[[5-(trifluoromethyl) pyrazin-2-yl]methyl]-2-azaspiro[3.5]nonane-2-carbonyl]-8-oxa-2,5-diazaspiro[3.5]nonan-6-one;
2-[6-(3-cyclopropyl-1,2,4-triazol-1-yl)-2-azaspiro[3.3]heptane-2-carbonyl]-8-oxa-2,5-diazaspiro[3.5]nonan-6-one;
2-[6-[[2-fluoro-4-(trifluoromethyl)phenyl]-2-azaspiro[3.3]heptane-2-carbonyl]-8-oxa-2,5-diazaspiro[3.5]nonan-6-one;
2-[6-[[4-(trifluoromethylsulfonyl)phenyl]methyl]-2,6-diazaspiro[3.3]heptane-2-carbonyl]-8-oxa-2,5-diazaspiro[3.5]nonan-6-one;
2-[6-[[4-(trifluoromethyl) pyrazol-1-yl]methyl]-2-azaspiro[3.3]heptane-2-carbonyl]-8-oxa-2,5-diazaspiro[3.5]nonan-6-one;
2-[6-[[6-(trifluoromethyl)pyridazin-3-yl]methyl]-2-azaspiro[3.3]heptane-2-carbonyl]-8-oxa-2,5-diazaspiro[3.5]nonan-6-one;
2-[6-[[2-(trifluoromethyl)pyrimidin-5-yl]methyl]-2-azaspiro[3.3]heptane-2-carbonyl]-8-oxa-2,5-diazaspiro[3.5]nonan-6-one;
2-[7-[[4-(trifluoromethylsulfonyl)phenyl]methyl]-2,7-diazaspiro[3.4]octane-2-carbonyl]-8-oxa-2,5-diazaspiro[3.5]nonan-6-one;
2-[(6S)-6-[[4-(trifluoromethylsulfonyl)phenyl]methyl]-2-azaspiro[3.4]octane-2-carbonyl]-8-oxa-2,5-diazaspiro[3.5]nonan-6-one;
2-[(6R)-6-[[4-(trifluoromethylsulfonyl)phenyl]methyl]-2-azaspiro[3.4]octane-2-carbonyl]-8-oxa-2,5-diazaspiro[3.5]nonan-6-one;
2-[(6S)-6-[[3-(trifluoromethylsulfonyl)phenyl]methyl]-2-azaspiro[3.4]octane-2-carbonyl]-8-oxa-2,5-diazaspiro[3.5]nonan-6-one;
2-[(6R)-6-[[3-(trifluoromethylsulfonyl)phenyl]methyl]-2-azaspiro[3.4]octane-2-carbonyl]-8-oxa-2,5-diazaspiro[3.5]nonan-6-one;
2-[6-[[3-methylsulfonyl-5-(trifluoromethyl)phenyl]methyl]-2-azaspiro[3.3]heptane-2-carbonyl]-8-oxa-2,5-diazaspiro[3.5]nonan-6-one;
2-[6-[[3-(trifluoromethyl) pyrazol-1-yl]methyl]-2-azaspiro[3.3]heptane-2-carbonyl]-8-oxa-2,5-diazaspiro[3.5]nonan-6-one;
2-[7-[[6-(trifluoromethyl)-3-pyridyl]methyl]-2,7-diazaspiro[3.4]octane-2-carbonyl]-8-oxa-2,5-diazaspiro[3.5]nonan-6-one;
2-[7-[[5-(trifluoromethyl)-2-pyridyl]methyl]-2,7-diazaspiro[3.4]octane-2-carbonyl]-8-oxa-2,5-diazaspiro[3.5]nonan-6-one;
2-[[2-(6-oxo-7-oxa-2,5-diazaspiro[3.4]octane-2-carbonyl)-2-azaspiro[3.3]heptan-6-yl]methyl]-5-(trifluoromethyl)benzonitrile;
5-[[2-(6-oxo-7-oxa-2,5-diazaspiro[3.4]octane-2-carbonyl)-2-azaspiro[3.3]heptan-6-yl]methyl]-2-(trifluoromethyl)pyridine-3-carbonitrile;
4-[[2-(6-oxo-7-oxa-2,5-diazaspiro[3.4]octane-2-carbonyl)-2-azaspiro[3.3]heptan-6-yl]methyl]-2-(trifluoromethoxy)benzamide;
5-[[2-(6-oxo-7-oxa-2,5-diazaspiro[3.4]octane-2-carbonyl)-2-azaspiro[3.3]heptan-6-yl]methyl]-2-(trifluoromethoxy)benzamide;
2-[6-[[2-methyl-5-(trifluoromethyl) pyrazol-3-yl]methyl]-2-azaspiro[3.3]heptane-2-carbonyl]-2,5-diazaspiro[3.4]octan-6-one;
1-[[2-(6-oxo-2,5-diazaspiro[3.4]octane-2-carbonyl)-2-azaspiro[3.3]heptan-6-yl]methyl]pyrazole-4-carbonitrile;
2-[6-[[1-(2,2,2-trifluoroethyl) pyrazol-4-yl]methyl]-2-azaspiro[3.3]heptane-2-carbonyl]-2,5-diazaspiro[3.4]octan-6-one;
2-[6-[[2-oxo-4-(trifluoromethyl)-1-pyridyl]methyl]-2-azaspiro[3.3]heptane-2-carbonyl]-2,5-diazaspiro[3.4]octan-6-one;
6-oxo-N-[[6-(trifluoromethyl)-3,4-diazatricyclo[7.1.1.03,7]undeca-4,6-dien-1-yl]methyl]-2,5-diazaspiro[3.4]octane-2-carboxamide;
3-fluoro-4-[[2-(6-oxo-2,5-diazaspiro[3.4]octane-2-carbonyl)-2-azaspiro[3.3]heptan-6-yl]methyl]benzonitrile;
2-[6-[[5-(trifluoromethylsulfonyl)-3-pyridyl]methyl]-2-azaspiro[3.3]heptane-2-carbonyl]-2,5-diazaspiro[3.4]octan-6-one;
2-[6-[[5-(trifluoromethylsulfonyl)-3-pyridyl]methyl]-2-azaspiro[3.3]heptane-2-carbonyl]-8-oxa-2,5-diazaspiro[3.5]nonan-6-one;
2-[6-[[3-fluoro-5-(trifluoromethylsulfonyl)phenyl]methyl]-2-azaspiro[3.3]heptane-2-carbonyl]-8-oxa-2,5-diazaspiro[3.5]nonan-6-one;
2-[6-[[3-(methylsulfonimidoyl)-5-(trifluoromethyl)phenyl]methyl]-2-azaspiro[3.3]heptane-2-carbonyl]-2,5-diazaspiro[3.4]octan-6-one;
2-[7-[[3-(trifluoromethylsulfonyl)phenyl]methyl]-2,7-diazaspiro[3.4]octane-2-carbonyl]-2,5-diazaspiro[3.4]octan-6-one;
2-[6-[[5-(difluoromethyl)-1H-pyrazol-3-yl]methyl]-2-azaspiro[3.3]heptane-2-carbonyl]-2,5-diazaspiro[3.4]octan-6-one;
2-[6-[[2-fluoro-4-(trifluoromethylsulfonyl)phenyl]methyl]-2,6-diazaspiro[3.3]heptane-2-carbonyl]-2,5-diazaspiro[3.4]octan-6-one;
2-[6-[[2-oxo-5-(trifluoromethyl)-1-pyridyl]methyl]-2-azaspiro[3.3]heptane-2-carbonyl]-2,5-diazaspiro[3.4]octan-6-one;
2-[7-[[3-(trifluoromethylsulfonyl)phenyl]methyl]-2,7-diazaspiro[3.4]octane-2-carbonyl]-8-oxa-2,5-diazaspiro[3.5]nonan-6-one;
5-[[2-(6-oxo-7-oxa-2,5-diazaspiro[3.4]octane-2-carbonyl)-2-azaspiro[3.3]heptan-6-yl]methyl]-3-(trifluoromethyl)pyridine-2-carbonitrile;
2-[6-[[5-(trifluoromethylsulfonyl)-2-pyridyl]methyl]-2-azaspiro[3.3]heptane-2-carbonyl]-2,5-diazaspiro[3.4]octan-6-one;
2-[6-[[5-(trifluoromethylsulfonyl)-2-pyridyl]methyl]-2-azaspiro[3.3]heptane-2-carbonyl]-2,5-diazaspiro[3.5]nonan-6-one;
2-[6-[[5-(trifluoromethylsulfonyl)-2-pyridyl]methyl]-2-azaspiro[3.3]heptane-2-carbonyl]-8-oxa-2,5-diazaspiro[3.5]nonan-6-one;
2-[6-[[2-fluoro-4-(trifluoromethylsulfonimidoyl)phenyl]methyl]-2-azaspiro[3.3]heptane-2-carbonyl]-2,5-diazaspiro[3.4]octan-6-one;
2-[7-[[3-(trifluoromethylsulfonyl)phenyl]methyl]-2,7-diazaspiro[3.4]octane-2-carbonyl]-2,5-diazaspiro[3.5]nonan-6-one;
2-[6-[[2-fluoro-4-(trifluoromethylsulfonyl)phenyl]methyl]-2,6-diazaspiro[3.3]heptane-2-carbonyl]-2,5-diazaspiro[3.5]nonan-6-one;
2-[6-[[3-methylsulfonyl-5-(trifluoromethyl)phenyl]methyl]-2-azaspiro[3.3]heptane-2-carbonyl]-2,5-diazaspiro[3.5]nonan-6-one;
2-[6-[(4-cyclopropylsulfonylphenyl)methyl]-2-azaspiro[3.3]heptane-2-carbonyl]-2,5-diazaspiro[3.5]nonan-6-one;
4-[[2-(6-oxo-2,5-diazaspiro[3.5]nonane-2-carbonyl)-2-azaspiro[3.3]heptan-6-yl]methyl]-2-(trifluoromethyl)benzonitrile;
5-[[2-(6-oxo-2,5-diazaspiro[3.5]nonane-2-carbonyl)-2-azaspiro[3.3]heptan-6-yl]methyl]-2-(trifluoromethyl)benzonitrile;
5-[[2-(6-oxo-2,5-diazaspiro[3.5]nonane-2-carbonyl)-2-azaspiro[3.3]heptan-6-yl]methyl]-2-(trifluoromethyl)pyridine-3-carbonitrile;
3-fluoro-4-[[2-(6-oxo-2,5-diazaspiro[3.5]nonane-2-carbonyl)-2-azaspiro[3.3]heptan-6-yl]methyl]benzonitrile;
2-[6-[[4-(trifluoromethyl) pyrazol-1-yl]methyl]-2-azaspiro[3.3]heptane-2-carbonyl]-2,5-diazaspiro[3.5]nonan-6-one;
2-[6-[[1-methyl-5-(trifluoromethyl) pyrazol-3-yl]methyl]-2-azaspiro[3.3]heptane-2-carbonyl]-2,5-diazaspiro[3.5]nonan-6-one;
2-[6-[[1-methyl-5-(trifluoromethyl) pyrazol-4-yl]methyl]-2-azaspiro[3.3]heptane-2-carbonyl]-2,5-diazaspiro[3.5]nonan-6-one;
2-[6-[[2-methyl-4-(trifluoromethyl) pyrazol-3-yl]methyl]-2-azaspiro[3.3]heptane-2-carbonyl]-2,5-diazaspiro[3.5]nonan-6-one;
2-[6-[[3-(trifluoromethyl)-1H-pyrazol-5-yl]methyl]-2-azaspiro[3.3]heptane-2-carbonyl]-2,5-diazaspiro[3.5]nonan-6-one;
2-[6-[[3-(difluoromethyl)-1H-pyrazol-5-yl]methyl]-2-azaspiro[3.3]heptane-2-carbonyl]-2,5-diazaspiro[3.5]nonan-6-one;
2-[6-[[3-(trifluoromethoxy)phenyl]sulfonyl-2-azaspiro[3.3]heptane-2-carbonyl]-2,5-diazaspiro[3.4]octan-6-one;
2-[7-[[4-(trifluoromethyl)phenyl]sulfonyl-2-azaspiro[3.5]nonane-2-carbonyl]-2,5-diazaspiro[3.4]octan-6-one;
2-[7-[3-(trifluoromethyl)phenyl]sulfonyl-2-azaspiro[3.5]nonane-2-carbonyl]-2,5-diazaspiro[3.4]octan-6-one;
2-[6-[[4-(trifluoromethylsulfonimidoyl)phenyl]methyl]-2-azaspiro[3.3]heptane-2-carbonyl]-2,5-diazaspiro[3.4]octan-6-one;
2-[6-[[4-(trifluoromethylsulfonimidoyl)phenyl]methyl]-2-azaspiro[3.3]heptane-2-carbonyl]-2,5-diazaspiro[3.5]nonan-6-one;
2-[6-[[4-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[4-(trifluoromethylsulfonimidoyl)phenyl]methyl]-2-azaspiro[3.3]heptane-2-carbonyl]-2,5-diazaspiro[3.4]octan-6-one;
2-[6-[[4-(trifluoromethylsulfonimidoyl)phenyl]methyl]-2-azaspiro[3.3]heptane-2-carbonyl]-2,5-diazaspiro[3.5]nonan-6-one;
2-[6-[[4-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[3-(trifluoromethylsulfonimidoyl)phenyl]methyl]-2-azaspiro[3.3]heptane-2-carbonyl]-2,5-diazaspiro[3.4]octan-6-one;
2-[6-[[3-(trifluoromethylsulfonimidoyl)phenyl]methyl]-2-azaspiro[3.3]heptane-2-carbonyl]-2,5-diazaspiro[3.5]nonan-6-one;
2-[6-[[3-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[3-(trifluoromethylsulfonimidoyl)phenyl]methyl]-2-azaspiro[3.3]heptane-2-carbonyl]-2,5-diazaspiro[3.4]octan-6-one;
2-[6-[[3-(trifluoromethylsulfonimidoyl)phenyl]methyl]-2-azaspiro[3.3]heptane-2-carbonyl]-2,5-diazaspiro[3.5]nonan-6-one;
2-[6-[[3-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[4-(trifluoromethylsulfonyl)phenyl]methyl]-2-azaspiro[3.3]heptane-2-carbonyl]-2,5-diazaspiro[3.4]octan-6-one;
2-[6-[[4-(trifluoromethylsulfonyl)phenyl]methyl]-2-azaspiro[3.3]heptane-2-carbonyl]-8-oxa-2,5-diazaspiro[3.5]nonan-6-one;
2-[6-[[3-(trifluoromethoxy)phenyl]sulfonimidoyl]-2-azaspiro[3.3]heptane-2-carbonyl]-2,5-diazaspiro[3.4]octan-6-one;
2-[6-[[3-fluoro-5-(trifluoromethylsulfonimidoyl)phenyl]methyl]-2-azaspiro[3.3]heptane-2-carbonyl]-2,5-diazaspiro[3.4]octan-6-one;
2-[6-[[5-(trifluoromethyl)-3-pyridyl]oxy]-2-azaspiro[3.3]heptane-2-carbonyl]-2,5-diazaspiro[3.4]octan-6-one;
2-[6-[[4-(trifluoromethyl)triazol-2-yl]methyl]-2-azaspiro[3.3]heptane-2-carbonyl]-2,5-diazaspiro[3.4]octan-6-one;
4-[[2-(6-oxo-2,5-diazaspiro[3.4]octane-2-carbonyl)-2-azaspiro[3.3]heptan-6-yl]oxy]-2-(trifluoromethyl)benzonitrile;
4-[[2-(6-oxo-8-oxa-2,5-diazaspiro[3.5]nonane-2-carbonyl)-2-azaspiro[3.3]heptan-6-yl]oxy]-2-(trifluoromethyl)benzonitrile;
2-[6-[(5-chloro-2-pyridyl)oxy]-2-azaspiro[3.3]heptane-2-carbonyl]-2,5-diazaspiro[3.4]octan-6-one;
2-[6-[[1-methyl-4-(trifluoromethyl) pyrazol-3-yl]methyl]-2-azaspiro[3.3]heptane-2-carbonyl]-2,5-diazaspiro[3.4]octan-6-one;
2-[6-[(5-chloro-1,3-dimethyl-pyrazol-4-yl)methyl]-2,6-diazaspiro[3.3]heptane-2-carbonyl]-7-oxa-2,5-diazaspiro[3.4]octan-6-one;
2-[6-[[4-(trifluoromethyl) thiazol-2-yl]methyl]-2,6-diazaspiro[3.3]heptane-2-carbonyl]-2,5-diazaspiro[3.4]octan-6-one;
5-[[2-(6-oxo-2,5-diazaspiro[3.4]octane-2-carbonyl)-2-azaspiro[3.3]heptan-6-yl]oxy]-2-(trifluoromethyl)benzonitrile;
2-[6-[[4-(trifluoromethyl) thiazol-2-yl]oxy-2-azaspiro[3.3]heptane-2-carbonyl]-2,5-diazaspiro[3.4]octan-6-one;
2-[6-[(5-fluoro-2-pyridyl)oxy]-2-azaspiro[3.3]heptane-2-carbonyl]-2,5-diazaspiro[3.4]octan-6-one;
2-[6-[[1-methyl-3-(trifluoromethyl) pyrazol-4-yl]oxy-2-azaspiro[3.3]heptane-2-carbonyl]-2,5-diazaspiro[3.4]octan-6-one;
2-[6-[[4-[1-(trifluoromethyl)cyclopropyl]pyrazol-1-yl]methyl]-2-azaspiro[3.3]heptane-2-carbonyl]-2,5-diazaspiro[3.4]octan-6-one;
2-[6-[6-(trifluoromethyl)pyrimidin-4-yl]methyl]-2-azaspiro[3.3]heptane-2-carbonyl]-2,5-diazaspiro[3.4]octan-6-one;
2-[6-[(3-chloro-5-fluoro-2-pyridyl)oxy]-2-azaspiro[3.3]heptane-2-carbonyl]-2,5-diazaspiro[3.4]octan-6-one;
2-[6-[[2-(trifluoromethyl)pyrimidin-4-yl]methyl]-2-azaspiro[3.3]heptane-2-carbonyl]-2,5-diazaspiro[3.4]octan-6-one;
2-[6-[[2-(trifluoromethyl) thiazol-5-yl]methyl]-2,6-diazaspiro[3.3]heptane-2-carbonyl]-2,5-diazaspiro[3.4]octan-6-one;
2-[6-[[5-(trifluoromethyl)-1H-pyrazol-4-yl]methyl]-2,6-diazaspiro[3.3]heptane-2-carbonyl]-2,5-diazaspiro[3.4]octan-6-one;
2-[7-[[6-(trifluoromethyl)pyridazin-3-yl]methyl]-2,7-diazaspiro[3.4]octane-2-carbonyl]-2,5-diazaspiro[3.4]octan-6-one;
2-[6-(5-fluoro-3-pyridyl)-2,6-diazaspiro[3.3]heptane-2-carbonyl]-7-oxa-2,5-diazaspiro[3.4]octan-6-one;
2-[6-[[4-(trifluoromethyl) thiazol-2-yl]methyl]-2-azaspiro[3.3]heptane-2-carbonyl]-2,5-diazaspiro[3.4]octan-6-one;
2-[6-([1,2,4]triazolo[1,5-a]pyridin-6-ylmethyl)-2-azaspiro[3.3]heptane-2-carbonyl]-2,5-diazaspiro[3.4]octan-6-one;
2-[6-(1H-pyrazolo[4,3-b]pyridin-5-ylmethyl)-2-azaspiro[3.3]heptane-2-carbonyl]-2,5-diazaspiro[3.4]octan-6-one;
2-[6-[[3-fluoro-5-(trifluoromethylsulfonimidoyl)phenyl]methyl]-2-azaspiro[3.3]heptane-2-carbonyl]-8-oxa-2,5-diazaspiro[3.5]nonan-6-one;
2-[7-[[5-(trifluoromethyl)-4H-1,2,4-triazol-3-yl]methyl]-2-azaspiro[3.5]nonane-2-carbonyl]-2,5-diazaspiro[3.4]octan-6-one;
2-[6-[[1-cyclopropyl-3-(trifluoromethyl) pyrazol-4-yl]methyl]-2-azaspiro[3.3]heptane-2-carbonyl]-2,5-diazaspiro[3.4]octan-6-one;
2-[7-[[2-methyl-4-(trifluoromethyl) pyrazol-3-yl]methyl]-2,7-diazaspiro[3.5]nonane-2-carbonyl]-2,5-diazaspiro[3.4]octan-6-one;
2-[6-[[5-methyl-3-(trifluoromethyl) pyrazol-1-yl]methyl]-2-azaspiro[3.3]heptane-2-carbonyl]-2,5-diazaspiro[3.4]octan-6-one;
2-[6-[[4-(trifluoromethyl)pyrimidin-2-yl]methyl]-2-azaspiro[3.3]heptane-2-carbonyl]-2,5-diazaspiro[3.4]octan-6-one;
2-[6-[[3-methyl-5-(trifluoromethyl) pyrazol-1-yl]methyl]-2-azaspiro[3.3]heptane-2-carbonyl]-2,5-diazaspiro[3.4]octan-6-one;
2-[6-[[2-keto-5-(trifluoromethyl)-1H-pyridin-3-yl]methyl]-2-azaspiro[3.3]heptane-2-carbonyl]-2,5-diazaspiro[3.4]octan-6-one;
2-[6-[[3-[1-(trifluoromethyl)cyclopropyl]-1,2,4-triazol-1-yl]methyl]-2-azaspiro[3.3]heptane-2-carbonyl]-2,5-diazaspiro[3.4]octan-6-one;
2-[6-[(2-tert-butylthiazol-5-yl)methyl]-2,6-diazaspiro[3.3]heptane-2-carbonyl]-2,5-diazaspiro[3.4]octan-6-one;
2-[7-[[5-(trifluoromethyl) isothiazol-3-yl]methyl]-2,7-diazaspiro[3.5]nonane-2-carbonyl]-2,5-diazaspiro[3.4]octan-6-one;
2-[6-[[5-(trifluoromethyl)-1H-1,2,4-triazol-3-yl]methyl]-2-azaspiro[3.3]heptane-2-carbonyl]-2,5-diazaspiro[3.4]octan-6-one;
2-[6-[(4-cyclobutylthiadiazol-5-yl)methyl]-2,6-diazaspiro[3.3]heptane-2-carbonyl]-2,5-diazaspiro[3.4]octan-6-one;
2-[6-[[4-(trifluoromethylsulfonimidoyl) phenoxy]-2-azaspiro[3.3]heptane-2-carbonyl]-2,5-diazaspiro[3.4]octan-6-one;
2-[6-(4-dimethylphosphoryl-2-fluoro-benzyl)-2-azaspiro[3.3]heptane-2-carbonyl]-2,5-diazaspiro[3.4]octan-6-one;
2-[7-[[2-methyl-5-(trifluoromethyl) pyrazol-3-yl]methyl]-2,7-diazaspiro[3.5]nonane-2-carbonyl]-2,5-diazaspiro[3.4]octan-6-one;
2-[7-[[5-(trifluoromethyl) isoxazol-3-yl]methyl]-2,7-diazaspiro[3.5]nonane-2-carbonyl]-2,5-diazaspiro[3.4]octan-6-one;
2-[6-(3-dimethylphosphoryl-5-fluoro-benzyl)-2-azaspiro[3.3]heptane-2-carbonyl]-2,5-diazaspiro[3.4]octan-6-one;
2-[6-[(3-cyclopropyl-1,2,4-thiadiazol-5-yl)methyl]-2,6-diazaspiro[3.3]heptane-2-carbonyl]-2,5-diazaspiro[3.4]octan-6-one;
2-[6-[[5-methyl-4-(trifluoromethyl) thiazol-2-yl]methyl]-2,6-diazaspiro[3.3]heptane-2-carbonyl]-2,5-diazaspiro[3.4]octan-6-one;
2-[6-[[5-(trifluoromethyl)-1,3,4-thiadiazol-2-yl]methyl]-2,6-diazaspiro[3.3]heptane-2-carbonyl]-2,5-diazaspiro[3.4]octan-6-one;
2-[6-[[2-methyl-4-(trifluoromethyl) thiazol-5-yl]methyl]-2,6-diazaspiro[3.3]heptane-2-carbonyl]-2,5-diazaspiro[3.4]octan-6-one;
2-[6-(1-methylpyrazol-4-yl)oxy-2-azaspiro[3.3]heptane-2-carbonyl]-2,5-diazaspiro[3.4]octan-6-one;
2-[6-[(4-cyclopropylthiadiazol-5-yl)methyl]-2,6-diazaspiro[3.3]heptane-2-carbonyl]-2,5-diazaspiro[3.4]octan-6-one;
2-[6-[[5-(difluoromethyl)-3-methyl-pyrazol-1-yl]methyl]-2-azaspiro[3.3]heptane-2-carbonyl]-2,5-diazaspiro[3.4]octan-6-one;
2-[6-[[3-(difluoromethyl)-5-methyl-pyrazol-1-yl]methyl]-2-azaspiro[3.3]heptane-2-carbonyl]-2,5-diazaspiro[3.4]octan-6-one;
2-[7-[[5-(difluoromethyl) thiazol-2-yl]methyl]-2,7-diazaspiro[3.5]nonane-2-carbonyl]-2,5-diazaspiro[3.4]octan-6-one;
2-[6-[[5-(trifluoromethyl)-1,2,4-thiadiazol-3-yl]methyl]-2-azaspiro[3.3]heptane-2-carbonyl]-2,5-diazaspiro[3.4]octan-6-one;
2-[7-[[5-(trifluoromethyl)-1H-pyrazol-3-yl]methyl]-2,7-diazaspiro[3.5]nonane-2-carbonyl]-2,5-diazaspiro[3.4]octan-6-one;
2-[6-[[4-(trifluoromethyl) thiazol-2-yl]methyl]-2-azaspiro[3.3]heptane-2-carbonyl]-7-oxa-2,5-diazaspiro[3.4]octan-6-one;
2-[6-[[1-methyl-5-(trifluoromethyl) pyrazol-4-yl]methyl]-2-azaspiro[3.3]heptane-2-carbonyl]-7-oxa-2,5-diazaspiro[3.4]octan-6-one;
4-[[2-(6-keto-7-oxa-2,5-diazaspiro[3.4]octane-2-carbonyl)-2-azaspiro[3.3]heptan-6-yl]methyl]-2-(trifluoromethyl)benzonitrile;
2-[6-[[5-(trifluoromethyl)-2-pyridyl]methyl]-2-azaspiro[3.3]heptane-2-carbonyl]-7-oxa-2,5-diazaspiro[3.4]octan-6-one;
2-[6-[[6-(trifluoromethoxy)-3-pyridyl]methyl]-2-azaspiro[3.3]heptane-2-carbonyl]-7-oxa-2,5-diazaspiro[3.4]octan-6-one;
2-[6-[[3-(trifluoromethylsulfonimidoyl)benzyl]-2-azaspiro[3.3]heptane-2-carbonyl]-7-oxa-2,5-diazaspiro[3.4]octan-6-one;
2-[6-[[3-(trifluoromethylsulfonimidoyl)benzyl]-2-azaspiro[3.3]heptane-2-carbonyl]-7-oxa-2,5-diazaspiro[3.4]octan-6-one;
2-[6-[[4-(trifluoromethylsulfonimidoyl)benzyl]-2-azaspiro[3.3]heptane-2-carbonyl]-7-oxa-2,5-diazaspiro[3.4]octan-6-one;
2-[6-[[4-(trifluoromethylsulfonimidoyl)phenyl]methyl]-2-azaspiro[3.3]heptane-2-carbonyl]-7-oxa-2,5-diazaspiro[3.4]octan-6-one;
2-[6-(3-triflylbenzyl)-2-azaspiro[3.3]heptane-2-carbonyl]-2,5-diazaspiro[3.4]octan-6-one;
2-[6-[[2-fluoro-4-(trifluoromethyl)benzyl]-2,6-diazaspiro[3.3]heptane-2-carbonyl]-2,5-diazaspiro[3.4]octan-6-one;
2-[6-[[4-(trifluoromethyl)triazol-2-yl]methyl]-2-azaspiro[3.3]heptane-2-carbonyl]-8-oxa-2,5-diazaspiro[3.5]nonan-6-one;
2-[6-[[3-(trifluoromethyl)-1,2,4-thiadiazol-5-yl]methyl]-2-azaspiro[3.3]heptane-2-carbonyl]-8-oxa-2,5-diazaspiro[3.5]nonan-6-one;
2-[6-[[5-(difluoromethyl)-3-methyl-pyrazol-1-yl]methyl]-2-azaspiro[3.3]heptane-2-carbonyl]-8-oxa-2,5-diazaspiro[3.5]nonan-6-one;
2-[6-[[1-(trifluoromethyl) pyrazol-4-yl]methyl]-2-azaspiro[3.3]heptane-2-carbonyl]-8-oxa-2,5-diazaspiro[3.5]nonan-6-one;
2-[6-[[2-methyl-4-(trifluoromethyl) pyrazol-3-yl]methyl]-2-azaspiro[3.3]heptane-2-carbonyl]-8-oxa-2,5-diazaspiro[3.5]nonan-6-one;
2-[6-[[1-methyl-5-(trifluoromethyl) pyrazol-3-yl]methyl]-2-azaspiro[3.3]heptane-2-carbonyl]-8-oxa-2,5-diazaspiro[3.5]nonan-6-one;
2-[6-[[1-methyl-5-(trifluoromethyl) pyrazol-4-yl]methyl]-2-azaspiro[3.3]heptane-2-carbonyl]-8-oxa-2,5-diazaspiro[3.5]nonan-6-one;
2-[6-[[4-(trifluoromethyl) thiazol-2-yl]methyl]-2-azaspiro[3.3]heptane-2-carbonyl]-8-oxa-2,5-diazaspiro[3.5]nonan-6-one;
2-[6-[[1-methyl-3-(trifluoromethyl) pyrazol-4-yl]methyl]-2-azaspiro[3.3]heptane-2-carbonyl]-8-oxa-2,5-diazaspiro[3.5]nonan-6-one;
2-[6-[(3,5-difluoro-2-pyridyl)methyl]-2-azaspiro[3.3]heptane-2-carbonyl]-8-oxa-2,5-diazaspiro[3.5]nonan-6-one;
2-[6-[[2-fluoro-4-(trifluoromethyl)benzyl]-2,6-diazaspiro[3.3]heptane-2-carbonyl]-8-oxa-2,5-diazaspiro[3.5]nonan-6-one;
2-[6-(2-fluoro-4-triflyl-benzyl)-2,6-diazaspiro[3.3]heptane-2-carbonyl]-8-oxa-2,5-diazaspiro[3.5]nonan-6-one;
2-[6-[(4-tert-butyloxazol-2-yl)methyl]-2,6-diazaspiro[3.3]heptane-2-carbonyl]-2,5-diazaspiro[3.4]octan-6-one; and
2-[6-[(2-keto-1H-pyridin-3-yl)methyl]-2,6-diazaspiro[3.3]heptane-2-carbonyl]-2,5-diazaspiro[3.4]octan-6-one.

In a preferred embodiment, the present invention provides a compound of formula (I) as described herein, or a pharmaceutically acceptable salt thereof, selected from:
2-[6-(4-mesylbenzyl)-2-azaspiro[3.3]heptane-2-carbonyl]-7-oxa-2,5-diazaspiro[3.4]octan-6-one;
2-[7-[[5-(trifluoromethyl)-2-pyridyl]amino]-2-azaspiro[3.5]nonane-2-carbonyl]-7-oxa-2,5-diazaspiro[3.4]octan-6-one;
2-[6-[[4-(trifluoromethylsulfonimidoyl)benzyl]-2-azaspiro[3.3]heptane-2-carbonyl]-2,5-diazaspiro[3.4]octan-6-one;
2-[6-[[3-(trifluoromethylsulfonimidoyl)benzyl]-2-azaspiro[3.3]heptane-2-carbonyl]-2,5-diazaspiro[3.4]octan-6-one;
2-[6-[[6-(trifluoromethyl)-3-pyridyl]methyl]-2-azaspiro[3.3]heptane-2-carbonyl]-7-oxa-2,5-diazaspiro[3.4]octan-6-one;
2-[7-[[6-(trifluoromethyl)pyridazin-3-yl]oxy-2-azaspiro[3.5]nonane-2-carbonyl]-7-oxa-2,5-diazaspiro[3.4]octan-6-one;
5-[2-(6-keto-2,5-diazaspiro[3.4]octane-2-carbonyl)-2-azaspiro[3.3]heptan-6-yl]methyl]-2-(trifluoromethyl)benzonitrile;
5-[[2-(6-keto-2,5,7-triazaspiro[3.4]octane-2-carbonyl)-2-azaspiro[3.3]heptan-6-yl]methyl]-2-(trifluoromethyl)benzonitrile;
5-[[2-(6-keto-7-oxa-2,5-diazaspiro[3.4]octane-2-carbonyl)-2-azaspiro[3.3]heptan-6-yl]methyl]-2-(trifluoromethyl)benzonitrile; and
2-[6-[[5-(trifluoromethyl)pyrimidin-2-yl]methyl]-2-azaspiro[3.3]heptane-2-carbonyl]-2,5-diazaspiro[3.4]octan-6-one.

In a particularly preferred embodiment, the present invention provides a compound of formula (I) as described herein, or a pharmaceutically acceptable salt thereof, wherein said compound is 2-[6-(4-mesylbenzyl)-2-azaspiro[3.3]heptane-2-carbonyl]-7-oxa-2,5-diazaspiro[3.4]octan-6-one.

In a particularly preferred embodiment, the present invention provides a compound of formula (I) as described herein, or a pharmaceutically acceptable salt thereof, wherein said compound is 2-[7-[[5-(trifluoromethyl)-2-pyridyl]amino]-2-azaspiro[3.5]nonane-2-carbonyl]-7-oxa-2,5-diazaspiro[3.4]octan-6-one.

In a particularly preferred embodiment, the present invention provides a compound of formula (I) as described herein, or a pharmaceutically acceptable salt thereof, wherein said compound is 2-[6-[[4-(trifluoromethylsulfonimidoyl)benzyl]-2-azaspiro[3.3]heptane-2-carbonyl]-2,5-diazaspiro[3.4]octan-6-one.

In a particularly preferred embodiment, the present invention provides a compound of formula (I) as described herein, or a pharmaceutically acceptable salt thereof, wherein said compound is 2-[6-[[3-(trifluoromethylsulfonimidoyl)benzyl]-2-azaspiro[3.3]heptane-2-carbonyl]-2,5-diazaspiro[3.4]octan-6-one.

In a particularly preferred embodiment, the present invention provides a compound of formula (I) as described herein, or a pharmaceutically acceptable salt thereof, wherein said compound is 2-[6-[[6-(trifluoromethyl)-3-pyridyl]methyl]-2-azaspiro[3.3]heptane-2-carbonyl]-7-oxa-2,5-diazaspiro[3.4]octan-6-one.

In a particularly preferred embodiment, the present invention provides a compound of formula (I) as described herein, or a pharmaceutically acceptable salt thereof, wherein said compound is 2-[7-[[6-(trifluoromethyl)pyridazin-3-yl]oxy-2-azaspiro[3.5]nonane-2-carbonyl]-7-oxa-2,5-diazaspiro[3.4]octan-6-one.

In a particularly preferred embodiment, the present invention provides a compound of formula (I) as described herein, or a pharmaceutically acceptable salt thereof, wherein said compound is 5-[2-(6-keto-2,5-diazaspiro[3.4]octane-2-carbonyl)-2-azaspiro[3.3]heptan-6-yl]methyl]-2-(trifluoromethyl)benzonitrile.

In a particularly preferred embodiment, the present invention provides a compound of formula (I) as described herein, or a pharmaceutically acceptable salt thereof, wherein said compound is 5-[[2-(6-keto-2,5,7-triazaspiro[3.4]octane-2-carbonyl)-2-azaspiro[3.3]heptan-6-yl]methyl]-2-(trifluoromethyl)benzonitrile.

In a particularly preferred embodiment, the present invention provides a compound of formula (I) as described herein, or a pharmaceutically acceptable salt thereof, wherein said compound is 5-[[2-(6-keto-7-oxa-2,5-diazaspiro[3.4]octane-2-carbonyl)-2-azaspiro[3.3]heptan-6-yl]methyl]-2-(trifluoromethyl)benzonitrile.

In a particularly preferred embodiment, the present invention provides a compound of formula (I) as described herein, or a pharmaceutically acceptable salt thereof, wherein said compound is 2-[6-[[5-(trifluoromethyl)pyrimidin-2-yl]methyl]-2-azaspiro[3.3]heptane-2-carbonyl]-2,5-diazaspiro[3.4]octan-6-one.

In a particular embodiment, the present invention provides pharmaceutically acceptable salts of the compounds according to formula (I) as described herein. In a further particular embodiment, the present invention provides compounds according to formula (I) as described herein in their free form (i.e., as free bases or acids).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)-insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations. 2nd Edition. Richard C. Larock. John Wiley & Sons. New York. NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:

AcOH=acetic acid, ACN=acetonitrile, Bn=benzyl, BINAP=(2,2′-bis(diphenylphosphino)-1,1′-binaphthyl), Boc=tert-butyloxycarbonyl, CAS RN=chemical abstracts registration number, Cbz=benzyloxycarbonyl, Cs₂CO₃=cesium carbonate, CO=carbon monoxide, CuCl=copper (I) chloride, CuCN=copper (I) cyanide, CuI=copper (I) iodide, DABCO=1,4-Diazabicyclo[2.2.2]octane; triethylenediamine, DAST=(diethylamino) sulfur trifluoride, DBU=1,8-diazabicyclo[5.4.0]undec-7-ene, DEAD=diethyl azodicarboxylate, DIAD=diisopropyl azodicarboxylate, DIBAL-H=diisobutyl aluminium hydride, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC·HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluoro-phosphate, HCl=hydrogen chloride, HOBt=1-hydroxy-1H-benzotriazole; HPLC=high performance liquid chromatography, iPrMgCl=isopropylmagnesium chloride, I2=iodine, IPA=2-propanol, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, mCPBA=meta-chloroperoxy benzoic acid, MgSO₄=magnesium sulfate, min=minute(s), mL=milliliter, MPLC=medium pressure liquid chromatography, MS=mass spectrum, nBuLi=n-butyllithium, NaBH; CN=sodium cyanoborohydride, NaH=sodium hydride, NBS=N-bromosuccinimide, NaHCO₃=sodium hydrogen carbonate, NaNO₂=sodium nitrite, NaBH(OAc)₃=sodium triacetoxy borohydride, NaOH=sodium hydroxide, Na₂CO₃=sodium carbonate, Na₂SO₄=sodium sulfate, Na₂S₂O₃=sodium thiosulfate, NEt₃=triethylamine (TEA), NH₄Cl=ammonium chloride, NMP=N-methyl-2-pyrrolidone, OAc=Acetoxy, T₃P=propylphosphonic anhydride, PE=petroleum ether, PG=protective group, Pd—C=palladium on activated carbon, PdCl2(dppf)-CH₂Cl₂=1,1′-bis(diphenylphosphino)ferrocene-palladium(II) dichloride dichloromethane complex, Pd₂(dba)₃=tris(dibenzylideneacetone)dipalladium(0)). Pd(OAc)₂=palladium(II) acetate, Pd(OH)₂=palladium hydroxide, Pd(PPh₃)₄=tetrakis(triphenylphosphine)palladium(0)), PMP=1,2,2,6,6-Pentamethylpiperidine, PTSA=p-toluenesulfonic acid, R=any group, RP=reverse phase, RT=room temperature, SFC=Supercritical Fluid Chromatography, S-PHOS=2-dicyclohexylphosphino-2′,6′-dimethoxybiphenyl, TBAI=tetra butyl ammonium iodine, TEA=triethylamine, TFA=trifluoroacetic acid, THF=tetrahydrofuran, TMEDA=N,N,N′,N′-tetramethylethylenediamine, TS-TPP=triphenylphospine-polymer bound, ZnCl₂=zinc chloride, Hal=halogen, prep-TLC=preparative thin layer chromatography.

The present compounds of formula I can be prepared by reacting an activated intermediate of formula 2 with the nucleophilic spirocyclic amine 1 by heating in a solvent such as DMF or CH₃CN in the presence of a base such as DIPEA. (Scheme 1) In some cases an alternative activated intermediate bearing a 4-nitrophenyl group instead of the 1,2,4-triazole was used. Alternatively the activated intermediate can be formed on the other coupling partner (1) that will make the urea of formula I.

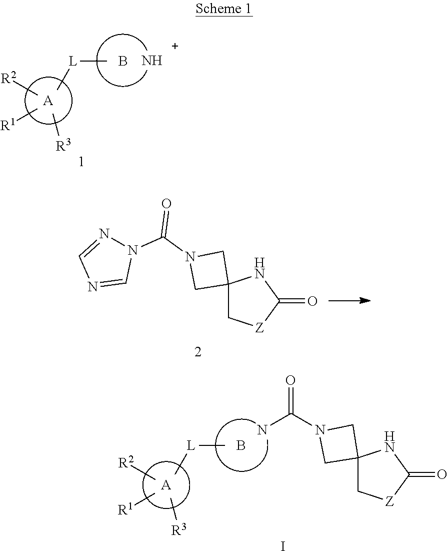

The activated intermediate 2 can be generated transiently in the reaction mixture, or by reacting an amine 3 with a coupling agent such as di(1H-1,2,4-triazol-1-yl) methanone in a solvent such as CH₂Cl₂ in the presence of a base such as DIPEA (Scheme 2). The related 4-nitrophenylcarbonate intermediates can be generated in a similar process using 4-nitrophenyl carbonochloridate. Alternatively, the same strategy as in Schemes 1 and 2 may be used, but with the activated intermediate being constructed initially on spirocyclic amine 1, before coupling with amine 3.

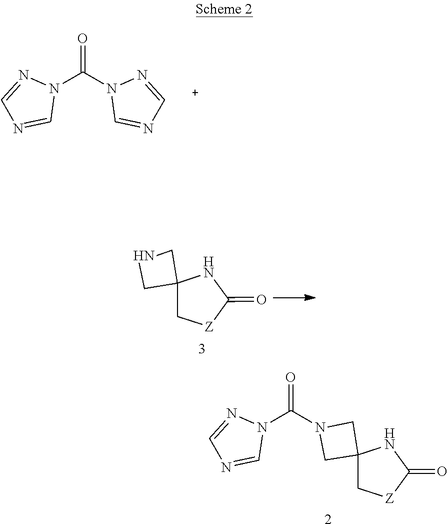

Building blocks of formula 4 where L=CH₂ can be generated by Suzuki reaction (e.g. (Pd(dppf)Cl₂, K₂CO₃, dioxane/H₂O), (X=Br, I) followed by hydrogenation (e.g. Pd/C, H₂). The required boronate intermediate 5 can be generated by reacting a ketone with 4,4,5,5-tetramethyl-2-[(tetramethyl-1,3,2-dioxaborolan-2-yl)methyl]-1,3,2-dioxaborolane (LiTMP, THF, −78° C.). (Scheme 3). Where A=N-linked heteroaryl, a Chan-Lam type coupling can be used in place of the Suzuki reaction, followed by the hydrogenation/deprotection.

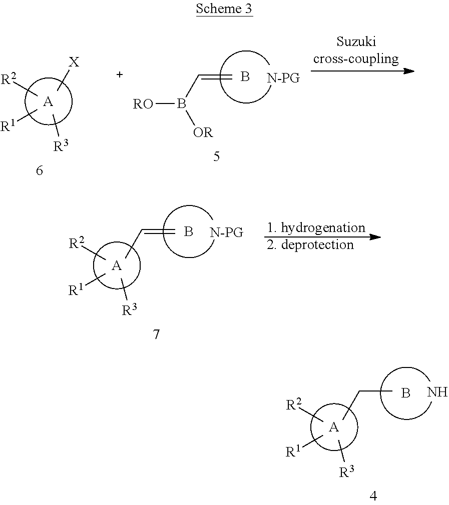

Building blocks of formula 8 with L=bond and A=C-linked (hetero) aryl can be generated by coupling a suitably protected boronic acid derivative 9 (X=B(OR)₂) with an iodide 10 under nickel or palladium catalysis. Alternatively a bromide 9 (X=Br) can be coupled directly with 10 in a photochemical reaction using Ir[dF(CF₃)ppy]₂(dtbbpy)PF₆, NiCl₂·DME, dtbbpy and (TMS)₃SiH. (Scheme 4) Alternatively C-linked heteroaryl rings A may be installed using standard heterocyclic ring syntheses, typically starting from acid or cyano derivatives of the spirocyclic amine.

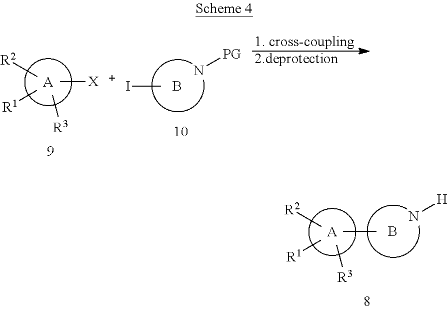

Alternatively, building blocks of formula 11 with L=oxygen and A is (hetero) aryl (where X is in a position suitable for S_{N}Ar displacement) can be prepared by reacting 12 (X is a leaving group such as Cl, Br, typically adjacent to aromatic N for S_{N}Ar reaction), with a suitably protected alcohol building block 13 in the presence of a base such as NaOtBu, followed by deprotection under standard conditions (e.g. with TsOH when PG=Boc). (Scheme 5) A similar scheme could be used for installing a small aliphatic unit or an aliphatic (hetero) cycle such as cyclopropyl as the A ring, via S_{N}2 displacement of a leaving group X (typically OMs, Br or I) using a base such as NaH.

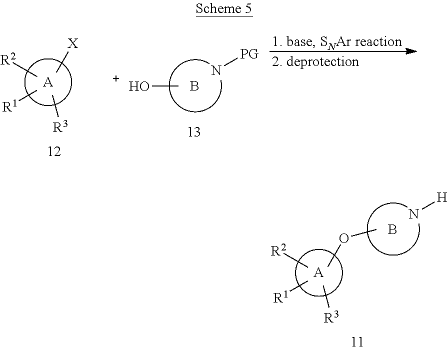

Alternatively, building blocks of formula 14 with L=NHCO could be prepared by reacting a suitably protected carboxylic acid 15 with an amine 16 to produce amide 17 using standard amide coupling techniques (e.g. HATU, Et₃N), followed by deprotection. The amide 17 could also be reduced prior to deprotection (e.g. using borane-methyl sulfide complex) to yield amine building blocks of formula 18. (Scheme 6).

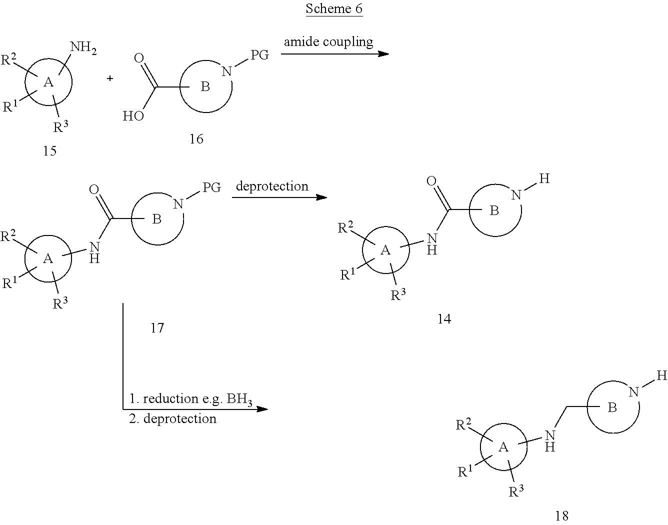

Alternatively, building blocks of formula 19 with L=SO₂NH can be prepared by reacting sulfonyl chloride 20 with a suitably protected amine building block 13 in the presence of a base such as DIPEA, followed by deprotection under standard conditions (e.g. with TsOH or TFA when PG=Boc). (Scheme 7)

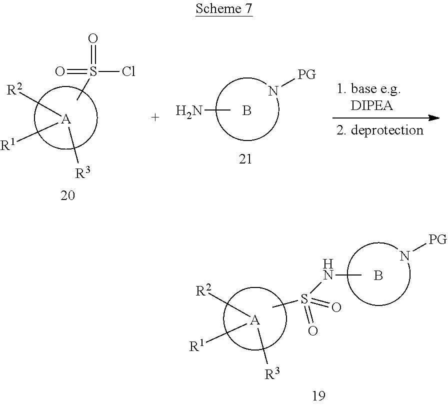

Alternatively, building blocks of formula 22 with L=N and A is (hetero) aryl (where X is in a position suitable for S_{N}Ar displacement) can be prepared by reacting 12 (X is a leaving group such as Cl, Br, often adjacent to aromatic N for S_{N}Ar reaction), with a suitably protected amine building block 21 in the presence of a base such as DIPEA, followed by deprotection under standard conditions (e.g. with TsOH when PG=Boc). (Scheme 8)

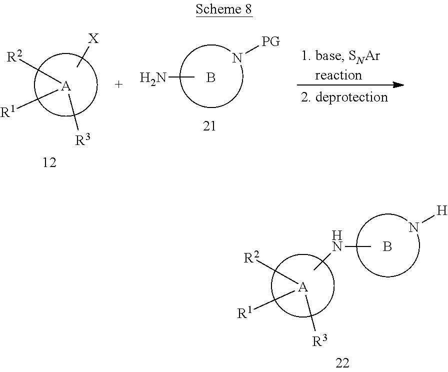

Alternatively, building blocks of formula 22 with L=bond and A is N-linked heterocyclic can be prepared by reacting nucleophilic hetrocycle A (24), with a suitably protected building block 25 (X=leaving group such as OMs, I, Br) in the presence of a base such as Cs₂CO₃, followed by deprotection under standard conditions (e.g. with TsOH when PG=Boc). (Scheme 9) Typically mesylate building blocks 25 were used (X=OMs), which can conveniently be generated from the hydroxyl analog by reacting with MsCl in the presence of a mild base such as Et₃N.

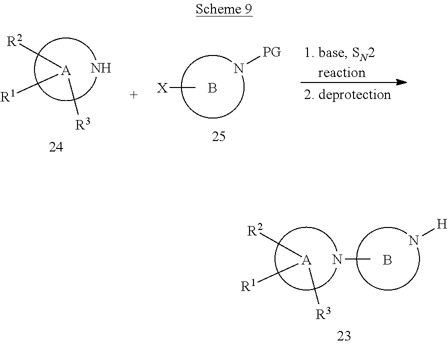

Alternatively, building blocks of formula 22 with L=bond and A is N-linked heterocyclic can be prepared by a reductive amination reaction of hetrocycle A (24) with a suitably protected ketone building block 26 in the presence of a reducing agent such as sodium triacetoxyborohydride or sodium cyanoborohydride, followed by deprotection under standard conditions (e.g. with TsOH when PG=Boc). (Scheme 10)

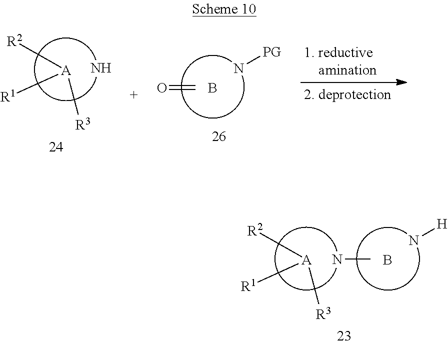

Alternatively, building blocks of formula 27 with L=CH₂N may be installed by a reductive amination reaction of amine 28 with a suitably protected ketone building block 26 in the presence of a reducing agent such as sodium triacetoxyborohydride or sodium cyanoborohydride, followed by deprotection under standard conditions (e.g. with TsOH when PG=Boc). (Scheme 11)

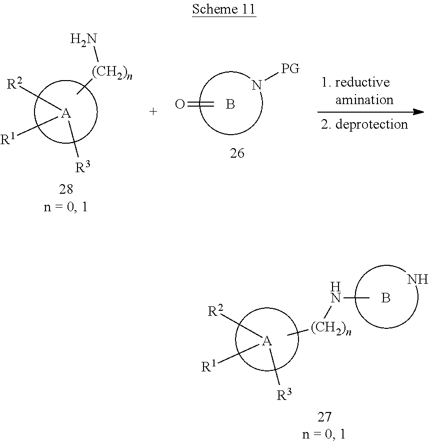

Alternatively, building blocks of formula 28 with L=CH₂ and B is N-linked can be prepared by a reductive amination reaction of aldehyde 29 with suitably protected hetrocycle B (30) in the presence of a reducing agent such as sodium triacetoxyborohydride or sodium cyanoborohydride, followed by deprotection under standard conditions (e.g. with TsOH when PG=Boc). (Scheme 12)

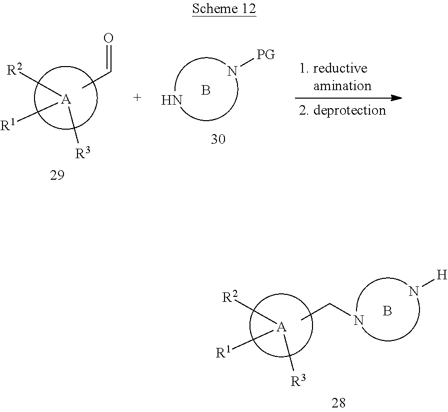

Alternatively, building blocks of formula 31 with L=SO₂ and B is N-linked can be prepared from a sulfonyl chloride 32 suitably protected hetrocycle B (30) in the presence of a base such as DIPEA, followed by deprotection under standard conditions (e.g. with TsOH when PG=Boc). (Scheme 13)

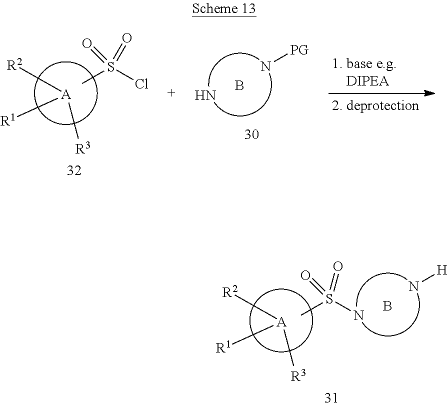

Alternatively, building blocks of formula 28 with L=CH₂ and A is N-linked can be prepared by a reductive amination reaction of (hetero) aryl methylhalide (X=Br, I, Cl) 33 with suitably protected heterocycle B (30) in the presence of a base such as K₂CO₃ in a solvent such as ACN, followed by deprotection under standard conditions (e.g. with TsOH when PG=Boc). (Scheme 14)

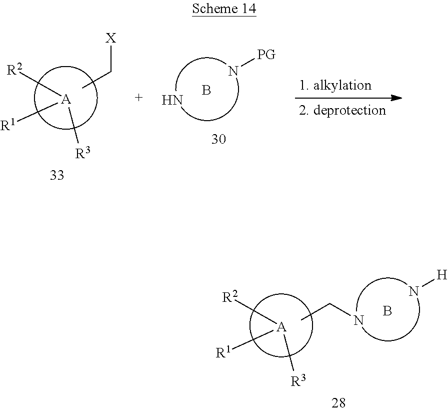

Alternatively, sulfonylurea building blocks of formula 34 with L=—NHSO₂— or —CH₂NHSO₂—, can be prepared by activating 2-methyl-1-(2-methylimidazol-1-yl) sulfonyl-imidazole by methylation with methyl trifluoromethanesulfonate, followed by reaction with suitably protected amine 30; a further sequence of activation by methylation with methyl trifluoromethanesulfonate followed by reaction with amine 52; and finally deprotection under standard conditions (e.g. with TsOH or TFA when PG=Boc). (Scheme 15) Alternatively sulfonylurea building blocks 34 can be generated from sulfuryl chloride followed by sequential additions of 30 and 52 in the presence of a base such as Et₃N or DIPEA, and finally deprotection under standard conditions.

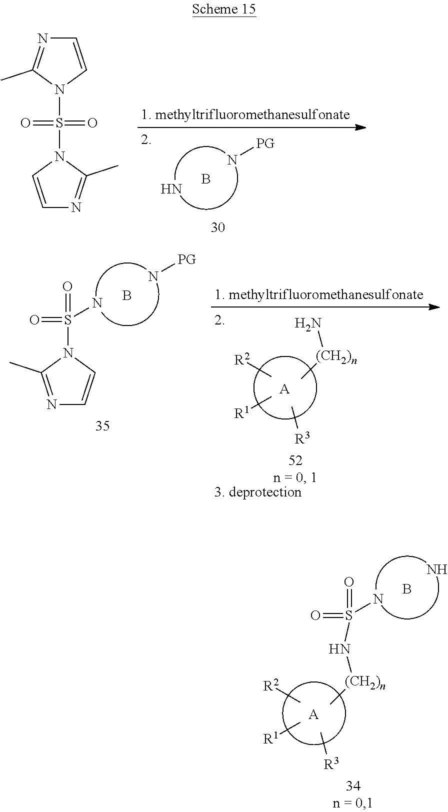

Alternatively, building blocks of formula 36 with L=bond, B is N-linked and A is (hetero) aryl can be prepared using a metal-catalysed cross-coupling reaction (e.g. Buchwald reaction, Pd-catalysis) between a suitably protected heterocycle B (30) and a (hetero) arylhalide (X=Br, I, Cl), followed by deprotection under standard conditions (e.g. with TsOH or TFA when PG=Boc). (Scheme 16) In certain cases, if the (hetero) aryl 37 is suitable for S_{N}Ar reaction (X=F) then building blocks of formula 37 can be generated by S_{N}Ar reaction of (spiro) cycle B 30 and 37, in the presence of a base (e.g. K₂CO₃ or Et₃N, DMSO, heating).

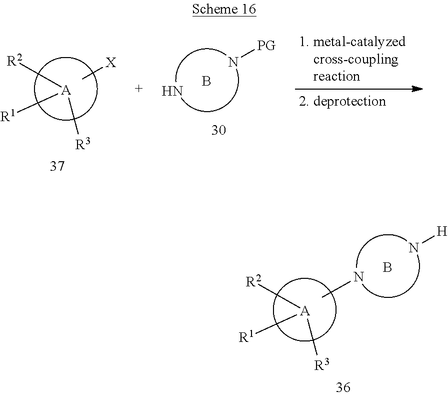

Alternatively, building blocks of formula 38 can be prepared by a Mitsunobu type reaction of heterocycle A (24) with a hydroxyl building block 39 (e.g. using diisopropyl azodicarboxylate and triphenylphosphine, or Tsunoda reagent (cyanomethylenetrimethylphosphorane)), followed by deprotection under standard conditions (e.g. with TsOH when PG=Boc). (Scheme 17) Alternatively, building blocks of formula 38 can be prepared by conversion of hydroxyl building block 39 to a mesylate (e.g. using MsCl, Et₃N) followed by an S_{N}2 reaction with the heterocycle A (24) in the presence of a base such as NaH.

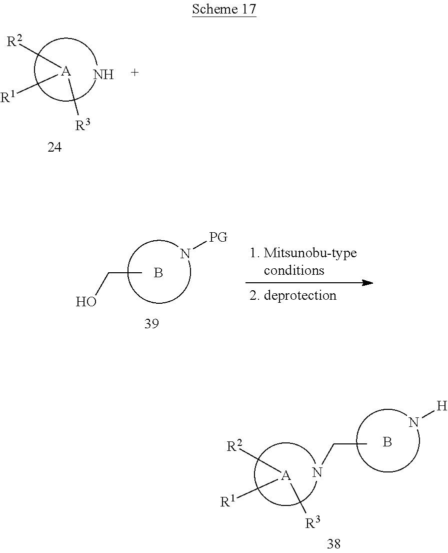

Alternatively, building blocks of formula 40 with L=oxygen and A is (hetero) aryl can be prepared by reacting a suitably protected (spiro) cyclic amine bearing a hydroxyl group (42) with a nucleophilic (hetero) aryl alcohol (41) under Mitsunobu-type conditions (e.g. using Tsunoda reagent, (tributylphosphoranylidene) acetonitrile or PPh₃/DIAD), followed by deprotection. (Scheme 18)

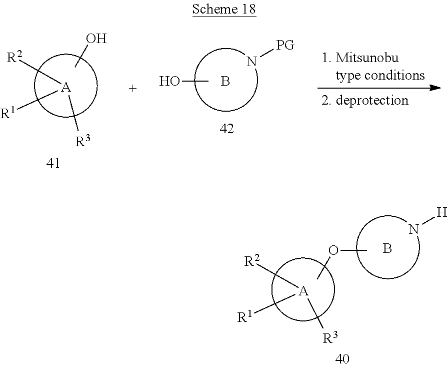

Alternatively, building blocks of formula 43 where B is C-linked and L=—SO₂— may be generated by S_{N}2 reaction of a thiol 44 with (spiro) cyclic amine 45 (Y=leaving group, such as OMs) under basic conditions (e.g. K₂CO₃) followed by oxidation of the thioether to the sulfone (e.g. with mCPBA) and deprotection under standard conditions (e.g. with TsOH when PG=Boc). (Scheme 19) The same sequence can be used to generate sulfoxime (L=—SNO—) using a modified oxidation step to install the sulfoximine from the thioether (e.g. using iodobenzene diacetate and ammonium carbamate).

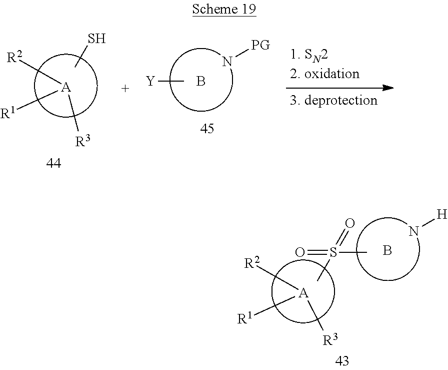

Alternatively, building blocks of formula 4 where A is a C-linked heteroaryl and L=—CH₂— may be generated using standard heterocyclic synthesis techniques starting from a suitable carboxylic acid (47) or nitrile (48) derivative. The nitrile derivatives can be generated from the hydroxyl derivatives (46) via conversion to a mesylate (e.g. using MsCl, Et₃N) followed by S_{N}2 displacement of the mesylate group with cyanide (e.g. using KCN). (Scheme 20)

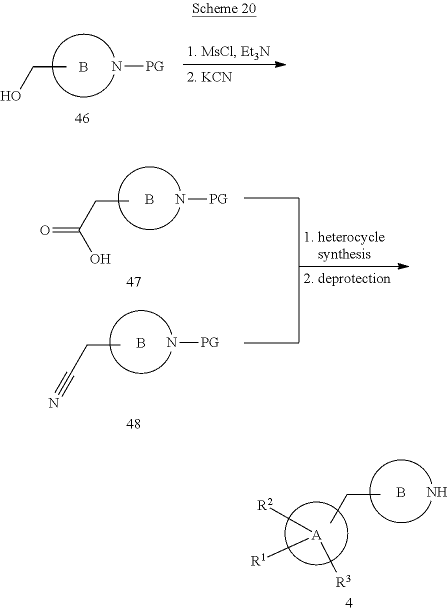

(Hetero) aryl trifluoromethylcyclopropyl building blocks 49 were not generally available, and were instead generated from halide building block 50 (X=I, Br) via Suzuki reaction with 1-(trifluoromethyl)vinylboronic acid to give 51. Cyclopropanation using diphenyl(methyl) sulfonium tetrafluoroborate and LiHMDS gave the required building block 49 (Scheme 21). This sequence could also be carried out inbetween steps in other synthetic schemes, e.g. for N-linked heteroaryl rings A, these functionalization steps would often be carried out while making building blocks of formula 4, after the Suzuki/hydrogenation sequence, but prior to final deprotection (see Scheme 3).

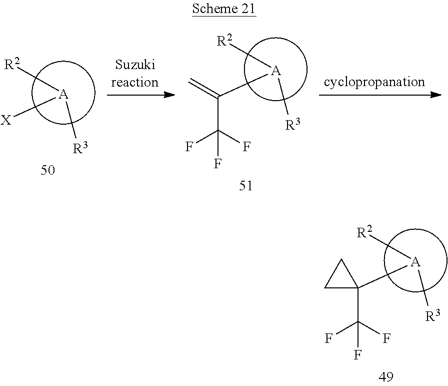

In some cases, compounds of formula I, could be further functionalized to give other compounds of formula I. For example, a compound of formula I bearing a (hetero) aryl bromide or iodide can be further functionalized with other groups e.g. small amine, small alkyl using metal catalyzed cross-coupling conditions such as Buchwald or Suzuki reactions.

In some cases, building blocks could be generated from commercially available fragments using standard functional group interconversion techniques (e.g. conversion of halides to other groups e.g. small amine, small alkyl using metal catalyzed cross-coupling conditions such as Buchwald or Suzuki reactions, conversion of boron-containing groups to hydroxyl using alkaline peroxide conditions, alkylation of hydroxyl or amine groups via S_{N}2 reaction or reductive amination, acylation using an activated carbonyl derivative, or installation of —SO₂Me or —SO₂CF₃ groups using literature techniques). Such techniques may also be used to elaborate commercially available fragments before, after, or intermediate within the synthetic sequences described above.

In one aspect, the present invention provides a process of manufacturing a compound of formula (I) described herein, or a pharmaceutically acceptable salt thereof, comprising:
(a) reacting nucleophilic spirocyclic amine 1

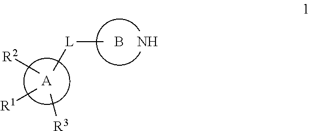

wherein the variables are as described herein;
with an activated intermediate of formula 2

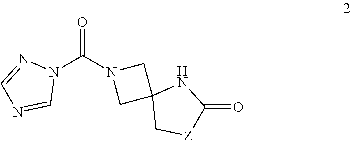

wherein Z is as described herein;
in a solvent and in the presence of a base to form said compound of formula (I); or
(b) reacting nucleophilic spirocyclic amine 1

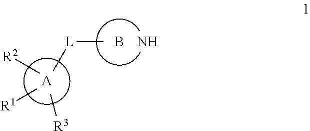

wherein the variables are as described herein;
with an activated intermediate of formula 2a

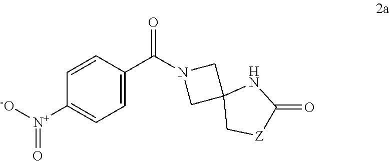

wherein Z is as described herein;
in a solvent and in the presence of a base to form said compound of formula (I); and
optionally
(c) contacting said compound of formula (I) with an acid to form a pharmaceutically acceptable salt thereof.

In one embodiment, said process is carried out at a temperature between room temperature and reflux of the solvent mixture, e.g. at about 30° C. to about 80° C., in particular at about 30° C., about 40° C., about 50° C., about 60° C., about 70° C., or about 80° C.

In one embodiment, the base used in said process is DIPEA.

In one embodiment, the solvent used in said process is DMF or CH₃CN.

In one aspect, the present invention provides a compound of formula (I) as described herein, or a pharmaceutically acceptable salt thereof, when manufactured according to any one of the processes described herein.

### MAGL Inhibitory Activity

Compounds of the present invention are MAGL inhibitors. Thus, in one aspect, the present invention provides the use of compounds of formula (I) as described herein for inhibiting MAGL in a mammal.

In a further aspect, the present invention provides compounds of formula (I) as described herein for use in a method of inhibiting MAGL in a mammal.

In a further aspect, the present invention provides the use of compounds of formula (I) as described herein for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds of formula (I) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”.

Compounds of formula (I) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”. The 2-AG assay was carried out in 384 well polypropylene assay plates. Compound dilutions were made in 100% DMSO in a polypropylene plate in 3-fold dilution steps to give a final concentration range in the assay from 12.5 μM to 0.8 μM. Compound dilutions were added to MAGL protein in assay buffer (50 mM TRIS, 1 mM EDTA, 0.01% (v/v) Tween-20, 2.5% (v/v) DMSO). After shaking, the plate was incubated for 15 min at RT. To start the reaction, 2-arachidonoylglycerol in assay buffer was added. The final concentrations in the assay was 50 μM for MAGL protein and 8 μM 2-arachidonoylglyerol. After shaking and 30 min incubation at RT, the reaction was quenched by the addition of two assay volumes of acetonitrile containing 4 μM of d8-arachidonic acid. The amount of arachidonic acid formed was traced by an online SPE system (Agilent Rapidfire) coupled to a triple quadrupole mass spectrometer. A C18 SPE cartridge (Agilent G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d 8-arachidonic acid].

TABLE 1

| | IC50 |
|---|---|
| | MAGL |
| Example | [μM] |
| 1 | 0.00091 |
| 2 | 0.00215 |
| 3 | 0.00285 |
| 4 | 0.01121 |
| 5 | 0.00115 |
| 6 | 0.00047 |
| 7 | 0.00035 |
| 8 | 0.01512 |
| 9 | 0.02046 |
| 10 | 0.02455 |
| 11 | 0.03115 |
| 12 | 0.03642 |
| 13 | 0.00093 |
| 14 | 0.00084 |
| 15 | 0.00030 |
| 16 | 0.00009 |
| 17 | 0.00329 |
| 18 | 0.00002 |
| 19 | 0.00700 |
| 20 | 0.00031 |
| 21 | 0.00001 |
| 22 | <5 nM |
| 23 | <5 nM |
| 24 | <5 nM |
| 25 | 0.00005 |
| 26 | 0.00011 |
| 27 | 0.00014 |
| 28 | 0.00023 |
| 29 | 0.00034 |
| 30 | 0.00045 |
| 31 | 0.00061 |
| 32 | 0.00067 |
| 33 | 0.00087 |
| 34 | 0.00109 |
| 35 | 0.00143 |
| 36 | 0.00143 |
| 37 | 0.00191 |
| 38 | 0.00234 |
| 39 | 0.00238 |
| 40 | 0.00304 |
| 41 | 0.00552 |
| 42 | 0.05337 |
| 43 | 0.64593 |
| 44 | 0.00032 |
| 45 | 0.02631 |
| 46 | 0.00015 |
| 47 | 0.00294 |
| 48 | 0.00015 |
| 49 | 0.00077 |
| 50 | 0.00078 |
| 51 | 0.02404 |
| 52 | 0.74192 |
| 53 | 0.00044 |
| 54 | 0.00937 |
| 55 | 0.00981 |
| 56 | 0.01289 |
| 57 | 0.01336 |
| 58 | 0.01728 |
| 59 | 0.03655 |
| 60 | 0.00074 |
| 61 | 0.00019 |
| 62 | 0.00098 |
| 63 | 0.00128 |
| 64 | 0.00128 |
| 65 | 0.00185 |
| 66 | 0.02987 |
| 67 | 0.08856 |
| 68 | 0.00213 |
| 69 | 0.00083 |
| 70 | 0.00368 |
| 71 | 0.00329 |
| 72 | 0.09823 |
| 73 | 0.08017 |
| 74 | 0.07768 |
| 75 | 0.27080 |
| 76 | 0.00833 |
| 77 | 0.00091 |
| 78 | 0.00230 |
| 79 | 0.00379 |
| 80 | 0.00619 |
| 81 | 0.00762 |
| 82 | 0.00558 |
| 83 | 0.02425 |
| 84 | 0.00099 |
| 85 | 0.00094 |
| 86 | 0.98388 |
| 87 | 0.00642 |
| 88 | 0.01347 |
| 89 | 0.02972 |
| 90 | 0.01907 |
| 91 | 0.63462 |
| 92 | 0.00065 |
| 93 | 0.14241 |
| 94 | 0.00108 |
| 95 | 0.00037 |
| 96 | 0.00188 |
| 97 | 0.00015 |
| 98 | 0.00175 |
| 99 | 0.00309 |
| 100 | 0.00342 |
| 101 | 0.00018 |
| 102 | 0.01080 |
| 103 | 0.00028 |
| 104 | 0.00285 |
| 105 | 0.00005 |
| 106 | 0.00005 |
| 107 | 0.00008 |
| 108 | 0.00018 |
| 109 | 0.00021 |
| 110 | 0.00024 |
| 111 | 0.00031 |
| 112 | 0.00057 |
| 113 | 0.00062 |
| 114 | 0.00063 |
| 115 | 0.00102 |
| 116 | 0.00173 |
| 117 | 0.00196 |
| 118 | 0.00249 |
| 119 | 0.00293 |
| 120 | 0.00295 |
| 121 | 0.00419 |
| 122 | 0.00433 |
| 123 | 0.00443 |
| 124 | 0.00454 |
| 125 | 0.00581 |
| 126 | 0.02134 |
| 127 | 0.03083 |
| 128 | 0.05188 |
| 129 | 0.06674 |
| 130 | 0.18600 |
| 131 | 0.20907 |
| 132 | 0.00079 |
| 133 | 0.01310 |
| 134 | 0.01905 |
| 135 | 0.00101 |
| 136 | 0.00466 |
| 137 | 0.00097 |
| 138 | 0.05103 |
| 139 | 0.00076 |
| 140 | 0.07889 |
| 141 | 0.21732 |
| 142 | 0.00045 |
| 143 | 0.00007 |
| 144 | 2.12103 |
| 145 | 0.00006 |
| 146 | 0.01094 |
| 147 | 0.02321 |
| 148 | 0.12285 |
| 149 | 0.00168 |
| 150 | 0.13964 |
| 151 | 0.19605 |
| 152 | 0.00307 |
| 153 | 0.00475 |
| 154 | 0.00411 |
| 155 | 0.23684 |
| 156 | 0.00507 |
| 157 | 0.00986 |
| 158 | 0.03385 |
| 159 | 0.10895 |
| 160 | 0.27902 |
| 161 | 0.05148 |
| 162 | 0.01514 |
| 163 | 0.00278 |
| 164 | 0.05248 |
| 165 | 0.00052 |
| 166 | 0.00131 |
| 167 | 0.00172 |
| 168 | 0.00245 |
| 169 | 0.00235 |
| 170 | 0.00782 |
| 171 | 0.00142 |
| 172 | 0.03819 |
| 173 | 0.02078 |
| 174 | 0.01666 |
| 175 | 0.00169 |
| 176 | 0.00300 |
| 177 | 0.00201 |
| 178 | 0.01543 |
| 179 | 0.00166 |
| 180 | 0.20247 |
| 181 | 0.00145 |
| 182 | 0.48492 |
| 183 | 0.00731 |
| 184 | 0.21045 |
| 185 | 0.03447 |
| 186 | 0.09499 |
| 187 | 0.04667 |
| 189 | 0.00172 |
| 190 | 0.01043 |
| 191 | 0.02998 |
| 192 | 0.01467 |
| 193 | 0.06529 |
| 194 | 0.04459 |
| 195 | 0.00251 |
| 196 | 0.03878 |
| 197 | 0.00439 |
| 198 | 0.23206 |
| 199 | 0.26568 |
| 200 | 0.11564 |
| 201 | 0.16484 |
| 202 | 0.06137 |
| 203 | 0.06158 |
| 204 | 0.01178 |
| 205 | 0.26451 |
| 206 | 0.27798 |
| 207 | 0.41356 |
| 208 | 0.06707 |
| 209 | 0.00628 |
| 210 | 0.00084 |
| 211 | 0.00039 |
| 212 | 0.00046 |
| 213 | 0.00055 |
| 214 | 0.38619 |
| 215 | 0.00320 |
| 216 | 0.01762 |
| 217 | 0.00078 |
| 218 | 0.08730 |
| 219 | 0.00043 |
| 220 | 0.00418 |
| 221 | 0.00131 |
| 222 | 0.00227 |
| 223 | 0.00096 |
| 224 | 0.00023 |
| 225 | 0.01701 |
| 226 | 0.04588 |
| 227 | 1.79711 |
| 228 | 0.01844 |
| 229 | 0.07842 |
| 230 | 0.05895 |
| 231 | 0.25539 |
| 232 | 0.23004 |
| 233 | 0.26481 |
| 234 | 0.00849 |
| 235 | 0.00735 |
| 236 | 0.00216 |
| 237 | 0.00315 |
| 238 | 0.01654 |
| 239 | 0.05279 |
| 240 | 1.24115 |
| 241 | 0.92779 |
| 242 | 0.00006 |
| 243 | 0.00060 |
| 244 | 0.00094 |
| 245 | 0.00151 |
| 246 | 0.00617 |
| 247 | 0.31077 |
| 248 | 0.03279 |
| 249 | 0.00216 |
| 251 | 0.00060 |
| 252 | 0.00983 |
| 253 | 0.03350 |
| 254 | 0.00484 |
| 255 | 0.00419 |
| 256 | 0.02690 |
| 257 | 0.01647 |
| 258 | 0.01865 |
| 259 | 0.02594 |
| 260 | 0.08065 |
| 261 | 0.00043 |
| 262 | 0.00106 |
| 263 | 0.18301 |
| 264 | 0.00551 |
| 265 | 0.00048 |
| 266 | 1.37582 |
| 267 | 1.24205 |
| 268 | 0.30098 |
| 269 | 0.47298 |
| 270 | 0.07244 |
| 271 | 0.03164 |
| 272 | 0.79497 |
| 273 | 0.06822 |
| 274 | 0.54014 |
| 275 | 0.10416 |
| 276 | 0.09272 |
| 277 | 0.19455 |
| 278 | 0.47857 |
| 279 | 1.03155 |
| 280 | 0.19523 |
| 281 | 0.00644 |
| 282 | 0.03724 |
| 283 | 0.00062 |
| 284 | 0.12008 |
| 285 | 0.00495 |
| 286 | 0.00289 |
| 287 | 0.43683 |
| 288 | 0.00891 |
| 289 | 0.00044 |
| 290 | 0.04564 |
| 291 | 0.00127 |
| 292 | 0.00129 |
| 293 | 0.06942 |
| 294 | 0.00215 |
| 295 | 0.00007 |
| 296 | 0.00015 |
| 297 | 0.07590 |
| 298 | 0.00083 |
| 299 | 0.06349 |
| 300 | 0.04737 |
| 301 | 0.01538 |
| 302 | 0.06880 |
| 303 | 0.03567 |
| 304 | 0.00373 |
| 305 | 0.02093 |
| 306 | 0.28666 |
| 307 | 0.04809 |
| 308 | 0.04904 |
| 309 | 0.09494 |
| 310 | 0.07690 |
| 311 | 0.00408 |
| 312 | 0.05495 |
| 313 | 0.02780 |
| 314 | 0.00862 |
| 315 | 0.20561 |
| 316 | 0.55302 |
| 317 | 1.03569 |
| 318 | 0.14551 |
| 319 | 0.01245 |
| 320 | 0.17105 |
| 321 | 0.08816 |
| 322 | 0.00432 |
| 323 | 0.00017 |
| 324 | 0.00335 |
| 325 | 0.00388 |
| 326 | 0.00653 |
| 327 | 0.00757 |
| 328 | 0.01587 |
| 329 | 0.01606 |
| 330 | 0.02457 |
| 331 | 0.04524 |
| 332 | 0.05268 |
| 333 | 0.08806 |
| 334 | 0.08823 |
| 335 | 0.10057 |
| 336 | 0.14617 |
| 337 | 0.15962 |
| 338 | 0.17838 |
| 339 | 0.21536 |
| 340 | 0.42761 |
| 341 | 0.72960 |
| 342 | 1.59739 |
| 343 | 1.80105 |
| 344 | 2.44739 |
| 345 | 3.33457 |
| 346 | 0.02909 |
| 347 | 0.00819 |
| 348 | 0.09088 |
| 349 | 0.02295 |
| 350 | 0.16509 |
| 351 | 0.00037 |
| 352 | 0.00015 |
| 353 | 0.0001 |
| 354 | 0.00028 |
| 355 | 0.00019 |
| 356 | 0.0002 |
| 357 | 0.00015 |
| 358 | 0.00026 |
| 359 | 0.00032 |
| 360 | 0.0002 |
| 361 | 0.0058 |
| 362 | 0.07825 |
| 363 | 0.05487 |
| 364 | 0.02724 |
| 365 | 0.04095 |
| 366 | 0.0224 |
| 367 | 0.00288 |
| 368 | 0.00411 |
| 369 | 0.02306 |
| 370 | 0.01184 |
| 371 | 0.00904 |
| 372 | 0.01564 |
| 373 | 0.05922 |
| 374 | 0.08872 |
| 375 | 1.85186 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 UM and 5 μM, as measured in the MAGL assay described herein.

### Using the Compounds of the Invention

In one aspect, the present invention provides compounds of formula (I), or pharmaceutically acceptable salts thereof, as described herein for use as therapeutically active substance.

In one embodiment, the compounds of the present invention are “peripherally” active, i.e., they are not penetrating the blood brain barrier.

In a further aspect, the present invention provides a method for the treatment or prophylaxis of diseases or conditions associated with MAGL in a mammal, which method comprises administering an effective amount of a compound of formula (I), or a pharmaceutically acceptable salt thereof, as described herein to the mammal.

In one embodiment, said diseases or conditions associated with MAGL are selected from neuroinflammation, neurodegenerative diseases, pain, cancer, mental disorders and inflammatory bowel disease.

In one embodiment, said diseases or conditions associated with MAGL are selected from neuroinflammation and neurodegenerative diseases.

In one embodiment, said diseases or conditions associated with MAGL are neurodegenerative diseases.

In one embodiment, said disease or condition associated with MAGL is cancer.

In a particularly preferred embodiment, said disease or condition associated with MAGL is inflammatory bowel disease.

In one embodiment, said disease or condition associated with MAGL is pain.

In one embodiment, said diseases or conditions associated with MAGL are selected from multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and visceral pain.

In one embodiment, said diseases or conditions associated with MAGL are selected from multiple sclerosis, Alzheimer's disease and Parkinson's disease.

In one embodiment, said diseases or conditions associated with MAGL are selected from inflammatory bowel disease, inflammatory bowel symptoms, gut motility, visceral pain, fibromyalgia, endometriosis, COPD, and asthma.

In one aspect, the present invention provides a compound of formula (I), or a pharmaceutically acceptable salt thereof, for use in a method described above.

In one aspect, the present invention provides the use of a compound of formula (I), or of a pharmaceutically acceptable salt thereof, in a method described above.

In one aspect, the present invention provides the use of a compound of formula (I), or of a pharmaceutically acceptable salt thereof, in the manufacture of a medicament for the treatment or prophylaxis of a disease or condition associated with MAGL described herein.

### Pharmaceutical Compositions and Administration

In one aspect, the present invention provides a pharmaceutical composition comprising a compound of formula (I) as described herein and a therapeutically inert carrier.

In one embodiment, there is provided a pharmaceutical composition according to Example 376 or 377.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Synthesis of Building Blocks

### Example A.5

### 8-oxa-2,5-diazaspiro[3.5]nonan-6-one; trifluoroacetic Acid

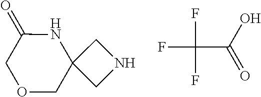

To a solution of 6-keto-8-oxa-2,5-diazaspiro[3.5]nonane-2-carboxylic acid tert-butyl ester (625 mg, 2.58 mmol) in dichloromethane (14 mL) was added TFA (2.94 g, 1.99 mL, 25.8 mmol) and the reaction mixture was stirred at r.t for 18 h. Volatiles were removed in vacuo to yield 955 mg of the crude title compound which was used without further purification. MS (ESI): m/z=143.1 [M−TFA+H]⁺

### Example A.6

### 2,5-diazaspiro[3.5]nonan-6-one; 4-methylbenzenesulfonic Acid

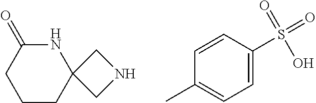

To a solution of tert-butyl 6-oxo-2,5-diazaspiro[3.5]nonane-2-carboxylate (1275 mg, 5.31 mmol) in Ethyl acetate (50 mL), p-toluenesulfonic acid monohydrate (2520 mg, 13.3 mmol) was added. The mixture was stirred at 20° C. for 15 h. The precipitated solid was filtered, washed with ACN and dried to afford the title compound (1655 mg, 3.42 mmol, 61% yield) as a light brown solid. MS (ESI): m/z=141.0 [M−TsOH+H]⁺

### Step a) tert-butyl 3-allyl-3-(prop-2-enoylamino) azetidine-1-carboxylate

To a solution of tert-butyl 3-allyl-3-amino-azetidine-1-carboxylate (CAS: 1440962-19-5) (6.0 g, 28.3 mmol) in DCM (150 mL) triethylamine (4.73 mL, 33.9 mmol) was added. The mixture was cooled to −30° C. and acryloyl chloride (2.4 mL, 29.7 mmol) was added dropwise. The mixture was warmed to 20° C. and stirred for 3 h. The reaction mixture was washed with water, the organic phase was dried over sodium sulfate and concentrated. The residue was purified by column chromatography (1:1 EtOAc/hexane) to give the title compound (4.55 g, 17.1 mmol, 58.6% yield) as an yellow oil. MS (ESI): m/z=211.0 [M−C₄H₈+H]⁺

### Step b) tert-butyl 6-oxo-2,5-diazaspiro[3.5]non-7-ene-2-carboxylate

A solution of tert-butyl 3-allyl-3-(prop-2-enoylamino) azetidine-1-carboxylate (4.45 g, 16.7 mmol) in toluene (500 mL) was degassed with Ar. (1,3-bis(2,4,6-trimethylphenyl)-2-imidazolidinylidene)dichloro(phenylmethylene)(tricyclohexylphosphine)ruthenium (709 mg, 0.84 mmol) was added and the mixture was heated at 90° C. for 10 h. After cooling, solvent was evaporated and the residue was purified by column chromatography (100% EtOAc) to give the title compound (1.35 g, 5.67 mmol, 33% yield) as a brown solid. MS (ESI): m/z=183.2 [M−C₄H₈+H]⁺

### Step c) tert-butyl 6-oxo-2,5-diazaspiro[3.5]nonane-2-carboxylate

The suspension of tert-butyl 6-oxo-2,5-diazaspiro[3.5]non-7-ene-2-carboxylate (1350.0 mg, 5.67 mmol, 1.0 eq), Pd/C 10% (301.41 mg, 0.28 mmol, 0.05 eq) in Ethanol (30 mL) was stirred under hydrogen atmosphere for 16 h. The mixture was filtered, the filtrate was concentrated. The residue was dried to afford tert-butyl 6-oxo-2,5-diazaspiro[3.5]nonane-2-carboxylate (1360.0 mg, 5.66 mmol, 98.6% yield) as a light brown solid. MS (ESI): m/z=185.0 [M−C₄H₈+H]⁺

### Example B.1

### [3-(2-azaspiro[3.3]heptan-6-ylmethyl)phenyl]-imino-oxo-(trifluoromethyl)-λ6-sulfane; 4-azaspiro[3.3]heptane-2-carboxylate

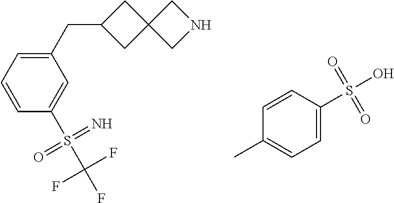 [SMILES: N=S(=O)(c1cccc(CC2CC3(CNC3)C2)c1)C(F)(F)F ; Cc1ccc(S(=O)(=O)O)cc1]

A mixture of p-toluenesulfonic acid (1.18 g, 6.83 mmol) and tert-butyl 6-[[3-(trifluoromethylsulfonimidoyl)phenyl]methyl]-2-azaspiro[3.3]heptane-2-carboxylate (1.3 g, 3.11 mmol) in ethyl acetate (30 mL) was stirred at 40° C. for 24 h. After the completion of the reaction, the reaction mixture was concentrated and purified by HPLC to afford the title compound (339 mg, 0.690 mmol, 15.6% yield) as brown viscous oil. MS (ESI): m/z=319.0 [M−TsOH+H]⁺

### Step a) tert-butyl 6-[(4,4,5,5-tetramethyl-1,3,2-dioxaborolan-2-yl)methylene]-2-azaspiro[3.3]heptane-2-carboxylate

A mixture of 2,2,6,6-tetramethylpiperidine (95.9 mL, 568 mmol) in THF (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THF (750 mL) was added dropwise. After stirring for 30 min, a solution of tert-butyl 6-oxo-2-azaspiro[3.3]heptane-2-carboxylate (100 g, 473 mmol) in THF (300 mL) was added in dropwise at −60° C. The reaction mixture was allowed to slowly warm up to 25° C. and stirred at 25° C. for 12 h. The mixture was added H₂O (80 mL) slowly and then purified together with an additional batch of equal size by silica gel column (PE/EA=1:0 to 3:1 gradient) to give the title compound (220 g, 656 mmol, approx 69% yield per batch) as a white solid which was confirmed by ¹H NMR (400 MHZ, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) tert-butyl 6-[[3-(trifluoromethylsulfonimidoyl)phenyl]methylene]-2-azaspiro[3.3]heptane-2-carboxylate

(3-bromophenyl)-imino-oxo-(trifluoromethyl)-λ6-sulfane (2.47 g, 8.59 mmol), tert-butyl 6-[(4,4,5,5-tetramethyl-1,3,2-dioxaborolan-2-yl)methylene]-2-azaspiro[3.3]heptane-2-carboxylate (2.4 g, 7.16 mmol), 1,1′-bis(diphenylphosphino)ferrocene-palladium(II) dichloride dichloromethane complex (1.17 g, 1.43 mmol) and potassium carbonate (1.98 g, 14.3 mmol) were dissolved in 1,4-Dioxane (40 mL) and water (8 mL). The reaction mixture was heated to 120° C. under argon for 16 h. The reaction mixture was concentrated under reduced pressure. The residue was partitioned between ethyl acetate and water. The organic layer was washed with brine. The extract was dried over sodium sulfate, filtered through a thin layer of silica gel and evaporated. The crude product was purified by column chromatography to afford the title compound (1 g, 2.4 mmol, 31.9% yield) as light yellow solid. MS (ESI): m/z=361.0 [M−tBu+H]⁺.

### Step c) tert-butyl 6-[[3-(trifluoromethylsulfonimidoyl)phenyl]methyl]-2-azaspiro[3.3]heptane-2-carboxylate

A mixture of tert-butyl 6-[[3-(trifluoromethylsulfonimidoyl)phenyl]methylene]-2-azaspiro[3.3]heptane-2-carboxylate (1.3 g, 3.12 mmol) and palladium on carbon (10%) (0.16 mL, 1.56 mmol) in EtOAc (35 mL) was stirred in an autoclave for 24 h under 30 bar of H₂. Then the reaction mixture was filtered and concentrated to afford the title compound (1.3 g, 3.11 mmol, 96.5% yield) a as grey oil. MS (ESI): m/z=319.0 [M−Boc+H]⁺.

In analogy to Example B.1, the following building blocks were generated using the relevant (hetero) aryl bromide or iodide building block for the Suzuki coupling in Step b. In some cases, alternative salts (e.g. trifluoroacetate, ditosylate, hydrochloride) were also used. To introduce different spiro-ring systems further building block substitutions can be made, for example Example B.25 used tert-butyl 7-oxo-2-azaspiro[3.5]nonane-2-carboxylate (CAS: 1363381-22-9) in place of tert-butyl 6-oxo-2-azaspiro[3.3]heptane-2-carboxylate in Step a), and Example B.26 and B.47 used tert-butyl 6-oxo-2-azaspiro[3.4]octane-2-carboxylate (CAS: 1363382-39-1) in place of tert-butyl 6-oxo-2-azaspiro[3.3]heptane-2-carboxylate in Step a).

| | | | | MS, ESI: |
|---|---|---|---|---|
| Ex. | Structure | Systematic Name | Building Blocks | m/z |
| B.2 | 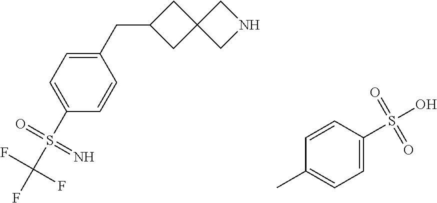 [SMILES: N=S(=O)(c1ccc(CC2CC3(CNC3)C2)cc1)C(F)(F)F ; Cc1ccc(S(=O)(=O)O)cc1] | [4-(2-azaspiro[3.3]- heptan-6-ylmethyl)- phenyl]imino-oxo- (trifluoromethyl)-λ⁶- sulfane; 4-methyl- benzenesulfonic acid | (4-bromophen- yl)(imino)(tri- fluoromethyl)- λ6-sulfanone (CAS: 1421133- 03-0) | 319.0 [M − TsOH + H]⁺ |
| B.3 | 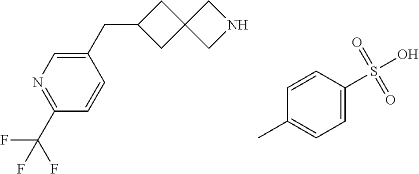 [SMILES: FC(F)(F)c1ccc(CC2CC3(CNC3)C2)cn1 ; Cc1ccc(S(=O)(=O)O)cc1] | 6-[[6-(trifluorometh- yl)-3-pyridyl]meth- yl]-2-azaspiro[3.3]- heptane; 4-methyl- benzenesulfonic acid | 5-bromo-2- (trifluorometh-yl)pyridine (CAS: 436799- 32-5) | 257.0 [M − TsOH + H]⁺ |
| B.4 | 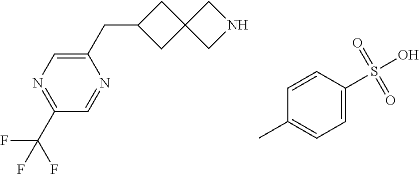 [SMILES: FC(F)(F)c1cnc(CC2CC3(CNC3)C2)cn1 ; Cc1ccc(S(=O)(=O)O)cc1] | 6-[[5-(trifluoro- methyl)pyrazin-2- yl]meth-yl]-2-aza- spiro[3.3]heptane; 4-methylbenzene- sulfonic acid | 2-bromo-5- (trifluorometh- yl)pyrazine (CAS: 799557- 87-2) | 258.2 [M − TsOH + H]⁺ |
| B.5 | 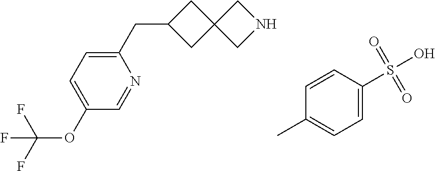 [SMILES: FC(F)(F)Oc1ccc(CC2CC3(CNC3)C2)nc1 ; Cc1ccc(S(=O)(=O)O)cc1] | 6-[[6-(trifluoro- methyl)-3-pyridyl]- methyl]-2-azaspiro- [3.3]heptane; 4- methylbenzenesul- fonic acid | 2-bromo-5- (trifluorometh- oxy)pyridine (CAS: 888327- 36-4) | 273.3 [M − TsOH + H]⁺ |
| B.6 | 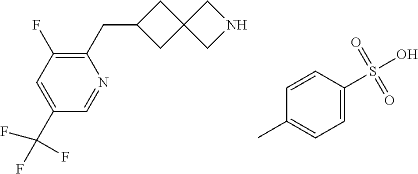 [SMILES: Fc1cc(C(F)(F)F)cnc1CC1CC2(CNC2)C1 ; Cc1ccc(S(=O)(=O)O)cc1] | 6-[[3-fluoro-5-(tri- fluoromethyl)-2-pyr- idyl]methyl]-2-aza- spiro[3.3]heptane; 4- methylbenzenesul- fonic acid | 2-bromo-3- fluoro-5-(tri- fluoromethyl)- pyridine (CAS: 89402-29-9) | 447.0 [M + H]⁺ |
| B.7 | 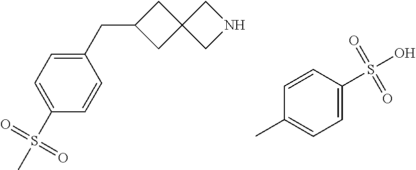 [SMILES: CS(=O)(=O)c1ccc(CC2CC3(CNC3)C2)cc1 ; Cc1ccc(S(=O)(=O)O)cc1] | 6-[(4-methylsulfon- ylphenyl)methyl]- 2-azaspiro[3.3]- heptane; 4-methyl- benzenesulfonic acid | 4-bromophenyl methyl sulfone (CAS: 3466-32- 8) | 266.0 [M − TsOH + H]⁺ |
| B.8 | 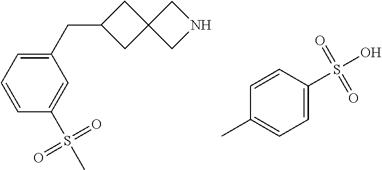 [SMILES: CS(=O)(=O)c1cccc(CC2CC3(CNC3)C2)c1 ; Cc1ccc(S(=O)(=O)O)cc1] | 6-[(3-methylsulfon- ylphenyl)methyl]-2- azaspiro[3.3]heptane; 4-methylbenzene- sulfonic acid | 3-bromophenyl- methylsulfone (CAS: 34896- 80-5) | 265.9 [M − TsOH + H]⁺ |
| B.9 | 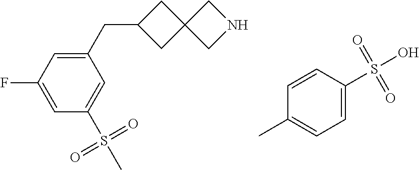 [SMILES: CS(=O)(=O)c1cc(F)cc(CC2CC3(CNC3)C2)c1 ; Cc1ccc(S(=O)(=O)O)cc1] | 6-[(3-fluoro-5-meth- ylsulfonylphenyl)- methyl]-2-azaspiro- [3.3]heptane; 4- methylbenzenesul- fonic acid | 1-bromo-3- fluoro-5-methyl- sulfonyl-benzene (CAS: 1207970- 78-2) | 284.0 [M − TsOH + H]⁺ |
| B.10 | 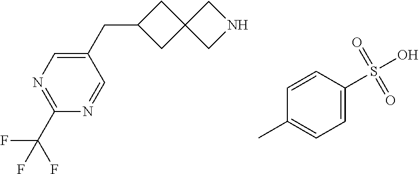 [SMILES: FC(F)(F)c1ncc(CC2CC3(CNC3)C2)cn1 ; Cc1ccc(S(=O)(=O)O)cc1] | 6-[[2-(trifluorometh- yl)pyrimidin-5-yl]- methyl]-2-azaspiro- [3.3]heptane; 4- methylbenzenesul- fonic acid | 5-bromo-2- (trifluorometh- yl)pyrimidine (CAS: 799557- 86-1) | 258.1 [M − TsOH + H]⁺ |
| B.11 | 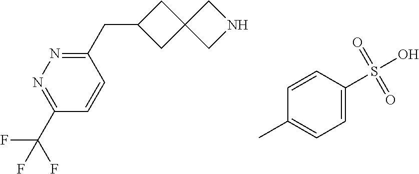 [SMILES: FC(F)(F)c1ccc(CC2CC3(CNC3)C2)nn1 ; Cc1ccc(S(=O)(=O)O)cc1] | 6-[[6-(trifluorometh- yl)pyridazin-3-yl]- methyl]-2-azaspiro- [3.3]heptane; 4- methylbenzenesul- fonic acid | 3-bromo-6- (trifluorometh- yl)pyridazine (CAS: 174607- 37-5) | 258.4 [M − TsOH + H]⁺ |
| B.12 | 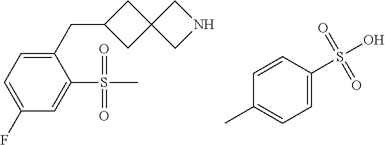 [SMILES: CS(=O)(=O)c1cc(F)ccc1CC1CC2(CNC2)C1 ; Cc1ccc(S(=O)(=O)O)cc1] | 6-[(4-fluoro-2-meth- ylsulfonyl-phenyl)- methyl]-2-azaspiro- [3.3]heptane; 4- methylbenzenesul- fonic acid | 1-bromo-4- fluoro-2-meth- anesulfonyl- benzene (CAS: 628692-10-4) | 284.4 [M − TsOH + H]⁺ |
| B.13 | 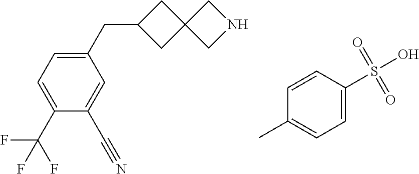 [SMILES: N#Cc1cc(CC2CC3(CNC3)C2)ccc1C(F)(F)F ; Cc1ccc(S(=O)(=O)O)cc1] | 5-(2-azaspiro[3.3]- heptan-6-ylmethyl)- 2-(trifluoromethyl)- benzonitrile; 4-meth- ylbenzenesulfonic acid | 5-bromo-2- (trifluorometh- yl)benzonitrile (CAS: 1208076- 28-1) | 281.1 [M − TsOH + H]⁺ |
| B.14 | 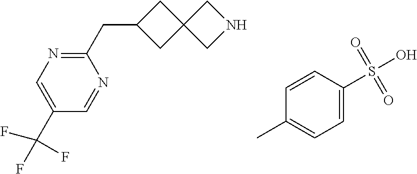 [SMILES: FC(F)(F)c1cnc(CC2CC3(CNC3)C2)nc1 ; Cc1ccc(S(=O)(=O)O)cc1] | 6-[[5-(trifluorometh- yl)pyrimidin-2-yl]- methyl]-2-azaspiro- [3.3]heptane; 4-methylbenzenesul- fonic acid | 2-bromo-5- (trifluorometh- yl)pyrimidine (CAS: 69034- 09-9) | 257.9 [M − TsOH + H]⁺ |
| B.15 | 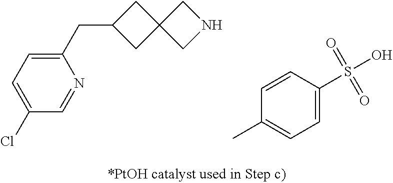 | 6-[(5-chloro-2- pyridyl)methyl]-2- azaspiro[3.3]heptane; 4-methylbenzene- sulfonic acid | 2-bromo-5- chloropyridine | 223.2 [M − TsOH + H]⁺ |
| B.16 | 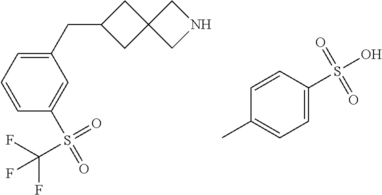 [SMILES: O=S(=O)(c1cccc(CC2CC3(CNC3)C2)c1)C(F)(F)F ; Cc1ccc(S(=O)(=O)O)cc1] | 6-[[3-(trifluorometh- ylsulfonyl)phenyl]- methyl]-2-azaspiro- [3.3]heptane; 4- methylbenzenesul- fonic acid | 1-bromo-3- ((trifluorometh- yl)sulfonyl) benzene (CAS: 2728-70-3) | 319.0 [M − TsOH + H]⁺ |
| B.17 | 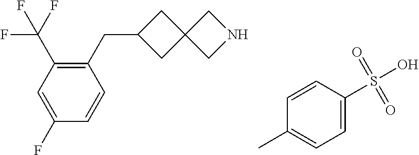 [SMILES: Fc1ccc(CC2CC3(CNC3)C2)c(C(F)(F)F)c1 ; Cc1ccc(S(=O)(=O)O)cc1] | 6-[[4-fluoro-2- (trifluoromethyl)- phenyl]methyl]-2- azaspiro[3.3]heptane; 4-methylbenzene- sulfonic acid | 2-bromo-5- fluorobenzo- trifluoride | 274.0 [M − TsOH + H]⁺ |
| B.18 | 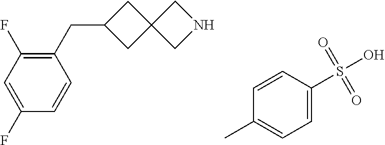 [SMILES: Fc1ccc(CC2CC3(CNC3)C2)c(F)c1 ; Cc1ccc(S(=O)(=O)O)cc1] | 6-[(2,4-difluoro- phenyl)methyl]-2- azaspiro[3.3]heptane; 4-methylbenzene- sulfonic acid | 1-bromo-2,4- difluorobenzene | 224.0 [M − TsOH + H]⁺ |
| B.19 | 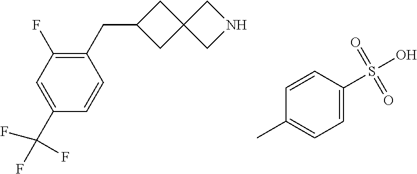 [SMILES: Fc1cc(C(F)(F)F)ccc1CC1CC2(CNC2)C1 ; Cc1ccc(S(=O)(=O)O)cc1] | 6-[[2-fluoro-4- (trifluoromethyl)- phenyl]methyl]-2- azaspiro[3.3]heptane; 4-methylbenzene- sulfonic acid | 4-bromo-3- fluorobenzo- trifluoride | 274.0 [M − TsOH + H]⁺ |
| B.20 | 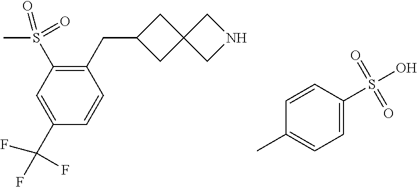 [SMILES: CS(=O)(=O)c1cc(C(F)(F)F)ccc1CC1CC2(CNC2)C1 ; Cc1ccc(S(=O)(=O)O)cc1] | 6-[[2-methylsulfon- yl-4-(trifluorometh- yl)phenyl]methyl]-2- azaspiro[3.3]heptane; 4-methylbenzene- sulfonic acid | 1-bromo-2- methanesulfon- yl-4-(trifluoro- methyl)benzene (CAS: 1215205- 98-3) | 334.1 [M − TsOH + H]⁺ |
| B.21 | 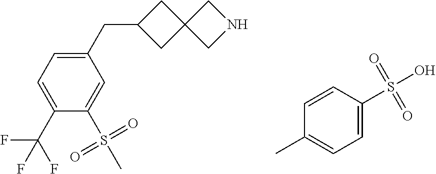 [SMILES: CS(=O)(=O)c1cc(CC2CC3(CNC3)C2)ccc1C(F)(F)F ; Cc1ccc(S(=O)(=O)O)cc1] | 6-[3-methylsulfon- yl-4-(trifluorometh- yl)phenyl]methyl]- 2-azaspiro[3.3]- heptane; 4-methyl- benzenesulfonic acid | 4-bromo-2- methylsulfon- yl-1-(trifluoro- methyl)benzene (C.1) | 334.0 [M − TsOH + H]⁺ |
| B.22 | 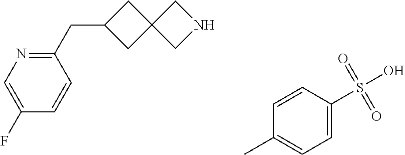 | 6-[(5-fluoro-2- pyridyl)methyl]-2- azaspiro[3.3]heptane; 4-methylbenzene- sulfonic acid | 2-bromo-5- fluoropyridine | 207.5 [M − TsOH + H]⁺ |
| B.23 | 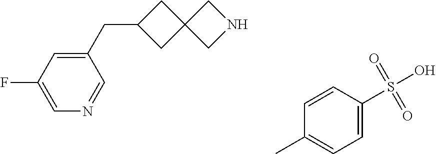 | 6-[(5-fluoro-3- pyridyl)methyl]-2- azaspiro[3.3]heptane; 4-methylbenzene- sulfonic acid | 3-bromo-5- fluoropyridine | 207.2 [M − TsOH + H]⁺ |
| B.24 | 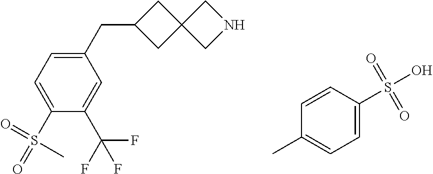 [SMILES: CS(=O)(=O)c1ccc(CC2CC3(CNC3)C2)cc1C(F)(F)F ; Cc1ccc(S(=O)(=O)O)cc1] | 6-[[4-methylsulfon- yl-3-(trifluorometh- yl)phenyl]methyl]-2- azaspiro[3.3]heptane; 4-methylbenzene- sulfonic acid | 4-bromo-1- methylsulfon- yl-2-(trifluoro- methyl)benzene (CAS: 300356- 32-5) | 334.2 [M − TsOH + H]⁺ |
| B.25 | 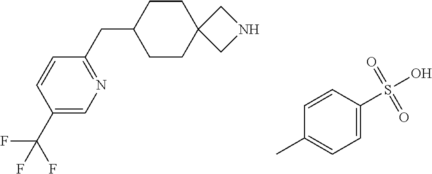 [SMILES: FC(F)(F)c1ccc(CC2CCC3(CC2)CNC3)nc1 ; Cc1ccc(S(=O)(=O)O)cc1] | 7-[[5-(trifluoro- methyl)-2-pyridyl]- methyl]-2-azaspiro- [3.5]nonane; 4-meth- ylbenzenesulfonic acid | 2-bromo-5- (trifluorometh- yl)pyridine | 285.2 [M − TsOH + H]⁺ |
| B.26 | 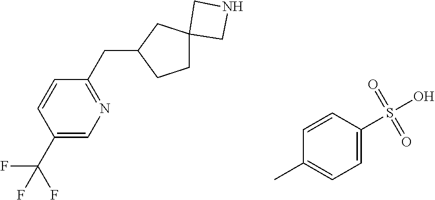 [SMILES: FC(F)(F)c1ccc(CC2CCC3(CNC3)C2)nc1 ; Cc1ccc(S(=O)(=O)O)cc1] | 6-[5-(trifluorometh- yl)-2-pyridyl]meth- yl]-2-azaspiro[3.4]- octane; 4-methyl- benzenesulfonic acid | 2-bromo-5- (trifluorometh- yl)pyridine | 271.2 [M − TsOH + H]⁺ |
| B.34 | 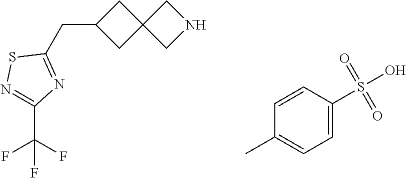 [SMILES: FC(F)(F)c1nsc(CC2CC3(CNC3)C2)n1 ; Cc1ccc(S(=O)(=O)O)cc1] | 5-(2-azaspiro[3.3]- heptan-6-ylmethyl)- 3-(trifluoromethyl)- 1,2,4-thiadiazole; 4- methylbenzenesul- fonic acid | 5-bromo-3- (trifluorometh- yl)-1,2,4- thiadiazole (CAS: 1781783- 65-0) | 264.0 [M − TsOH + H]⁺ |
| B.35 | 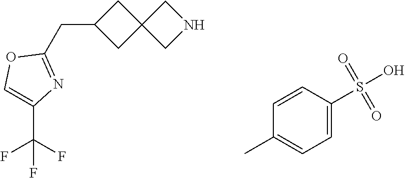 [SMILES: FC(F)(F)c1coc(CC2CC3(CNC3)C2)n1 ; Cc1ccc(S(=O)(=O)O)cc1] | 2-(2-azaspiro[3.3]- heptan-6-ylmethyl)- 4-(trifluorometh- yl)oxazole; 4-meth- ylbenzenesulfonic acid | 2-bromo-4- (trifluorometh- yl)oxazole (CAS: 1060816- 14-9) | 247.2 [M − TsOH + H]⁺ |
| B.47 | 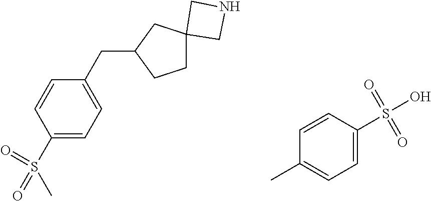 [SMILES: CS(=O)(=O)c1ccc(CC2CCC3(CNC3)C2)cc1 ; Cc1ccc(S(=O)(=O)O)cc1] | 6-[(4-methylsulfon- ylphenyl)methyl]-2- azaspiro[3.4]octane; 4-methylbenzene- sulfonic acid | 4-bromophenyl- methyl sulfone | 280.0 [M − TsOH + H]⁺ |
| B.69 | 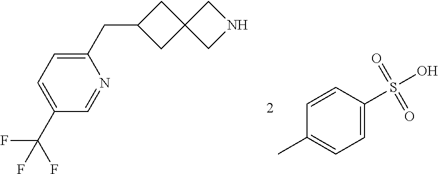 [SMILES: FC(F)(F)c1ccc(CC2CC3(CNC3)C2)nc1 ; Cc1ccc(S(=O)(=O)O)cc1] | 6-[5-(trifluorometh- yl)-2-pyridyl]meth- yl]-2-azaspiro[3.3]- heptane; di 4-methyl- benzenesulfonic acid | 2-bromo-5- (trifluorometh- yl)pyridine | 257.4 [M − 2TsOH + H]⁺ |
| B.70 | 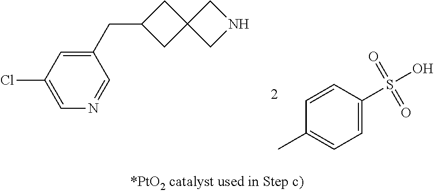 | 6-[(5-chloro-3- pyridyl)methyl]-2- azaspiro[3.3]heptane; di 4-methylbenzene- sulfonic acid | 3-bromo-5- chloropyridine | 223.1 [M − 2TsOH + H]₊ |
| B.71 | 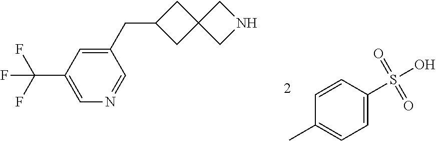 [SMILES: FC(F)(F)c1cncc(CC2CC3(CNC3)C2)c1 ; Cc1ccc(S(=O)(=O)O)cc1] | 6-[[5-(trifluorometh- yl)-3-pyridyl]meth- yl]-2-azaspiro[3.3]- heptane; di 4-meth- ylbenzenesulfonic acid | 3-bromo-5- (trifluorometh- yl)pyridine | 257.4 [M − 2TsOH + H]⁺ |
| B.72 | 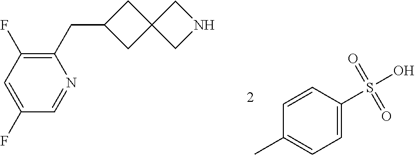 [SMILES: Fc1cnc(CC2CC3(CNC3)C2)c(F)c1 ; Cc1ccc(S(=O)(=O)O)cc1] | 6-[(3,5-difluoro-2- pyridyl)methyl]-2- azaspiro[3.3]heptane; di 4-methylbenzene- sulfonic acid | 2-bromo-3,5- difluoropyridine | 225.0 [M − 2TsOH + H]⁺ |
| B.73 | 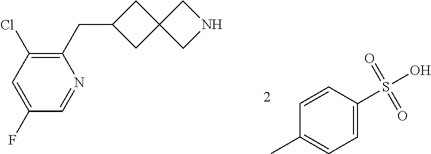 [SMILES: Fc1cnc(CC2CC3(CNC3)C2)c(Cl)c1 ; Cc1ccc(S(=O)(=O)O)cc1] | 6-[(3-chloro-5- fluoro-2-pyridyl)- methyl]-2-azaspiro- [3.3]heptane; di 4- methylbenzenesul- fonic acid | 2-bromo-3- chloro-5-fluoro- pyridine | 241.1 [M − 2TsOH + H]⁺ |
| B.74 | 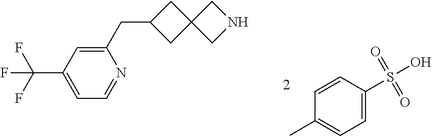 [SMILES: FC(F)(F)c1ccnc(CC2CC3(CNC3)C2)c1 ; Cc1ccc(S(=O)(=O)O)cc1] | 6-[[4-(trifluorometh- yl)-2-pyridyl]meth- yl]-2-azaspiro[3.3]- heptane; di 4-methyl- benzenesulfonic acid; | 2-bromo-4- (trifluorometh- yl)pyridine | 257.5 [M − 2TsOH + H]⁺ |
| B.77 | 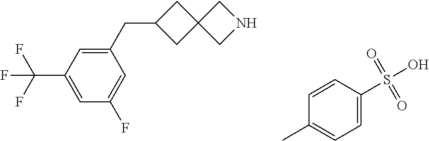 [SMILES: Fc1cc(CC2CC3(CNC3)C2)cc(C(F)(F)F)c1 ; Cc1ccc(S(=O)(=O)O)cc1] | 6-[[3-fluoro-5- (trifluoromethyl)- phenyl]methyl]-2- azaspiro[3.3]heptane; 4-methylbenzene- sulfonic acid | 3-bromo-5- fluorobenzo- trifluoride | 274.0 [M − TsOH + H]⁺ |
| B.86 | 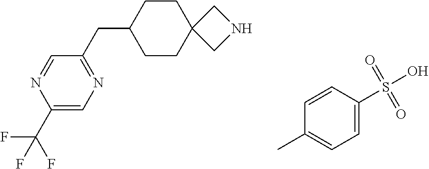 [SMILES: FC(F)(F)c1cnc(CC2CCC3(CC2)CNC3)cn1 ; Cc1ccc(S(=O)(=O)O)cc1] | 7-[[5-(trifluorometh- yl)pyrazin-2-yl]- methyl]-2-azaspiro- [3.5]nonane; 4-meth- ylbenzenesulfonic acid | 2-bromo-5- (trifluorometh- yl)pyrazine | 285.0 [M − TsOH + H]⁺ |
| B.87 | 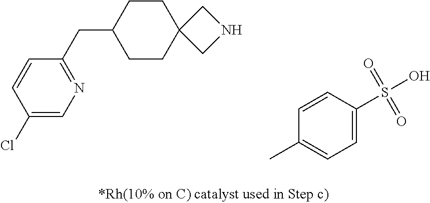 [SMILES: Clc1ccc(CC2CCC3(CC2)CNC3)nc1 ; Cc1ccc(S(=O)(=O)O)cc1] | 7-[(5-chloro-2- pyridyl)methyl]-2- azaspiro[3.5]nonane; 4-methylbenzene- sulfonic acid | 2-bromo-5- chloropyridine | 251.2 [M − TsOH + H]⁺ |
| B.88 | 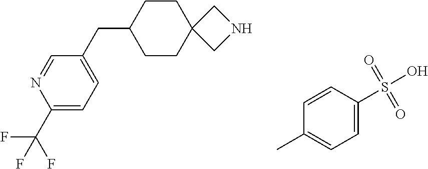 [SMILES: FC(F)(F)c1ccc(CC2CCC3(CC2)CNC3)cn1 ; Cc1ccc(S(=O)(=O)O)cc1] | 7-[[6-(trifluorometh- yl)-3-pyridyl]meth- yl]-2-azaspiro[3.5]- nonane; 4-methyl- benzenesulfonic acid | 5-bromo-2- (trifluorometh- yl)pyridine | 285.2 [M − TsOH + H]⁺ |
| B.89 | 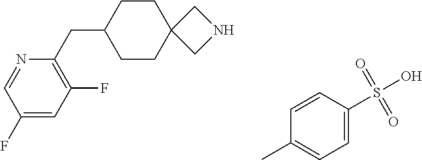 [SMILES: Fc1cnc(CC2CCC3(CC2)CNC3)c(F)c1 ; Cc1ccc(S(=O)(=O)O)cc1] | 7-[(3,5-difluoro-2- pyridyl)methyl]-2- azaspiro[3.5]nonane; 4-methylbenzene- sulfonic acid | 2-bromo-3,5- difluoropyridine | 253.2 [M − TsOH + H]⁺ |
| B.92 | 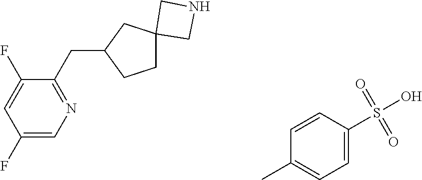 [SMILES: Fc1cnc(CC2CCC3(CNC3)C2)c(F)c1 ; Cc1ccc(S(=O)(=O)O)cc1] | 6-[(3,5-difluoro-2- pyridyl)methyl]-2- azaspiro[3.4]octane; 4-methylbenzene- sulfonic acid | 2-bromo-3,5- difluoropyridine | 239.2 [M − TsOH + H]⁺ |
| B.93 | 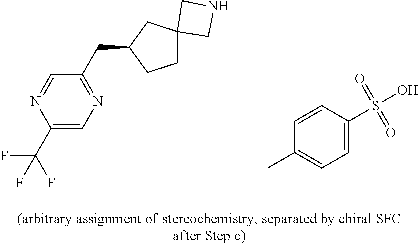 [SMILES: FC(F)(F)c1cnc(C[C@@H]2CCC3(CNC3)C2)cn1 ; Cc1ccc(S(=O)(=O)O)cc1] | (6R)-6-[[5-(trifluoro- methyl)pyrazin-2- yl]methyl]-2- azaspiro[3.4]octane; 4-methylbenzene- sulfonic acid | 2-bromo-5- (trifluorometh- yl)pyrazine | 272.2 [M − TsOH + H]⁺ |
| B.94 | 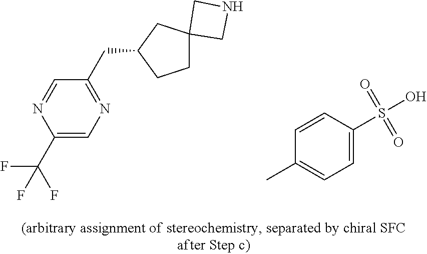 [SMILES: FC(F)(F)c1cnc(C[C@H]2CCC3(CNC3)C2)cn1 ; Cc1ccc(S(=O)(=O)O)cc1] | 6-[[5-(trifluoro- methyl)pyrazin-2- yl]methyl]-2- azaspiro[3.4]octane; 4-methylbenzene- sulfonic acid | 2-bromo-5- (trifluorometh- yl)pyrazine | 272.2 [M − TsOH + H]⁺ |
| B.95 | 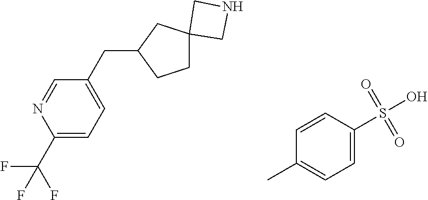 [SMILES: FC(F)(F)c1ccc(CC2CCC3(CNC3)C2)cn1 ; Cc1ccc(S(=O)(=O)O)cc1] | 6-[[6-(trifluoro- methyl)-3-pyridyl]- methyl]-2-azaspiro- [3.4]octane; 4-meth- ylbenzenesulfonic acid | 5-bromo-2- (trifluorometh- yl)pyridine | 271.1 [M − TsOH + H]⁺ |
| B.96 | 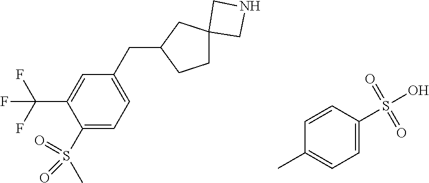 [SMILES: CS(=O)(=O)c1ccc(CC2CCC3(CNC3)C2)cc1C(F)(F)F ; Cc1ccc(S(=O)(=O)O)cc1] | 6-[[4-methylsulfon- yl-3-(trifluorometh- yl)phenyl]methyl]-2- azaspiro[3.4]octane; 4-methylbenzene- sulfonic acid | 4-bromo-1-mes- yl-2-(trifluoro- methyl)benzene (CAS: 300356- 32-5) | 348.3 [M − TsOH + H]⁺ |
| B.302 | 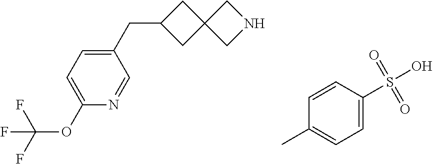 [SMILES: FC(F)(F)Oc1ccc(CC2CC3(CNC3)C2)cn1 ; Cc1ccc(S(=O)(=O)O)cc1] | 6-[[6-(trifluorometh- oxy)-3-pyridyl]meth- yl]-2-azaspiro[3.3]- heptane; 4-methyl- benzenesulfonic acid | 5-bromo-2- (trifluorometh- oxy)pyridine (CAS: 886371- 77-3) | 273.3 [M − TsOH + H]⁺ |
| B.305 | 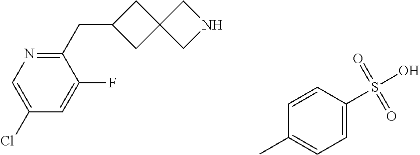 [SMILES: Fc1cc(Cl)cnc1CC1CC2(CNC2)C1 ; Cc1ccc(S(=O)(=O)O)cc1] | 6-[(5-chloro-3- fluoro-2-pyridyl)- methyl]-2-azaspiro- [3.3]heptane; di 4- methylbenzenesul- fonic acid | 2-bromo-5- chloro-3-fluoro- pyridine (CAS: 514797-97-8) | 241.1 [M − 2TsOH + H]⁺ |
| B.306 | 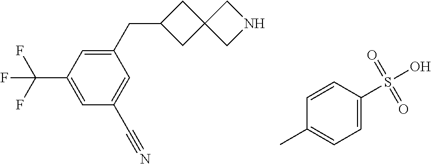 [SMILES: N#Cc1cc(CC2CC3(CNC3)C2)cc(C(F)(F)F)c1 ; Cc1ccc(S(=O)(=O)O)cc1] | 3-(2-azaspiro[3.3]- heptan-6-ylmethyl)- 5-(trifluoromethyl)- benzonitrile; 4-meth- ylbenzenesulfonic acid | 3-bromo-5- (trifluorometh- yl)benzonitrile (CAS: 691877- 03-9) | 281.2 [M − TsOH + H]⁺ |
| B.307 | 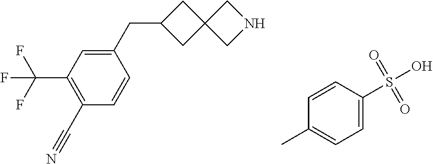 [SMILES: N#Cc1ccc(CC2CC3(CNC3)C2)cc1C(F)(F)F ; Cc1ccc(S(=O)(=O)O)cc1] | 4-(2-azaspiro[3.3]- heptan-6-ylmethyl)- 2-(trifluorometh- yl)benzonitrile; di 4-methylbenzene- sulfonic acid | 4-bromo-2- (trifluorometh- yl)benzonitrile (CAS: 191165- 13-6) | 281.1 [M − 2TsOH + H]⁺ |
| B.308 | 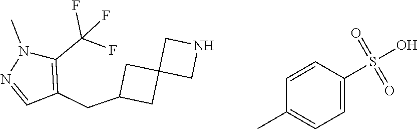 [SMILES: Cn1ncc(CC2CC3(CNC3)C2)c1C(F)(F)F ; Cc1ccc(S(=O)(=O)O)cc1] | 6-[[1-methyl-5- (trifluoromethyl)pyr- azol-4-yl]methyl]-2- azaspiro[3.3]heptane; 4-methylbenzene- sulfonic acid | 4-bromo-1- methyl-5-(tri- fluoromethyl)- pyrazole | 260.2 [M − TsOH + H]⁺ |
| B.309 | 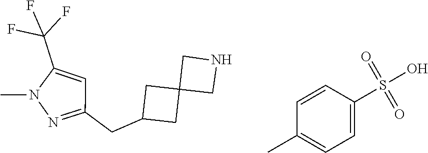 [SMILES: Cn1nc(CC2CC3(CNC3)C2)cc1C(F)(F)F ; Cc1ccc(S(=O)(=O)O)cc1] | 6-[[1-methyl-5- (trifluoromethyl)pyr- azol-3-yl]methyl]-2- azaspiro[3.3]heptane; 4-methylbenzene- sulfonic acid | 3-bromo-1- methyl-5-(tri- fluoromethyl)- pyrazole | 260.0 [M − TsOH + H]⁺ |
| B.310 | 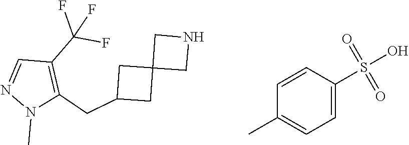 [SMILES: Cn1ncc(C(F)(F)F)c1CC1CC2(CNC2)C1 ; Cc1ccc(S(=O)(=O)O)cc1] | 6-[[2-methyl-4- (trifluoromethyl)- pyrazol-3-yl]meth- yl]-2-azaspiro[3.3]- heptane; 4-methyl- benzenesulfonic acid; | 5-bromo-1- methyl-4-(tri- fluoromethyl)- pyrazole (CAS: 1783749-68-7) | 260.0 [M − TsOH + H]⁺ |
| B.312 | 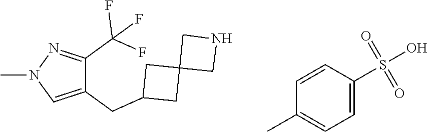 [SMILES: Cn1cc(CC2CC3(CNC3)C2)c(C(F)(F)F)n1 ; Cc1ccc(S(=O)(=O)O)cc1] | 6-[[1-methyl-3- (trifluoromethyl)- pyrazol-4-yl]meth- yl]-2-azaspiro[3.3]- heptane; 4-methyl- benzenesulfonic acid | 4-bromo-1- methyl-3-(tri- fluoromethyl)- 1H-pyrazole (CAS: 497832- 99-2) | 260.2 [M − TsOH + H]⁺ |
| B.313 | 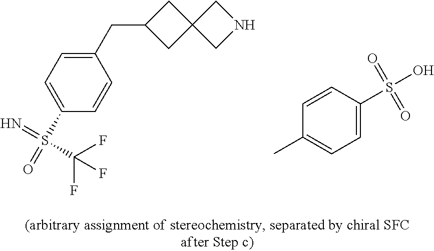 | [4-(2-azaspiro[3.3]- heptan-6-ylmethyl)- phenyl]-imino-oxo- (trifluoromethyl)- λ⁶-sulfane; 4-methyl- benzenesulfonic acid | (4-bromophen- yl)(imino)(tri- fluoromethyl)- λ⁶-sulfanone (CAS: 1421133- 03-0) | 319.1 [M − TsOH + H]⁺ |
| B.314 | 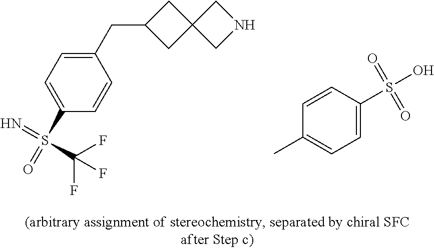 [SMILES: N=[S@@](=O)(c1ccc(CC2CC3(CNC3)C2)cc1)C(F)(F)F ; Cc1ccc(S(=O)(=O)O)cc1] | [4-(2-azaspiro[3.3]- heptan-6-ylmeth- yl)phenyl]-imino- oxo-(trifluorometh- yl)-λ⁶-sulfane; 4- methylbenzenesul- fonic acid | (4-bromophen- yl)(imino)(tri- fluoromethyl)- λ6-sulfanone (CAS: 1421133- 03-0) | 319.3 [M − TsOH + H]⁺ |
| P.77 | 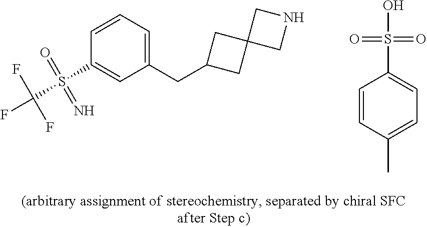 [SMILES: N=[S@@](=O)(c1cccc(CC2CC3(CNC3)C2)c1)C(F)(F)F ; Cc1ccc(S(=O)(=O)O)cc1] | [3-(2-azaspiro[3.3]- heptan-6-ylmethyl)- phenyl]-imino-oxo- (trifluoromethyl)-λ⁶- sulfane; 4-methyl- benzenesulfonic acid | (3-bromophen- yl)-imino-oxo- (trifluorometh- yl)-λ6-sulfane | 319.1 [M + H]⁺ |
| P.78 | 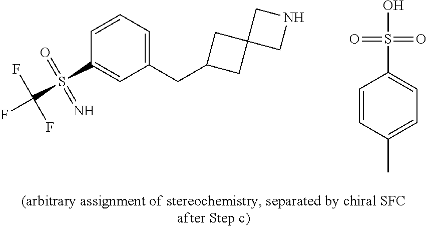 [SMILES: N=[S@](=O)(c1cccc(CC2CC3(CNC3)C2)c1)C(F)(F)F ; Cc1ccc(S(=O)(=O)O)cc1] | [3-(2-azaspiro[3.3]- heptan-6-ylmethyl)- phenyl]-imino-oxo- (trifluoromethyl)-λ⁶- sulfane; 4-methyl- benzenesulfonic acid | (3-bromophen- yl)-imino-oxo- (trifluorometh- yl)-λ6-sulfane | 319.1 [M + H]⁺ |
| P.41 | 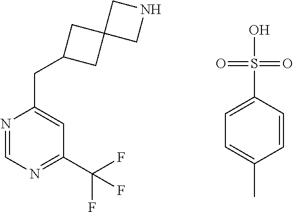 [SMILES: FC(F)(F)c1cc(CC2CC3(CNC3)C2)ncn1 ; Cc1ccc(S(=O)(=O)O)cc1] | 4-methylbenzene- sulfonic acid; 6-[[6- (trifluoromethyl)pyr- imidin-4-yl]methyl]- 2-azaspiro[3.3]- heptane | 4-chloro-6-(tri- fluoromethyl)- pyrimidine | 258.0 [M + H]⁺ |
| P.42 | 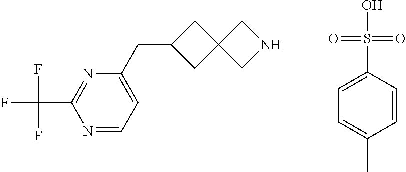 [SMILES: FC(F)(F)c1nccc(CC2CC3(CNC3)C2)n1 ; Cc1ccc(S(=O)(=O)O)cc1] | 4-methylbenzene- sulfonic acid; 6-[[2- (trifluoromethyl)pyr- imidin-4-yl]methyl]- 2-azaspiro[3.3]- heptane | 4-chloro-2- (trifluorometh- yl)pyrimidine | 258.1 [M + H]⁺ |
| P.7 | 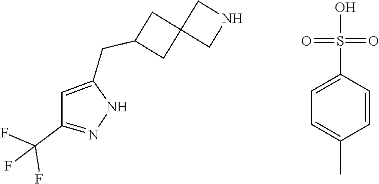 [SMILES: FC(F)(F)c1cc(CC2CC3(CNC3)C2)[nH]n1 ; Cc1ccc(S(=O)(=O)O)cc1] | 6-[[3-(trifluorometh- yl)-1H-pyrazol-5- yl]methyl]-2-aza- spiro[3.3]heptane; 4-methylbenzene- sulfonic acid | 5-bromo-3- (trifluorometh- yl)-1H-pyrazole | 246.0 [M + H]⁺ |
| P.10 | 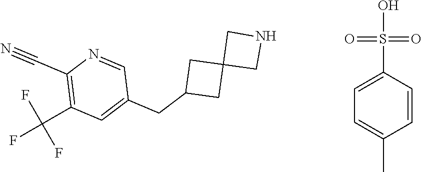 [SMILES: N#Cc1ncc(CC2CC3(CNC3)C2)cc1C(F)(F)F ; Cc1ccc(S(=O)(=O)O)cc1] | 5-(2-azaspiro[3.3]- heptan-6-ylmethyl)- 3-(trifluoromethyl)- picolinonitrile; 4- methylbenzenesul- fonic acid | 5-bromo-3- (trifluorometh- yl)picolinonitrile (CAS 1214377- 57-7) | 282.2 [M + H]⁺ |
| P.11 | 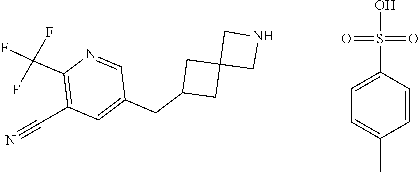 [SMILES: N#Cc1cc(CC2CC3(CNC3)C2)cnc1C(F)(F)F ; Cc1ccc(S(=O)(=O)O)cc1] | 5-(2-azaspiro[3.3]- heptan-6-ylmethyl)- 2-(trifluoromethyl)- nicotinonitrile; 4- methylbenzenesul- fonic acid | 5-bromo-2- (trifluorometh- yl)nicotinonitrile (CAS 1245914- 98-0) | 282.2 [M + H]⁺ |
| P.12 | 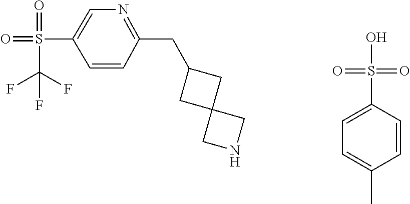 [SMILES: O=S(=O)(c1ccc(CC2CC3(CNC3)C2)nc1)C(F)(F)F ; Cc1ccc(S(=O)(=O)O)cc1] | 6-[[5-(trifluorometh- ylsulfonyl)-2- pyridyl]methyl]-2- azaspiro[3.3]heptane; 4-methylbenzene- sulfonic acid | 5-chloro-2- (trifluorometh- ylsulfonyl)pyr- idine (CAS: 2091447-89-9) | 321.1 [M + H]⁺ |
| P.13 | 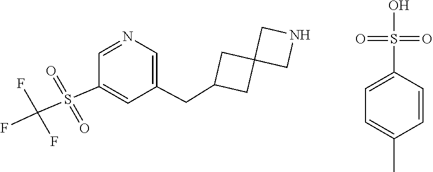 [SMILES: O=S(=O)(c1cncc(CC2CC3(CNC3)C2)c1)C(F)(F)F ; Cc1ccc(S(=O)(=O)O)cc1] | 4-methylbenzene- sulfonic acid; 6-[[5- (trifluoromethylsul- fonyl)-3-pyridyl]- methyl]-2-azaspiro- [3.3]heptane | 3-bromo-5- (trifluorometh- ylsulfonyl)pyr- idine (CAS: 1936542-46-9) | 321.1 [M + H]⁺ |
| P.31 | 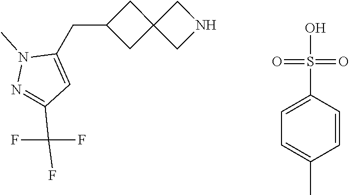 [SMILES: Cn1nc(C(F)(F)F)cc1CC1CC2(CNC2)C1 ; Cc1ccc(S(=O)(=O)O)cc1] | 6-[[2-methyl-5- (trifluoromethyl)- pyrazol-1-3-yl]meth- yl]-2-azaspiro[3.3]- heptane; 4-methyl- benzenesulfonic acid | 5-bromo-1- methyl-3-(tri- fluoromethyl)- pyrazole | 260.2 [M + H]⁺ |
| P.34 | 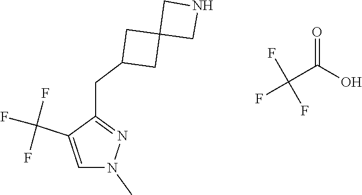 [SMILES: Cn1cc(C(F)(F)F)c(CC2CC3(CNC3)C2)n1 ; O=C(O)C(F)(F)F] | 6-[[1-methyl-4- (trifluoromethyl)- pyrazol-3-yl]meth- yl]-2-azaspiro[3.3]- heptane; 2,2,2- trifluoroacetic acid | 3-bromo-1- methyl-4-(tri- fluoromethyl)- pyrazole | 260.1 [M + H]⁺ |
| P.37 | 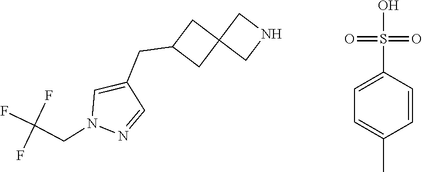 [SMILES: FC(F)(F)Cn1cc(CC2CC3(CNC3)C2)cn1 ; Cc1ccc(S(=O)(=O)O)cc1] | 6-[[1-(2,2,2-tri- fluoroethyl)pyrazol- 4-yl]methyl]-2- azaspiro[3.3]heptane; 4-methylbenzene- sulfonic acid | 4-bromo-1- (2,2,2-trifluoro- ethyl)pyrazole | 246.0 [M + H]⁺ |
| P.66 | 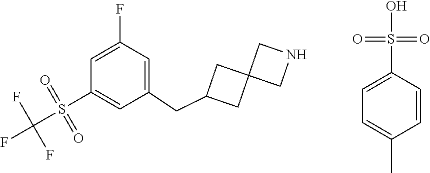 [SMILES: O=S(=O)(c1cc(F)cc(CC2CC3(CNC3)C2)c1)C(F)(F)F ; Cc1ccc(S(=O)(=O)O)cc1] | 6-[[3-fluoro-5-(tri- fluoromethylsulfon- yl)phenyl]methyl]-2- azaspiro[3.3]heptane; 4-methylbenzene- sulfonic acid | 1-bromo-3- fluoro-5-(tri- fluoromethylsul- fonyl)benzene | 338.0 [M + H]⁺ |
| P.74 | 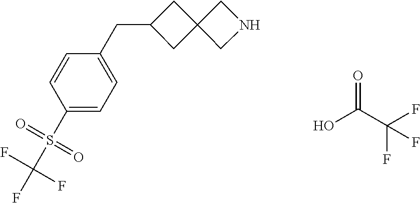 [SMILES: O=S(=O)(c1ccc(CC2CC3(CNC3)C2)cc1)C(F)(F)F ; O=C(O)C(F)(F)F] | 6-[[4-(trifluorometh- ylsulfonyl)phenyl]- methyl]-2-azaspiro- [3.3]heptane; 2,2,2- trifluoroacetic acid | 1-bromo-4- (trifluorometh- ylsulfonyl)- benzene | 320.1 [M + H]⁺ |
| P.90 | 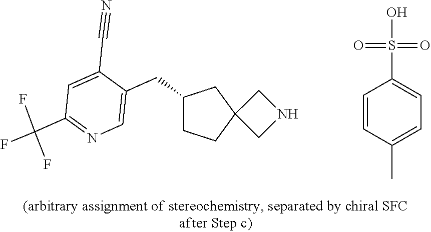 [SMILES: N#Cc1cc(C(F)(F)F)ncc1C[C@H]1CCC2(CNC2)C1 ; Cc1ccc(S(=O)(=O)O)cc1] | 5-[[rac-(6S)-2- azaspiro[3.4]octan- 6-yl]methyl]-2-(tri- fluoromethyl)pyr- idine-4-carbonitrile; 4-methylbenzene- sulfonic acid | 5-bromo-2- (trifluorometh- yl)pyridine-4- carbonitrile (CAS: 1070892- 04-4) | 296.2 [M + H]⁺ |
| P.91 | 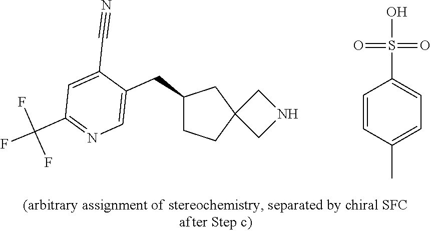 [SMILES: N#Cc1cc(C(F)(F)F)ncc1C[C@@H]1CCC2(CNC2)C1 ; Cc1ccc(S(=O)(=O)O)cc1] | 5-[[rac-(6R)-2- azaspiro[3.4]octan- 6-yl]methyl]-2-(tri- fluoromethyl)pyr- idine-4-carbonitrile; 4-methylbenzene- sulfonic acid | 5-bromo-2- (trifluorometh- yl)pyridine-4- carbonitrile (CAS: 1070892- 04-4) | 296.2 [M + H]⁺ |
| P.92 | 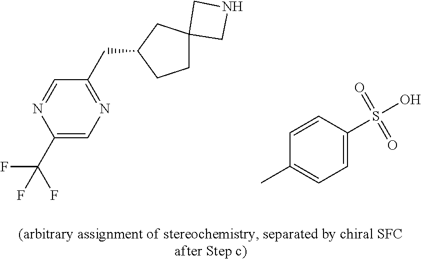 [SMILES: FC(F)(F)c1cnc(C[C@H]2CCC3(CNC3)C2)cn1 ; Cc1ccc(S(=O)(=O)O)cc1] | (6R)-6-[[5-(tri- fluoromethyl)pyr- azin-2-yl]methyl]-2- azaspiro[3.4]octane; 4-methylbenzene- sulfonic acid | 2-bromo-5- (trifluorometh- yl)pyrazine | 272.2 [M + H]⁺ |
| P.93 | 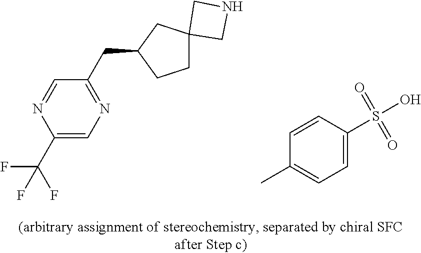 [SMILES: FC(F)(F)c1cnc(C[C@@H]2CCC3(CNC3)C2)cn1 ; Cc1ccc(S(=O)(=O)O)cc1] | (6R)-6-[[5-(trifluoro- methyl)pyrazin-2- yl]methyl]-2-aza- spiro[3.4]octane; 4- methylbenzenesul- fonic acid | 2-bromo-5- (trifluorometh- yl)pyrazine | 272.2 [M + H]⁺ |
| P.95 | 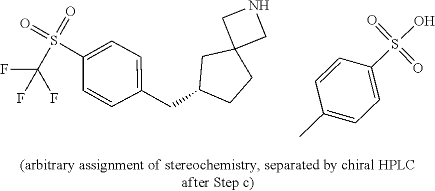 [SMILES: O=S(=O)(c1ccc(C[C@H]2CCC3(CNC3)C2)cc1)C(F)(F)F ; Cc1ccc(S(=O)(=O)O)cc1] | (6R)-6-[[4-(trifluoro- methylsulfonyl)phen- yl]methyl]-2-aza- spiro[3.4]octane; 4- methylbenzenesul- fonic acid | 1-bromo-4- (trifluorometh- ylsulfonyl)- benzene | 334.2 [M + H]⁺ |
| P.96 | 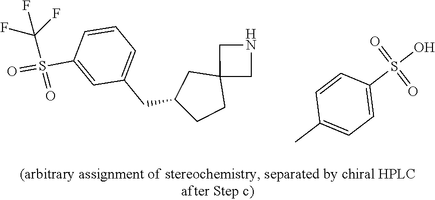 [SMILES: O=S(=O)(c1cccc(C[C@H]2CCC3(CNC3)C2)c1)C(F)(F)F ; Cc1ccc(S(=O)(=O)O)cc1] | (6R)-6-[[3-(trifluoro- methylsulfonyl)phen- yl]methyl]-2-aza- spiro[3.4]octane; 4- methylbenzenesul- fonic acid; | 1-bromo-3- (trifluorometh- ylsulfonyl)- benzene | 334.1 [M + H]⁺ |
| B.315 | 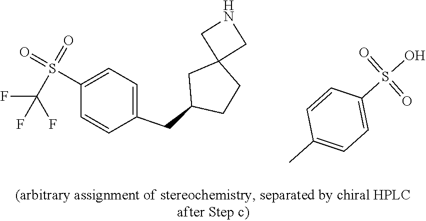 [SMILES: O=S(=O)(c1ccc(C[C@@H]2CCC3(CNC3)C2)cc1)C(F)(F)F ; Cc1ccc(S(=O)(=O)O)cc1] | (6S)-6-[[4-(trifluoro- methylsulfonyl)phen- yl]methyl]-2-aza- spiro[3.4]octane; 4- methylbenzenesul- fonic acid | 1-bromo-4- (trifluorometh- ylsulfonyl)- benzene | 334.2 [M + H]⁺ |
| B.316 | 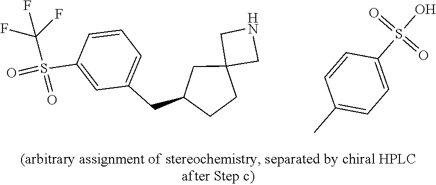 [SMILES: O=S(=O)(c1cccc(C[C@@H]2CCC3(CNC3)C2)c1)C(F)(F)F ; Cc1ccc(S(=O)(=O)O)cc1] | (6S)-6-[[3-(trifluoro- methylsulfonyl)phen- yl]methyl]-2-aza- spiro[3.4]octane; 4- methylbenzenesul- fonic acid; | 1-bromo-3- (trifluorometh- ylsulfonyl)- benzene | 334.1 [M + H]⁺ |
| P.71 | 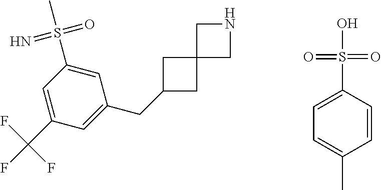 [SMILES: CS(=N)(=O)c1cc(CC2CC3(CNC3)C2)cc(C(F)(F)F)c1 ; Cc1ccc(S(=O)(=O)O)cc1] | [3-(2-azaspiro[3.3]- heptan-6-ylmethyl)- 5-(trifluorometh- yl)phenyl]-imino- methyl-oxo-26-sulfane; 4-methyl- benzenesulfonic acid | C.6 [3-bromo- 5-(trifluoro- methyl)phenyl]- imino-methyl- oxo-16-sulfane | 333.2 [M + H]⁺ |
| B.318 | 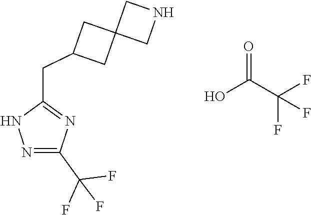 [SMILES: FC(F)(F)c1n[nH]c(CC2CC3(CNC3)C2)n1 ; O=C(O)C(F)(F)F] | 6-[[3-(trifluorometh- yl)-1H-1,2,4-triazol- 5-yl]methyl]-2-aza- spiro[3.3]heptane; 2,2,2-trifluoroacetic acid | 3-Bromo-5- (trifluorometh- yl)-1H-1,2,4- triazole (CAS: 1185320-36-8) | 247.5 [M − TFA + H]⁺ |
| B.320 | 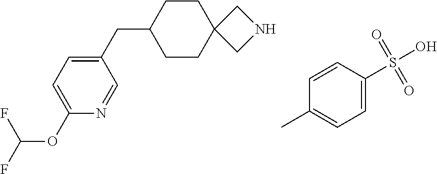 [SMILES: FC(F)Oc1ccc(CC2CCC3(CC2)CNC3)cn1 ; Cc1ccc(S(=O)(=O)O)cc1] | 7-[[6-(difluorometh- oxy)-3-pyridyl]meth- yl]-2-azaspiro[3.5]- nonane; 4-methyl- benzenesulfonic acid | 5-bromo-2- (difluorometh- oxy)pyridine (CAS: 899452- 26-7) | 283.2 [M − TsOH + H]⁺ |
| B.321 | 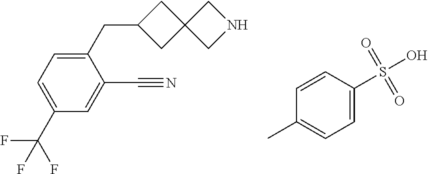 [SMILES: N#Cc1cc(C(F)(F)F)ccc1CC1CC2(CNC2)C1 ; Cc1ccc(S(=O)(=O)O)cc1] | 2-(2-azaspiro[3.3]- heptan-6-ylmethyl)- 5-(trifluoromethyl)- benzonitrile; 4-meth- ylbenzenesulfonic acid | 2-bromo-5- (trifluorometh- yl)benzonitrile (CAS: 1483-55- 2) | 281.1 [M − TsOH + H]⁺ |
| B.322 | 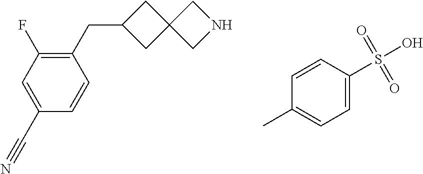 [SMILES: N#Cc1ccc(CC2CC3(CNC3)C2)c(F)c1 ; Cc1ccc(S(=O)(=O)O)cc1] | 4-(2-azaspiro[3.3]- heptan-6-ylmethyl)- 3-fluoro-benzonitrile; 4-methylbenzene- sulfonic acid | 4-bromo-3- fluorobenzo- nitrile | 231.2 [M − TsOH + H]⁺ |
| B.330 | 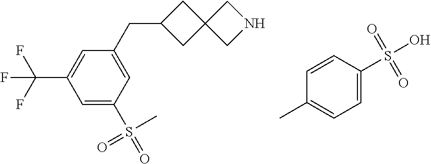 [SMILES: CS(=O)(=O)c1cc(CC2CC3(CNC3)C2)cc(C(F)(F)F)c1 ; Cc1ccc(S(=O)(=O)O)cc1] | 6-[[3-methylsulfon- yl-5-(trifluorometh- yl)phenyl]methyl]-2- azaspiro[3.3]heptane; 4-methylbenzene- sulfonic acid | 1-bromo-3- methylsulfonyl- 5-(trifluoro- methyl)benzene (CAS: 1215205- 96-1) | 334.1 [M − TsOH + H]⁺ |
| B.331 | 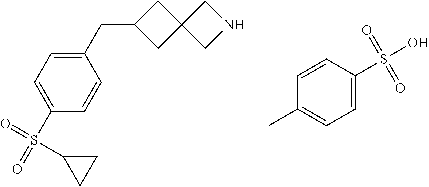 [SMILES: O=S(=O)(c1ccc(CC2CC3(CNC3)C2)cc1)C1CC1 ; Cc1ccc(S(=O)(=O)O)cc1] | 6-[(4-cyclopropyl- sulfonylphenyl)- methyl]-2-azaspiro- [3.3]heptane; 4- methylbenzenesul- fonic acid | 1-bromo-4- cyclopropyl- sulfonyl- benzene (CAS: 648906-28-9) | 292.2 [M − TsOH + H]⁺ |
| B.335 | 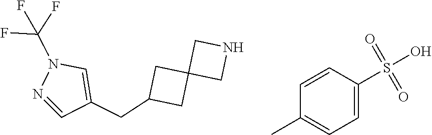 [SMILES: FC(F)(F)n1cc(CC2CC3(CNC3)C2)cn1 ; Cc1ccc(S(=O)(=O)O)cc1] | 6-[[1-(trifluorometh- yl)pyrazo1-4-yl]- methyl]-2-azaspiro- [3.3]heptane; 4- methylbenzenesul- fonic acid | 4-bromo-1- (trifluorometh- yl)pyrazole (CAS: 1046831- 97-3) | 246.1 [M − TsOH + H]⁺ |
| B.344 | 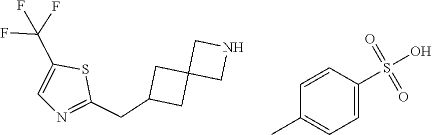 [SMILES: FC(F)(F)c1cnc(CC2CC3(CNC3)C2)s1 ; Cc1ccc(S(=O)(=O)O)cc1] | 2-(2-azaspiro[3.3]- heptan-6-ylmethyl)- 5-(trifluoromethyl)- thiazole; 4-methyl- benzenesulfonic acid | 2-bromo-4- (trifluorometh- yl)thiazole (CAS: 41731- 39-9) | 263.0 [M − TsOH + H]⁺ |
| B.345 | 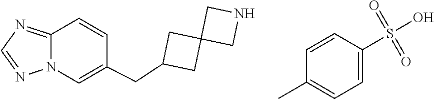 [SMILES: c1nc2ccc(CC3CC4(CNC4)C3)cn2n1 ; Cc1ccc(S(=O)(=O)O)cc1] | 6-(2-azaspiro[3.3]- heptan-6-ylmethyl)- [1,2,4]triazolo[1,5- a]pyridine; di 4- methylbenzenesul- fonic acid | 6-bromo-[1,2,4]- triazolo[1,5-a]- pyridine (CAS: 356560-80-0) | 229.1 [M − 2TsOH + H]⁺ |
| B.346 | 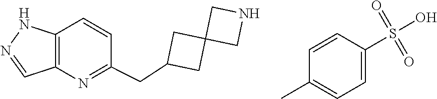 [SMILES: c1cc2[nH]ncc2nc1CC1CC2(CNC2)C1 ; Cc1ccc(S(=O)(=O)O)cc1] | 5-(2-azaspiro[3.3]- heptan-6-ylmethyl)- 1H-pyrazolo[4,3-b]- pyridine;4-methyl- benzenesulfonic acid | 5-bromo-1H- pyrazolo[4,3-b]- pyridine (CAS: 1227628-78-5) | 229.1 [M − 2TsOH + H]⁺ |
| B.353 | 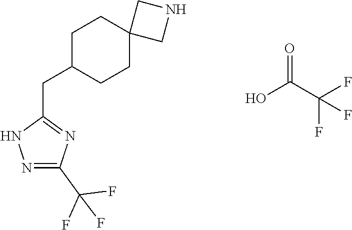 [SMILES: FC(F)(F)c1n[nH]c(CC2CCC3(CC2)CNC3)n1 ; O=C(O)C(F)(F)F] | 7-[[3-(trifluorometh- yl)-1H-1,2,4-triazol- 5-yl]methyl]-2-aza- spiro[3.5]nonane; 2,2,2-trifluoroacetic acid | 3-Bromo-5- (trifluorometh- yl)-1H-1,2,4- triazole (CAS: 1185320-36-8) | 275.2 [M − TFA + H]⁺ |
| B.355 | 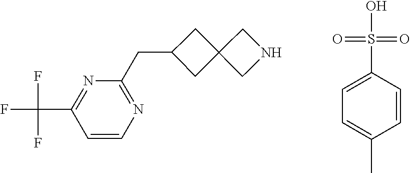 [SMILES: FC(F)(F)c1ccnc(CC2CC3(CNC3)C2)n1 ; Cc1ccc(S(=O)(=O)O)cc1] | 6-[[4-(trifluorometh- yl)pyrimidin-2-yl]- methyl]-2-azaspiro- [3.3]heptane; 4- methylbenzenesul- fonic acid | 2-chloro-4- (trifluorometh- yl)pyrimidine | 258.2 [M − TsOH + H]⁺ |
| B.356 | 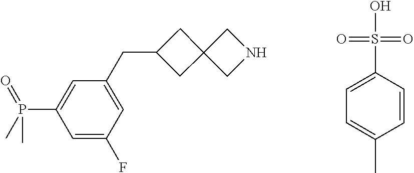 [SMILES: CP(C)(=O)c1cc(F)cc(CC2CC3(CNC3)C2)c1 ; Cc1ccc(S(=O)(=O)O)cc1] | 6-[(3-dimethylphos- phoryl-5-fluoro- phenyl)methyl]-2- azaspiro[3.3]heptane; 4-methylbenzene- sulfonic acid | 1-bromo-3- dimethylphos- phoryl-5-fluoro- benzene (C.7) | 282.0 [M − TsOH + H]⁺ |
| B.357 | 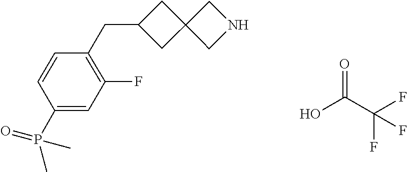 [SMILES: CP(C)(=O)c1ccc(CC2CC3(CNC3)C2)c(F)c1 ; O=C(O)C(F)(F)F] | 6-[(4-dimethylphos- phoryl-2-fluoro- phenyl)methyl]-2- azaspiro[3.3]heptane; 4-methylbenzene- sulfonic acid | 1-chloro-4- dimethylphos- phoryl-2-fluoro- benzene (C.8) | 282.1 [M − TFA + H]⁺ |
| B.373 | 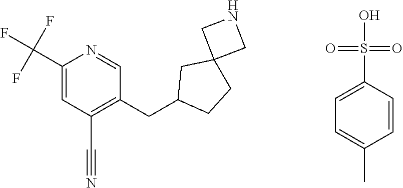 [SMILES: N#Cc1cc(C(F)(F)F)ncc1CC1CCC2(CNC2)C1 ; Cc1ccc(S(=O)(=O)O)cc1] | 5-(2-azaspiro[3.4]- octan-6-ylmethyl)- 2-(trifluoromethyl)- pyridine-4-car- bonitrile; 4-meth- ylbenzenesulfonic acid | 5-Bromo-2- (trifluorometh- yl)isonicotino- nitrile (CAS: 1070892-04-4) | 296.2 [M − TsOH + H]⁺ |
| B.374 | 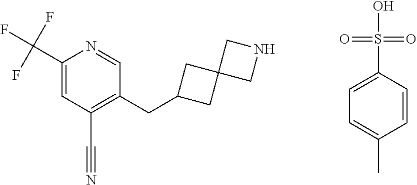 [SMILES: N#Cc1cc(C(F)(F)F)ncc1CC1CC2(CNC2)C1 ; Cc1ccc(S(=O)(=O)O)cc1] | 5-(2-azaspiro[3.3]- heptan-6-ylmethyl)- 2-(trifluoromethyl)- pyridine-4-car- bonitrile; 4-methyl- benzenesulfonic acid | 5-Bromo-2- (trifluorometh- yl)isonicotino- nitrile (CAS: 1070892-04-4) | 282.1 [M − TsOH + H]⁺ |
| B.383 | 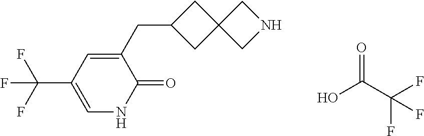 [SMILES: O=c1[nH]cc(C(F)(F)F)cc1CC1CC2(CNC2)C1 ; O=C(O)C(F)(F)F] | 3-(2-azaspiro[3.3]- heptan-6-ylmethyl)- 5-(trifluoromethyl)- 1H-pyridin-2-one; 2,2,2-trifluoroacetic acid | 3-bromo-2- hydroxy-5- (trifluorometh- yl)pyridine (CAS: 76041- 73-1) | 273.2 [M − TFA + H]⁺ |

### Example B.27

### 7-[6-(trifluoromethyl)pyridazin-3-yl]oxy-2-azaspiro[3.5]nonane; 4-methylbenzenesulfonic Acid

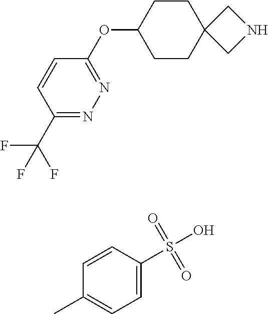 [SMILES: FC(F)(F)c1ccc(OC2CCC3(CC2)CNC3)nn1 ; Cc1ccc(S(=O)(=O)O)cc1]

To a suspension of 7-[6-(trifluoromethyl)pyridazin-3-yl]oxy-2-azaspiro[3.5]nonane-2-carboxylic acid tert-butyl ester (350 mg, 0.903 mmol) in isopropyl acetate (4 mL) and was added p-toluenesulfonic acid monohydrate (258 mg, 1.36 mmol). The mixture was stirred at 100° C. for 6 h. The reaction mixture was concentrated in vacuo. Et₂O was added, and the mixture filtered through sintered glass. The white solid was washed with Et₂O (2×) and dried in vacuo to afford the title compound (0.420 g, 88%) as a white solid. MS (ESI): m/z=288.0 [M−TsOH+H]⁺

### Step a) 7-[6-(trifluoromethyl)pyridazin-3-yl]oxy-2-azaspiro[3.5]nonane-2-carboxylic acid tert-butyl Ester

To a solution of 7-hydroxy-2-azaspiro[3.5]nonane-2-carboxylic acid tert-butyl ester (CAS: 1363383-18-9) (350 mg, 1.45 mmol) and potassium t-butoxide (195 mg, 1.74 mmol) in N,N-dimethylformamide (3.5 mL) was added 3-fluoro-6-(trifluoromethyl)pyridazine (248 mg, 1.49 mmol). The mixture was stirred at 80° C. for 15 h. The reaction mixture was poured into EtOAc and washed with water and brine. The organic layer was dried over Na₂SO₄ and concentrated in vacuo. The crude material was purified by flash chromatography (eluting with 0% to 40% EtOAc in heptane) to afford the title compound (353 mg, 59.69%) as white solid. MS (ESI): m/z=332.1 [M−tBu+H]⁺

In analogy to Example B.27, the following building blocks were generated using the relevant (hetero) aryl halide and hydroxy-spirocyclic building blocks. In some cases alternative solvents and bases were used for the S_{N}Ar reaction e.g. DMSO solvent, or NaH in DMF in Step a) were used.

| | | | | MS, |
|---|---|---|---|---|
| | | | Building | ESI: |
| Ex. | Structure | Systematic Name | Blocks | m/z |
| B.43 | 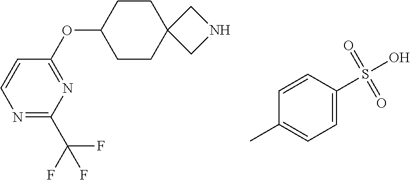 [SMILES: FC(F)(F)c1nccc(OC2CCC3(CC2)CNC3)n1 ; Cc1ccc(S(=O)(=O)O)cc1] | 7-[2- (trifluoromethyl)pyrimidin- 4-yl]oxy-2- azaspiro[3.5]nonane; 4- methylbenzenesulfonic acid | 4-chloro-2- (trifluoromethyl) pyrimidine, 7-hydroxy-2- azaspiro[3.5] nonane-2- carboxylic acid tert-butyl ester | 288.1 [M − TsOH + H]⁺ |
| B.44 | 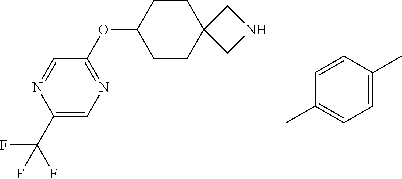 | 7-[5- (trifluoromethyl)pyrazin- 2-yl]oxy-2- azaspiro[3.5]nonane; 4- methylbenzenesulfonic acid | fluoro-5- (trifluoromethyl) pyrazine, 7- hydroxy-2- azaspiro[3.5] nonane-2- carboxylic acid tert-butyl ester | 288.1 [M − TsOH + H]⁺ |
| B.45 | 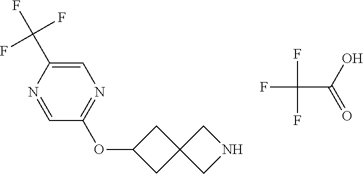 [SMILES: FC(F)(F)c1cnc(OC2CC3(CNC3)C2)cn1 ; O=C(O)C(F)(F)F] | 6-[5- (trifluoromethyl)pyrazin- 2-yl]oxy-2- azaspiro[3.3]heptane; trifluoroacetic acid | 2-chloro-5- (trifluoromethyl) pyrazine, 6- hydroxy-2- azaspiro[3.3] heptane-2- carboxylic acid tert-butyl ester (CAS: 1147557-97-8) | 260.1 [M − TFA + H]⁺ |
| B.46 | 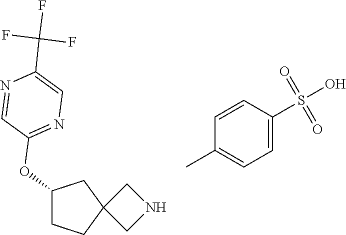 [SMILES: FC(F)(F)c1cnc(O[C@H]2CCC3(CNC3)C2)cn1 ; Cc1ccc(S(=O)(=O)O)cc1] | (6S)-6-[5- (trifluoromethyl)pyrazin- 2-yl]oxy-2- azaspiro[3.4]octane; 4- methylbenzenesulfonic acid | 2-fluoro-5- (trifluoromethyl) pyrazine, (6S)-6- hydroxy-2- azaspiro[3.4] octane-2- carboxylic acid tert-butyl ester (CAS: 2376903-72-7) | 274.0 [M − TsOH + H]⁺ |
| B.48 | 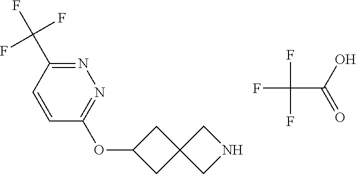 [SMILES: FC(F)(F)c1ccc(OC2CC3(CNC3)C2)nn1 ; O=C(O)C(F)(F)F] | 6-[6- (trifluoromethyl)pyridazin- 3-yl]oxy-2- azaspiro[3.3]heptane; trifluoroacetic acid | 3-chloro-6- (trifluoromethyl) pyridazine; 6-hydroxy-2- azaspiro[3.3] heptane-2- carboxylic acid tert-butyl ester (CAS: 1147557-97-8) | 260.1 [M − TFA + H]⁺ |
| B.85 | 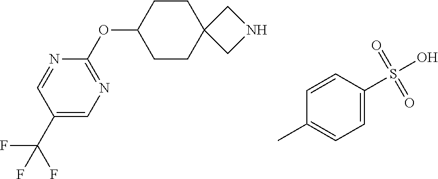 [SMILES: FC(F)(F)c1cnc(OC2CCC3(CC2)CNC3)nc1 ; Cc1ccc(S(=O)(=O)O)cc1] | 7-[5- (trifluoromethyl)pyrimidin- 2-yl]oxy-2- azaspiro[3.5]nonane; 4- methylbenzenesulfonic acid | 2-chloro-5- (trifluoromethyl) pyrimidine, 7-hydroxy-2- azaspiro[3.5] nonane-2- carboxylic acid tert-butyl ester | 288.1 [M − TsOH + H]+ |
| B.97 | 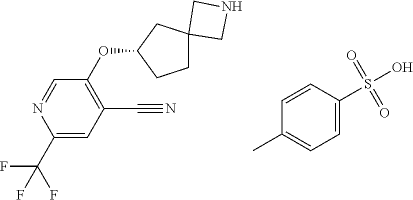 [SMILES: N#Cc1cc(C(F)(F)F)ncc1O[C@H]1CCC2(CNC2)C1 ; Cc1ccc(S(=O)(=O)O)cc1] | 5-[[(6S)-2- azaspiro[3.4]octan-6- yl]oxy]-2- (trifluoromethyl)pyridine- 4-carbonitrile; 4- methylbenzenesulfonic acid | 5-bromo-2- (trifluoromethyl) isonicotinonitrile (CAS: 1070892-04-4) and (6S)-6- hydroxy-2- azaspiro[3.4] octane-2- carboxylic acid tert-butyl ester (CAS: 2376903-72-7) | 298.2 [M − TsOH + H]⁺ |
| B.98 | 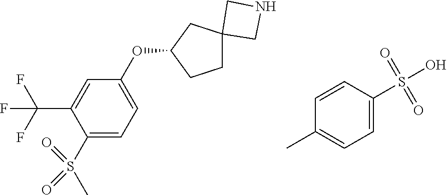 [SMILES: CS(=O)(=O)c1ccc(O[C@H]2CCC3(CNC3)C2)cc1C(F)(F)F ; Cc1ccc(S(=O)(=O)O)cc1] | (6S)-6-[4- methylsulfonyl-3- (trifluoromethyl)phenoxy]- 2-azaspiro[3.4]octane; 4-methylbenzenesulfonic acid | 4-bromo-1- mesyl-2- (trifluoromethyl) benzene and (6S)-6- hydroxy-2- azaspiro[3.4] octane-2- carboxylic acid tert-butyl ester (CAS: 2376903-72-7) and | 350.2 [M − TsOH + H]⁺ |
| B.260 | 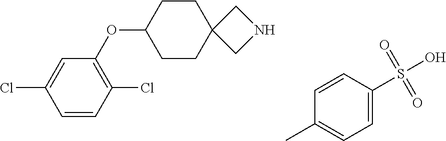 [SMILES: Clc1ccc(Cl)c(OC2CCC3(CC2)CNC3)c1 ; Cc1ccc(S(=O)(=O)O)cc1] | 7-(2,5-dichlorophenoxy)- 2-azaspiro[3.5]nonane; 4- methylbenzenesulfonic acid | 1,4-dichloro-2- methylsulfonyl benzene (CAS RN: 66640-63-9), 7-hydroxy- 2- azaspiro[3.5] nonane-2- carboxylic acid tert-butyl ester (CAS RN: 1363383-18-9) | 286.2 [M + H]⁺ |
| B.264 | 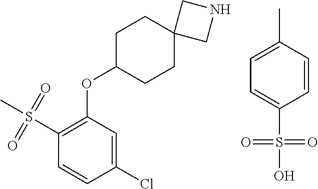 [SMILES: CS(=O)(=O)c1ccc(Cl)cc1OC1CCC2(CC1)CNC2 ; Cc1ccc(S(=O)(=O)O)cc1] | 7-(5-chloro-2- methylsulfonyl- phenoxy)-2- azaspiro[3.5]nonane; 4- methylbenzenesulfonic acid | 4-chloro-2- fluoro-1- mesyl-benzene (CAS RN: 1261284-27- 8), 7-hydroxy- 2- azaspiro[3.5] nonane-2- carboxylic acid tert-butyl ester (CAS RN: 1363383-18-9) | 330.1 [M + H]⁺ |
| B.269 | 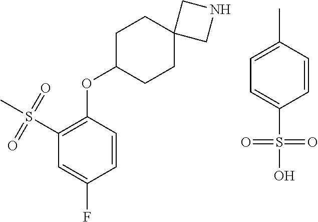 [SMILES: CS(=O)(=O)c1cc(F)ccc1OC1CCC2(CC1)CNC2 ; Cc1ccc(S(=O)(=O)O)cc1] | 7-(4-fluoro-2- methylsulfonyl- phenoxy)-2- azaspiro[3.5]nonane; 4- methylbenzenesulfonic acid | 1,4-difluoro-2- mesyl-benzene (CAS RN: 61655-69-4), 7-hydroxy-2- azaspiro[3.5] nonane-2- carboxylic acid tert-butyl ester (CAS RN: 1363383-18-9) | 314.1 [M + H]⁺ |
| B.319 | 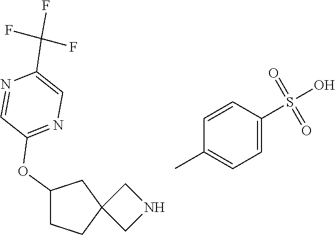 | 6-[5- (trifluoromethyl)pyrazin- 2-yl]oxy-2- azaspiro[3.4]octane; 4- methylbenzenesulfonic acid | 2-fluoro-5- (trifluoromethyl) pyrazine, 6- hydroxy-2- azaspiro[3.4] octane-2- carboxylic acid tert-butyl ester | 274.0 [M − TsOH + H]⁺ |
| B.332 | 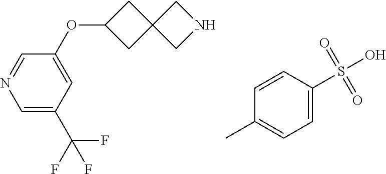 [SMILES: FC(F)(F)c1cncc(OC2CC3(CNC3)C2)c1 ; Cc1ccc(S(=O)(=O)O)cc1] | 6-[[5-(trifluoromethyl)-3- pyridyl]oxy]-2- azaspiro[3.3]heptane; 4- methylbenzenesulfonic acid | 3-fluoro-5- (trifluoromethyl) pyridine; 6- hydroxy-2- azaspiro[3.3] heptane-2- carboxylic acid tert-butyl ester | 260.1 [M − TsOH + H]⁺ |
| B.333 | 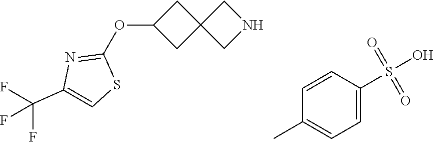 [SMILES: FC(F)(F)c1csc(OC2CC3(CNC3)C2)n1 ; Cc1ccc(S(=O)(=O)O)cc1] | 2-(2-azaspiro[3.3]heptan- 6-yloxy)-4- (trifluoromethyl)thiazole; 4-methylbenzenesulfonic acid | 2-bromo-4- (trifluoromethyl) thiazole; 6- hydroxy-2- azaspiro[3.3] heptane-2- carboxylic acid tert-butyl ester | 265.0 [M − TsOH + H]⁺ |
| B.340 | 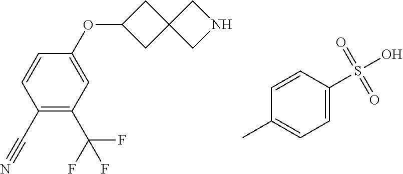 [SMILES: N#Cc1ccc(OC2CC3(CNC3)C2)cc1C(F)(F)F ; Cc1ccc(S(=O)(=O)O)cc1] | 4-(2-azaspiro[3.3]heptan- 6-yloxy)-2- (trifluoromethyl) benzonitrile; 4- methylbenzenesulfonic acid | 4-fluoro-2- (trifluoromethyl) benzonitrile; 6-hydroxy-2- azaspiro[3.3] heptane-2- carboxylic acid tert-butyl ester | 283.0 [M − TsOH + H]⁺ |
| B.341 | 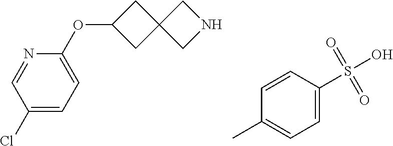 | 6-[(5-chloro-2- pyridyl)oxy]-2- azaspiro[3.3]heptane; 4- methylbenzenesulfonic acid | 5-chloro-2- fluoropyridine; 6-hydroxy-2- azaspiro[3.3] heptane-2- carboxylic acid tert-butyl ester | 225.0 [M − TsOH + H]⁺ |
| B.342 | 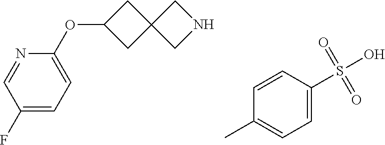 | 6-[(5-fluoro-2- pyridyl)oxy]-2- azaspiro[3.3]heptane; 4- methylbenzenesulfonic acid | 2,5- difluoropyridine; 6-hydroxy- 2- azaspiro[3.3] heptane-2- carboxylic acid tert-butyl ester | 209.1 [M − TsOH + H]⁺ |
| B.343 | 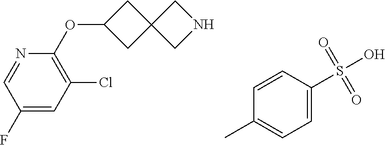 [SMILES: Fc1cnc(OC2CC3(CNC3)C2)c(Cl)c1 ; Cc1ccc(S(=O)(=O)O)cc1] | 6-[(3-chloro-5-fluoro-2- pyridyl)oxy]-2- azaspiro[3.3]heptane; 4- methylbenzenesulfonic acid | 5-chloro-2- fluoropyridine; 6-hydroxy-2- azaspiro[3.3] heptane-2- carboxylic acid tert-butyl ester | 243.0 [M − TsOH + H]⁺ |

### Example B.28

### 6-[4-(trifluoromethylsulfonyl)phenyl]-2-azaspiro[3.3]heptane; 4-methylbenzenesulfonic Acid

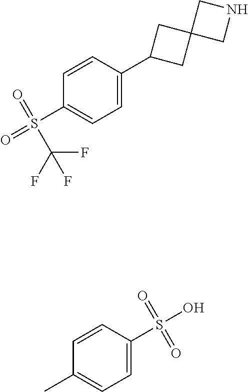 [SMILES: O=S(=O)(c1ccc(C2CC3(CNC3)C2)cc1)C(F)(F)F ; Cc1ccc(S(=O)(=O)O)cc1]

A solution of 6-(4-triflylphenyl)-2-azaspiro[3.3]heptane-2-carboxylic acid tert-butyl ester (313 mg. 0.772 mmol) and p-toluenesulfonic acid monohydrate (162 mg, 0.849 mmol) was stirred at reflux in ethyl acetate (4.26 mL) for 4 h. The crude was filtered and the solid phase was washed with EtOAc and diethylether to afford the title compound as a white solid (assumed purity 95%), which was used directly without further purification. MS (ESI): m/z=306.2 [M−TsOH+H]⁺

### Step a) 6-(4-triflylphenyl)-2-azaspiro[3.3]heptane-2-carboxylic acid tert-butyl Ester

To a 25 mL Radley tube equipped with a stir bar and argon flushed, was added tert-butyl 6-iodo-2-azaspiro[3.3]heptane-2-carboxylate (CAS: 2059140-61-1) (500 mg, 1.55 mmol), (IR[DF(CF₃)PPY]₂(DTBPY))PF₆ (17.4 mg, 0.015 mmol) photocatalyst, TRIS(TRIMETHYLSILYL)SILANE (403 mg, 500 μL, 1.62 mmol), anhydrous sodium carbonate (328 mg, 3.09 mmol) and 1-bromo-4-[(trifluoromethyl) sulfonyl]benzene (CAS: 312-20-9) (492 mg, 1.7 mmol). DME (14.7 mL) was added and the mixture was stirred for 5 min with argon bubbling through the mixture. The vial was sealed.

To a separate vial (flushed with argon) was added 4,4′-DI-TERT-BUTYL-2,2′-BIPYRIDINE (4.15 mg, 0.015 mmol) and NICKEL(II) CHLORIDE ETHYLENE GLYCOL DIMETHYL ETHER COMPLEX (3.4 mg, 0.015 mmol) then DME (737 μL) was added. The precatalyst vial was sealed, purged with argon again. The precatalyst vial was sonicated for 5 min and then poured into the reaction vessel. The reaction was stirred and irradiated with a 465 nm lamp, under argon atmosphere for 14 h. The sodium carbonate was filtered off, washed with ethylacetate and the filtrate was evaporated. The crude was absorbed with Isolute HM-N, dried and purified by flash chromatography, eluting with heptane/EtOAc 0 to 30% to afford the title compound as white solid (313 mg, 47.4%, 95% purity) which was used directly for the next step without further purification. MS (ESI): m/z=350.0 [M−tBu+H]⁺

In analogy to Example B.28, the following building blocks were generated using the relevant (hetero) aryl bromide building block for the photochemical coupling in Step a. In some cases, alternative salts (e.g. trifluoroacetate, ditosylate, hydrochloride) were also used.

| | | | | MS, |
|---|---|---|---|---|
| | | | Building | ESI: |
| Ex. | Structure | Systematic Name | Blocks | m/z |
| B.38 | 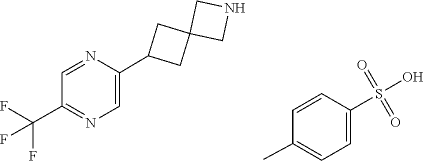 [SMILES: FC(F)(F)c1cnc(C2CC3(CNC3)C2)cn1 ; Cc1ccc(S(=O)(=O)O)cc1] | 6-[5- (trifluoromethyl) pyrazin-2-yl]-2- azaspiro[3.3]heptane; 4- methylbenzenesulfonic acid | 2-bromo- 5- (trifluoro- methyl) pyrazine | 244.1 [M − TsOH + H]⁺ |
| B.49 | 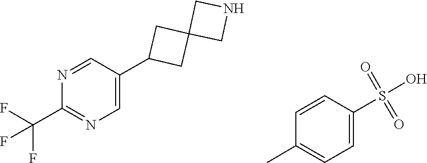 [SMILES: FC(F)(F)c1ncc(C2CC3(CNC3)C2)cn1 ; Cc1ccc(S(=O)(=O)O)cc1] | 6-[2- (trifluoromethyl) pyrimidin-5-yl]-2- azaspiro[3.3]heptane; 4- methylbenzenesulfonic acid | 5-bromo- 2- (trifluoro- methyl) pyrimidine | 244.2 [M − TsOH + H]⁺ |
| B.324 | 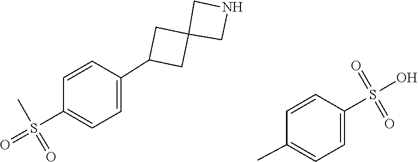 [SMILES: CS(=O)(=O)c1ccc(C2CC3(CNC3)C2)cc1 ; Cc1ccc(S(=O)(=O)O)cc1] | 6-(4- methylsulfonylphenyl)- 2-azaspiro[3.3]heptane; 4- methylbenzenesulfonic acid | 4- bromophenyl methyl sulfone | 252.1 [M − TsOH + H]⁺ |
| B.328 | 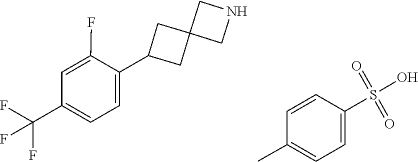 [SMILES: Fc1cc(C(F)(F)F)ccc1C1CC2(CNC2)C1 ; Cc1ccc(S(=O)(=O)O)cc1] | 6-[2-fluoro-4- (trifluoromethyl)phenyl 1-2- azaspiro[3.3]heptane; 4- methylbenzenesulfonic acid | 4-bromo- 3- fluorobenzotrifluoride | 260.0 [M − TsOH + H]⁺ |
| B.370 | 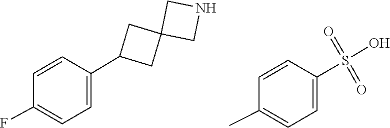 | 6-(4-fluorophenyl)-2- azaspiro[3.3]heptane; 4- methylbenzenesulfonic acid | 4- bromofluorobenzene | 192.1 [M − TsOH + H]⁺ |
| B.371 | 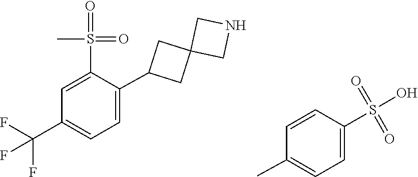 [SMILES: CS(=O)(=O)c1cc(C(F)(F)F)ccc1C1CC2(CNC2)C1 ; Cc1ccc(S(=O)(=O)O)cc1] | 6-[2-methylsulfonyl-4- (trifluoromethyl)phenyl 1-2- azaspiro[3.3]heptane; 4- methylbenzenesulfonic acid | 1-bromo- 2- methylsulfonyl- 4- (trifluoromethyl) benzene (CAS: 1215205-98-3) | 192.1 [M − TsOH + H]⁺ |

### Example B.325

### 6-(5-fluoro-3-pyridyl)-2-azaspiro[3.3]heptane; 4-methylbenzenesulfonic Acid

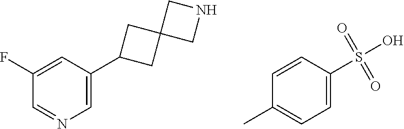

The solution of tert-butyl 6-(5-fluoro-3-pyridyl)-2-azaspiro[3.3]heptane-2-carboxylate (4.0 g, 13.68 mmol) and p-toluenesulfonic acid monohydrate (6.51 g, 34.21 mmol) in EtOAc (150 mL) was stirred at 25° C. for 18 h. Then RM was evaporated and obtained residue (as an oil) was stirred with TBME (150 mL) for 6 h. The obtained precipitate was filtered, washed with TBME and dried to give the title compound (5.66 g, 10.6 mmol, 73.2% yield) as white solid. MS (ESI): m/z=193.2 [M−TsOH+H]⁺

### Step a) tert-butyl 6-(5-fluoro-3-pyridyl)-2-azaspiro[3.3]heptane-2-carboxylate

To the stirred mixture of 5-fluoropyridine-3-boronic acid (CAS: 872041-86-6) (6.1 g, 43.3 mmol), (1S,2S)-2-aminocyclohexanol (249 mg, 2.17 mmol), Nickel(II) iodide (677 mg, 2.17 mmol) in iPrOH (140 mL) under Argon atmosphere, 2 M sodium bis(trimethylsilyl)amide solution in THF (21.7 mL, 43.3 mmol) was added via syringe at RT. Then RM was stirred for 10 min at ambient temperature, before tert-butyl 6-iodo-2-azaspiro[3.3]heptane-2-carboxylate (CAS: 2059140-61-1) (7.0 g, 21.7 mmol) was added. The reaction mixture was refluxed for 4 h and then stirred overnight at RT. The obtained mixture was filtered through SiO₂ and filter-cake washed with IPA. The filterate was evaporated and residue was partioned between TBME and water. The organic layer was dried over Na₂SO₄ and evaporated in vacuum. The obtained crude product was purified with flash column chromatography to give the title compounds (3.95 g, 13.5 mmol, 59.3% yield) as white solid. MS (ESI): m/z=293.2 [M−TsOH+H]⁺

### Example B.29

### N-(2-azaspiro[3.3]heptan-6-ylmethyl)-1-(trifluoromethyl)cyclopropanamine;4-methylbenzenesulfonic Acid

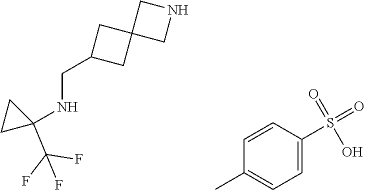 [SMILES: FC(F)(F)C1(NCC2CC3(CNC3)C2)CC1 ; Cc1ccc(S(=O)(=O)O)cc1]

p-toluenesulfonic acid monohydrate (1024 mg, 5.38 mmol) was added to a stirred solution of tert-butyl 6-[[1-(trifluoromethyl)cyclopropyl]amino methyl]-2-azaspiro[3.3]heptane-2-carboxylate (600 mg, 1.79 mmol) in acetonitrile (20 mL). The reaction mixture was stirred for 16 h. The solvent was evaporated under reduced pressure and the residue was triturated with MTBE to give the title compound (668 mg, 1.15 mmol, 64% yield) as a white solid. MS (ESI): m/z=235.2 [M−TsOH+H]⁺

### Step a) tert-butyl 6-[[1-(trifluoromethyl)cyclopropyl]carbamoyl]-2-azaspiro[3.3]heptane-2-carboxylate

To a stirred solution of 2-tert-butoxy carbonyl-2-azaspiro[3.3]heptane-6-carboxylic acid (CAS: 1211526-53-2) (2.0 g, 8.29 mmol) and 1-(trifluoromethyl)cyclopropanamine hydrochloride (CAS: 112738-67-7) (1340 mg, 8.29 mmol) in DMF (5 mL) were added O-(7-azabenzotriazol-1-yl)-N,N,N′,N′-tetramethyluronium hexafluorophosphate (3782 mg, 9.95 mmol) and N,N-diisopropylethylamine (5.05 mL, 29.0 mmol). The mixture was stirred overnight at room temp. and then poured onto water and extracted with EtOAc (2×50 mL). The combined organic fractions were washed three times with water, dried under anhydrous sodium sulfate, and the solvent was removed under vacuum. The residue was triturated with 25 ml of MTBE to give the title compound (1.7 g, 4.88 mmol, 59% yield) as white solid. MS (ESI): m/z=347.2 [M−H]⁻

### Step b) tert-butyl 6-[[[1-(trifluoromethyl)cyclopropyl]amino]methyl]-2-azaspiro[3.3]heptane-2-carboxylate

Tert-butyl 6-[[1-(trifluoromethyl)cyclopropyl]carbamoyl]-2-azaspiro[3.3]heptane-2-carboxylate (1.1 g, 3.16 mmol) was dissolved in THF (30 mL). Borane-methyl sulfide complex (0.48 g. 6.32 mmol) was added at 0 C. The reaction mixture was stirred at reflux for 6 h and then cooled to 0 C and quenched with the drop-wise addition of methanol (5 mL) and then concentrated in vacuo.

The residue was diluted with brine and then extracted with EtOAc (3 times). The organic layers were combined, dried over anhydrous sodium sulfate, filtered and concentrated to afford the title compound (600 mg, 1.79 mmol, 57% yield) as colorless oil. MS (ESI): m/z=279.0 [M−tBu+H]⁺

### Example B.30

### N-(2-azaspiro[3.3]heptan-6-yl)-3-(trifluoromethyl)benzenesulfonamide; trifluoroacetic Acid

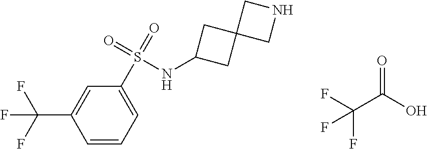 [SMILES: O=S(=O)(NC1CC2(CNC2)C1)c1cccc(C(F)(F)F)c1 ; O=C(O)C(F)(F)F]

To a solution of 6-[[3-(trifluoromethyl)phenyl]sulfonylamino]-2-azaspiro[3.3]heptane-2-carboxylic acid tert-butyl ester (1245 mg, 2.96 mmol) in dichloromethane (8 mL) was added TFA (3.38 g, 2.28 mL, 29.6 mmol) and the reaction mixture was then stirred at room temp for 18 h. Volatiles were removed in vacuo to yield 1910 mg of the crude title compound, purity of roughly 65% and major contaminant excess of TFA, which was used without further purification. MS (ESI): m/z=321.1 [M−TFA+H]⁺

### Step a) 6-[[3-(trifluoromethyl)phenyl]sulfonylamino]-2-azaspiro[3.3]heptane-2-carboxylic Acid Tert-Butyl Ester

To a solution of 6-amino-2-azaspiro[3.3]heptane-2-carboxylic acid tert-butyl ester (750 mg, 3.53 mmol) in dichloromethane (15 mL) cooled down to 0° C. was added DIPEA (685 mg, 926 μL, 5.3 mmol) and 3-(trifluoromethyl)benzenesulfonyl chloride (907 mg, 3.71 mmol) after which the reaction mixture was stirred at 0° C. for 30 min and at r.t for 1 h. The reaction mixture was poured into a separating funnel containing dichloromethane and aq. sol. Na₂CO₃ μM. The organic phase was collected and the aqueous phase was back-extracted with dichloromethane. The combined organic phases were dried over sodium sulfate and evaporated down to dryness. The crude material was purified by flash chromatography with an eluent mixture of heptane and ethyl acetate (10% to 90%) to yield 775 mg of the title compound. MS (ESI): m/z=365.1 [M−tBu+H]⁺

### Example B.31

### N-[6-(trifluoromethyl)pyridazin-3-yl]-2-azaspiro[3.3]heptan-6-amine; trifluoroacetic Acid

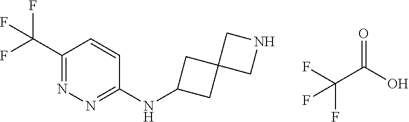 [SMILES: FC(F)(F)c1ccc(NC2CC3(CNC3)C2)nn1 ; O=C(O)C(F)(F)F]

To a solution of 6-[[6-(trifluoromethyl)pyridazin-3-yl]amino]-2-azaspiro[3.3]heptane-2-carboxylic acid tert-butyl ester (708 mg, 1.94 mmol) in dichloromethane (8 mL) was added TFA (2.21 g, 1.49 mL, 19.4 mmol) and the reaction mixture was stirred at r.t for 18 h. Volatiles were removed in vacuo to yield 1310 mg of the crude title compound, purity roughly 55% with major contaminant excess TFA, which was used without further purification. MS (ESI): m/z=259.1 [M−TFA+H]⁺

### Step a) 6-[[6-(trifluoromethyl)pyridazin-3-yl]amino]-2-azaspiro[3.3]heptane-2-carboxylic Acid Tert-Butyl Ester

To a solution of 6-amino-2-azaspiro[3.3]heptane-2-carboxylic acid tert-butyl ester (832 mg, 3.92 mmol) in N,N-dimethylformamide (12 mL) was added DIPEA (690 mg, 932 μL, 5.34 mmol) and 3-chloro-6-(trifluoromethyl)pyridazine (650 mg, 3.56 mmol) after which the reaction mixture was stirred at 80° C. for 18 h. Volatiles were removed in vacuo and the crude residue was partitioned between ethyl acetate and sat. aq. NH₄Cl solution. The organic phase was collected and the aqueous phase was back-extracted with ethyl acetate. The combined organic phases were dried over sodium sulfate and evaporated down to dryness. The crude material was purified by flash chromatography, using an eluent mixture of dichloromethane and methanol (0% to 10%) to yield 708 mg of the title compound. MS (ESI): m/z=359.2 [M+H]⁺

### Example B.32

### 6-[[4-(trifluoromethyl) pyrazol-1-yl]methyl]-2-azaspiro[3.3]heptane; 4-methylbenzenesulfonic Acid

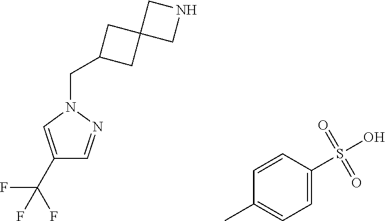 [SMILES: FC(F)(F)c1cnn(CC2CC3(CNC3)C2)c1 ; Cc1ccc(S(=O)(=O)O)cc1]

A solution of tert-butyl 6-[[4-(trifluoromethyl) pyrazol-1-yl]methyl]-2-azaspiro[3.3]heptane-2-carboxylate (675 mg, 1.95 mmol) and p-toluenesulfonic acid (404 mg, 2.35 mmol) in EtOAc (6 mL) was stirred at 80° C. for 12 h. The mixture was concentrated under vacuum to give a residue. To the residue was added deionized water and the mixture was lyophilized to give the title compound (794 mg, 96% yield) as a white solid. MS (ESI): m/z=246.2 [M−TsOH+H]⁺

### Step a) tert-butyl 6-[[4-(trifluoromethyl) pyrazol-1-yl]methylene]-2-azaspiro[3.3]heptane-2-carboxylate

To a solution of 4-(trifluoromethyl)-1H-pyrazole (2435 mg, 17.9 mmol), tert-butyl 6-[(4,4,5,5-tetramethyl-1,3,2-dioxaborolan-2-yl)methylene]-2-azaspiro[3.3]heptane-2-carboxylate (Example B.1, Step a)) (2000 mg, 5.97 mmol) and pyridine (1.45 mL, 17.9 mmol) in DMSO (80 mL) was added copper diacetate (2380 mg, 11.9 mmol) under O₂ atmosphere, then stirred at 100° C. for 12 h under O₂ (balloon) condition. The aqueous phase was extracted with ethyl acetate (200 mL×3). The combined organic phase was washed with brine (200 mL×3), dried with anhydrous Na₂SO₄, filtered and concentrated in vacuum. The residue was purified by flash silica gel chromatography (eluent of 0 to 30% ethyl acetate/petroleum ether) to give a crude product which was purified by reversed-phase HPLC (0.1% FA condition) to give the title compound (640 mg, 31% yield) as a brown solid. MS (ESI): m/z=288.1 [M−tBu+H]⁺

### Step b) tert-butyl 6-[[4-(trifluoromethyl) pyrazol-1-yl]methyl]-2-azaspiro[3.3]heptane-2-carboxylate

To a solution of tert-butyl 6-[[4-(trifluoromethyl) pyrazol-1-yl]methylene]-2-azaspiro[3.3]heptane-2-carboxylate (690 mg, 2.01 mmol) in EtOAc (7 mL) was added wet Pd/C (230 mg, 0.200 mmol), the mixture was stirred at 25° C. under H₂ atmosphere (balloon) for 2 h. The mixture was then filtered and the filtrate was concentrated to give the title compound (690 mg, 99% yield) as yellow solid. MS (ESI): m/z=346.1 [M+H]⁺

In analogy to Example B.32, the following building blocks were generated using the relevant (hetero) aryl building block for the Chan Lam-type coupling in Step 1. In some cases, alternative salts (e.g. trifluoroacetate, ditosylate, hydrochloride) were also used.

| | | | | MS, |
|---|---|---|---|---|
| | | | Building | ESI: |
| Ex. | Structure | Systematic Name | Blocks | m/z |
| B.36 | 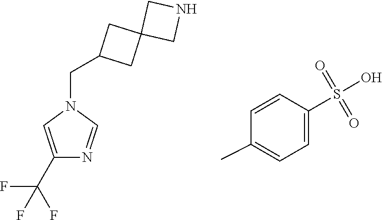 [SMILES: FC(F)(F)c1cn(CC2CC3(CNC3)C2)cn1 ; Cc1ccc(S(=O)(=O)O)cc1] | 6-[]4- (trifluoromethyl) imidazol- 1-yl]methyl]-2- azaspiro[3.3]heptane; 4- methylbenzenesulfonic acid | 4- (trifluoromethyl)- 1H- imidazole (CAS: 33468- 69-8) | 246.3 [M − TsOH + H]⁺ |
| B.41 | 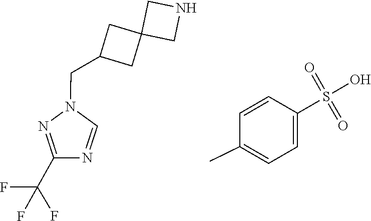 [SMILES: FC(F)(F)c1ncn(CC2CC3(CNC3)C2)n1 ; Cc1ccc(S(=O)(=O)O)cc1] | 6-[[3-(trifluoromethyl)- 1,2,4-triazol-1- yl]methyl]-2- azaspiro[3.3]heptane; 4- methylbenzenesulfonic acid | 3- (trifluoromethyl)- 1H-1,2,4- triazole (CAS: 60406- 75-9) | 247.1 [M − TsOH + H]⁺ |

### Example B.33

### 6-(3-cyclopropyl-1,2,4-triazol-1-yl)-2-azaspiro[3.3]heptane; trifluoroacetic Acid

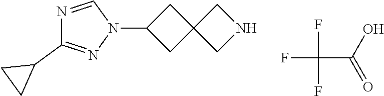 [SMILES: c1nc(C2CC2)nn1C1CC2(CNC2)C1 ; O=C(O)C(F)(F)F]

To a solution of tert-butyl 6-(3-cyclopropyl-1,2,4-triazol-1-yl)-2-azaspiro[3.3]heptane-2-carboxylate (6.00 g, 19.7 mmol) in DCM (120 mL) was added TFA (46.2 g, 405 mmol, 30 mL) at 25° C. The mixture was stirred at 30° C. for 16 h. The reaction mixture was filtered and concentrated under reduced pressure to give a residue. The crude product was used into the next step without further purification. The title compound (14.0 g, crude) was used in the next step without further purification. MS (ESI): m/z=205.2 [M+H]⁺

### Step a) tert-butyl 6-methylsulfonyloxy-2-azaspiro[3.3]heptane-2-carboxylate

To a solution of tert-butyl 6-hydroxy-2-azaspiro[3.3]heptane-2-carboxylate (CAS: 1147557-97-8) (10.0 g, 46.9 mmol) in DCM (200 mL) was added TEA (7.12 g, 70.3 mmol, 9.79 mL) and MsCl (6.90 g, 60.2 mmol, 4.66 mL) dropwise at 0° C., The mixture was stirred at 30° C. for 2 h. The reaction mixture was quenched by addition of aq NaHCO₃ solution (200 mL), and then extracted with DCM (300 mL×2). The combined organic layers were dried over Na₂SO₄, filtered and concentrated under reduced pressure to give a residue (13.5 g crude, 46.3 mmol, 98.8% yield), which was used into the next step without further purification. MS (ESI): m/z=236.2 [M+H]⁺

### Step b) tert-butyl 6-(3-cyclopropyl-1,2,4-triazol-1-yl)-2-azaspiro[3.3]heptane-2-carboxylate

To a solution of tert-butyl 6-methylsulfonyloxy-2-azaspiro[3.3]heptane-2-carboxylate (12.0 g, 41.2 mmol, 90.0% purity) in ACN (200 mL) was added 3-cyclopropyl-1H-1,2,4-triazole (CAS: 1211390-33-8) (4.50 g, 41.2 mmol) and Cs₂CO₃ (26.8 g, 82.4 mmol) at 25° C. The mixture was stirred at 100° C. for 16 h. The reaction mixture was filtered and concentrated under reduced pressure to give a residue. The residue was further separated by SFC to obtain the title compound (6.77 g, 22.2 mmol, 54.0% yield) as a brown solid. MS (ESI): m/z=305.2 [M+H]⁺

### Example B.37

### 7-[[1-(trifluoromethyl)cyclopropyl]methoxy]-2-azaspiro[3.5]nonane; 4-methylbenzenesulfonic Acid

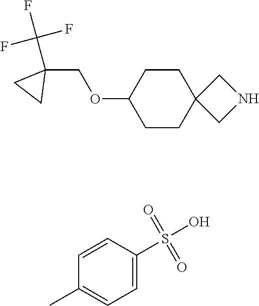 [SMILES: FC(F)(F)C1(COC2CCC3(CC2)CNC3)CC1 ; Cc1ccc(S(=O)(=O)O)cc1]

To a solution of 7-[[1-(trifluoromethyl)cyclopropyl]methoxy]-2-azaspiro[3.5]nonane-2-carboxylic acid tert-butyl ester (675 mg, 1.86 mmol) in isopropyl acetate (14 mL) was added p-toluenesulfonic acid monohydrate (424 mg, 2.23 mmol). The mixture was stirred at 80° C. for 5 h. The reaction mixture was concentrated in vacuo. Et₂O was added, and the mixture filtered through sintered glass. The white solid was washed twice with Et₂O and dried in vacuo to afford the title compound (752 mg, 88%) as a white solid MS (ESI): m/z=264.4 [M+H]⁺

### Step a) 7-[[1-(trifluoromethyl)cyclopropyl]methoxy]-2-azaspiro[3.5]nonane-2-carboxylic Acid Tert-Butyl Ester

To a solution of 7-hydroxy-2-azaspiro[3.5]nonane-2-carboxylic acid tert-butyl ester (CAS: 1363383-18-9) (0.600 g, 2.49 mmol) in N,N-dimethylformamide, extra dry (6 mL) was added sodium hydride (114 mg, 2.86 mmol). The mixture was stirred at room temp. for 1 h. 1-(bromomethyl)-1-(trifluoromethyl)cyclopropane (505 mg, 2.49 mmol) was added. The mixture was stirred at 80° C. for 16 h.

The reaction mixture was poured into EtOAc and washed with water and brine. The organic layer was dried over Na₂SO₄ and concentrated in vacuo. The crude material was purified by flash chromatography (eluting with 0% to 40% AcOEt in heptane) to afford the title compound (681 mg, 68%). MS (ESI): m/z=308.1 [M−tBu+H]⁺

### Example B.39

### 6-[[1-(trifluoromethyl)cyclopropyl]methoxy]-2-azaspiro[3.3]heptane; 4-methylbenzenesulfonic Acid

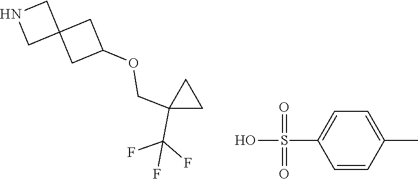 [SMILES: FC(F)(F)C1(COC2CC3(CNC3)C2)CC1 ; Cc1ccc(S(=O)(=O)O)cc1]

A solution of 6-[[1-(trifluoromethyl)cyclopropyl]methoxy]-2-azaspiro[3.3]heptane-2-carboxylic acid tert-butyl ester (633 mg, 1.89 mmol) in ethyl acetate (20 mL) was treated with p-toluenesulfonic acid monohydrate (366 mg, 1.93 mmol), at 23° C. The mixture was then heated to 80° C. for 18 h, before being cooled down to 23° C. and evaporated, to give 6-[[1-(trifluoromethyl)cyclopropyl]methoxy]-2-azaspiro[3.3]heptane 4-methylbenzenesulfonate (769 mg, 95.0%) as light yellow solid. MS (ESI): m/z=236.2 [M−C₇H₈O₃S+H]⁺

### Step a) 6-[[1-(trifluoromethyl)cyclopropyl]methoxy]-2-azaspiro[3.3]heptane-2-carboxylic Acid Tert-Butyl Ester

A solution of 6-hydroxy-2-azaspiro[3.3]heptane-2-carboxylic acid tert-butyl ester (500 mg, 2.34 mmol) in N,N-dimethylformamide, extra dry (10 mL) was treated with 1-(bromomethyl)-1-(trifluoromethyl)cyclopropane (476 mg, 2.34 mmol), at 23° C. under Ar. The mixture was stirred for another 30 min at this temperature, before being heated to 80° C. and stirred for 21.5 h. The mixture was then cooled down to 23° C., diluted with EtOAc, and the organic layer was washed with 1 M NaHCO₃ solution (1×), water (2×), and brine (1×). The organic layer was then dried over Na₂SO₄, filtered, and evaporated, to give the title compound (633 mg, 73%) as a crude colorless oil which was used directly without further purification. MS (ESI): m/z=280.2 [M+H−tBu]⁺

### Example B.40

### N-[1-(trifluoromethyl)cyclopropyl]-2-azaspiro[3.3]heptan-6-amine; 4-methylbenzenesulfonic Acid

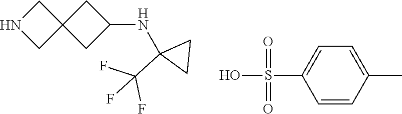

A solution of tert-butyl 6-[[1-(trifluoromethyl)cyclopropyl]amino]-2-azaspiro[3.3]heptane-2-carboxylate (880 mg, 2.75 mmol) and p-toluenesulfonic acid monohydrate (1568 mg, 8.24 mmol) in EtOAc (30 mL) was heated at reflux for 3 h, then cooled to room temp. and stirred for another 16 h. The obtained precipitate was collected by filtration, washed with EtOAc (15 mL) and dried under vacuum to provide the title compound (1335 mg, 84% yield) as a white solid. MS (ESI): m/z=221.2 [M−TsOH+H]⁺

### Step a) tert-butyl 6-[[1-(trifluoromethyl)cyclopropyl]amino]-2-azaspiro[3.3]heptane-2-carboxylate

To a stirred mixture of 1-(trifluoromethyl)cyclopropanamine hydrochloride (918 mg, 5.68 mmol) and tert-butyl 6-oxo-2-azaspiro[3.3]heptane-2-carboxylate (CAS: 1181816-12-5) (1.2 g, 5.68 mmol) in DCM (70 mL), triethylamine (2.38 mL, 17.0 mmol) was added. The reaction mixture was stirred at room temp. for 20 min. Then sodium triacetoxy borohydride (2410 mg, 11.4 mmol) was added to the solution in one portion and obtained mixture was stirred for 18 h at 23° C. Then reaction mixture was diluted with DCM (50 mL), and 5% NaHCO₃ (aq. sol.) (80 mL) was added. The organic phase was separated, and the aqueous layer was extracted with DCM (50 mL). The organic layers were combined, washed with brine (50 mL), dried over Na₂SO₄ and evaporated. Purification by FC (SiO₂; PE/MTBE) gave the title compound (70 mg, 3.7% yield) as a white solid. MS (ESI): m/z=321.2 [M+H]⁺

### Example B.42

### N-[1-(trifluoromethyl)cyclopropyl]-2-azaspiro[3.3]heptane-6-carboxamide; hydrochloride

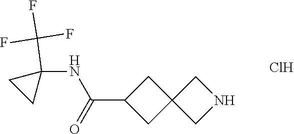 [SMILES: O=C(NC1(C(F)(F)F)CC1)C1CC2(CNC2)C1 ; Cl]

To a solution of benzyl 6-[[1-(trifluoromethyl)cyclopropyl]carbamoyl]-2-azaspiro[3.3]heptane-2-carboxylate (200 mg, 0.520 mmol) in methanol (10 mL) was added palladium (10% on carbon) (0.02 mL, 0.190 mmol). The reaction mixture was stirred for 24 h at room temperature under a hydrogen atmosphere. The solids were removed by filtration and the filtrate was concentrated in vacuo. Then the residue was dissolved in THF (10 mL) and treated with 4 N HCl in dioxane, and stirred for 10 min at 23° C. The precipitate was collected by filtration to afford the title compound (60 mg, 40% yield) as a white solid. MS (ESI): m/z=249.2 [M+H]⁺

### Step a) benzyl 6-[[1-(trifluoromethyl)cyclopropyl]carbamoyl]-2-azaspiro[3.3]heptane-2-carboxylate

To a stirred solution of 2-benzyloxycarbonyl-2-azaspiro[3.3]heptane-6-carboxylic acid (CAS: 1291487-33-6) (1.3 g, 4.72 mmol) and 1-(trifluoromethyl)cyclopropanamine hydrochloride (0.76 g, 4.72 mmol) in DMF (10 mL) were added HATU (2.33 g, 6.14 mmol) and TEA (2.3 mL, 16.5 mmol). The mixture was stirred overnight at 23° C. and then poured onto water and extracted with EtOAc (2×100 mL). The combined organic fractions were washed three times with water, dried under anhydrous sodium sulfate, and the solvent was removed under vacuum. The residue was triturated with 50 mL of MTBE to give the title compound (1 g, 55% yield) as a white solid. MS (ESI): m/z=383.2 [M+H]⁺

### Example B.50

### 6-[6-(trifluoromethyl)-3-pyridyl]-2-azaspiro[3.3]heptane; 4-methylbenzenesulfonic Acid

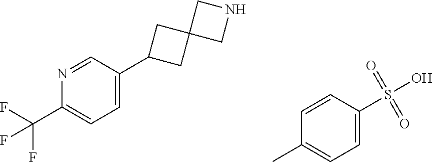 [SMILES: FC(F)(F)c1ccc(C2CC3(CNC3)C2)cn1 ; Cc1ccc(S(=O)(=O)O)cc1]

The mixture of tert-butyl 6-[6-(trifluoromethyl)-3-pyridyl]-2-azaspiro[3.3]heptane-2-carboxylate (1450 mg, 4.24 mmol) and p-toluenesulfonic acid (1605 mg, 9.32 mmol) in ethyl acetate (10 mL) was stirred at 80° C. for 16 h. The reaction mixture was filtered and the cake was concentrated to give the title compound (2110 mg, 3.6 mmol, 84% yield) as a off-white solid. MS (ESI): m/z=243.3 [M−TsOH+H]⁺

### Step a) tert-butyl 6-[6-(trifluoromethyl)-3-pyridyl]-2-azaspiro[3.3]heptane-2-carboxylate

Two batches were set up in parallel. Tert-butyl 6-iodo-2-azaspiro[3.3]heptane-2-carboxylate (1.0 g, 3.09 mmol), 2-trifluoromethylpyridine-5-boronic acid (1180 mg, 6.19 mmol), Sodium bis(trimethylsilyl)amide in THF (6.19 mL, 6.19 mmol), trans-2-aminocyclohexanol hydrochloride (28.2 mg, 0.190 mmol) and nickel(II) iodide (58.0 mg, 0.190 mmol) were taken up into a microwave tube in 2-propanol (10 mL). The sealed tube was heated at 110° C. for 2.5 h under microwave. The reaction was quenched by H₂O slowly. The residue was purified by flash silica gel chromatography (eluent of 0 to 20% ethyl acetate/petroleum ether gradient) to give the title compound (1.5 g, 4.38 mmol, 71% yield) as yellow solid. MS (ESI): m/z=287.2 [M−tBu+H]⁺

### Example B.51

### 6-[3-(trifluoromethyl) azetidin-1-yl]-2-azaspiro[3.3]heptane; 4-methylbenzenesulfonic Acid

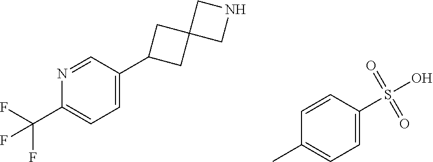 [SMILES: FC(F)(F)c1ccc(C2CC3(CNC3)C2)cn1 ; Cc1ccc(S(=O)(=O)O)cc1]

A solution of tert-butyl 6-[3-(trifluoromethyl) azetidin-1-yl]-2-azaspiro[3.3]heptane-2-carboxylate (550 mg, 1.72 mmol) and p-toluenesulfonic acid monohydrate (980 mg, 5.15 mmol) in EtOAc (50 mL) was heated at reflux for 2 h, then cooled to room temp. and stirred for another 16 h. The precipitate was collected by filtration, washed with ethyl acetate (20 mL) and dried under vacuum to provide the title compound (486 mg, 49% yield) as a white solid. MS (ESI): m/z=221.2 [M−TsOH+H]⁺

### Step a) tert-butyl 6-[3-(trifluoromethyl) azetidin-1-yl]-2-azaspiro[3.3]heptane-2-carboxylate

Tert-butyl 6-oxo-2-azaspiro[3.3]heptane-2-carboxylate (CAS: 1181816-12-5) (800 mg, 3.79 mmol), 3-(trifluoromethyl) azetidine; hydrochloride (CAS: 1221272-90-7) (612 mg, 3.79 mmol), and triethylamine (1.58 mL, 11.4 mmol) were mixed in DCM (40 mL) and stirred for 10 min at room temperature. Then sodium triacetoxyborohydride (1.61 g, 7.57 mmol) was added in one portion, and the reaction mixture was stirred for 18 h at room temperature. Then reaction mixture was diluted with DCM (50 mL), and 5% NaHCO₃ aq. sol. (80 mL) was added. The organic phase was separated, and the aqueous layer was extracted with DCM (50 mL). The organic layers were combined, washed with brine (50 mL), dried over Na₂SO₄ and evaporated. The crude product was purified by HPLC to obtain the title compound (550 mg, 44% yield) as a light yellow solid. MS (ESI): m/z=321.0 [M+H]⁺

In analogy to Example B.51, the following building blocks were generated using the relevant amine building block in Step a).

| | | | | MS, |
|---|---|---|---|---|
| | | | Building | ESI: |
| Ex. | Structure | Systematic Name | Blocks | m/z |
| B.52 | 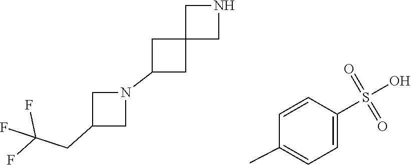 [SMILES: FC(F)(F)CC1CN(C2CC3(CNC3)C2)C1 ; Cc1ccc(S(=O)(=O)O)cc1] | 6-[3-(2,2,2- trifluoroethyl)azetidin- 1-yl]-2- azaspiro[3.3]heptane; 4- methylbenzenesulfonic acid | 3-(2,2,2- trifluoroethyl) azetidine; hydrochloride (CAS: 1394041-81-6) | 235.2 [M − TsOH + H]⁺ |
| B.156 | 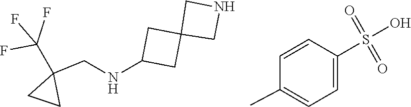 [SMILES: FC(F)(F)C1(CNC2CC3(CNC3)C2)CC1 ; Cc1ccc(S(=O)(=O)O)cc1] | N-[[1- (trifluoromethyl) cyclopropyl]methyl]-2- azaspiro[3.3]heptan-6- amine; 4- methylbenzenesulfonic acid | [1- (trifluoromethyl) cyclopropyl] methanamine; hydrochloride | 235.2 [M − TsOH + H]⁺ |

### Example B.53

### 7-[[4-(trifluoromethylsulfonyl)phenyl]methyl]-2,7-diazaspiro[3.5]nonane; trifluoroacetic Acid

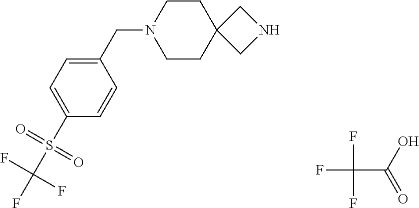 [SMILES: O=S(=O)(c1ccc(CN2CCC3(CC2)CNC3)cc1)C(F)(F)F ; O=C(O)C(F)(F)F]

To a solution of 7-(4-triflylbenzyl)-2,7-diazaspiro[3.5]nonane-2-carboxylic acid tert-butyl ester (360 mg, 0.803 mmol) in isopropyl acetate (6 mL) was added p-toluenesulfonic acid monohydrate (336 mg, 1.77 mmol). The mixture was stirred at 80° C. for 5 h. Et₂O was added, and the mixture filtered through sintered glass. The white solid was washed twice with Et₂O and dried in vacuo to afford the title compound (537 mg, 92%) as white solid. MS (ESI): m/z=349.1 [M−TFA+H]⁺

### Step a) 7-(4-triflylbenzyl)-2,7-diazaspiro[3.5]nonane-2-carboxylic Acid Tert-Butyl Ester

To a solution of 4-triflylbenzaldehyde (CAS: 650-89-5) (316 mg, 1.33 mmol) and 2,7-diazaspiro[3.5]nonane-2-carboxylic acid tert-butyl ester (CAS: 236406-55-6) (0.300 g, 1.33 mmol) in 1,2-dichloroethane (2.5 mL) was added sodium triacetoxy borohydride (309 mg, 1.46 mmol) and acetic acid (159 mg, 152 μL, 2.65 mmol). The mixture was stirred at room temp for 2 h. The reaction mixture was poured into EtOAc:THF 2:1 and washed with NaHCO₃ sat. aq. sol., water and brine. The organic layer was dried over Na₂SO₄ and evaporated. Purification by FC (SiO₂; DCM/MeOH) gave the title compound (364 mg, 58%) as a white solid. MS (ESI): m/z=449.5 [M+H]⁺

In analogy to Example B.53, the following building blocks were generated using the relevant building blocks in Step a).

| | | | | MS, |
|---|---|---|---|---|
| | | | Building | ESI: |
| Ex. | Structure | Systematic Name | Blocks | m/z |
| B.54 | 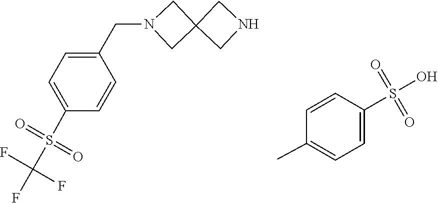 [SMILES: O=S(=O)(c1ccc(CN2CC3(CNC3)C2)cc1)C(F)(F)F ; Cc1ccc(S(=O)(=O)O)cc1] | 2-[[4- (trifluoromethyl- sulfonyl) phenyl]methyl]- 2,6- diazaspiro[3.3]heptane; 4- methyl- benzenesulfonic acid | 4- triflyl- benzaldehyde, tert-Butyl 2,6-diazaspiro [3.3]heptane-2- carboxylate oxalate(2:1) (CAS 1041026- 71-4) | 321.1 [M − TsOH + H]⁺ |
| B.100 | 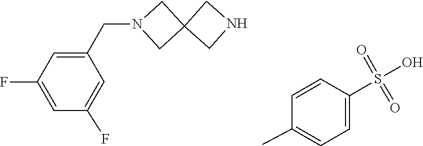 [SMILES: Fc1cc(F)cc(CN2CC3(CNC3)C2)c1 ; Cc1ccc(S(=O)(=O)O)cc1] | 2-[(3,5- difluorophenyl) methyl]-2,6- diazaspiro[3.3] heptane; 4- methylbenzenesulfonic acid | tert-Butyl2,6- diazaspiro[3.3] heptane-2- carboxylate oxalate(2:1); 3,5- difluor- obenzaldehyde | 225.0 [M − TsOH + H]⁺ |
| B.106 | 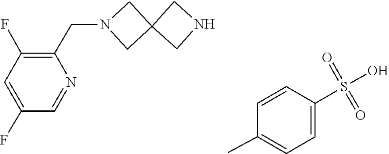 [SMILES: Fc1cnc(CN2CC3(CNC3)C2)c(F)c1 ; Cc1ccc(S(=O)(=O)O)cc1] | 2-[(3,5-difluoro-2- pyridyl)methyl]-2,6- diazaspiro[3.3] heptane; 4- methylbenzenesulfonic acid | tert-Butyl 2,6- diazaspiro[3.3] heptane-2- carboxylate oxalate(2:1); 3,5- difluoropyridine- 2- carbaldehyde (CAS: 780801-58-3) | 226.2 [M − TsOH + H]⁺ |
| B.107 | 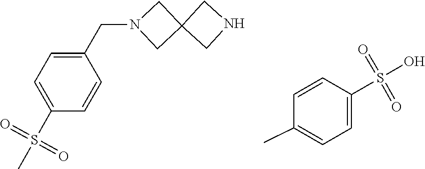 [SMILES: CS(=O)(=O)c1ccc(CN2CC3(CNC3)C2)cc1 ; Cc1ccc(S(=O)(=O)O)cc1] | 2-[(4- methylsulfonylphenyl) methyl]-2,6- diazaspiro[3.3]heptane; di 4- methylbenzenesulfonic acid | tert-Butyl 2,6- diazaspiro[3.3] heptane- 2- carboxylate oxalate(2:1); 4- mesyl- benzaldehyde | 267.1 [M − TsOH + H]⁺ |
| B.108 | 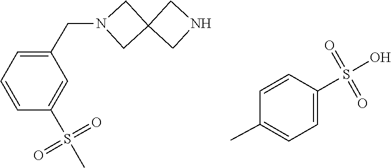 [SMILES: CS(=O)(=O)c1cccc(CN2CC3(CNC3)C2)c1 ; Cc1ccc(S(=O)(=O)O)cc1] | 2-[(3- methylsulfonylphenyl) methyl]-2,6- diazaspiro[3.3]heptane; di 4- methylbenzenesulfonic acid | tert-Butyl 2,6-diazaspiro [3.3]heptane-2- carboxylate oxalate(2:1); 3- mesyl- benzaldehyde | 267.1 [M − TsOH + H]⁺ |
| B.109 | 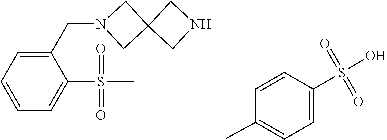 [SMILES: CS(=O)(=O)c1ccccc1CN1CC2(CNC2)C1 ; Cc1ccc(S(=O)(=O)O)cc1] | 2-[(2- methylsulfonylphenyl) methyl]-2,6- diazaspiro[3.3]heptane; di 4- methylbenzenesulfonic acid | tert-Butyl 2,6- diazaspiro[3.3] heptane-2- carboxylate oxalate(2:1); 2- mesyl- benzaldehyde | 267.1 [M − TsOH + H]⁺ |
| B.116 | 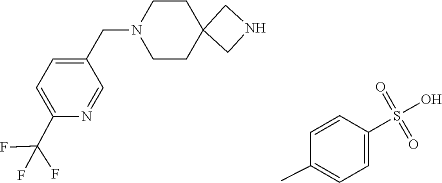 [SMILES: FC(F)(F)c1ccc(CN2CCC3(CC2)CNC3)cn1 ; Cc1ccc(S(=O)(=O)O)cc1] | 7-[[6- (trifluoromethyl)-3- pyridyl]methyl]-2,7- diazaspiro[3.5] nonane; 4- methylbenzenesulfonic acid | 2,7- diazaspiro[3.5] nonane-2- carboxylic acid tert- butyl ester; 6- (trifluoromethyl) nicotinaldehyde | 286.1 [M − TsOH + H]⁺ |
| B.117 | 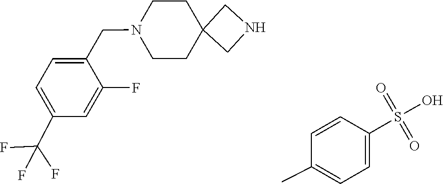 [SMILES: Fc1cc(C(F)(F)F)ccc1CN1CCC2(CC1)CNC2 ; Cc1ccc(S(=O)(=O)O)cc1] | 7-[[2-fluoro-4- (trifluoromethyl) phenyl]methyl]-2,7- diazaspiro[3.5] nonane;4- methylbenzenesulfonic acid | 2,7- diazaspiro[3.5] nonane-2- carboxylic acid tert- butyl ester; 2-fluoro-4- (trifluoromethyl) benzaldehyde | 303.1 [M − TsOH + H]⁺ |
| B.118 | 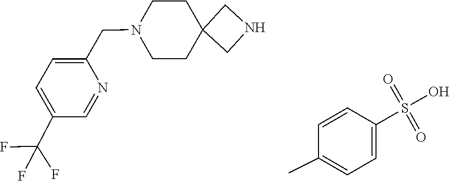 [SMILES: FC(F)(F)c1ccc(CN2CCC3(CC2)CNC3)nc1 ; Cc1ccc(S(=O)(=O)O)cc1] | 7-[[5- (trifluoromethyl)-2- pyridyl]methyl]-2,7- diazaspiro[3.5]nonane; 4- methylbenzenesulfonic acid | 2,7- diazaspiro[3.5] nonane-2- carboxylic acid tert- butyl ester; 5- (trifluoromethyl) picolinaldehyde | 286.1 [M − TsOH + H]⁺ |
| B.119 | 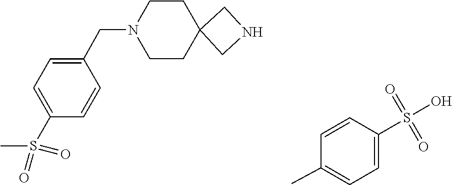 [SMILES: CS(=O)(=O)c1ccc(CN2CCC3(CC2)CNC3)cc1 ; Cc1ccc(S(=O)(=O)O)cc1] | 7-[(4- methylsulfonylphenyl) methyl]-2,7- diazaspiro[3.5]nonane; di 4- methylbenzenesulfonic acid | 2,7-diazaspiro [3.5] nonane-2- carboxylic acid tert- butyl ester; 4- mesylbenzal dehyde | 295.2 [M − TsOH + H]⁺ |
| B.120 | 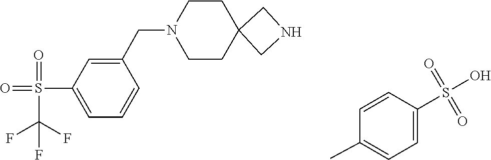 [SMILES: O=S(=O)(c1cccc(CN2CCC3(CC2)CNC3)c1)C(F)(F)F ; Cc1ccc(S(=O)(=O)O)cc1] | 7-[[3- (trifluoromethylsulfonyl) phenyl]methyl]- 2,7- diazaspiro[3.5] nonane; di 4- methylbenzenesulfonic acid | 2,7-diazaspiro [3.5] nonane-2- carboxylic acid tert- butyl ester; 3- triflylbenzal dehyde (CAS: 1274904-33-4) | 349.1 [M − TsOH + H]+ |
| B.121 | 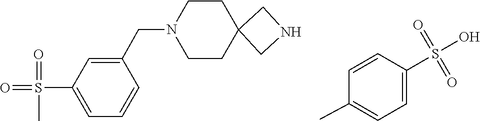 [SMILES: CS(=O)(=O)c1cccc(CN2CCC3(CC2)CNC3)c1 ; Cc1ccc(S(=O)(=O)O)cc1] | 7-[(3- methylsulfonylphenyl) methyl]-2,7- diazaspiro[3.5]nonane; 4- methylbenzenesulfonic acid | 2,7- diazaspiro[3.5] nonane-2- carboxylic acid tert- butyl ester; 3- mesyl- benzaldehyde | 295.2 [M − 2TsOH + H]⁺ |
| B.122 | 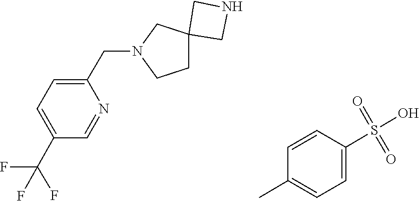 [SMILES: FC(F)(F)c1ccc(CN2CCC3(CNC3)C2)nc1 ; Cc1ccc(S(=O)(=O)O)cc1] | 6-[[5- (trifluoromethyl)-2- pyridyl]methyl]-2,6- diazaspiro[3.4]octane; 4- methylbenzenesulfonic acid | tert-butyl 2,6- diazaspiro [3.4]octane-2- carboxylate; 5- (trifluoromethyl) pyridine-2- carbaldehyde | 272.0 [M − TsOH + H]⁺ |
| B.123 | 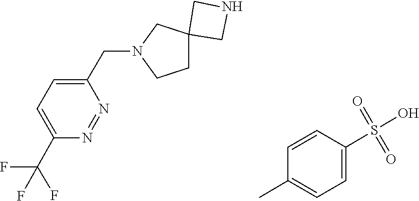 [SMILES: FC(F)(F)c1ccc(CN2CCC3(CNC3)C2)nn1 ; Cc1ccc(S(=O)(=O)O)cc1] | 6-[[6- (trifluoromethyl) pyridazin-3-yl]methyl]- 2,6- diazaspiro[3.4]octane; 4- methylbenzenesulfonic acid | tert-butyl 2,6- diazaspiro[3.4] octane-2- carboxylate; 6- (trifluoromethyl) pyridazine-3- carbaldehyde (CAS: 1245643- 49-5) | 273.0 [M − 2TsOH + H]⁺ |
| B.124 | 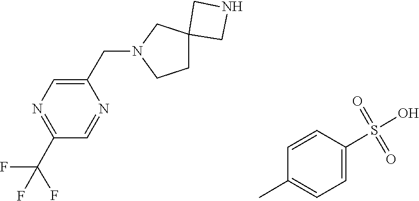 [SMILES: FC(F)(F)c1cnc(CN2CCC3(CNC3)C2)cn1 ; Cc1ccc(S(=O)(=O)O)cc1] | 6-[[5- (trifluoromethyl) pyrazin-2-yl]methyl]- 2,6- diazaspiro[3.4]octane; 4- methylbenzenesulfonic acid | tert-butyl 2,6- diazaspiro[3.4] octane-2- carboxylate; 5- (trifluorome thyl)pyrazine-2- carbaldehyde (CAS: 1196151- 36-6) | 273.2 [M − 2TsOH + H]⁺ |
| B.125 | 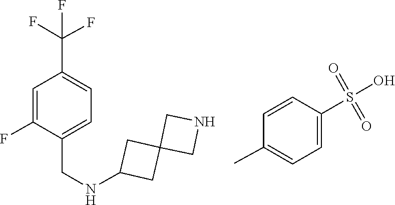 | N-[[2-fluoro-4- (trifluoromethyl) phenyl]methyl]-2- azaspiro[3.3]heptan- 6-amine; 4- methylbenzenesulfonic acid | 6-amino-2- azaspiro[3.3] heptane-2- carboxylic acid tert- butyl ester; 2-fluoro-4- (trifluoromethyl) benzaldehyde | 289.1 [M − TsOH + H]⁺ |
| B.303 | 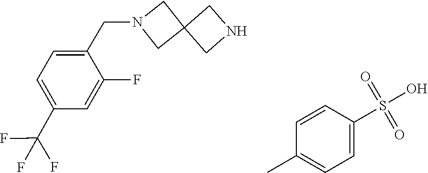 [SMILES: Fc1cc(C(F)(F)F)ccc1CN1CC2(CNC2)C1 ; Cc1ccc(S(=O)(=O)O)cc1] | 2-[[2-fluoro-4- (trifluoromethyl) phenyl]methyl]-2,6- diazaspiro[3.3] heptane; 4- methylbenzenesulfonic acid | tert-Butyl 2,6- diazaspiro[3.3] heptane-2- carboxylate oxalate(2:1) (CAS: 1374659- 01-4); 2-fluoro-4- (trifluoromethyl) benzaldehyde (CAS: 89763-93-9) | 275.2 [M − TsOH + H]⁺ |
| B.326 | 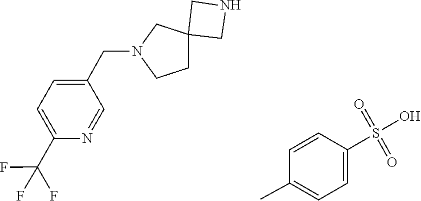 [SMILES: FC(F)(F)c1ccc(CN2CCC3(CNC3)C2)cn1 ; Cc1ccc(S(=O)(=O)O)cc1] | 6-[[6- (trifluoromethyl)-3- pyridyl]methyl]-2,6- diazaspiro[3.4]octane; di 4- methylbenzenesulfonic acid | tert-butyl 2,6- diazaspiro[3.4] octane-2- carboxylate; 6- (trifluoromethyl) pyridine-3- carboxaldehyde | 272.4 [M − 2TsOH + H]⁺ |
| B.347 | 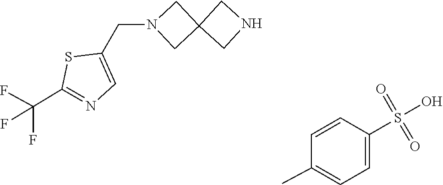 [SMILES: FC(F)(F)c1ncc(CN2CC3(CNC3)C2)s1 ; Cc1ccc(S(=O)(=O)O)cc1] | 5-(2,6- diazaspiro[3.3]heptan- 2-ylmethyl)-2- (trifluoromethyl) thiazole; di 4- methylbenzenesulfonic acid | tert-Butyl2,6- diazaspiro[3.3] heptane-2- carboxylate oxalate(2:1) (CAS: 1374659- 01-4); 2- (trifluoromethyl) thiazole-5- carbaldehyde (CAS: 903130-38-1) | 264.0 [M − 2TsOH + H]⁺ |
| B.350 | 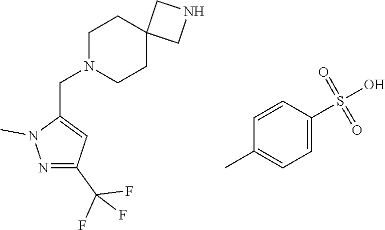 [SMILES: Cn1nc(C(F)(F)F)cc1CN1CCC2(CC1)CNC2 ; Cc1ccc(S(=O)(=O)O)cc1] | 7-[[3- (trifluoromethyl)- 1H-pyrazol-5- yl]methyl]-2,7- diazaspiro[3.5]nonane; di 4- methylbenzenesulfonic acid | 2-(tert- butoxycarbonyl)- 2,7-diazaspiro [3.5] nonane; 2-methyl-5- (trifluoromethyl) pyrazole-3- carbaldehyde (CAS: 1414962-92-7) | 289.1 [M − 2TsOH + H]⁺ |
| B.351 | 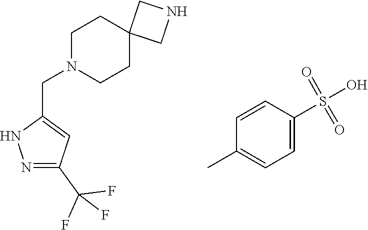 [SMILES: FC(F)(F)c1cc(CN2CCC3(CC2)CNC3)[nH]n1 ; Cc1ccc(S(=O)(=O)O)cc1] | 7-[[3- (trifluoromethyl)- 1H-pyrazol-5- yl]methyl]-2,7- diazaspiro[3.5]nonane; di 4- methylbenzenesulfonic acid | 2-(tert- butoxycarbonyl)- 2,7-diazaspiro [3.5] nonane; 3- (trifluoromethyl)- 1H-pyrazole-5- carbaldehyde (CAS: 591234-14-9) | 275.2 [M − 2TsOH + H]⁺ |
| B.352 | 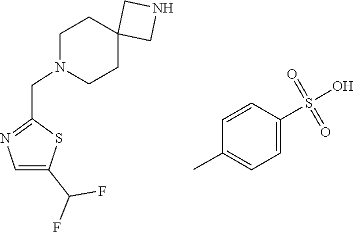 [SMILES: FC(F)c1cnc(CN2CCC3(CC2)CNC3)s1 ; Cc1ccc(S(=O)(=O)O)cc1] | 2-(2,7- diazaspiro[3.5]nonan- 7-ylmethyl)-5- (difluoromethyl) thiazole; 4- methylbenzenesulfonic acid | 2-(tert- butoxycarbonyl)- 2,7-diazaspiro [3.5] nonane; 5- (difluorome thyl)thiazole-2- carbaldehyde (CAS: 1780912- 17-5 ) | 274.0 [M − TsOH + H]⁺ |
| B.362 | 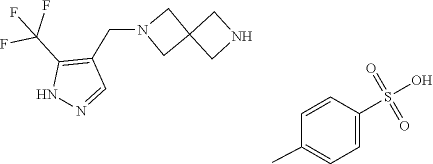 [SMILES: FC(F)(F)c1[nH]ncc1CN1CC2(CNC2)C1 ; Cc1ccc(S(=O)(=O)O)cc1] | 2-[[5- (trifluoromethyl)- 1H-pyrazol-4- yl]methyl]-2,6- diazaspiro[3.3] heptane; 4- methylbenzenesulfonic acid | tert-Butyl 2,6- diazaspiro[3.3] heptane-2- carboxylate oxalate(2:1) (CAS: 1374659-01-4); 5- (trifluoromethyl)- 1H-pyrazole-4- carbaldehyde | 247.0 [M − TsOH + H]⁺ |
| | | | (CAS: | |
| | | | 1001020-14-9) | |
| B.363 | 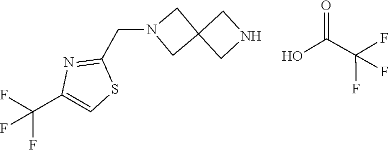 [SMILES: FC(F)(F)c1csc(CN2CC3(CNC3)C2)n1 ; O=C(O)C(F)(F)F] | 2-(2,6- diazaspiro[3.3]heptan- 2-ylmethyl)-4- (trifluoromethyl) thiazole; 2,2,2- trifluoroacetic acid | tert-butyl 2,6- diazaspiro[3.3] heptane-2- carboxylate hydrochloride; 4- (trifluoromethyl) thiazole-2- carbaldehyde (CAS: 354587-75-0) | 264.0 [M − TFA + H]⁺ |
| B.364 | 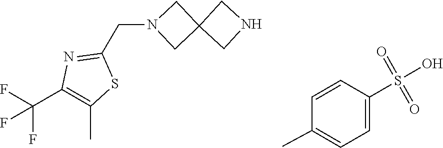 [SMILES: Cc1sc(CN2CC3(CNC3)C2)nc1C(F)(F)F ; Cc1ccc(S(=O)(=O)O)cc1] | 2-(2,6- diazaspiro[3.3]heptan- 2-ylmethyl)- 5-methyl-4- (trifluoromethyl) thiazole; 4- methylbenzenesulfonic acid | tert-butyl 2,6- diazaspiro[3.3] heptane-2- carboxylate hydrochloride; 5- methyl-4- (trifluoromethyl)- 1,3-thiazole-2- carbaldehyde (CAS: 1379099-99-6) | 278.0 [M − TFA + H]⁺ |
| B.365 | 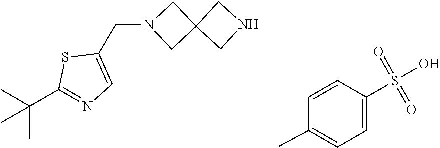 [SMILES: CC(C)(C)c1ncc(CN2CC3(CNC3)C2)s1 ; Cc1ccc(S(=O)(=O)O)cc1] | 2-tert-butyl-5-(2,6- diazaspiro[3.3]heptan- 2-ylmethyl)thiazole; 4- methylbenzenesulfonic acid | tert-butyl 2,6-diazaspiro [3.3] heptane-2- carboxylate; oxalic acid; 2-tert- butylthiazole-5- carbaldehyde (CAS: 1211695-52-1) | 252.0 [M − TsOH + H]⁺ |
| B.375 | 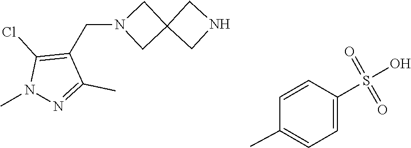 [SMILES: Cc1nn(C)c(Cl)c1CN1CC2(CNC2)C1 ; Cc1ccc(S(=O)(=O)O)cc1] | 2-[(5-chloro-1,3- dimethyl-pyrazol-4- yl)methyl]-2,6- diazaspiro[3.3]heptane; 4- methylbenzenesulfonic acid | 2,6-diazaspiro [3.3]heptane-2- carboxylic acid tert- butyl ester; 5-chloro-1,3- dimethyl- pyrazole-4- carbaldehyde (CAS: 27006-76-4) | 241.1 [M − TsOH + H]⁺ |
| B.380 | 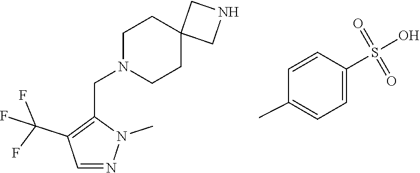 [SMILES: Cn1ncc(C(F)(F)F)c1CN1CCC2(CC1)CNC2 ; Cc1ccc(S(=O)(=O)O)cc1] | 7-[[2-methyl-4- (trifluoromethyl) pyrazol-3-yl]methyl]- 2,7-diazaspiro[3.5] nonane; 4- methylbenzenesulfonic acid | 2-(tert- butoxycarbonyl)- 2,7-diazaspiro [3.5]nonane; 1-methyl-4- (trifluoromethyl)- 1H-pyrazole-5- carbaldehyde (CAS: 1780459-24-6) | 289.2 [M − TsOH + H]⁺ |
| B.385 | 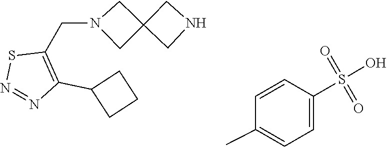 [SMILES: C1CC(c2nnsc2CN2CC3(CNC3)C2)C1 ; Cc1ccc(S(=O)(=O)O)cc1] | 4-cyclobutyl-5-(2,6- diazaspiro[3.3] heptan-2- ylmethyl)thiadiazole; 4- methylbenzenesulfonic acid | tert-butyl 2,6- diazaspiro[3.3] heptane-2- carboxylate hydrochloride; 4-cyclo- butylthiadiazole- 5-carbaldehyde (CAS: 2639443-82-4) | 251.2 [M − TsOH + H]⁺ |
| B.391 | 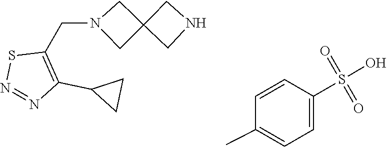 [SMILES: C1CC1c1nnsc1CN1CC2(CNC2)C1 ; Cc1ccc(S(=O)(=O)O)cc1] | 4-cyclopropyl-5-(2,6- diazaspiro[3.3]heptan- 2- ylmethyl)thiadiazole; 4- methylbenzenesulfonic acid | tert-butyl 2,6- diazaspiro[3.3] heptane-2- carboxylate hydrochloride; 4-cyclopropyl thiadiazole- 5-carbaldehyde (CAS: 1000878-41-0) | 237.0 [M − TsOH + H]⁺ |
| B.395 | 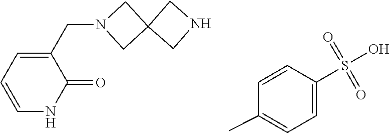 | 3-(2,6- diazaspiro[3.3]heptan- 2-ylmethyl)-1H- pyridin-2-one; 4- methylbenzenesulfonic acid | tert-butyl 2,6- diazaspiro[3.3] heptane-2- carboxylate hydrochloride; | 206.2 [M − TsOH + H]⁺ |

### Example B.55

### 2-[3-(trifluoromethoxy)phenyl]sulfonyl-2,6-diazaspiro[3.3]heptane; trifluoroacetic Acid

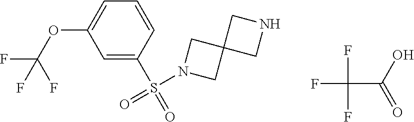 [SMILES: O=S(=O)(c1cccc(OC(F)(F)F)c1)N1CC2(CNC2)C1 ; O=C(O)C(F)(F)F]

To a solution of 2-[3-(trifluoromethoxy)phenyl]sulfonyl-2,6-diazaspiro[3.3]heptane-6-carboxylic acid tert-butyl ester (1350 mg, 3.2 mmol) in dichloromethane (13.5 mL) was added TFA (3.64 g, 2.46 mL, 32.0 mmol) and the reaction mixture was stirred at room temperature for 18 h. Volatiles were removed in vacuo to yield 1855 mg of the crude title compound (purity roughly 70% major contaminant excess of TFA), which was used without further purification. MS (ESI): m/z=323.1 [M−TFA+H]⁺

### Step a) 2-[3-(trifluoromethoxy)phenyl]sulfonyl-2,6-diazaspiro[3.3]heptane-6-carboxylic Acid Tert-Butyl Ester

To a suspension of 2,6-diazaspiro[3.3]heptane-2-carboxylic acid tert-butyl ester (790 mg, 3.98 mmol) in dichloromethane (18 mL) cooled down to 0° C. was added DIPEA (773 mg, 1.04 mL, 5.98 mmol) and 3-(trifluoromethoxy)benzenesulfonyl chloride (1.04 g, 3.98 mmol) after which the reaction mixture was stirred at 0° C. for 10 min and at room temp. for 1 h. The reaction mixture was diluted with dichloromethane and extracted with aq. Na₂CO₃ 1M solution. The organic phase was collected and the aqueous phase was back-extracted with dichloromethane. The combined organic phases were dried over sodium sulfate and evaporated down to dryness. The residue was purified by FC (SiO₂; heptane/EtOAc) to yield 1350 mg of the title compound. MS (ESI): m/z=367.1 [M−tBu+H]⁺

In analogy to Example B.55, the following building blocks were generated using the relevant building blocks in Step a). For Examples B.167 and B.168, 2,7-diazaspiro[3.5]nonane-2-carboxylic acid tert-butyl ester was used in place of 2,6-diazaspiro[3.3]heptane-2-carboxylic acid tert-butyl ester.

| | | | | MS, |
|---|---|---|---|---|
| | | | Building | ESI: |
| Ex. | Structure | Systematic Name | Blocks | m/z |
| B.56 | 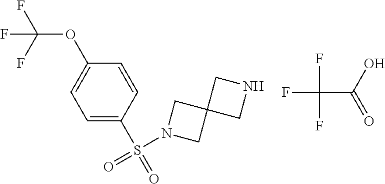 | 2-[4- (trifluoromethoxy)phe- nyl]sulfonyl-2,6- diazaspiro[3.3]heptane; trifluoroacetic acid | 4- (trifluorometh- oxy)benzene- sulfonyl chloride | 323.2 [M − TFA + H]⁺ |
| B.57 | 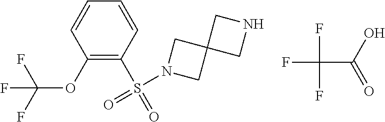 [SMILES: O=S(=O)(c1ccccc1OC(F)(F)F)N1CC2(CNC2)C1 ; O=C(O)C(F)(F)F] | 2-[2- (trifluoromethoxy)phe- nyl]sulfonyl-2,6- diazaspiro[3.3]heptane; trifluoroacetic acid | 2- (trifluorometh- oxy)benzene- sulfonyl chloride | 323.2 [M − TFA + H]⁺ |
| B.59 | 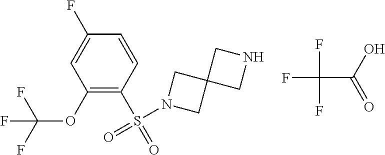 | 2-[4-fluoro-2- (trifluoromethyl)phe- nyl]sulfonyl-2,6- diazaspiro[3.3]heptane; trifluoroacetic acid | 4-fluoro-2- (trifluorometh- yl)benzene- sulfonyl chloride | 325.1 [M − TFA + H]⁺ |
| B.68 | 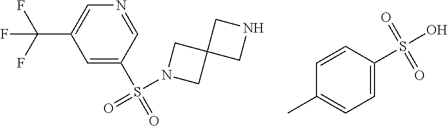 [SMILES: O=S(=O)(c1cncc(C(F)(F)F)c1)N1CC2(CNC2)C1 ; Cc1ccc(S(=O)(=O)O)cc1] | 2-[[5- (trifluoromethyl)-3- pyridyl]sulfonyl]- 2,6- diazaspiro[3.3]heptane; 4- methylbenzenesulfo nic acid | 5- (trifluorometh- yl)pyridine- 3-sulfonyl chloride (CAS: 1060802- 03-0) | 308.0 [M − TsOH + H]⁺ |
| B167 | 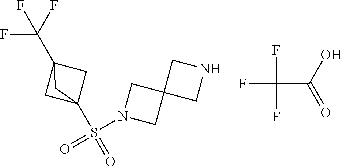 | 7-[[3- (trifluoromethyl)-1- bicyclo[1.1.1]pentan- yl]sulfonyl]-2,7- diazaspiro[3.5]nonane; trifluoroacetic acid | 3- (trifluorometh- yl)bicyclo [1.1.1]pentane- 1- sulfonyl chloride | 325.1 [M − TFA + H]⁺ |
| B.168 | 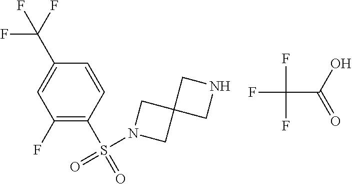 | 7-[2-fluoro-4- (trifluoromethyl)phe- nyl]sulfonyl-2,7- diazaspiro[3.5]nonane; 2,2,2- trifluoroacetic acid | 2-fluoro-4- (trifluorometh- yl)benzene- sulfonyl chloride | 353.1 [M − TFA + H]⁺ |
| B.262 | 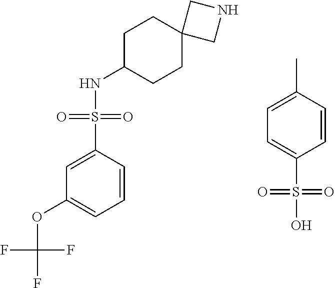 [SMILES: O=S(=O)(NC1CCC2(CC1)CNC2)c1cccc(OC(F)(F)F)c1 ; Cc1ccc(S(=O)(=O)O)cc1] | N-(2- azaspiro[3.5]nonan- 7-yl)-3- (trifluoromethoxy)ben- zenesulfonamide; 4- methylbenzenesulfo- nic acid | 7-amino-2- azaspiro[3.5] nonane-2- carboxylic acid tert- butyl ester (CAS RN: 1408075- 19-3) and 3- (trifluorometh- oxy)benzene- sulfonyl chloride (CAS RN: 220227-84- 9) | 409.1 [M − tBu + H]⁺ |

### Example B.101

### 2-[[6-(trifluoromethyl)pyridazin-3-yl]methyl]-2,6-diazaspiro[3.3]heptane; di 4-methylbenzenesulfonic Acid

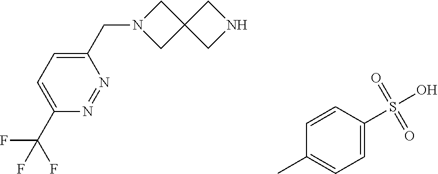 [SMILES: FC(F)(F)c1ccc(CN2CC3(CNC3)C2)nn1 ; Cc1ccc(S(=O)(=O)O)cc1]

A mixture of p-toluenesulfonic acid (1010 mg, 5.86 mmol), tert-butyl 6-[[6-(trifluoromethyl)pyridazin-3-yl]methyl]-2,6-diazaspiro[3.3]heptane-2-carboxylate (1000 mg, 2.79 mmol) in EtOAc (10 mL) was stirred at 80° C. for 12 h. The mixture was filtered and cake was concentrated to give the title compound (1450 mg, 86% yield). MS (ESI): m/z=259.2 [M−2TsOH+H]⁺

### Step a) 3-(chloromethyl)-6-(trifluoromethyl)pyridazine

A mixture of 3-methyl-6-(trifluoromethyl)pyridazine (2.0 g, 12.3 mmol) in 1,2-dichloroethane (40 mL) was added trichloroisocyanuric acid (958 mg, 4.12 mmol). The mixture was heated to 80° C. and stirred for 12 h. The residue was purified by FC to give the title compound (1.3 g, 54% yield) as a white solid. MS (ESI): m/z=197.1 [M+H]⁺

### Step b) tert-butyl 6-[[6-(trifluoromethyl)pyridazin-3-yl]methyl]-2,6-diazaspiro[3.3]heptane-2-carboxylate

To a solution of 3-(chloromethyl)-6-(trifluoromethyl)pyridazine (1.3 g, 6.61 mmol), tert-butyl 2,6-diazaspiro[3.3]heptane-2-carboxylate; oxalic acid (3.22 g, 6.61 mmol) in ACN (10 mL) was added K₂CO₃ (1.83 g, 13.2 mmol) at 25° C. The mixture was stirred at 25° C. for 12 h. The mixture was stirred at 50° C. for 2 h. The residue was purified by silica column (petroleum ether:ethyl acetate=10:1 to 0:1) and concentrated under reduced pressure to give the title compound (1.7 g. 71.7% yield) as a white solid. MS (ESI): m/z=359.3 [M+H]⁺

In analogy to Example B. 101, the following building blocks were generated using the relevant commercial building blocks in Step b). For 4,6 or 4,5 spiro ring systems, 2,7-diazaspiro[3.5]nonane-2-carboxylic acid tert-butyl ester and tert-butyl 2,6-diazaspiro[3.4]octane-2-carboxylate respectively can be used in place of 2,6-diazaspiro[3.3]heptane-2-carboxylic acid tert-butyl ester. In some cases Cs₂CO₃ was used in place of K₂CO₃.

| | | | | MS, |
|---|---|---|---|---|
| | | | Building | ESI: |
| Ex. | Structure | Systematic Name | Blocks | m/z |
| B.103 | 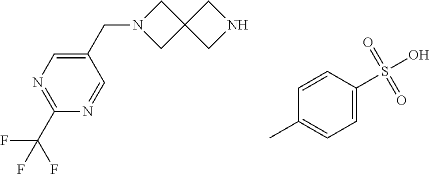 [SMILES: FC(F)(F)c1ncc(CN2CC3(CNC3)C2)cn1 ; Cc1ccc(S(=O)(=O)O)cc1] | 2-[[2- (trifluoromethyl)pyri- midin-5-yl]methyl]- 2,6- diazaspiro[3.3]heptane; 4- methylbenzenesulfo- nic acid | 5- (bromometh- yl)-2- (trifluorometh- yl)pyrimi- dine | 259.1 [M + H]⁺ |
| B.112 | 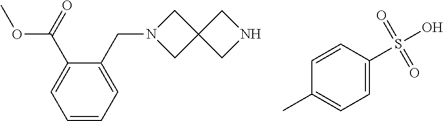 [SMILES: COC(=O)c1ccccc1CN1CC2(CNC2)C1 ; Cc1ccc(S(=O)(=O)O)cc1] | methyl 2-(2,6- diazaspiro[3.3]heptan- 2- ylmethyl)benzoate; 4- methylbenzenesulo- nic acid | methyl 2- (bromometh- yl)benzoate | 247.2 [M + H]⁺ |
| B323 | 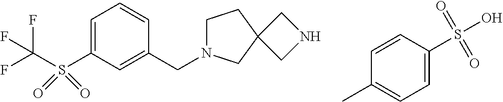 [SMILES: O=S(=O)(c1cccc(CN2CCC3(CNC3)C2)c1)C(F)(F)F ; Cc1ccc(S(=O)(=O)O)cc1] | 6-[[3- (trifluoromethylsulfo- nyl)phenyl]methyl]- 2,6- diazaspiro[3.4]octane; 4- methylbenzenesulfo- | 1- (bromometh- yl)-3- (trifluorometh- ylsulfonyl) benzene (CAS: | 335.0 [M + H]⁺ |
| | | nic acid | 45767-66-6) | |
| B327 | 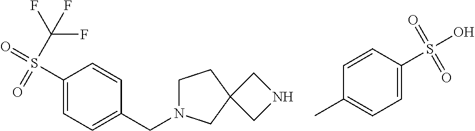 | 6-[[4- (trifluoromethylsulfo- nyl)phenyl]methyl]- 2,6- diazaspiro[3.4]octane; 4- methylbenzenesulfo- nic acid | 1- (bromometh- yl)-4- (trifluorometh- ylsulfonyl) benzene (CAS: 118089-89-7) | 335.1 [M + H]⁺ |
| B.360 | 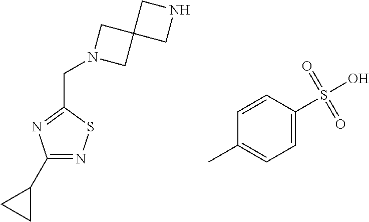 [SMILES: C1CC1c1nsc(CN2CC3(CNC3)C2)n1 ; Cc1ccc(S(=O)(=O)O)cc1] | 3-cyclopropyl-5- (2,6- diazaspiro[3.3]heptan- 2-ylmethyl)-1,2,4- thiadiazole; 4- methylbenzenesulfo- nic acid | 5- (chlorometh- yl)-3- cyclopropyl- 1,2,4- thiadiazole (CAS: 2090447- 99-5) | 237.0 [M + H]⁺ |
| B.361 | 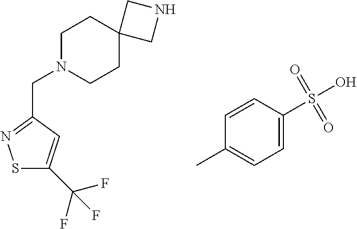 [SMILES: FC(F)(F)c1cc(CN2CCC3(CC2)CNC3)ns1 ; Cc1ccc(S(=O)(=O)O)cc1] | 3-(2,7- diazaspiro[3.5]nonan- 7-ylmethyl)-5- (trifluoromethyl)isothi- azole; 4- methylbenzenesulfo- nic acid | 3- (bromometh- yl)-5- (trifluorometh- yl)isothiazole (CAS: 2253638- 60-5) | 292.2 [M + H]⁺ |
| B.387 | 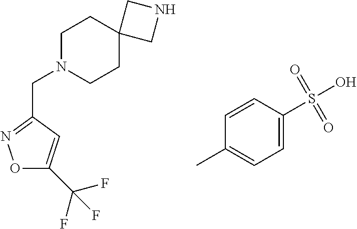 [SMILES: FC(F)(F)c1cc(CN2CCC3(CC2)CNC3)no1 ; Cc1ccc(S(=O)(=O)O)cc1] | 3-(2,7- diazaspiro[3.5]nonan- 7-ylmethyl)-5- (trifluoromethyl)isox- azole; 4- methylbenzenesulfo- nic acid | 3- (bromometh- yl)-5- (trifluorometh- yl)isoxazole (CAS: 861135-68-4) | 276.2 [M + H]⁺ |
| B.388 | 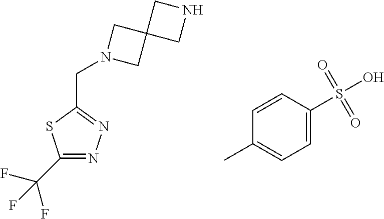 [SMILES: FC(F)(F)c1nnc(CN2CC3(CNC3)C2)s1 ; Cc1ccc(S(=O)(=O)O)cc1] | 2-(2,6- diazaspiro[3.3]heptan- 2-ylmethyl)-5- (trifluoromethyl)- 1,3,4-thiadiazole; 4- methylbenzenesulfo- nic acid | 2- (chlorometh- yl)-5- (trifluorometh- yl)-1,3,4- thiadiazole (CAS: 1260672- 42-1) | 265.0 [M + H]⁺ |
| B.389 | 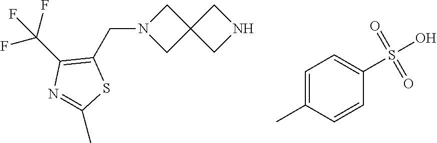 [SMILES: Cc1nc(C(F)(F)F)c(CN2CC3(CNC3)C2)s1 ; Cc1ccc(S(=O)(=O)O)cc1] | 5-(2,6- diazaspiro[3.3]heptan- 2-ylmethyl)-2- methyl-4- (trifluoromethyl)thia- zole; 4- methylbenzenesulfo- nic acid | 2-methyl-4- (trifluorometh- yl)thiazole- 5- carbaldehyde (CAS: 1034566- 13-6) | 278.0 [M + H]⁺ |
| B.394 | 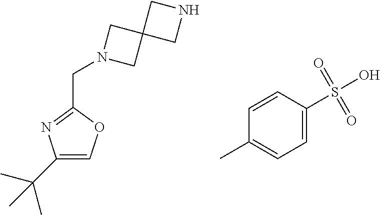 [SMILES: CC(C)(C)c1coc(CN2CC3(CNC3)C2)n1 ; Cc1ccc(S(=O)(=O)O)cc1] | 4-tert-butyl-2-(2,6- diazaspiro[3.3]heptan- 2- ylmethyl)oxazole; 4- methylbenzenesulfo- nic acid | 2- (bromometh- yl)-4-tert- butyl- oxazole (CAS: 2126162- 62-5) | 236.0 [M + H]⁺ |
| B.396 | 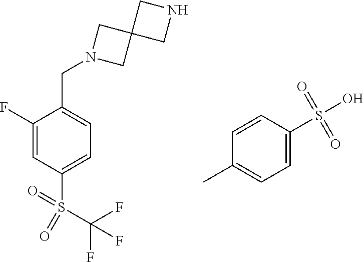 [SMILES: O=S(=O)(c1ccc(CN2CC3(CNC3)C2)c(F)c1)C(F)(F)F ; Cc1ccc(S(=O)(=O)O)cc1] | 2-[[2-fluoro-4- (trifluoromethylsulfo- nyl)phenyl]methyl]- 2,6- diazaspiro[3.3]heptane; 4- methylbenzenesulfo- nic acid | 2-fluoro-1- methyl-4- (trifluorometh- ylsulfonyl) benzene (C.9) | 339.0 [M + H]⁺ |

### Example B.110

### 2-[[4-fluoro-2-(trifluoromethyl)phenyl]methyl]-2,6-diazaspiro[3.3]heptane; trifluoroacetic Acid

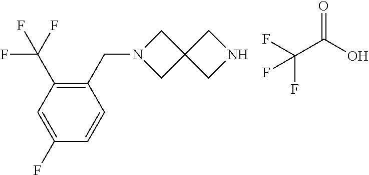

To a solution of text-butyl 6-(4-fluoro-2-(trifluoromethyl)benzyl)-2,6-diazaspiro[3.3]heptane-2-carboxylate (455 mg, 1.09 mmol) in dichloromethane (4 mL) was added TFA (843 μL, 10.9 μL) and the reaction mixture was stirred at RT for 18 h. Volatiles were removed in vacuo to yield 685 mg of the crude title compound (purity roughly 80%) which was used without further purification. MS (ESI): m/z=275.2 [M−TFA+H]⁺

### Step a) tert-butyl 6-(4-fluoro-2-(trifluoromethyl)benzoyl)-2,6-diazaspiro[3.3]heptane-2-carboxylate

To a solution of tert-butyl 2,6-diazaspiro[3.3]heptane-2-carboxylate (400 mg, 2.02 mmol) in CH₂Cl₂ (9 mL) cooled down to 0° C. was added DIPEA (652 mg, 881 μL, 5.04 mmol) and 4-fluoro-2-(trifluoromethyl)benzoyl chloride (503 mg, 2.22 mmol). The reaction mixture was stirred at 0° C. for 10 min and at RT for 18 h. The reaction mixture was diluted with dichloromethane and extracted with aq. Na₂CO₃ 1M solution, the organic phase was collected and the aqueous phase was back-extracted with dichloromethane. The combined organic phases were dried over sodium sulfate and evaporated down to dryness. The crude material was purified by flash chromatography, using an eluent mixture of heptane and ethyl acetate (5% to 80%) to give the title compound (569 mg). MS (ESI): m/z=389.3 [M+H]⁺

### Step b) tert-butyl 6-(4-fluoro-2-(trifluoromethyl)benzyl)-2,6-diazaspiro[3.3]heptane-2-carboxylate

To a solution of tert-butyl 6-(4-fluoro-2-(trifluoromethyl)benzoyl)-2,6-diazaspiro[3.3]heptane-2-carboxylate (565 mg, 1.45 mmol) in dry THF (5 mL) was slowly added borane tetrahydrofuran complex 1.0 M (3.64 mL, 3.64 mmol) and the reaction mixture was then refluxed for 20 h. The reaction was cooled down to 0° C. followed by addition of slow addition of methanol to quench excess borane after which it was stirred at 23° C. for 15 min followed by stirring at 55° C. for 18 h. Volatiles were removed in vacuo and the crude residue was directly purified by flash chromatography using an eluent mixture of dichloromethane and methanol (0% to 10%) to yield 417 mg of the title compound. MS (ESI): m/z=375.2 [M+H]⁺

### Example B.113

### 2-(2,6-diazaspiro[3.3]heptan-2-ylmethyl)-N-methyl-benzamide; 4-methylbenzenesulfonic Acid

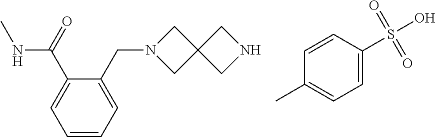 [SMILES: CNC(=O)c1ccccc1CN1CC2(CNC2)C1 ; Cc1ccc(S(=O)(=O)O)cc1]

A solution of tert-butyl 6-[[2-(methylcarbamoyl)phenyl]methyl]-2,6-diazaspiro[3.3]heptane-2-carboxylate (800 mg, 2.32 mmol) and p-toluenesulfonic acid monohydrate (1320 mg, 6.95 mmol) in EtOAc (60 mL) was heated at reflux for 2 h, then cooled to RT and stirred for another 16 h. The precipitate was collected by filtration, washed with ethyl acetate (20 mL) and dried under vacuum to provide the title compound (1069 mg, 1.81 mmol, 77% yield) as light yellow solid. MS (ESI): m/z=246.2 [M+H]⁺

### Step a) tert-butyl 6-[[2-(methylcarbamoyl)phenyl]methyl]-2,6-diazaspiro[3.3]heptane-2-carboxylate

tert-butyl 6-[(2-methoxycarbonylphenyl)methyl]-2,6-diazaspiro[3.3]heptane-2-carboxylate (Example B.112) (1000 mg, 2.89 mmol) was mixed with a solution of monomethylamine (43.3 mL, 866 mmol) (20% in MeOH) in a vial. Then the vial was sealed and heated at 70° C. for 24 h. The reaction mixture was then cooled to room temperature and concentrated in vacuo to give the title compound (970 mg, 92% yield) as light yellow viscous oil. The product was used in the next step without further purification. MS (ESI): m/z=346.2 [M+H]⁺

### Example B.149

### N-(1-methylcyclopropyl)-2,6-diazaspiro[3.3]heptane-2-sulfonamide;2,2,2-trifluoroacetic Acid

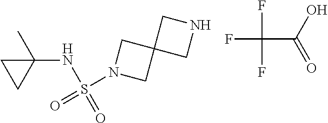

To a solution of 2-[(1-methylcyclopropyl) sulfamoyl]-2,6-diazaspiro[3.3]heptane-6-carboxylic acid tert-butyl ester (448 mg, 1.35 mmol) in dichloromethane (5 mL) was added TFA (1.54 g, 1.04 mL, 13.52 mmol, 10 eq) and the reaction mixture was stirred at room temperature for 18 h. Volatiles were removed in vacuo to yield the crude title compound (736 mg), roughly 63% purity with excess TFA as major contaminant, which was used directly without further purification. MS (ESI): m/z=232.2 [M−TFA+H]⁺

### Step a) 2-(2-methylimidazol-1-yl) sulfonyl-2,6-diazaspiro[3.3]heptane-6-carboxylic Acid Tert-Butyl Ester

To a solution of 2-methyl-1-(2-methylimidazol-1-yl) sulfonyl-imidazole (1.5 g, 6.63 mmol) in dichloromethane (27 mL) under an inert atmosphere cooled down to 0° C. was slowly added methyl trifluoromethanesulfonate (1.09 g, 730 μL, 6.63 mmol). Upon completion of reagent addition a white precipitate began to form and the reaction mixture was allowed to stir at 0° C. and slowly warm up to room temperature overnight. Volatiles were removed in vacuo to give 2.60 g of the crude intermediate as a white solid which was used without further purification.

The crude solid was dissolved in acetonitrile, extra dry (27 mL) followed by addition of 2,6-diazaspiro[3.3]heptane-2-carboxylic acid tert-butyl ester (1.31 g, 6.63 mmol) after which the reaction mixture was stirred at 80° C. for 64 h. The reaction mixture was diluted with ethyl acetate, poured into a separating funnel and extracted with aq. sol. Na₂CO₃ 1 M. The organic phase was collected and the aqueous phase was back-extracted with ethyl acetate. The combined organic phases were dried over sodium sulfate and evaporated down to dryness. The crude material was submitted for SFC purification to yield 1372 mg of the title compound. MS (ESI): m/z=343.2 [M−TFA+H]⁺

### Step b) 2-[(1-methylcyclopropyl) sulfamoyl]-2,6-diazaspiro[3.3]heptane-6-carboxylic Acid Tert-Butyl Ester

To a solution of 2-(2-methylimidazol-1-yl) sulfonyl-2,6-diazaspiro[3.3]heptane-6-carboxylic acid tert-butyl ester (778 mg, 2.27 mmol) in dichloromethane (10 mL) cooled down to 0° C. was added methyl trifluoromethanesulfonate (392 mg, 263 μL, 2.39 mmol) and the reaction mixture was stirred at 0° C. for 3 h. Volatiles were removed in vacuo and the crude white solid was re-dissolved in acetonitrile, extra dry (10 mL) followed by addition of (1-methylcyclopropyl)amine (242 mg, 3.41 mmol) after which the reaction mixture was stirred at 70° C. for 18 h. Volatiles were removed in vacuo. The crude residue was dissolved in ethyl acetate, transferred into a separating funnel and extracted with sat. aq. solution Na₂CO₃. The organic phase was collected and the aqueous phase was back-extracted with ethyl acetate. The combined organic phases were dried over sodium sulfate and evaporated down to dryness. Purification by FC (SiO₂: DCM/MeOH) gave the title compound (448 mg). MS (ESI): m/z=330.3 [M+H]⁺

### Example B.152

### N-[[1-(trifluoromethyl)cyclopropyl]methyl]-2,6-diazaspiro[3.3]heptane-2-sulfonamide; 4-methylbenzenesulfonic Acid

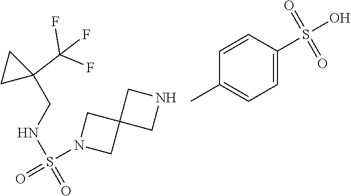

A solution of tert-butyl 2-[[1-(trifluoromethyl)cyclopropyl]methylsulfamoyl]-2,6-diazaspiro[3.3]heptane-6-carboxylate (450 mg, 1.13 mmol) and p-toluenesulfonic acid monohydrate (429 mg, 2.25 mmol) in EtOAc (15 mL) was heated at reflux for 2 h, then cooled to RT and stirred for another 16 h. The precipitate was collected by filtration, washed with EtOAc (5 mL) and dried under vacuum to provide the title compound (298 mg, 53% yield). MS (ESI): m/z=300.2 [M−TsOH+H]⁺

### Step a) tert-butyl 2-chlorosulfonyl-2,6-diazaspiro[3.3]heptane-6-carboxylate

To a stirred solution of sulfuryl chloride (0.63 g, 4.69 mmol) in DCM (15 mL) at 0° C. was added a mixture of triethylamine (1.19 mL, 8.52 mmol) and tert-butyl 2,6-diazaspiro[3.3]heptane-2-carboxylate hydrochloride (1.0 g, 4.26 mmol) (as a solution in 15 mL of DCM) at such a rate as to keep the temperature below 20° C. The reaction mixture was stirred at room temperature for 18 h, then evaporated to dryness. The crude sulfamoyl chloride (30% purity) was used directly in next step without further purification.

### Step b) tert-butyl 2-[[1-(trifluoromethyl)cyclopropyl]methylsulfamoyl]-2,6-diazaspiro[3.3]heptane-6-carboxylate

To a stirred mixture of tert-butyl 2-chlorosulfonyl-2,6-diazaspiro[3.3]heptane-6-carboxylate (310 mg, 1.04 mmol) and [1-(trifluoromethyl)cyclopropyl]methanamine; hydrochloride (238 mg, 1.36 mmol) in ACN (10 mL), N,N-diisopropylethylamine (0.55 mL, 3.13 mmol) was added. Then the tube was was sealed and stirred at 40° C. for 18 h. Then the reaction mixture was concentrated to dryness and the residue was taken up in DCM (20 mL) and the organics washed with water (2×5 mL) and saturated brine solution (5 mL). The organic layer was dried (Na₂SO₄) before being concentrated to dryness in vacuo. The (290 mg, 0.730 mmol, 66% yield) was used to the next step without further purification. MS (ESI): m/z=398.2 [M−H]⁻

### Example B.153

### 2-[5-(trifluoromethyl) pyrazin-2-yl]-2,6-diazaspiro[3.3]heptane; 4-methylbenzenesulfonic Acid

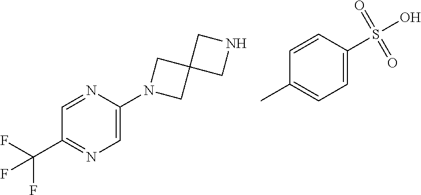

In a flask was added 6-[5-(trifluoromethyl) pyrazin-2-yl]-2,6-diazaspiro[3.3]heptane-2-carboxylic acid tert-butyl ester (312 mg, 0.906 mmol), p-toluenesulfonic acid monohydrate (361.96 mg, 1.9 mmol) in ethyl acetate (7.64 mL) the mixture was stirred at reflux overnight. The crude residue was washed with EtOAc and diethylether to afford the title compound as an off-white solid with an assumed purity of 95%, which was used without further purification. MS (ESI): m/z=245.1 [M−TsOH+H]⁺

### Step a) 6-[5-(trifluoromethyl) pyrazin-2-yl]-2,6-diazaspiro[3.3]heptane-2-carboxylic Acid Tert-Butyl Ester

In a flask was added 2-bromo-5-(trifluoromethyl) pyrazine (280 mg, 1.23 mmol), 2-Boc-2,6-diazaspiro[3.3]heptane (269 mg, 1.36 mmol) and cesium carbonate (804 mg, 2.47 mmol) in 1,4-dioxane (6. mL). The suspension was bubbled with N₂ for 5 min and chloro(2-dicyclohexylphosphino-2′,6′-diisopropoxy-1,1′-biphenyl) [2-(2′-amino-1,1′-biphenyl)]palladium(II) (47.9 mg, 0.062 mmol) was added. The mixture was heated at 110° C. for 2 h. The mixture was diluted in EtOAc and filtered through celite, the filtrate was concentrated. Purification by FC (SiO₂; heptane/EtOAc) gave the title compound as a yellow solid which was directly used in the next step. MS (ESI): m/z=345.1 [M+H]⁺

In analogy to Example B.153, the following building blocks were generated using the relevant commercial building blocks.

| | | | | MS, |
|---|---|---|---|---|
| | | | Building | ESI: |
| Ex. | Structure | Systematic Name | Blocks | m/z |
| B.372 | 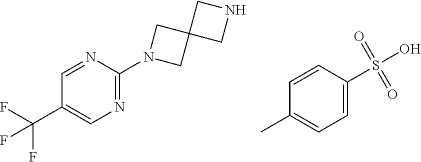 [SMILES: FC(F)(F)c1cnc(N2CC3(CNC3)C2)nc1 ; Cc1ccc(S(=O)(=O)O)cc1] | 2-[5- (trifluoromethyl)pyri- midin-2-yl]-2,6- diazaspiro[3.3]heptane; 4- methylbenzenesulfo- nic acid | 2-bromo-5- (trifluorometh- yl)pyrimi- dine | 245.1 [M − TsOH + H]⁺ |

### Example B.265

### N-[5-(trifluoromethyl) pyrazin-2-yl]-2-azaspiro[3.5]nonan-7-amine; 4-methylbenzenesulfonic Acid

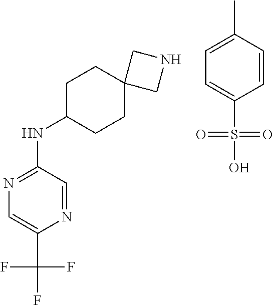

To a solution of 7-[[5-(trifluoromethyl) pyrazin-2-yl]amino]-2-azaspiro[3.5]nonane-2-carboxylic acid tert-butyl ester (525 mg, 1.36 mmol) in isopropyl acetate (12 mL) was added p-toluenesulfonic acid monohydrate (568.57 mg, 2.99 mmol). The mixture was stirred at 80° C. for 5 h, before being evaporated. Et₂O was added, and the resulting precipitate was filtered off, washed with Et₂O (2×), and dried, to give the title compound (755 mg, 83.7%) as a white solid MS (ESI) m/z=287.1 [M+H]⁺

### Step a): tert-butyl 7-[[5-(trifluoromethyl) pyrazin-2-yl]amino]-2-azaspiro[3.5]nonane-2-carboxylate

To a solution of 7-amino-2-azaspiro[3.5]nonane-2-carboxylic acid tert-butyl ester (CAS RN: 1408075-19-3; 1.33 g, 5.53 mmol) and DIPEA (1.45 mL, 8.3 mmol) in DMSO (10 mL) was added 2-fluoro-5-(trifluoromethyl) pyrazine (CAS RN: 1220799-65-4; 919.06 mg, 5.53 mmol). The mixture was stirred at 60° C. for 5 h, before being poured into EtOAc. The mixture was washed with water and brine. The organic layer was dried over Na₂SO₄, filtered, and evaporated. Purification by FC (SiO₂; heptane/EtOAc) gave the title compound (1.66 g, 73.75% yield) as a white solid. MS (ESI): m/z=331.2 [M+H−tBu]⁺

In analogy to Example B.265, the following building block was generated using the relevant commercial building block in Step a).

| | | | Building | MS, ESI: |
|---|---|---|---|---|
| Ex. | Structure | Systematic Name | Blocks | m/z |
| B.369 | 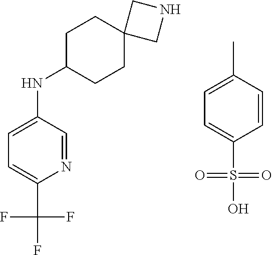 [SMILES: FC(F)(F)c1ccc(NC2CCC3(CC2)CNC3)cn1 ; Cc1ccc(S(=O)(=O)O)cc1] | N-[6- (trifluoromethyl)-3- pyridyl]-2- azaspiro[3.5]nonan-7- amine; 4- methylbenzenesulfonic acid | 2-chloro-5- (trifluoromethyl) pyridine | 286.1 [M + H]⁺ |

### Example B.368

### N-methyl-N-[5-(trifluoromethyl) pyrazin-2-yl]-2-azaspiro[3.5]nonan-7-amine; 4-methylbenzenesulfonic Acid

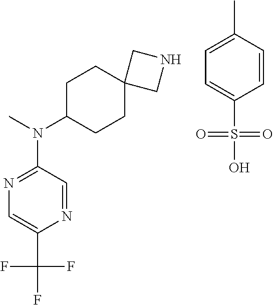

To a solution of 7-[methyl-[5-(trifluoromethyl) pyrazin-2-yl]amino]-2-azaspiro[3.5]nonane-2-carboxylic acid tert-butyl ester (460 mg, 1.15 mmol) in isopropyl acetate (10 mL) was added p-toluenesulfonic acid monohydrate (481 mg, 2.53 mmol). The mixture was stirred at 80° C. for 5 h. Et₂O was added, and the mixture filtered through sintered glass. The white solid was washed with Et₂O (2×) and dried in vacuo to afford the title compound (678 mg, 87%) as white solid. MS (ESI) m/z=301.1 [M+H]⁺

### Step a) 7-[methyl-[5-(trifluoromethyl) pyrazin-2-yl]amino]-2-azaspiro[3.5]nonane-2-carboxylic Acid Tert-Butyl Ester

To a solution of tert-butyl 7-[[5-(trifluoromethyl) pyrazin-2-yl]amino]-2-azaspiro[3.5]nonane-2-carboxylate (B.265, Step a) (0.610 g, 1.5 mmol) in N,N-dimethylformamide (3 mL) was added sodium hydride (72.0 mg, 1.8 mmol). The mixture was stirred at 40° C. for 15 min. The reaction mixture was cooled to room temp. Iodomethane (213 mg, 93.8 μL, 1.5 mmol) was added in one portion. The reaction mixture was stirred at 60° C. for 2 h, then poured into EtOac/THF 2:1 and washed with water, and brine. The organic layer was dried over Na₂SO₄ and concentrated in vacuo. The crude material was purified by flash chromatography (eluting with 0% to 2% methanol in dichloromethane) to afford the title compound (462 mg, 73.1%) as white solid. MS (ESI): m/z=345.2 [M+H−tBu]⁺

### Example B.311

### 6-[[5-(trifluoromethyl) pyrazol-1-yl]methyl]-2-azaspiro[3.3]heptane; 4-methylbenzenesulfonic Acid

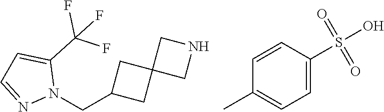 [SMILES: FC(F)(F)c1ccnn1CC1CC2(CNC2)C1 ; Cc1ccc(S(=O)(=O)O)cc1]

To a solution of tert-butyl 6-[[5-(trifluoromethyl) pyrazol-1-yl]methyl]-2-azaspiro[3.3]heptane-2-carboxylate (200 mg, 0.58 mmol) in EtOAc (2 mL) was added p-toluenesulfonic acid (110 mg, 0.64 mmol) and stirred for 12 h at 80° C. The reaction mixture was filtered and concentrated under reduced pressure to give the title compound (230 mg, 0.55 mmol, 95% yield) as a yellow oil. MS (ESI): m/z=246.1 [M+H]⁺.

### Step a) tert-butyl 6-[[5-(trifluoromethyl) pyrazol-1-yl]methyl]-2-azaspiro[3.3]heptane-2-carboxylate

To the mixture of tert-butyl 6-(hydroxymethyl)-2-azaspiro[3.3]heptane-2-carboxylate (CAS: 1363381-93-4) (10.0 g, 44.0 mmol), 5-(trifluoromethyl)-1H-pyrazole (5.99 g, 44.0 mmol), triphenylphosphine (14.4 g, 55.0 mmol) in THF (100 mL) was added diisopropyl azodicarboxylate (10.4 mL, 52.8 mmol) at 0° C., then the reaction mixture was stirred at 20° C. for 12 h under N₂. The reaction mixture was diluted with water 100 mL and extracted with EtOAc 300 mL (100 mL×3). The combined organic layers were washed with aq. sat. NaCl solution (50 mL), dried over Na₂SO₄, filtered and concentrated under reduced pressure to give a residue. The residue was purified by normal phase preparative HPLC to give the title compound (3.7 g, 10.7 mmol, 24.4% yield) as a yellow solid. MS (ESI): m/z=346.1 [M+H]⁺

In analogy to Example B.311, the following building block was generated using the relevant commercial building block in Step a).

| | | | | MS, |
|---|---|---|---|---|
| | | | | ESI: |
| Ex. | Structure | Systematic Name | Bruilding Blocks | m/z |
| P.51 | 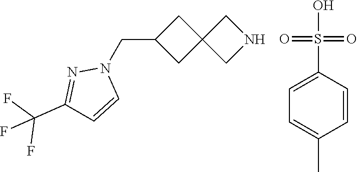 | 6-[[3- (trifluoromethyl)pyrazol- 1-yl]methyl]-2- azaspiro[3.3]heptane; 4- methylbenzenesulfonic acid | 5- (trifluoromethyl)- 1H-pyrazole | 246.2 [M − TsOH + H]⁺ |

### Example P.23

### 1-(2-Azaspiro[3.3]heptan-6-ylmethyl)-4-(trifluoromethyl)pyridin-2-one; 4-methylbenzenesulfonic Acid

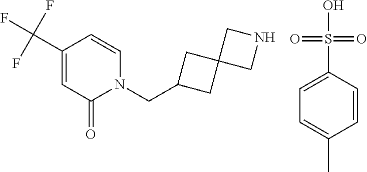

To a solution of tert-butyl 6-[[2-oxo-4-(trifluoromethyl)-1-pyridyl]methyl]-2-azaspiro[3.3]heptane-2-carboxylate (5 g, 13.4 mmol) in ethyl acetate (150 mL) was added p-toluenesulfonic acid monohydrate (5.1 g, 26.8 mmol) and stirred at 25° C. for 72 h. The reaction mixture was filtered and washed with diethyl ether to afford 1-(2-azaspiro[3.3]heptan-6-ylmethyl)-4-(trifluoromethyl)pyridin-2-one; 4-methylbenzenesulfonic acid (5.34 mg, 85% yield) as light yellow solid. MS (ESI): m/z=273.0 [M−TsOH+H]⁺.

### Step a) tert-butyl 6-(methylsulfonyloxymethyl)-2-azaspiro[3.3]heptane-2-carboxylate

To a stirred solution of tert-butyl 6-(hydroxymethyl)-2-azaspiro[3.3]heptane-2-carboxylate (CAS: 1363381-93-4) (14.9 g, 65.7 mmol) in DCM (299 mL) was added triethylamine (13.7 mL, 98.6 mmol), cooled the reaction mixture to 0° C. followed by dropwise addition of methanesulfonyl chloride (6.1 mL, 78.9 mmol) then reaction mixture was stirred at room temperature for 4 h. The reaction mixture was diluted with water and extracted with DCM, washed with water, brine, dried over anhydrous sodium sulphate and evaporate under reduced pressure to give the title compound (19.8 g, 64.8 mmol, 93.7% yield) as a light yellow solid. MS (ESI): m/z=250.0 [M−Bu+H]⁺.

Step b) tert-butyl 6-[[2-oxo-4-(trifluoromethyl)-1-pyridyl]methyl]-2-azaspiro[3.3]heptane-2-carboxylate 4-(trifluoromethyl)-1H-pyridin-2-one (CAS: 50650-59-4) (5.34 g, 32.8 mmol) was added in small portions under argon at 0° C. to a suspension of sodium hydride 60% in oil (2.14 g. 49.1 mmol) in DMF (100 mL). The mixture was stirred at 0° C. for 10 min and at room temperature for 30 min. The reaction mixture was cooled to 0° C. and tert-butyl 6-(methylsulfonyloxymethyl)-2-azaspiro[3.3]heptane-2-carboxylate (10 g, 32.7 mmol), sodium iodide (4.91 g, 32.7 mmol) were added in one portion. The mixture was stirred at 0° C. for 1 hour then at 80° C. for 18 hours. The reaction mixture was poured into EtOAc and washed with water and brine. The organic layer was dried over Na₂SO₄ and concentrated in vacuo. The crude material was purified by flash chromatography (silica gel, 120 g, 0% to 100% tert-butylmethylether in heptane) to afford the title compound (3.6 g, 29% yield) as yellow solid. MS (ESI): m/z=273.0 [M+H]⁺.

In analogy to Example P.23, the following building block was generated using the relevant commercial building block in Step b).

| | | | | MS, |
|---|---|---|---|---|
| | | | | ESI: |
| Ex. | Structure | Systematic Name | Building Blocks | m/z |
| P.24 | 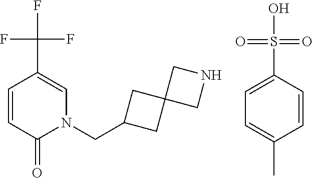 | 1-(2- azaspiro[3.3]heptan-6- ylmethyl)-5- (trifluoromethyl)pyridin- 2-one; 4- methylbenzenesulfonic acid | 5- (trifluoromethyl)- 1H-pyridin-2-one (CAS: 33252-63- 0) | 273.0 [M − TsOH + H]⁺ |

### Example P.50

### 1-(2-Azaspiro[3.3]heptan-6-ylmethyl)-4-(trifluoromethyl)pyridin-2-one; 4-methylbenzenesulfonic Acid

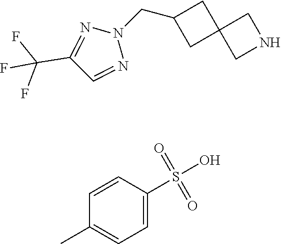 [SMILES: FC(F)(F)c1cnn(CC2CC3(CNC3)C2)n1 ; Cc1ccc(S(=O)(=O)O)cc1]

To a solution of tert-butyl 6-[[4-(trifluoromethyl)triazol-2-yl]methyl]-2-azaspiro[3.3]heptane-2-carboxylate (2.5 g, 7.2 mmol) in EtOAc (100 mL) was added p-toluenesulfonic acid monohydrate (4.12 g, 21.6 mmol). The reaction mixture was stirred at 25° C. for 18 h, filtered and washed with diethyl ether to afford the title compound (3.63 g, 81% yield) as white powder. MS (ESI): m/z=247.2 [M+H]⁺.

### Step a) tert-butyl 6-[[4-(trifluoromethyl)triazol-2-yl]methyl]-2-azaspiro[3.3]heptane-2-carboxylate

To a mixture of 4-(trifluoromethyl)-1H-triazole (2.69 g, 19.7 mmol), lithium bromide (3.41 g, 39.3 mmol) in acetonitrile (300 mL) was added tert-butyl 6-(methylsulfonyloxymethyl)-2-azaspiro[3.3]heptane-2-carboxylate (P.23, Step a) (6.0 g, 19.65 mmol, 1 eq, CAS 2740574-92-7).

The reaction mixture was stirred at 50° C. for 18 h, filtered and concentrated in vacuo. The crude material was purified by flash chromatography (silica gel, 220 g, 0% to 100% MTBE in heptane) to afford the title compound (2.5 g, 36% yield) as light yellow solid. MS (ESI): m/z=247.0 [M+H−Boc]⁺.

In analogy to Example P.50, the following building block was generated using the relevant commercial building block in Step a).

| | | | | MS, |
|---|---|---|---|---|
| | | | | ESI: |
| Ex. | Structure | Systematic Name | Building Blocks | m/z |
| B.354 | 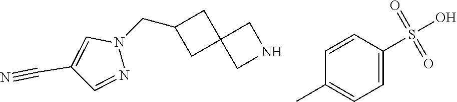 | 1-(2- azaspiro[3.3]heptan-6- ylmethyl)pyrazole-4- carbonitrile; 4- methylbenzenesulfonic acid | 1H-pyrazole-4- carbonitrile (CAS: 31108-57-3) | 203.0 [M − TsOH + H]⁺ |

### Example P.62

### 6-[[3-(Difluoromethyl)-1H-pyrazol-5-yl]methyl]-2-azaspiro[3.3]heptane; 2,2,2-trifluoroacetic Acid

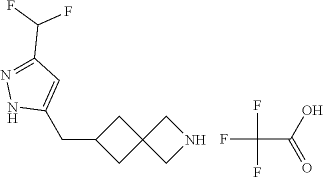

To a solution of tert-butyl 6-[[5-(difluoromethyl)-2-(2-trimethylsilylethoxymethyl) pyrazol-3-yl]methyl]-2-azaspiro[3.3]heptane-2-carboxylate (6 g, 13.1 mmol) in dichloromethane (40 mL) was added 2,2,2-trifluoroacetic acid (20 mL) at 0° C. The mixture was stirred at 20° C. for 16 h. The reaction mixture was concentrated in vacuo. The residue was purified by prep-HPLC to afford the title compound (3.94 g, 87% yield) as white solid. MS (ESI): m/z=228.2 [M+H]⁺.

### Step a) tert-Butyl 6-[[5-(difluoromethyl)-2-(2-trimethylsilylethoxymethyl) pyrazol-3-yl]methylene]-2-azaspiro[3.3]heptane-2-carboxylate

To a mixture of 2-[[5-bromo-3-(difluoromethyl) pyrazol-1-yl]methoxy]ethyl-trimethyl-silane (5.3 g, 16.2 mmol, CAS 2416163-95-4), tert-butyl 6-[(4,4,5,5-tetramethyl-1,3,2-dioxaborolan-2-yl)methylene]-2-azaspiro[3.3]heptane-2-carboxylate (5.97 g, 17.8 mmol, CAS 2763647-64-7) and potassium carbonate (4.47 g, 32.4 mmol) in 1,4-dioxane (50 mL) and water (5 mL) was added cyclopenta-2,4-dien-1-yl(diphenyl)phosphane; dichloromethane; dichloropalladium; iron (2+) (1.32 g, 1.62 mmol, 0.1 eq, CAS 95464 May 4) under N₂. The mixture was stirred at 100° C. for 2 h under N₂ atmosphere. The reaction mixture was poured into water (500 mL). The aqueous phase was extracted with EtOAc (300 mL×2). The organic phase was washed with brine (600 mL), dried with anhydrous Na₂SO₄, filtered and concentrated in vacuo. The residue was purified by column chromatography (SiO₂, petroleum ether/ethyl acetate=100/1 to 1/1) to afford the title compound (6.1 g, 13.4 mmol, 79% yield) as alight yellow solid. MS (ESI): m/z=456.3 [M+H]⁺

### Step b) tert-butyl 6-[[5-(difluoromethyl)-2-(2-trimethylsilylethoxymethyl) pyrazol-3-yl]methyl]-2-azaspiro[3.3]heptane-2-carboxylate

To a solution of tert-butyl 6-[[5-(difluoromethyl)-2-(2-trimethylsilylethoxymethyl) pyrazol-3-yl]methylene]-2-azaspiro[3.3]heptane-2-carboxylate (7 g, 15.4 mmol) in EtOAc (100 mL) was added Pd/C 10% (2 g, 4.61 mmol, 0.3 eq) under N₂ atmosphere. The mixture was stirred at 25° C. for 0.5 h under H₂ (15 PSI) atmosphere. The reaction mixture was filtered and concentrated in vacuo to afford tert-butyl 6-[[5-(difluoromethyl)-2-(2-trimethylsilylethoxymethyl) pyrazol-3-yl]methyl]-2-azaspiro[3.3]heptane-2-carboxylate (6 g, 85% yield) as a colorless oil.

### Example B.329

### [4-(2-azaspiro[3.3]heptan-6-ylmethyl)phenyl]-imino-oxo-(trifluoromethyl)-λ6-sulfane; 4-methylbenzenesulfonic Acid

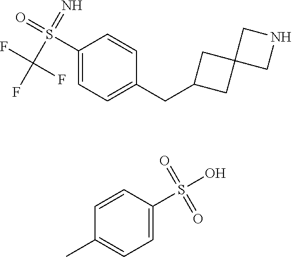 [SMILES: N=S(=O)(c1ccc(CC2CC3(CNC3)C2)cc1)C(F)(F)F ; Cc1ccc(S(=O)(=O)O)cc1]

To a mixture of tert-butyl 6-[[2-fluoro-4-(trifluoromethylsulfonimidoyl)phenyl]methyl]-2-azaspiro[3.3]heptane-2-carboxylate (2.03 g, 4.65 mmol) in EtOAc (20 mL) was added p-toluenesulfonic acid (0.96 g, 5.58 mmol) at 20° C. Then the mixture was stirred at 80° C. for 12 h. The mixture was concentrated to remove the solvent, then added deionized water and lyophilized to give the title compound (2.05 g, 4.03 mmol, 83.2% yield) as yellow gum. MS (ESI): m/z=337.1 [M−TsOH+H]⁺

### Step a) tert-butyl 6-[[2-fluoro-4-(trifluoromethylsulfanyl)phenyl]methylene]-2-azaspiro[3.3]heptane-2-carboxylate

To a solution of tert-butyl 6-[(4,4,5,5-tetramethyl-1,3,2-dioxaborolan-2-yl)methylene]-2-azaspiro[3.3]heptane-2-carboxylate (15.6 g, 46.5 mmol), 1-bromo-2-fluoro-4-(trifluoromethylsulfanyl)benzene (CAS: 1520947-39-0) (12.8 g, 46.5 mmol) and POTASSIUM CARBONATE (12.9 g, 93.1 mmol) in 1,4-Dioxane (170 mL) and water (34 mL) was added 1,1′-bis(diphenylphosphino) ferrocene-palladium(II) dichloride dichloromethane complex (3.8 g, 4.65 mmol) at 25° C., then the mixture was stirred at 80° C. under N₂ atmosphere for 12 h. The mixture was purified by chromatography on silica gel (PE:EA=5:1) and concentrated under vacuum to give a crude product, which was further purified by PREP-HPLC to give the title compound (5.3 g, 13.1 mmol, 28% yield) as a yellow oil. MS (ESI): m/z=348.0 [M−C₄H₈+H]⁺

### Step b) of tert-butyl 6-[[2-fluoro-4-(trifluoromethylsulfanyl)phenyl]methyl]-2-azaspiro[3.3]heptane-2-carboxylate

To a solution of tert-butyl 6-[[2-fluoro-4-(trifluoromethylsulfanyl)phenyl]methylene]-2-azaspiro[3.3]heptane-2-carboxylate (5.3 g, 13.1 mmol) in EtOAc (80 mL) was added wet Pd/C (1.8 g, 1.31 mmol) at 25° C. under N₂, the mixture was stirred at 25° C. under H₂ atmosphere (balloon) for 12 h. The mixture was then filtered and the filtrate was concentrated to give the title compound (5.5 g, 13.6 mmol, 95.0% yield) as colorless oil. MS (ESI): m/z=350.0 [M−C₄H₈+H]⁺

### Step c) tert-butyl 6-[[2-fluoro-4-(trifluoromethylsulfonimidoyl)phenyl]methyl]-2-azaspiro[3.3]heptane-2-carboxylate

To a solution of tert-butyl 6-[[2-fluoro-4-(trifluoromethylsulfanyl)phenyl]methyl]-2-azaspiro[3.3]heptane-2-carboxylate (5.3 g, 13.1 mmol) in trifluoroethanol (55.0 mL) was added PhI(OAc)₂ (17.7 g, 54.9 mmol) and NH₂COONH₄ (3.06 g, 39.2 mmol) at 25° C. Then the mixture was stirred at 60° C. for 12 h. The mixture was purified by PREP-HPLC (Phenomenex luna C18 150*40 mm*15 μm water (FA)-ACN) to give the title compound (2.03 g, 4.65 mmol, 36% yield) as yellow oil. MS (ESI): m/z=381.1 [M−C₄H₈+H]⁺

### Example B.334

### 5-(2-azaspiro[3.3]heptan-6-yloxy)-2-(trifluoromethyl)benzonitrile; 4-methylbenzenesulfonic Acid

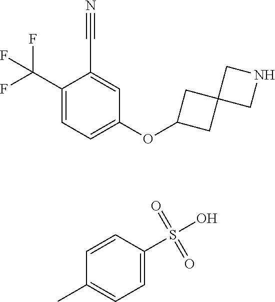 [SMILES: N#Cc1cc(OC2CC3(CNC3)C2)ccc1C(F)(F)F ; Cc1ccc(S(=O)(=O)O)cc1]

To a solution of tert-butyl 6-[3-cyano-4-(trifluoromethyl) phenoxy]-2-azaspiro[3.3]heptane-2-carboxylate (452 mg, 1.18 mmol) in Ethyl acetate (10 mL) p-toluenesulfonic acid monohydrate (292 mg, 1.54 mmol) was added. The mixture was stirred at 20° C. for 16 h, the precipitated solid was filtered, washed with ACN and dried to give the title compound (283 mg, 0.62 mmol, 53% yield) as a white solid. MS (ESI): m/z=283.2 [M−TsOH+H]⁺

### Step a) tert-butyl 6-[3-cyano-4-(trifluoromethyl) phenoxy]-2-azaspiro[3.3]heptane-2-carboxylate

A solution of tert-butyl 6-hydroxy-2-azaspiro[3.3]heptane-2-carboxylate (500 mg, 2.34 mmol) and 5-hydroxy-2-(trifluoromethyl)benzonitrile (483 mg, 2.58 mmol) in toluene (10 mL) was cooled to 0° C. Triphenylphosphine (799 mg, 3.05 mmol) and diisopropyl azodicarboxylate (0.6 mL, 3.05 mmol) were added under Ar. The mixture was warmed to 20° C. and stirred for 16 h. The mixture was concentrated, the residue was triturated with TBME. The precipitated solid was filtered off, the filtrate was concentrated. The residue was purified by FC (silica, 20% EtOAc in hexane) to afford the title compound (430 mg, 1.12 mmol, 48% yield) as a white solid. LCMS: molecular peak is not shown.

In analogy to Example B.334, the following building block was generated using the relevant commercial building block in Step a).

| | | | | MS, |
|---|---|---|---|---|
| | | | | ESI: |
| Ex. | Structure | Systematic Name | Building Blocks | m/z |
| B.376 | 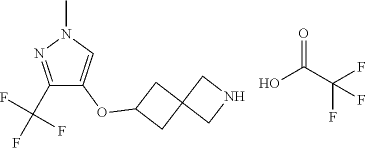 [SMILES: Cn1cc(OC2CC3(CNC3)C2)c(C(F)(F)F)n1 ; O=C(O)C(F)(F)F] | 6-[1-methyl-3- (trifluoromethyl)pyrazol- 4-yl]oxy-2- azaspiro[3.3]heptane; 2,2,2-trifluoroacetic acid | 1-methyl-3- (trifluoromethyl)py- razol-4-ol (CAS: 172834-79-6) | 262.2 [M − TFA + H]⁺ |

### Example B.336

### 6-[3-(trifluoromethoxy)phenyl]sulfonyl-2-azaspiro[3.3]heptane; 2,2,2-trifluoroacetic Acid

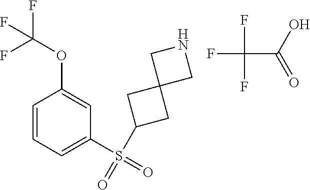

To a solution of 7-[3-(trifluoromethyl)phenyl]sulfonyl-2-azaspiro[3.5]nonane-2-carboxylic acid tert-butyl ester (267 mg, 0.585 mmol) in dichloromethane (3 mL) was added TFA (667 mg, 451 μL, 5.85 mmol) and the reaction mixture was stirred at RT for 18 h. Volatiles were removed in vacuo to give 431 mg of the crude title compound as colorless viscous oil (purity ˜60% major contaminant excess of TFA), which was used without further purification. MS (ESI): m/z=322.1 [M−TsOH+H]⁺

### Step a) tert-butyl 6-[[2-fluoro-4-(trifluoromethylsulfonimidoyl)phenyl]methyl]-2-azaspiro[3.3]heptane-2-carboxylate

To a solution of 6-methylsulfonyloxy-2-azaspiro[3.3]heptane-2-carboxylic acid tert-butyl ester (1000 mg, 3.26 mmol) in tetrahydrofuran (15 mL) was added 3-(trifluoromethoxy)benzenethiol (696 mg, 3.59 mmol) and cesium carbonate (1.06 g, 3.26 mmol) after which the reaction mixture was stirred at 60° C. for 18 h. A further addition of of 3-(trifluoromethoxy)benzenethiol (348 mg) and cesium carbonate (503 mg) was made, after which the reaction mixture was stirred again at 60° C. for 6 h. The reaction mixture was poured into a separating funnel containing ethyl acetate and aq. Na₂CO₃ 1 M solution for extraction. The organic phase was collected and the aqueous phase was back-extracted with ethyl acetate. The combined organic phases were dried over sodium sulfate and evaporated down to dryness. The crude material was purified by flash chromatography (silica gel, ethyl acetate in heptane (5% to 25%) to yield 1.11 g of the title compound. MS (ESI): m/z=334.0 [M−C₄H₈+H]⁺

### Step b) 7-[3-(trifluoromethyl)phenyl]sulfonyl-2-azaspiro[3.5]nonane-2-carboxylic Acid Tert-Butyl Ester

To a suspension of 6-[[3-(trifluoromethoxy)phenyl]thio]-2-azaspiro[3.3]heptane-2-carboxylic acid tert-butyl ester (250 mg, 0.629 mmol) in a mixture of methanol (3 mL)/water (3 mL) was added oxone (812 mg, 1.32 mmol) and sodium bicarbonate (159 mg, 1.89 mmol) after which the reaction mixture was stirred at RT for 18 h. The reaction mixture was poured into a separating funnel containing ethyl acetate and aq. sol. 1 M NaHCO₃, after extraction the organic phase was collected and the aqueous phase was back-extracted with ethyl acetate. The combined organic phases were dried over sodium sulfate and evaporated down to dryness to 265 mg of the crude title compound as a white solid. MS (ESI): m/z=366.1 [M−C₄H₈+H]⁺

In analogy to Example B.336, the following building block was generated using the relevant commercial building blocks.

| | | | | MS, |
|---|---|---|---|---|
| | | | | ESI: |
| Ex. | Structure | Systematic Name | Building Blocks | m/z |
| B.337 | 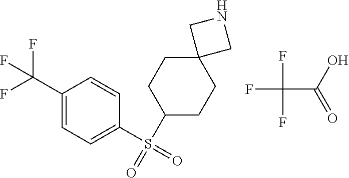 | 7-[4- (trifluoromethyl)phenyl] sulfonyl-2- azaspiro[3.5]nonane; 2,2,2-trifluoroacetic acid | 4- (trifluoromethyl)ben- zenethiol; 7- methylsulfonyloxy- 2- azaspiro[3.5]nonane- 2-carboxylic acid tert-butyl ester (CAS: 14212270-78-1) | 334.1 [M − TsOH + H]⁺ |
| B.338 | 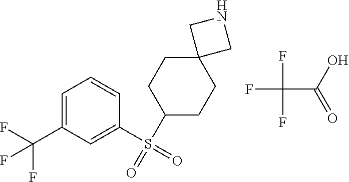 | 7-[3- (trifluoromethyl)phenyl] sulfonyl-2- azaspiro[3.5]nonane; 2,2,2-trifluoroacetic acid | 3- (trifluoromethyl)ben- zenethiol; 7- methylsulfonyloxy- 2- azaspiro[3.5]nonane- 2-carboxylic acid tert-butyl ester (CAS: 1421270-78-1) | 334.1 [M − TsOH + H]⁺ |

### Example B.339

### 2-azaspiro[3.3]heptan-6-yl-imino-oxo-[3-(trifluoromethoxy)phenyl]-λ6-sulfane; 2,2,2-trifluoroacetic Acid

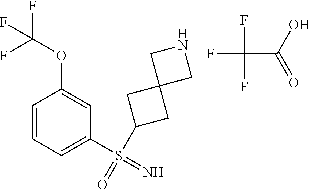

To a solution of 6-[[3-(trifluoromethoxy)phenyl]sulfonimidoyl]-2-azaspiro[3.3]heptane-2-carboxylic acid tert-butyl ester (225 mg, 0.508 mmol) in dichloromethane (2 mL) was added TFA (580 mg, 392 μL, 5.08 mmol) and the reaction mixture was stirred at RT for 18 h. Volatiles were removed in vacuo to give 440 mg of the crude title compound (purity ˜50%, major contaminant excess of TFA) as a yellow viscous oil, which was used without further purification. MS (ESI): m/z=321.1 [M−TsOH+H]⁺

### Step a) 6-[[3-(trifluoromethoxy)phenyl]sulfonimidoyl]-2-azaspiro[3.3]heptane-2-carboxylic Acid Tert-Butyl Ester

To a solution of 6-[[3-(trifluoromethoxy)phenyl]thio]-2-azaspiro[3.3]heptane-2-carboxylic acid tert-butyl ester (B.336, Step a) (300 mg, 0.755 mmol) in methanol (1.5 mL) was added iodobenzene diacetate (608 mg, 1.89 mmol) and ammonium carbamate (58.9 mg, 0.755 mmol) after which the reaction mixture was stirred at RT for 3 h. The crude reaction solution was absorbed onto H-MN isolate and dried under vacuum followed by direct purification by flash chromatography with a SiO₂ column (eluent mixture of heptane and a solution (EtOAc:EtOH 3:1) (5% to 50%)) to give 228 mg of the title compound as a colorless gum. MS (ESI): m/z=421.1 [M+H]⁺

### Example B.349

### 3-(2-azaspiro[3.3]heptan-6-ylmethyl)-5-(trifluoromethyl)-1,2,4-thiadiazole; 4-methylbenzenesulfonic Acid

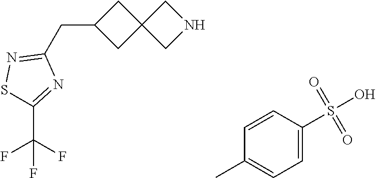 [SMILES: FC(F)(F)c1nc(CC2CC3(CNC3)C2)ns1 ; Cc1ccc(S(=O)(=O)O)cc1]

A mixture of tert-butyl 6-[[5-(trifluoromethyl)-1,2,4-thiadiazol-3-yl]methyl]-2-azaspiro[3.3]heptane-2-carboxylate (400 mg, 1.1 mmol) and TsOH (227 mg, 1.32 mmol) in EtOAc (5 mL) was stirred at 80° C. for 3 h. The reaction mixture was concentrated under reduced pressure and lyophilized to afford the title compound (415 mg, 0.95 mmol, 85.3% yield) as a white solid. MS (ESI): m/z=264.0 [M−TsOH+H]⁺

### Step a) tert-butyl 6-(cyanomethyl)-2-azaspiro[3.3]heptane-2-carboxylate

To a solution of tert-butyl 6-(methylsulfonyloxymethyl)-2-azaspiro[3.3]heptane-2-carboxylate (2740574-92-7) (5.0 g, 16.4 mmol) in DMSO (70 mL) was added potassium cyanide (2.45 g, 37.7 mmol) at room temperature. The resulting solution was stirred for 18 h at 80° C. The reaction was quenched by water (50 mL) and then extracted with TBME (3×100 mL). The organic layers were washed with brine, dried over Na₂SO₄, filtered and concentrated in vacuum to give the title compound (3.4 g, 14.4 mmol, 83.6% yield) as white solid. MS (ESI): m/z=181.0 [M−C₄H₈+H]⁺.

### Step b) tert-butyl 6-[2-(hydroxyamino)-2-imino-ethyl]-2-azaspiro[3.3]heptane-2-carboxylate

To a mixture of hydroxylamine; hydrochloride (2353 mg, 33.9 mmol) in ethanol (25 mL) was added TEA (3426 mg, 33.9 mmol) at 25° C. After 1 h, tert-butyl 6-(cyanomethyl)-2-azaspiro[3.3]heptane-2-carboxylate (4000 mg, 16.9 mmol) added to the reaction mixture above, then the reaction was stirred at 50° C. for further 12 h. The reaction was concentrated under reduced pressure to give a residue. The residue was dissolved in water (50.0 mL), extracted with ethyl acetate (50.0 mL×3), the combined extracts were concentrated under reduced pressure to give the title compound (4.5 g, 16.7 mmol, 98.7% yield) as a colorless oil, which was used for next step without further purification. MS (ESI): m/z=270.1 [M+H]⁺

### Step c) tert-butyl 6-(2-amino-2-imino-ethyl)-2-azaspiro[3.3]heptane-2-carboxylate; acetic Acid

To a solution of tert-butyl 6-[2-(hydroxyamino)-2-imino-ethyl]-2-azaspiro[3.3]heptane-2-carboxylate (4.5 g, 16.7 mmol) and Ac₂O (2.56 g, 25.1 mmol) in Acetic acid (20 mL) was added Pd/C (wet) (1.2 g) at 25° C. Then the reaction was stirred at 25° C. under H₂ atmosphere (H₂ balloon) for 12 h. The reaction mixture was filtered, the filtrate was concentrated under reduced pressure to give the crude title compound (5.2 g, 16.6 mmol, 99.3% yield) as a light yellow oil. MS (ESI): m/z=254.0 [M−AcOH+H]⁺

### Step d) tert-butyl 6-[(5-amino-1,2,4-thiadiazol-3-yl)methyl]-2-azaspiro[3.3]heptane-2-carboxylate

To a solution of tert-butyl 6-(2-amino-2-imino-ethyl)-2-azaspiro[3.3]heptane-2-carboxylate; acetic acid (3000 mg, 9.57 mmol) in water (30 mL) was added sodium hypochlorite (15.0 mL, 10.5 mmol) dropwise at 0° C., then the mixture was stirred at 20° C. for 1 h, then diluted with water and extracted with EtOAc (20 mL×3), the combined organic phase was dried over Na₂SO₄ and concentrated, the residue was dissolved in Methanol (30 mL), potassium thiocyanate (1023 mg, 10.5 mmol) was added at 0° C., then the solution was stirred at 20° C. for 11 h. The reaction mixture was filtered, the filtrate was concentrated under reduced pressure to give a residue. The residue was purified by column on silica (ethyl acetate:petroleum ether 0-80%) and concentrated under reduced pressure to give the title compound (1400 mg, 4.51 mmol, 47.1% yield) as a brown solid. MS (ESI): m/z=311.0 [M+H]⁺

### Step e) tert-butyl 6-[(5-iodo-1,2,4-thiadiazol-3-yl)methyl]-2-azaspiro[3.3]heptane-2-carboxylate

To a mixture of tert-butyl 6-[(5-amino-1,2,4-thiadiazol-3-yl)methyl]-2-azaspiro[3.3]heptane-2-carboxylate (1400 mg, 4.51 mmol and CuI (1718 mg, 9.02 mmol) in MeCN (50 mL) was added a solution of tert-butyl nitrite (930 mg, 9.02 mmol, 2.0 eq) in MeCN (5 mL) at 25° C., then the reaction was stirred at 70° C. under N₂ atmosphere for 12 h. The reaction mixture was filtered, the filtrate was concentrated under reduced pressure to give a residue. The residue was purified by column on silica (ethyl acetate:petroleum ether 0-40%) and concentrated under reduced pressure to give the title compound (900 mg, 2.14 mmol, 47.4% yield) as a yellow solid. MS (ESI): m/z=421.9 [M+H]⁺

### Step f) tert-butyl 6-[[5-(trifluoromethyl)-1,2,4-thiadiazol-3-yl]methyl]-2-azaspiro[3.3]heptane-2-carboxylate

A mixture of tert-butyl 6-[(5-iodo-1,2,4-thiadiazol-3-yl)methyl]-2-azaspiro[3.3]heptane-2-carboxylate (890 mg, 2.11 mmol), Diphenyl(trifluoromethyl) sulfonium trifluoromethanesulfonate (1025 mg, 2.54 mmol) and Cu (1207 mg, 6.34 mmol) in DMF (8 mL) was stirred at 60° C. under N₂ atmosphere for 12 h. The reaction mixture was filtered, the filtrate was concentrated under reduced pressure to give a residue. The residue was purified by reverse flash (FA condition; MeCN:H₂O=0-70%) and lyophilized to afford a residue. This residue was purified by column on silica (ethyl acetate:petroleum ether 0-30%) and concentrated under reduced pressure to give the title compound (400 mg, 1.1 mmol, 52.1% yield) as a colorless oil. MS (ESI): m/z=307.9 [M−C₄H₈+H]⁺

### Example B.358

### 4-(2-azaspiro[3.3]heptan-6-ylmethyl)-2-(trifluoromethoxy)benzamide; 4-methylbenzenesulfonic Acid

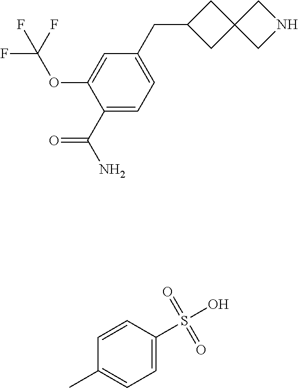 [SMILES: NC(=O)c1ccc(CC2CC3(CNC3)C2)cc1OC(F)(F)F ; Cc1ccc(S(=O)(=O)O)cc1]

To a solution of 6-[4-carbamoyl-3-(trifluoromethoxy)benzyl]-2-azaspiro[3.3]heptane-2-carboxylic acid tert-butyl ester (63 mg, 0.152 mmol) in ethyl acetate (1.01 mL) was added p-toluenesulfonic acid monohydrate (30.4 mg, 0.160 mmol). The reaction mixture was stirred overnight at reflux. Solvents were removed and product used without further purification for next step. MS (ESI): m/z=315.1 [M+H]⁺

### Step a) 6-[4-carbomethoxy-3-(trifluoromethoxy)benzylidene]-2-azaspiro[3.3]heptane-2-carboxylic Acid Tert-Butyl Ester

4-bromo-2-(trifluoromethoxy)benzoic acid methyl ester (981 mg, 603 μL, 3.28 mmol) and 6-[(4,4,5,5-tetramethyl-1,3,2-dioxaborolan-2-yl)methylene]-2-azaspiro[3.3]heptane-2-carboxylic acid tert-butyl ester (1000 mg, 2.98 mmol) were dissolved in a degassed solution of 1,4-dioxane (149 mL) and water (14.9 mL). The reaction mixture was degassed for 5 min again before addition of 1,1′-bis(di-tert-butylphosphino) ferrocene-palladium dichloride (97.2 mg, 0.149 mmol) followed by tripotassium phosphate (1.27 g, 5.97 mmol). The reaction mixture was then stirred at room temp. for 3.5 h. The reaction mixture was poured into EtOAc, washed with water. The aqueous layer was extracted back twice. Combined organic layers were washed with brine, dried over Na₂SO₄, filtered and concentrated under reduced pressure. The resulting crude was then purified by flash chromatography eluting with a gradient Heptane/EtOAc 0-35% to afford the title compound as a white solid. MS (ESI): m/z=372.1 [M−C₄H₈+H]⁺

### Step b) 6-[4-carbomethoxy-3-(trifluoromethoxy)benzyl]-2-azaspiro[3.3]heptane-2-carboxylic Acid Tert-Butyl Ester

6-[4-carbomethoxy-3-(trifluoromethoxy)benzylidene]-2-azaspiro[3.3]heptane-2-carboxylic acid tert-butyl ester (1050 mg, 2.46 mmol) was dissolved in methanol (81.9 mL) and tetrahydrofuran (40.9 mL). The reaction mixture was degassed for 10 min with argon. Then, still under argon, platinum (IV) oxide (112 mg, 0.491 mmol) was added to the mixture. The argon atmosphere was replaced by hydrogen (via balloon), and the reaction mixture stirred under hydrogen atmosphere for 1 h. The reaction mixture was filtrated, and the resulting solution was concentrated under reduced pressure to afford the crude title compound as an oil, which was used directly in the next step without further purification. MS (ESI): m/z=374.1 [M−C₄H₈+H]⁺

### Step c) 4-[(2-tert-butoxycarbonyl-2-azaspiro[3.3]heptan-6-yl)methyl]-2-(trifluoromethoxy)benzoic Acid

To a solution of 6-[4-carbomethoxy-3-(trifluoromethoxy)benzyl]-2-azaspiro[3.3]heptane-2-carboxylic acid tert-butyl ester (460 mg, 1.04 mmol) in tetrahydrofuran (2.6 mL) and methanol (2.6 mL) was added 1 M NaOH aqueous solution (4.16 mL, 4.16 mmol) at room temperature. The mixture was then heated at 70° C. for 48 h. Organic solvent was removed under reduced pressure and then the resulting crude material was acidified until pH 2. Then the aqueous layer was extracted three times with EtOAc. Organic layers were combined, dried over Na₂SO₄, filtered off and concentrated under reduced pressure to yield a crude product that was used directly in the next step without further purification. MS (ESI): m/z=360.1 [M−C₄H₈+H]⁺

### Step d) 6-[4-carbamoyl-3-(trifluoromethoxy)benzyl]-2-azaspiro[3.3]heptane-2-carboxylic Acid Tert-Butyl Ester

A solution of 4-[(2-tert-butoxycarbonyl-2-azaspiro[3.3]heptan-6-yl)methyl]-2-(trifluoromethoxy)benzoic acid (440 mg, 1.06 mmol) in dichloromethane (5.3 mL) was cooled at 0° C. CDI (177 mg, 1.06 mmol) was added and the resulting mixture was stirred 15 min. 2 M NH₃ in isopropanol solution (2.65 mL, 5.3 mmol) was then added and the reaction mixture was stirred overnight at room temp. A further addition of 2 M NH₃ in isopropanol solution (2.65 mL, 5.3 mmol) was made and the reaction stirred for 48 h. The reaction mixture was washed with 1

M HCl (aq) twice, and with water. The aqueous layer was back-extracted with dichloromethane. The combined organic layers were dried over Na₂SO₄ and concentrated under reduced pressure. The crude material was then purified by flash chromatography using hexane/EtOAc to afford the title compound as a viscous oil. MS (ESI): m/z=359.1 [M−C₄H₈+H]⁺

In analogy to Example B.358, the following building block was generated using the relevant commercial building blocks.

| | | | | MS, |
|---|---|---|---|---|
| | | | | ESI: |
| Ex. | Structure | Systematic Name | Building Blocks | m/z |
| B.359 | 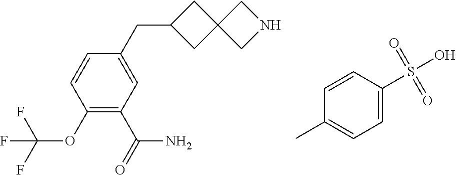 [SMILES: NC(=O)c1cc(CC2CC3(CNC3)C2)ccc1OC(F)(F)F ; Cc1ccc(S(=O)(=O)O)cc1] | 5-(2- azaspiro[3.3]heptan-6- ylmethyl)-2- (trifluoromethoxy)benz- amide; 4- methylbenzenesulfonic acid | 5-bromo-2- (trifluoromethoxy) benzoic acid methyl ester | 315.1 [M − TsOH + H]⁺ |

### Example B.366

### 2-[5-(trifluoromethyl)-2-pyridyl]-2,6-diazaspiro[3.3]heptane; 4-methylbenzenesulfonic Acid

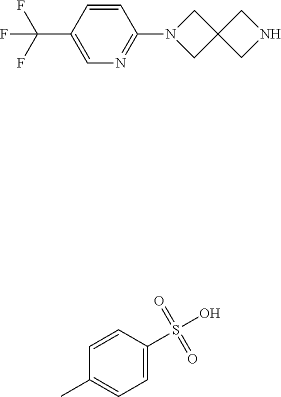 [SMILES: FC(F)(F)c1ccc(N2CC3(CNC3)C2)nc1 ; Cc1ccc(S(=O)(=O)O)cc1]

In a flask was added 6-[5-(trifluoromethyl)-2-pyridyl]-2,6-diazaspiro[3.3]heptane-2-carboxylic acid tert-butyl ester (285 mg, 0.830 mmol) and p-toluenesulfonic acid monohydrate (174 mg, 0.913 mmol) in ethyl acetate (7 mL), and the mixture was stirred at reflux overnight

Another equivalent of p-toluenesulfonic acid monohydrate (174 mg, 0.913 mmol) was added and the reaction stirred for a further 6 h. The solvent was evaporated and the crude was washed with diethyl ether to afford the crude title compound as an orange solid. MS (ESI): m/z=244.1 [M+H]⁺

### Step a) 6-[5-(trifluoromethyl)-2-pyridyl]-2,6-diazaspiro[3.3]heptane-2-carboxylic Acid Tert-Butyl Ester

In a flask was added 2-bromo-5-(trifluoromethyl)pyridine (250 mg, 1.11 mmol), 2-Boc-2,6-diazaspiro[3.3]heptane (241 mg, 1.22 mmol) and cesium carbonate (721 mg, 2.21 mmol) in 1,4-dioxane (5.38 mL) The suspension was bubbled with N₂ for 5 min and chloro(2-dicyclohexylphosphino-2′,6′-diisopropoxy-1,1′-biphenyl) [2-(2′-amino-1,1′-biphenyl)]palladium(II) (43.0 mg, 0.055 mmol) was added. The mixture was heated at 100° C. for 2 h. The mixture was diluted in EtOAc and filtered through celite, the filtrate was concentrated. Purification was performed by flash chromatography (heptane/EtOAc with gradient from 0 to 40% EtOAc to afford the title compound as a yellow solid. MS (ESI): m/z=344.1 [M+H]⁺

In analogy to Example B.367, the following building block was generated using the relevant commercial building blocks. In some cases different palladium/ligand catalysts were used in Step a) (e.g. Pd₂dba₃/Xantphos).

| | | | | MS, |
|---|---|---|---|---|
| | | | | ESI: |
| Ex. | Structure | Systematic Name | Building Blocks | m/z |
| B.367 | 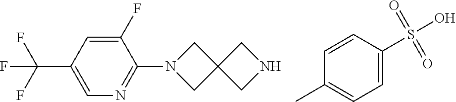 | 2-[3-fluoro-5- (trifluoromethyl)-2- pyridyl]-2,6- diazaspiro[3.3]heptane; 4- methylbenzenesulfonic acid | 2-bromo-3- fluoro-5- (trifluoromethyl)py- ridine (CAS: 89402-29-9) | 262.1 [M − TsOH + H]⁺ |
| B.378 | 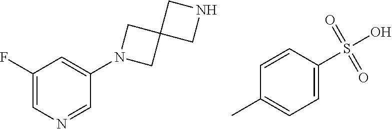 | 2-(5-fluoro-3- pyridyl)-2,6- diazaspiro[3.3]heptane; 4- methylbenzenesulfonic acid | 3-bromo-5- fluoropyridine | 194.0 [M − TsOH + H]⁺ |

### Example B.377

### 6-[[4-[1-(trifluoromethyl)cyclopropyl]pyrazol-1-yl]methyl]-2-azaspiro[3.3]heptane; 4-methylbenzenesulfonic Acid

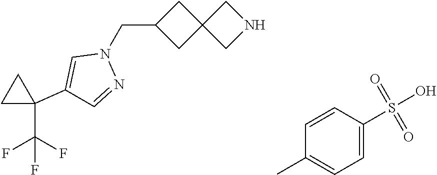 [SMILES: FC(F)(F)C1(c2cnn(CC3CC4(CNC4)C3)c2)CC1 ; Cc1ccc(S(=O)(=O)O)cc1]

A solution of tert-butyl 6-[[4-[1-(trifluoromethyl)cyclopropyl]pyrazol-1-yl]methyl]-2-azaspiro[3.3]heptane-2-carboxylate (400 mg, 1.04 mmol) and p-toluenesulfonic acid (214 mg, 1.25 mmol) in EtOAc (8 mL) was stirred at 80° C. for 12 h. The reaction mixture was concentrated and lyophilized to give the title compound (467 mg, 1.02 mmol, 91% yield) as a light brown solid. MS (ESI): m/z=286.2 [M+H]⁺

### Step a) tert-butyl 6-[(4-bromopyrazol-1-yl)methylene]-2-azaspiro[3.3]heptane-2-carboxylate

To a solution of 4-bromopyrazole (2000 mg, 13.6 mmol) in DCE (40 mL) was added tert-butyl 6-[(4,4,5,5-tetramethyl-1,3,2-dioxaborolan-2-yl)methylene]-2-azaspiro[3.3]heptane-2-carboxylate (6840 mg, 20.4 mmol), pyridine (2153 mg, 27.2 mmol), boric acid (841 mg, 13.6 mmol) and Cu(OAc)₂ (3670 mg, 18.4 mmol). The mixture was stirred at 70° C. for 12 h under 02. The reaction mixture was purified by prep-HPLC and lyophilized. The residue was triturated in petroleum ether (10 mL) and stirred for 10 min. The solid was collected by filtration to give the title compound (2867 mg, 8.09 mmol, 59% yield) as an off-white solid. MS (ESI): m/z=298.1 [M+H]⁺

### Step b) tert-butyl 6-[(4-bromopyrazol-1-yl)methyl]-2-azaspiro[3.3]heptane-2-carboxylate

To a solution of tert-butyl 6-[(4-bromopyrazol-1-yl)methylene]-2-azaspiro[3.3]heptane-2-carboxylate (2300 mg, 6.49 mmol) in EtOAc (25 mL) was added PtO₂ (920 mg, 4.05 mmol) at 20° C. under N₂, then the mixture was stirred at 20° C. under H₂ atmosphere (balloon) for 1 h. The precipitate was filtered off and the filtrate was dried in vacuo. The residue was purified over column chromatography (hexane/EtOAc, 1:1) to give the title compound (2200 mg, 6.18 mmol, 95% yield) as a light yellow solid.

MS (ESI): m/z=302.0 [M−C₄H₈+H]⁺

### Step c) tert-butyl 6-[[4-[1-(trifluoromethyl)vinyl]pyrazol-1-yl]methyl]-2-azaspiro[3.3]heptane-2-carboxylate

To a solution of tert-butyl 6-[(4-bromopyrazol-1-yl)methyl]-2-azaspiro[3.3]heptane-2-carboxylate (1700 mg, 4.77 mmol) in 1,4-Dioxane (20 mL) and water (4 mL) was added 1-(trifluoromethyl)vinylboronic acid hexylene glycol ester (1270 mg, 5.73 mmol), K₂CO₃ (1980 mg, 14.3 mmol) and [1,1′-BIS(DIPHENYLPHOSPHINO)FERROCENE]PALLADIUM(II) CHLORIDE (390 mg, 0.48 mmol) under N₂. The mixture was stirred at 80° C. for 12 h under N₂ atmosphere. The precipitate was filtered off and the filtrate was dried in vacuo. The residue was purified over column chromatography (PE/EA, 0-60%). The reaction mixture was purified by prep-HPLC, and lyophilized to give the title compound (1134.0 mg, 3.05 mmol, 64% yield) as light brown solid. MS (ESI): m/z=372.1 [M+H]⁺

### Step d) tert-butyl 6-[[4-[1-(trifluoromethyl)cyclopropyl]pyrazol-1-yl]methyl]-2-azaspiro[3.3]heptane-2-carboxylate

To a solution of tert-butyl 6-[[4-[1-(trifluoromethyl)vinyl]pyrazol-1-yl]methyl]-2-azaspiro[3.3]heptane-2-carboxylate (1000 mg, 2.69 mmol) in THF (20 mL) was added diphenyl(methyl) sulfonium tetrafluoroborate (1008 mg, 3.5 mmol). The suspension was cooled to 0° C. and NaHMDS/THF (1 M) (10.8 mL, 10.8 mmol) was added dropwise. The reaction mixture was warmed to 20° C. for and stirred for 12 h. The reaction mixture was purified by prep-HPLC and lyophilized to give the title compound (432 mg, 1.12 mmol, 42% yield) as light yellow solid. MS (ESI): m/z=386.1 [M+H]⁺

In analogy to Example B.377, the following building block was generated using the relevant commercial building blocks.

| | | | | MS, |
|---|---|---|---|---|
| | | | | ESI: |
| Ex. | Structure | Systematic Name | Building Blocks | m/z |
| B.384 | 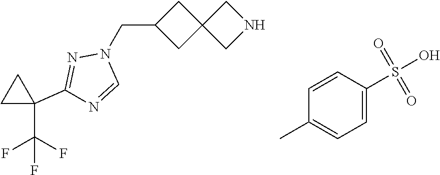 [SMILES: FC(F)(F)C1(c2ncn(CC3CC4(CNC4)C3)n2)CC1 ; Cc1ccc(S(=O)(=O)O)cc1] | 6-[[3-[1- (trifluoromethyl)cyclo- propyl]-1,2,4-triazol- 1-yl]methyl]-2- azaspiro[3.3]heptane; 4- methylbenzenesulfonic acid | 3-bromo-1H- 1,2,4-triazole | 287.0 [M − TsOH + H]⁺ |

### Example B.379

### 6-[[1-cyclopropyl-3-(trifluoromethyl) pyrazol-4-yl]methyl]-2-azaspiro[3.3]heptane; 4-methylbenzenesulfonic Acid

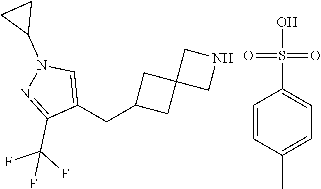

To the mixture of tert-butyl 6-[[1-cyclopropyl-3-(trifluoromethyl) pyrazol-4-yl]methyl]-2-azaspiro[3.3]heptane-2-carboxylate (790 mg, 2.05 mmol) in EtOAc (8 mL) was added p-toluenesulfonic acid (388 mg, 2.25 mmol) at 25° C., then the reaction mixture was stirred at 80° C. for 12 h. The reaction mixture was concentrated under reduced pressure to give a residue. 20 mL deionized water and 2 mL ACN was added to the residue, which was then lyophilized to give the title compound (811 mg, 1.77 mmol, 85% yield) as a yellow oil. MS (ESI): m/z=286.1 [M−TsOH+H]⁺

### Step a) tert-butyl 6-[[5-(trifluoromethyl)-1H-pyrazol-4-yl]methylene]-2-azaspiro[3.3]heptane-2-carboxylate

To the solution of tert-butyl 6-[(4,4,5,5-tetramethyl-1,3,2-dioxaborolan-2-yl)methylene]-2-azaspiro[3.3]heptane-2-carboxylate (5009 mg, 14.9 mmol), 4-bromo-5-(trifluoromethyl)-1H-pyrazole (2920 mg, 13.6 mmol) in 1,4-dioxane (73 mL), water (14.6 mL) was added potassium carbonate (3750 mg, 27.2 mmol), 1,1′-bis(diphenylphosphino) ferrocene-palladium(II) dichloride dichloromethane complex (554 mg, 0.68 mmol) at 20° C., then the reaction was stirred at 100° C. for 12 h under N₂. The reaction mixture was filtered and concentrated under reduced pressure to give a residue which was purified by flash silica gel chromatography (eluent of 0-40% ethyl acetate/petroleum ether) to give a crude product which was further purified by flash silica gel chromatography (eluent of 0-40% ethyl acetate/petroleum ether) to give the title compound (790 mg, 2.3 mmol, 17% yield) as a yellow oil. MS (ESI): m/z=288.0 [M−C₄H₈+H]⁺.

### Step b) tert-butyl 6-[[1-cyclopropyl-3-(trifluoromethyl) pyrazol-4-yl]methylene]-2-azaspiro[3.3]heptane-2-carboxylate

To a mixture of tert-butyl 6-[[5-(trifluoromethyl)-1H-pyrazol-4-yl]methylene]-2-azaspiro[3.3]heptane-2-carboxylate (600 mg, 1.75 mmol), cyclopropylboronic acid (600 mg, 6.99 mmol) in DCE (6 mL) was added pyridine (0.42 mL, 5.24 mmol), boric acid (108 mg, 1.75 mmol), copper diacetate (698 mg, 3.5 mmol) at 20° C., then the reaction mixture was stirred at 100° C. for 16 h under 02 (balloon). The reaction mixture was filtered and then diluted with water 50 mL and extracted with EtOAc 150 mL (50 mL×3). The combined organic layers were washed with brine (40 mL) dried over Na₂SO₄, filtered and concentrated under reduced pressure to give a residue which was purified by silica gel chromatography (0-28% ethyl acetate/petroleum ether) to give the title compound (350 mg, 0.91 mmol, 52%) as a colorless oil. MS (ESI): m/z=328.0 [M−C₄H₈+H]⁺

### Step c) tert-butyl 6-[[1-cyclopropyl-3-(trifluoromethyl) pyrazol-4-yl]methyl]-2-azaspiro[3.3]heptane-2-carboxylate

To the mixture of tert-butyl 6-[[1-cyclopropyl-3-(trifluoromethyl) pyrazol-4-yl]methylene]-2-azaspiro[3.3]heptane-2-carboxylate (720 mg, 1.88 mmol) in EtOAc (15 mL) was added Pd/C (wet, 216 mg, 10%) at 25° C., then the reaction mixture was stirred at 25° C. for 0.5 h under H₂ (15 Psi). The reaction mixture was filtered and the filter liquor was concentrated under reduced pressure to give the title compound (640 mg, 1.66 mmol, 88.4% yield) as a colorless oil. MS (ESI): m/z=330.0 [M−C₄H₈+H]⁺.

### Example B.381

### 6-[[5-methyl-3-(trifluoromethyl) pyrazol-1-yl]methyl]-2-azaspiro[3.3]heptane; 4-methylbenzenesulfonic Acid

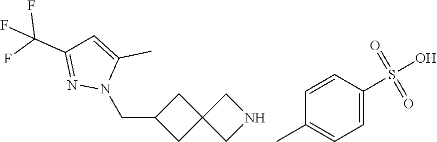

A mixture of tert-butyl 6-[[5-methyl-3-(trifluoromethyl) pyrazol-1-yl]methyl]-2-azaspiro[3.3]heptane-2-carboxylate (3.8 g, 10.57 mmol, 1.0 eq) and p-toluenesulfonic acid (4.55 g, 26.43 mmol, 2.5 eq) in EtOAc (70 mL) was stirred at 25° C. for 24 h. Then the reaction mixture was concentrated and crystallized from MTBE to give the title compound (3940 mg, 9.13 mmol, 86% yield) as white solid. MS (ESI): m/z=260.2 [M+H]⁺

### Step a) tert-butyl 6-[[5-methyl-3-(trifluoromethyl) pyrazol-1-yl]methyl]-2-azaspiro[3.3]heptane-2-carboxylate

To a solution of tert-butyl 6-(hydroxymethyl)-2-azaspiro[3.3]heptane-2-carboxylate (1000 mg, 4.4 mmol) in toluene (20 mL) was added 3-methyl-5-(trifluoromethyl)-1H-pyrazole (CAS: 10010-93-2) (660 mg, 4.4 mmol), and (Cyanomethylene)tributylphosphorane (1590 mg, 6.6 mmol). The reaction mixture was stirred at 100° C. for 12 h under N₂ atmosphere. The reaction mixture was filtered, and the filtrate was concentrated under reduced pressure to give a residue. The residue was purified by prep-HPLC and lyophilized to give the title compound (485 mg, 1.35 mmol, 31% yield) as dark brown powder. MS (ESI): m/z=304.0 [M+H]⁺

Note: Regioisomer tert-butyl 6-[[3-methyl-5-(trifluoromethyl) pyrazol-1-yl]methyl]-2-azaspiro[3.3]heptane-2-carboxylate (735 mg, 2.05 mmol, 46.5% yield) was also generated as a dark brown oil. MS (ESI): m/z=304.0 [M+H]⁺

### Example B.382

### 6-[[3-methyl-5-(trifluoromethyl) pyrazol-1-yl]methyl]-2-azaspiro[3.3]heptane; 4-methylbenzenesulfonic Acid

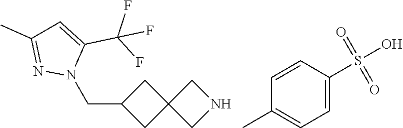

A mixture of tert-butyl 6-[[3-methyl-5-(trifluoromethyl) pyrazol-1-yl]methyl]-2-azaspiro[3.3]heptane-2-carboxylate (generated as regioisomer in B.381, Step a)) (2.3 g, 6.4 mmol) and p-toluenesulfonic acid (2755 mg, 16.0 mmol) in EtOAc (50 mL) was stirred at 25° C. for 24 h. Then the reaction mixture was concentrated and crystallized from MTBE to give the title compound (1468 mg, 3.4 mmol, 53% yield) as white solid. MS (ESI): m/z=260.2 [M+H]⁺

In analogy to Example B.381/B.382, the following regioisomeric pairs of building blocks were generated using the relevant commercial building blocks.

| | | | | MS, |
|---|---|---|---|---|
| | | | | ESI: |
| Ex. | Structure | Systematic Name | Building Blocks | m/z |
| B.392 | 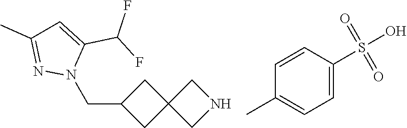 | 6-[[5- (difluoromethyl)-3- methyl-pyrazol-1- yl]methyl]-2- azaspiro[3.3]heptane; 4- methylbenzenesulfonic acid | 5- (difluoromethyl)- 3-methyl-1H- pyrazole (CAS: 936033-61-3) | 242.1 [M − TsOH + H]⁺ |
| B.393 | 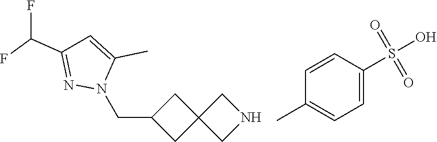 | 6-[[3- (difluoromethyl)-5- methyl-pyrazol-1- yl]methyl]-2- azaspiro[3.3]heptane; 4- methylbenzenesulfonic acid | 5- (difluoromethyl)- 3-methyl-1H- pyrazole (CAS: 936033-61-3) | 242.1 [M − TsOH + H]⁺ |

### Example B.386

### [4-(2-azaspiro[3.3]heptan-6-yloxy)phenyl]-imino-oxo-(trifluoromethyl)-2′-sulfane; 4-methylbenzenesulfonic Acid

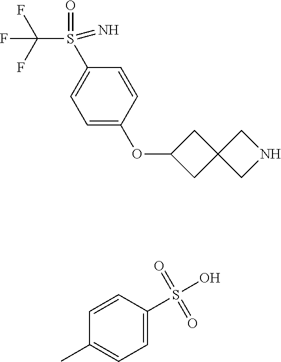 [SMILES: N=S(=O)(c1ccc(OC2CC3(CNC3)C2)cc1)C(F)(F)F ; Cc1ccc(S(=O)(=O)O)cc1]

To solution of tert-butyl 6-[4-(trifluoromethylsulfonimidoyl) phenoxy]-2-azaspiro[3.3]heptane-2-carboxylate (380 mg, 0.9 mmol) in EtOAc (5 mL) was added p-toluenesulfonic acid monohydrate (206 mg, 1.08 mmol) and stirred at 25° C. for 18 h, then reaction mixture was evaporated and purified by HPLC to give the title compound (143 mg, 0.29 mmol, 31% yield) as a yellow solid. MS (ESI): m/z=321.0 [M+H]⁺

### Step a) 4-(trifluoromethylsulfonimidoyl)phenol

To a stirred solution of imino-oxo-[4-(4,4,5,5-tetramethyl-1,3,2-dioxaborolan-2-yl)phenyl]-(trifluoromethyl)-λ6-sulfane (CAS: 1798336-50-1) (1.5 g, 4.48 mmol) in THF (75 mL) at 0° C. under air was added sodium hydrogen carbonate (376 mg, 4.48 mmol) in water (7.5 mL), followed by slow addition of hydrogen peroxide (1522 mg, 44.8 mmol). The reaction mixture was stirred at room temperature for 4 h. The resultant mixture was washed with water and aqueous sodium hydrogensulfite before being dried and concentrated to afford 4-(trifluoromethylsulfonimidoyl)phenol (1.0 g, 4.44 mmol, 94% yield) as yellow solid. MS (ESI): m/z=226.0 [M+H]⁺

### Step b) tert-butyl 6-[4-(trifluoromethylsulfonimidoyl) phenoxy]-2-azaspiro[3.3]heptane-2-carboxylate

To a mixture of 4-(trifluoromethylsulfonimidoyl)phenol (1.0 g, 4.44 mmol) and tert-butyl 6-hydroxy-2-azaspiro[3.3]heptane-2-carboxylate (947 mg, 4.44 mmol) was added cyanomethylenetributylphosphorane (2140 mg, 8.88 mmol) in Toluene (30 mL), and the resulting mixture was heated at 120° C. and stirred for 48 h. The reaction solution was concentrated. The residue was purified by flash chromatography (hexane/MTBE (10-100%)) to obtain the title compound (320 mg, 0.76 mmol, 16% yield) as yellow solid. MS (ESI): m/z=365.0 [M−C₄H₈+H]⁺.

In analogy to Example B.386, the following building block was generated using the relevant commercial building blocks.

| | | | | MS, |
|---|---|---|---|---|
| | | | | ESI: |
| Ex. | Structure | Systematic Name | Building Blocks | m/z |
| B.390 | 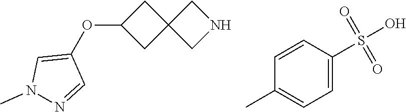 | 6-(1-methylpyrazol-4- yl)oxy-2- azaspiro[3.3]heptane; 4- methylbenzenesulfonic acid | 1-methylpyrazol- 4-ol | 194.2 [M − TsOH + H]⁺ |

### Example C.1

### 4-bromo-2-methylsulfonyl-1-(trifluoromethyl)benzene

To a solution of 4-chloro-2-methylsulfanyl-1-(trifluoromethyl)benzene (1950 mg, 8.6 mmol) in 1,2-dichloroethane (20 mL), CH₃CN (20 mL) and water (40 mL) cooled with a water bath was added sodium periodate (3680 mg, 17.2 mmol) and ruthenium (III) chloride hydrate (19.4 mg, 0.090 mmol) at 0° C., then the mixture was stirred at 25° C. for 12 h. The reaction mixture was diluted with 40 mL water, and extracted with 50 mL DCM and 2×50 mL EtOAc. The combined organic layers were washed with 75 mL brine, dried over Na₂SO₄, filtered, and evaporated. Purification by FC (SiO₂; PE/EtOAc) gave the title compound (1800 mg, 73% yield) as a white solid. ¹H NMR (400 MHZ, CHLOROFORM-d) δ ppm 8.32 (d, J=1.96 Hz, 1H) 7.87 (d, J=8.44 Hz, 1H) 7.76 (dd, J=8.44, 1.22 Hz, 1H) 3.22 ppm (s, 3H).

### Step a) 4-chloro-2-methylsulfanyl-1-(trifluoromethyl)benzene

A mixture of 4-chloro-2-fluoro-1-(trifluoromethyl)benzene (CAS: 94444-59-4) (5.0 g, 25.2 mmol) in DMF (50 mL) was added sodium methanethiolate (2.12 g, 30.2 mmol). The mixture was stirred at 50° C. for 2 h. The reaction mixture was diluted with 150 mL water, extracted with 2×75 mL EtOAc. The combined organic layers were washed with 100 mL brine, dried over Na₂SO₄, filtered, and evaporated. Purification by FC (SiO₂; PE/) gave the title compound (2 g, 31.5% yield) as a yellow solid. 1H NMR (400 MHZ, CHLOROFORM-d) δ ppm 7.55 (d, J=8.44 Hz, 1H) 7.30 (s, 1H) 7.20 (dd, J=8.38, 0.92 Hz, 1H) 2.52-2.57 ppm (m, 3H).

### Example C.6

### [3-bromo-5-(trifluoromethyl)phenyl]-imino-methyl-oxo-λ6-sulfane

Under argon atmosphere, a 200 mL sealed tube was charged with 1-bromo-3-methylsulfinyl-5-(trifluoromethyl)benzene (10.0 g, 34.8 mmol), amino 4-nitrobenzoate; trifluoromethanesulfonic acid (28.9 g, 87.1 mmol), ferrous sulfate (1058 mg, 6.97 mmol), 1,10-phenanthroline (2511 mg, 13.9 mmol) and ACN (80 mL). Then, the reaction mixture was stirred at 40° C. for 72 h. After cooling to room temperature, the reaction mixture was quenched with a saturated NaHCO₃ solution (100 mL). The mixture was extracted with CH₂Cl₂ (150 mL×3). The combined organic layers were dried over anhydrous Na₂SO₄ and concentrated in vacuo. The product was purified by column chromatography to give the title compound (2.2 g, 7.28 mmol, 19.9% yield) as a light yellow solid. MS (ESI): m/z=302.0/304.0 [M+H]⁺

### Step a) 1-bromo-3-methylsulfinyl-5-(trifluoromethyl)benzene

1-bromo-3-methylsulfanyl-5-(trifluoromethyl)benzene (11.0 g, 40.6 mmol) was dissolved in trifluoroacetic acid (62.5 mL, 812 mmol), and the resulting mixture was cooled at 0° C. with an ice bath. Next, hydrogen peroxide (3.77 mL, 44.6 mmol) in water (4 mL) was added, and the mixture was stirred 12 h at room temperature. After completion, the mixture was concentrated and partitioned between DCM and NaHCO₃ sat. aq. solution. The organic layer was dried and concentrated to obtain the title compound (10.0 g, 34.83 mmol, 81.55% yield) as white solid. MS (ESI): m/z=287.0/289.0 [M+H]⁺

### Example C.7

### 1-chloro-4-dimethylphosphoryl-2-fluoro-benzene

To a solution of 4-chloro-3-fluoroiodobenzene (2000 mg, 7.8 mmol) in 1,4-dioxane (20 mL) was added TEA (2.17 mL, 15.6 mmol), dimethylphosphine oxide (CAS: 7211-39-4) (730 mg, 9.36 mmol), Xantphos (903 mg, 1.56 mmol) and Pd₂(dba)₃ (714 mg, 0.78 mmol) at 25° C. The reaction mixture was stirred at 70° C. for 12 h under N₂ atmosphere. The reaction mixture was filtered, the filtrate was concentrated under reduced pressure to give a residue. The crude product was purified by reverse flash (FA condition; MeCN:H₂O 0-30%) and lyophilized to give 1-chloro-4-dimethylphosphoryl-2-fluoro-benzene (1387 mg, 6.71 mmol, 86% yield) as light yellow powder. MS (ESI): m/z=207.0 [M+H]⁺

### Example C.8

### 1-bromo-3-dimethylphosphoryl-5-fluoro-benzene

To a solution of 1-bromo-3-fluoro-5-iodo-benzene (2.0 g, 6.65 mmol) in 1,4-dioxane (20 mL) was added TEA (2.32 mL, 16.6 mmol), dimethylphosphine oxide (CAS: 7211-39-4) (545 mg, 6.98 mmol), Xantphos (1923 mg, 3.32 mmol) and Pd₂(dba)₃ (609 mg, 0.66 mmol). The reaction mixture was stirred at 60° C. for 16 h under N₂ atmosphere. The reaction mixture was filtered and the filter was concentrated. The crude product was purified by reversed-phase HPLC (0.1% FA condition) and lyophilized to give 1-bromo-3-dimethylphosphoryl-5-fluoro-benzene (1.36 g, 5.42 mmol, 81.5% yield) as brown solid which confirmed by ¹H NMR. MS (ESI): m/z=251.1 [M+H]⁺: ¹H NMR (400 MHZ, CHLOROFORM-d) δ=7.68-7.62 (m, 1H), 7.45-7.37 (m, 2H), 1.78 (s, 3H), 1.75 (s, 3H)

### Example C.9

### 1-(bromomethyl)-2-fluoro-4-(trifluoromethylsulfonyl)benzene

To a mixture of 2-fluoro-1-methyl-4-(trifluoromethylsulfonyl)benzene (4.2 g, 17.3 mmol) in trifluoromethylbenzene (80.0 mL, 646 mmol) was added N-BROMOSUCCINIMIDE (6.33 g, 35.6 mmol) and 2,2-AZOBIS(2-METHYLPROPIONITRILE) (5.84 g, 35.6 mmol). The reaction mixture was stirred at 90° C. for 12 h under N₂ atmosphere. The reaction mixture was concentrated under reduced pressure to give a residue. The residue was purified by flash chromatography on silica gel eluting with PE/EtOAc (9:1) to afford the title compound (2.5 g, 7.79 mmol, 44.9% yield) as a yellow oil. ¹H NMR (400 MHZ, CHLOROFORM-d) δ=4.55 (s, 2H), 7.73-7.79 (m, 2H), 7.85 (dd, J=8.01, 1.28 Hz, 1H).

### Example 1

### 2-[6-[[3-(trifluoromethylsulfonimidoyl)benzyl]-2-azaspiro[3.3]heptane-2-carbonyl]-2,5-diazaspiro[3.4]octan-6-one

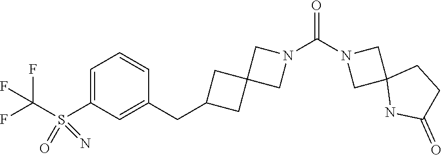 [SMILES: N=S(=O)(c1cccc(CC2CC3(C2)CN(C(=O)N2CC4(CCC(=O)N4)C2)C3)c1)C(F)(F)F]

To a solution of 2,5-diazaspiro[3.4]octan-6-one hydrochloride (A.1: CAS: 1630906-86-3) (23 mg, 0.141 mmol) in N,N-dimethylformamide (0.746 mL) cooled down to 0° C. was added DIPEA (128 mg, 173 μL, 0.990 mmol) followed by addition of bis(1,2,4-triazol-1-yl) methanone (24.4 mg. 0.149 mmol) after which the reaction mixture was stirred at 0° C. for 30 min. [3-(2-azaspiro[3.3]heptan-6-ylmethyl)phenyl]-imino-keto-(trifluoromethyl)-λ6-sulfane; tosylic acid (B.1) (72.9 mg, 0.149 mmol) was added to the reaction mixture, which was then stirred at 50° C. for 18 h. The crude reaction mixture was directly submitted for reversed-phase HPLC purification to yield 28.9 mg of the title compound as a colorless oil. MS (ESI): m/z=471.4 [M+H]⁺

In analogy to Example 1, Examples in the following table were generated, using the respective building blocks A.X and B.X.

| | | | | MS, |
|---|---|---|---|---|
| | | | Building | ESI: |
| Ex. | Structure | Systematic Name | Blocks | m/z |
| 2 | 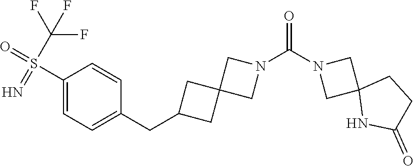 [SMILES: N=S(=O)(c1ccc(CC2CC3(C2)CN(C(=O)N2CC4(CCC(=O)N4)C2)C3)cc1)C(F)(F)F] | 2-[6-[4- (trifluoromethylsulfoni- midoyl)benzyl]-2- azaspiro[3.3]heptane-2- carbonyl]-2,5- diazaspiro[3.4]octan-6- one | A.1 and B.2 | 471.4 [M + H]⁺ |
| 5 | 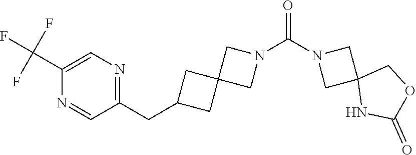 [SMILES: O=C1NC2(CO1)CN(C(=O)N1CC3(CC(Cc4cnc(C(F)(F)F)cn4)C3)C1)C2] | 2-[6-[[5- (trifluoromethyl)pyrazin- 2-y1]methyl]-2- azaspiro[3.3]heptane-2- carbonyl]-7-oxa-2,5- diazaspiro[3.4]octan-6- one | A.2 (CAS: 1780174- 72-2) and B.4 | 412.1 [M + H]⁺ |
| 6 | 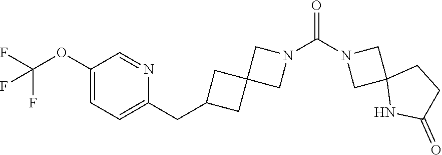 [SMILES: O=C1CCC2(CN(C(=O)N3CC4(CC(Cc5ccc(OC(F)(F)F)cn5)C4)C3)C2)N1] | 2-[6-[[5- (trifluoromethoxy)-2- pyridyl]methyl]-2- azaspiro[3.3]heptane-2- carbonyl]-2,5- diazaspiro[3.4]octan-6- one | A.1 and B.5 | 425.3 [M + H]⁺ |
| 7 | 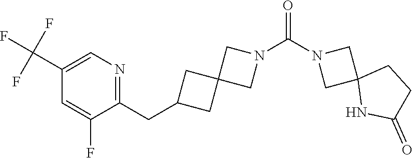 [SMILES: O=C1CCC2(CN(C(=O)N3CC4(CC(Cc5ncc(C(F)(F)F)cc5F)C4)C3)C2)N1] | 2-[6-[[3-fluoro-5- (trifluoromethyl)-2- pyridyl]methyl]-2- azaspiro[3.3]heptane-2- carbonyl]-2,5- diazaspiro[3.4]octan-6- one | A.1 and B.6 | 427.3 [M + H]⁺ |
| 10 | 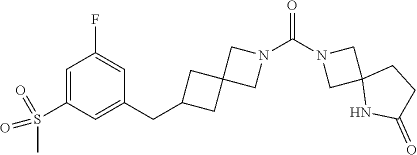 [SMILES: CS(=O)(=O)c1cc(F)cc(CC2CC3(C2)CN(C(=O)N2CC4(CCC(=O)N4)C2)C3)c1] | 2-[6-(3-fluoro-5-mesyl- benzyl)-2- azaspiro[3.3]heptane-2- carbonyl]-2,5- diazaspiro[3.4]octan-6- one | A.1 and B.9 | 436.3 [M + H]⁺ |
| 11 | 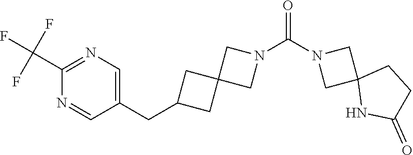 [SMILES: O=C1CCC2(CN(C(=O)N3CC4(CC(Cc5cnc(C(F)(F)F)nc5)C4)C3)C2)N1] | 2-[6-[[2- (trifluoromethyl)pyrimi- din-5-y1]methyl]-2- azaspiro[3.3]heptane-2- carbonyl]-2,5- diazaspiro[3.4]octan-6- one | A.1 and B.10 | 410.2 [M + H]⁺ |
| 12 | 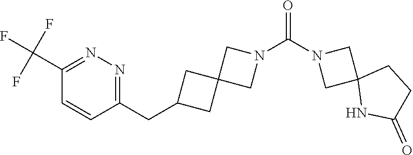 [SMILES: O=C1CCC2(CN(C(=O)N3CC4(CC(Cc5ccc(C(F)(F)F)nn5)C4)C3)C2)N1] | 2-[6-[[6- (trifluoromethyl)pyrida- zin-3-y1]methyl]-2- azaspiro[3.3]heptane-2- carbonyl]-2,5- diazaspiro[3.4]octan-6- one | A.1 and B.11 | 409.4 [M + H]⁺ |
| 13 | 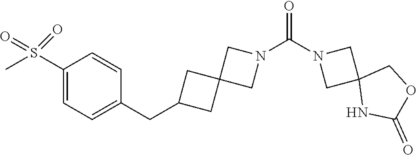 [SMILES: CS(=O)(=O)c1ccc(CC2CC3(C2)CN(C(=O)N2CC4(COC(=O)N4)C2)C3)cc1] | 2-[6-(4-mesylbenzyl)-2- azaspiro[3.3]heptane-2- carbonyl]-7-oxa-2,5- diazaspiro[3.4]octan-6- one | A.2 and B.7 | 419.5 [M + H]⁺ |
| 14 | 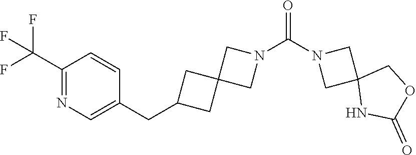 [SMILES: O=C1NC2(CO1)CN(C(=O)N1CC3(CC(Cc4ccc(C(F)(F)F)nc4)C3)C1)C2] | 2-[6-[[6- (trifluoromethyl)-3- pyridyl]methyl]-2- azaspiro[3.3]heptane-2- carbonyl]-7-oxa-2,5- diazaspiro[3.4]octan-6- one | A.2 and B.3 | 410.4 [M + H]⁺ |
| 15 | 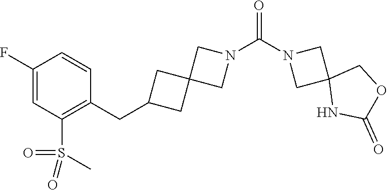 [SMILES: CS(=O)(=O)c1cc(F)ccc1CC1CC2(C1)CN(C(=O)N1CC3(COC(=O)N3)C1)C2] | 2-[6-(4-fluoro-2-mesyl- benzyl)-2- azaspiro[3.3]heptane-2- carbonyl]-7-oxa-2,5- diazaspiro[3.4]octan-6- one | A.2 and B.12 | 437.5 [M+H]⁺ |
| 16 | 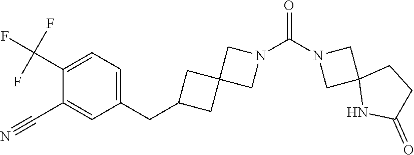 [SMILES: N#Cc1cc(CC2CC3(C2)CN(C(=O)N2CC4(CCC(=O)N4)C2)C3)ccc1C(F)(F)F] | 5-[[2-(6-keto-2,5- diazaspiro[3.4]octane- 2-carbonyl)-2- azaspiro[3.3]heptan-6- yl]methyl]-2- (trifluoromethyl)benzoni- trile | A.1 and B.13 | 433.2 [M + H]⁺ |
| 17 | 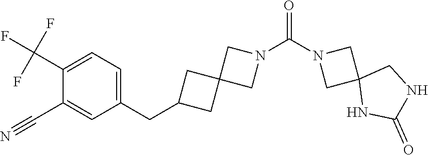 [SMILES: N#Cc1cc(CC2CC3(C2)CN(C(=O)N2CC4(CNC(=O)N4)C2)C3)ccc1C(F)(F)F] | 5-[[2-(6-keto-2,5,7- triazaspiro[3.4]octane- 2-carbonyl)-2- azaspiro[3.3]heptan-6- yl]methyl]-2- (trifluoromethyl)benzoni- trile | A.3 (CAS: 1609406- 92-9) and B.13 | 434.3 [M + H]⁺ |
| 18 | 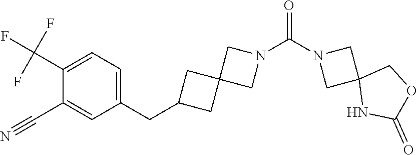 [SMILES: N#Cc1cc(CC2CC3(C2)CN(C(=O)N2CC4(COC(=O)N4)C2)C3)ccc1C(F)(F)F] | 5-[[2-(6-keto-7-oxa- 2,5- diazaspiro[3.4]octane- 2-carbonyl)-2- azaspiro[3.3]heptan-6- yl]methyl]-2- (trifluoromethyl)benzoni- trile | A.2 and B.13 | 435.3 [M + H]⁺ |
| 19 | 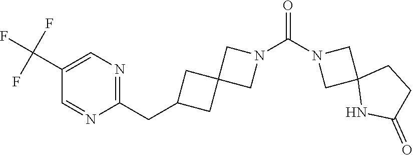 [SMILES: O=C1CCC2(CN(C(=O)N3CC4(CC(Cc5ncc(C(F)(F)F)cn5)C4)C3)C2)N1] | 2-[6-[[5- (trifluoromethyl)pyrimi- din-2-yl]methyl]-2- azaspiro[3.3]heptane-2- carbonyl]-2,5- diazaspiro[3.4]octan-6- one | A.1 and B.14 | 410.3 [M + H]⁺ |
| 20 | 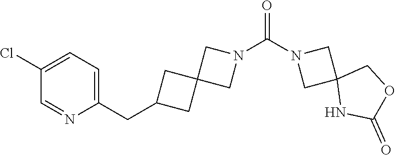 [SMILES: O=C1NC2(CO1)CN(C(=O)N1CC3(CC(Cc4ccc(Cl)cn4)C3)C1)C2] | 2-[6-[(5-chloro-2- pyridyl)methyl]-2- azaspiro[3.3]heptane-2- carbonyl]-7-oxa-2,5- diazaspiro[3.4]octan-6- one | A.2 and B.15 | 377.1 [M + H]⁺ |
| 21 | 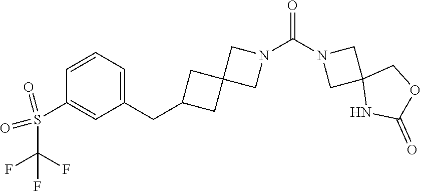 [SMILES: O=C1NC2(CO1)CN(C(=O)N1CC3(CC(Cc4cccc(S(=O)(=O)C(F)(F)F)c4)C3)C1)C2] | 2-[6-(3-triflylbenzyl)-2- azaspiro[3.3]heptane-2- carbonyl]-7-oxa-2,5- diazaspiro[3.4]octan-6- one | A.2 and B.16 | 474.2 [M + H]⁺ |
| 22 | 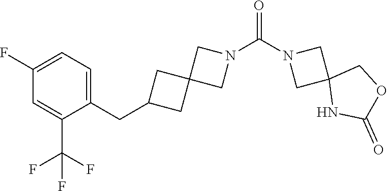 [SMILES: O=C1NC2(CO1)CN(C(=O)N1CC3(CC(Cc4ccc(F)cc4C(F)(F)F)C3)C1)C2] | 2-[6-[4-fluoro-2- (trifluoromethyl)benzyl]- 2- azaspiro[3.3]heptane-2- carbonyl]-7-oxa-2,5- diazaspiro[3.4]octan-6- one | A.2 and B.17 | 428.2 [M + H]⁺ |
| 23 | 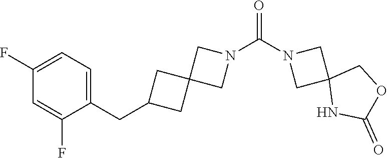 [SMILES: O=C1NC2(CO1)CN(C(=O)N1CC3(CC(Cc4ccc(F)cc4F)C3)C1)C2] | 2-[6-(2,4- difluorobenzyl)-2- azaspiro[3.3]heptane-2- carbonyl]-7-oxa-2,5- diazaspiro[3.4]octan-6- one | A.2 and B.18 | 378.1 [M + H]⁺ |
| 24 | 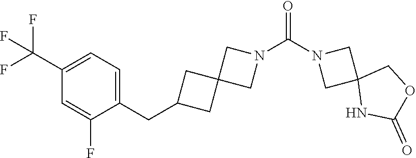 [SMILES: O=C1NC2(CO1)CN(C(=O)N1CC3(CC(Cc4ccc(C(F)(F)F)cc4F)C3)C1)C2] | 2-[6-[2-fluoro-4- (trifluoromethyl)benzyl]- 2- azaspiro[3.3]heptane-2- carbonyl]-7-oxa-2,5- diazaspiro[3.4]octan-6- one | A.2 and B.19 | 428.2 [M + H]⁺ |
| 25 | 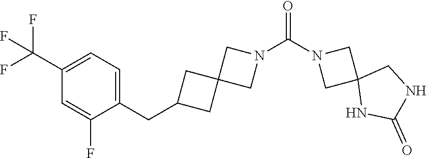 [SMILES: O=C1NCC2(CN(C(=O)N3CC4(CC(Cc5ccc(C(F)(F)F)cc5F)C4)C3)C2)N1] | 2-[6-[2-fluoro-4- (trifluoromethyl)benzyl]- 2- azaspiro[3.3]heptane-2- carbonyl]-2,5,7- triazaspiro[3.4]octan-6- one | A.3 and B.19 | 427.2 [M + H]⁺ |
| 26 | 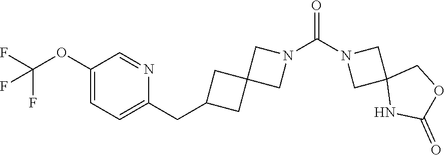 [SMILES: O=C1NC2(CO1)CN(C(=O)N1CC3(CC(Cc4ccc(OC(F)(F)F)cn4)C3)C1)C2] | 2-[6-[[5- (trifluoromethoxy)-2- pyridyl]methyl]-2- azaspiro[3.3]heptane-2- carbonyl]-7-oxa-2,5- diazaspiro[3.4]octan-6- one | A.2 and B.5 | 427.3 [M + H]⁺ |
| 27 | 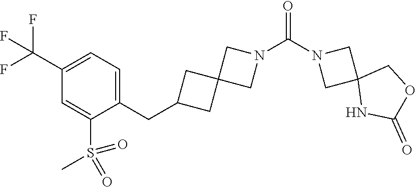 [SMILES: CS(=O)(=O)c1cc(C(F)(F)F)ccc1CC1CC2(C1)CN(C(=O)N1CC3(COC(=O)N3)C1)C2] | 2-[6-[2-mesy1-4- (trifluoromethyl)benzyl]- 2- azaspiro[3.3]heptane-2- carbonyl]-7-oxa-2,5- diazaspiro[3.4]octan-6- one | A.2 and B.20 | 488.3 [M + H]⁺ |
| 28 | 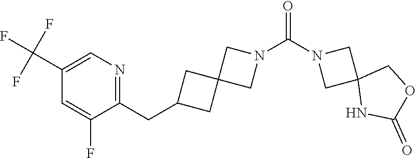 [SMILES: O=C1NC2(CO1)CN(C(=O)N1CC3(CC(Cc4ncc(C(F)(F)F)cc4F)C3)C1)C2] | 2-[6-[[3-fluoro-5- (trifluoromethyl)-2- pyridyl]methyl]-2- azaspiro[3.3]heptane-2- carbonyl]-7-oxa-2,5- diazaspiro[3.4]octan-6- one | A.2 and B.6 | 429.2 [M + H]⁺ |
| 29 | 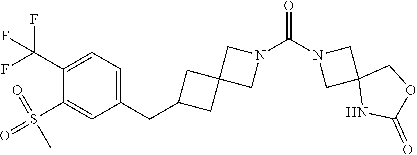 [SMILES: CS(=O)(=O)c1cc(CC2CC3(C2)CN(C(=O)N2CC4(COC(=O)N4)C2)C3)ccc1C(F)(F)F] | 2-[6-[3-mesyl-4- (trifluoromethyl)benzyl]- 2- azaspiro[3.3]heptane-2- carbonyl]-7-oxa-2,5- diazaspiro[3.4]octan-6- one | A.2 and B.21 | 488.3 [M + H]⁺ |
| 30 | 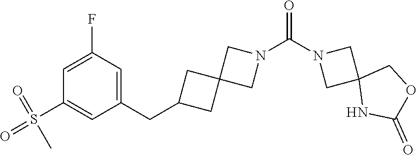 [SMILES: CS(=O)(=O)c1cc(F)cc(CC2CC3(C2)CN(C(=O)N2CC4(COC(=O)N4)C2)C3)c1] | 2-[6-(3-fluoro-5-mesyl- benzyl)-2- azaspiro[3.3]heptane-2- carbonyl]-7-oxa-2,5- diazaspiro[3.4]octan-6- one | A.2 and B.9 | 438.3 [M + H]⁺ |
| 31 | 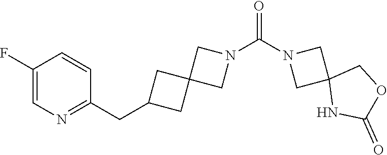 [SMILES: O=C1NC2(CO1)CN(C(=O)N1CC3(CC(Cc4ccc(F)cn4)C3)C1)C2] | 2-[6-[(5-fluoro-2- pyridyl)methyl]-2- azaspiro[3.3]heptane-2- carbonyl]-7-oxa-2,5- diazaspiro[3.4]octan-6- one | A.2 and B.22 | 361.2 [M + H]⁺ |
| 32 | 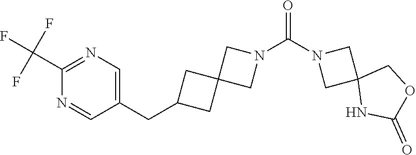 [SMILES: O=C1NC2(CO1)CN(C(=O)N1CC3(CC(Cc4cnc(C(F)(F)F)nc4)C3)C1)C2] | 2-[6-[[2- (trifluoromethyl)pyrimi- din-5-y1]methyl]-2- azaspiro[3.3]heptane-2- carbonyl]-7-oxa-2,5- diazaspiro[3.4]octan-6- one | A.2 and B.10 | 412.2 [M + H]⁺ |
| 33 | 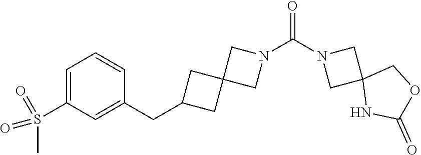 [SMILES: CS(=O)(=O)c1cccc(CC2CC3(C2)CN(C(=O)N2CC4(COC(=O)N4)C2)C3)c1] | 2-[6-(3-mesylbenzyl)-2- azaspiro[3.3]heptane-2- carbonyl]-7-oxa-2,5- diazaspiro[3.4]octan-6- one | A.2 and B.8 | 420.2 [M + H]⁺ |
| 34 | 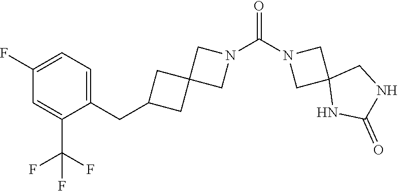 [SMILES: O=C1NCC2(CN(C(=O)N3CC4(CC(Cc5ccc(F)cc5C(F)(F)F)C4)C3)C2)N1] | 2-[6-[4-fluoro-2- (trifluoromethyl)benzyl]- 2- azaspiro[3.3]heptane-2- carbonyl]-2,5,7- triazaspiro[3.4]octan-6- one | A.3 and B.17 | 427.2 [M + H]⁺ |
| 35 | 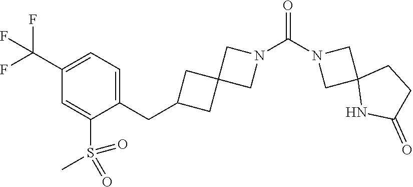 [SMILES: CS(=O)(=O)c1cc(C(F)(F)F)ccc1CC1CC2(C1)CN(C(=O)N1CC3(CCC(=O)N3)C1)C2] | 2-[6-[2-mesyl-4- (trifluoromethyl)benzyl]- 2- azaspiro[3.3]heptane-2- carbonyl]-2,5- diazaspiro[3.4]octan-6- one | A.1 and B.20 | 486.3 [M + H]⁺ |
| 36 | 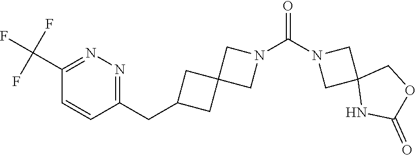 [SMILES: O=C1NC2(CO1)CN(C(=O)N1CC3(CC(Cc4ccc(C(F)(F)F)nn4)C3)C1)C2] | 2-[6-[[6- (trifluoromethyl)pyrida- zin-3-yl]methyl]-2- azaspiro[3.3]heptane-2- carbonyl]-7-oxa-2,5- diazaspiro[3.4]octan-6- one | A.2 and B.11 | 412.2 [M + H]⁺ |
| 37 | 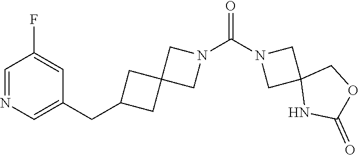 [SMILES: O=C1NC2(CO1)CN(C(=O)N1CC3(CC(Cc4cncc(F)c4)C3)C1)C2] | 2-[6-[(5-fluoro-3- pyridyl)methyl]-2- azaspiro[3.3]heptane-2- carbonyl]-7-oxa-2,5- diazaspiro[3.4]octan-6- one | A.2 and B.23 | 361.1 [M + H]⁺ |
| 38 | 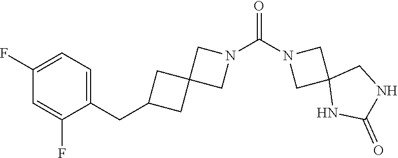 [SMILES: O=C1NCC2(CN(C(=O)N3CC4(CC(Cc5ccc(F)cc5F)C4)C3)C2)N1] | 2-[6-(2,4- difluorobenzyl)-2- azaspiro[3.3]heptane-2- carbonyl]-2,5,7- triazaspiro[3.4]octan-6- one | A.3 and B.18 | 377.2 [M + H]⁺ |
| 39 | 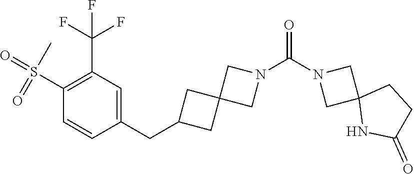 [SMILES: CS(=O)(=O)c1ccc(CC2CC3(C2)CN(C(=O)N2CC4(CCC(=O)N4)C2)C3)cc1C(F)(F)F] | 2-[6-[4-mesy1-3- (trifluoromethyl)benzyl]- 2- azaspiro[3.3]heptane-2- carbonyl]-2,5- diazaspiro[3.4]octan-6- one | A.1 and B.24 | 486.3 [M + H]⁺ |
| 40 | 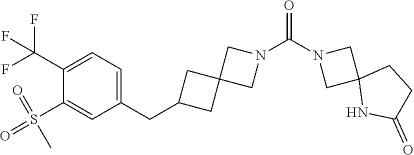 [SMILES: CS(=O)(=O)c1cc(CC2CC3(C2)CN(C(=O)N2CC4(CCC(=O)N4)C2)C3)ccc1C(F)(F)F] | 2-[6-[3-mesyl-4- (trifluoromethyl)benzyl]- 2- azaspiro[3.3]heptane-2- carbonyl]-2,5- diazaspiro[3.4]octan-6- one | A.1 and B.21 | 486.3 [M + H]⁺ |
| 42 | 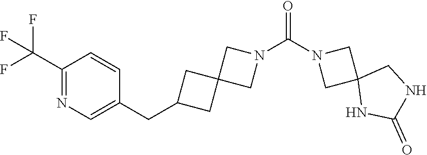 [SMILES: O=C1NCC2(CN(C(=O)N3CC4(CC(Cc5ccc(C(F)(F)F)nc5)C4)C3)C2)N1] | 2-[6-[[6- (trifluoromethyl)-3- pyridyl]methyl]-2- azaspiro[3.3]heptane-2- carbonyl]-2,5,7- triazaspiro[3.4]octan-6- one | A.3 and B.3 | 410.2 [M + H]⁺ |
| 43 | 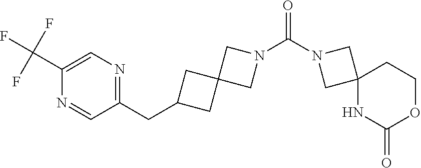 [SMILES: O=C1NC2(CCO1)CN(C(=O)N1CC3(CC(Cc4cnc(C(F)(F)F)cn4)C3)C1)C2] | 2-[6-[[5- (trifluoromethyl)pyrazin- 2-yl]methyl]-2- azaspiro[3.3]heptane-2- carbonyl]-7-oxa-2,5- diazaspiro[3.5]nonan-6- one | A. (CAS: 1955505- 70-0, Enamine, ENA508 624399) and B.4 | 426.2 [M + H]⁺ |
| 44 | 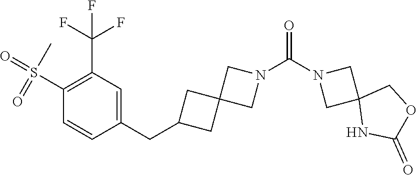 [SMILES: CS(=O)(=O)c1ccc(CC2CC3(C2)CN(C(=O)N2CC4(COC(=O)N4)C2)C3)cc1C(F)(F)F] | 2-[6-[4-mesyl-3- (trifluoromethyl)benzyl]- 2- azaspiro[3.3]heptane-2- carbonyl]-7-oxa-2,5- diazaspiro[3.4]octan-6- one | A.2 and B.24 | 488.3 [M + H]⁺ |
| 45 | 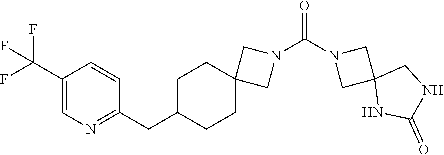 [SMILES: O=C1NCC2(CN(C(=O)N3CC4(CCC(Cc5ccc(C(F)(F)F)cn5)CC4)C3)C2)N1] | 2-[7-[[5- (trifluoromethyl)-2- pyridyl]methyl]-2- azaspiro[3.5]nonane-2- carbonyl]-2,5,7- triazaspiro[3.4]octan-6- one | A.3 and B.25 | 438.3 [M + H]⁺ |
| 46 | 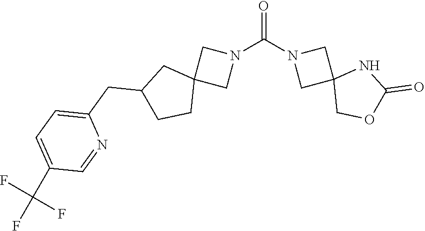 [SMILES: O=C1NC2(CO1)CN(C(=O)N1CC3(CCC(Cc4ccc(C(F)(F)F)cn4)C3)C1)C2] | 2-[6-[[5- (trifluoromethyl)-2- pyridyl]methyl]-2- azaspiro[3.4]octane-2- carbonyl]-7-oxa-2,5- diazaspiro[3.4]octan-6- one | A.2 and B.26 | 425.4 [M + H]⁺ |
| 47 | 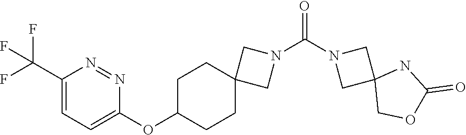 [SMILES: O=C1NC2(CO1)CN(C(=O)N1CC3(CCC(Oc4ccc(C(F)(F)F)nn4)CC3)C1)C2] | 2-[7-[6- (trifluoromethyl)pyrida- zin-3-y1]oxy-2- azaspiro[3.5]nonane-2- carbonyl]-7-oxa-2,5- diazaspiro[3.4]octan-6- one | A.2 and B.27 | 442.3 [M + H]⁺ |
| 48 | 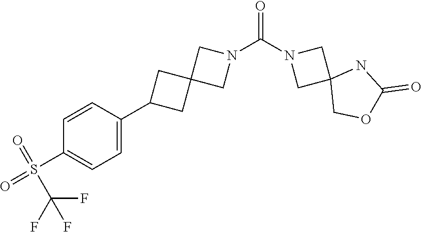 [SMILES: O=C1NC2(CO1)CN(C(=O)N1CC3(CC(c4ccc(S(=O)(=O)C(F)(F)F)cc4)C3)C1)C2] | 2-[6-(4-triflylphenyl)-2- azaspiro[3.3]heptane-2- carbonyl]-7-oxa-2,5- diazaspiro[3.4]octan-6- one | A.2 and B.28 | 401.1 [M + H]⁺ |
| 49 | 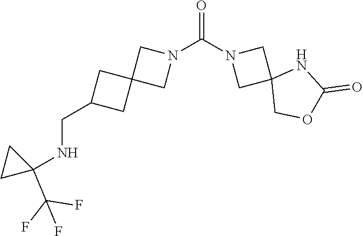 [SMILES: O=C1NC2(CO1)CN(C(=O)N1CC3(CC(CNC4(C(F)(F)F)CC4)C3)C1)C2] | 2-[6-[[[1- (trifluoromethyl)cyclopro- ropyl]amino]methyl]-2- azaspiro[3.3]heptane-2- carbonyl]-7-oxa-2,5- diazaspiro[3.4]octan-6- one | A.2 and B.29 | 389.2 [M + H]⁺ |
| 50 | 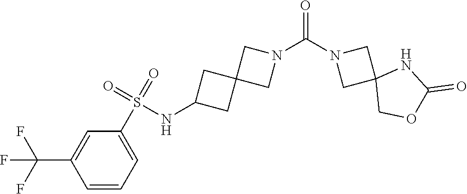 [SMILES: O=C1NC2(CO1)CN(C(=O)N1CC3(CC(NS(=O)(=O)c4cccc(C(F)(F)F)c4)C3)C1)C2] | N-[2-(6-keto-7-oxa-2,5- diazaspiro[3.4]octane- 2-carbonyl)-2- azaspiro[3.3]heptan-6- yl]-3- (trifluoromethyl)benzene- sulfonamide | A.2 and B.30 | 475.2 [M + H]⁺ |
| 51 | 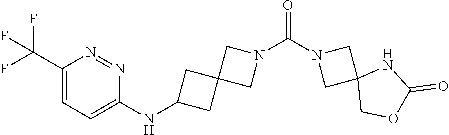 [SMILES: O=C1NC2(CO1)CN(C(=O)N1CC3(CC(Nc4ccc(C(F)(F)F)nn4)C3)C1)C2] | 2-[6-[[6- (trifluoromethyl)pyrida- zin-3-yl]amino]-2- azaspiro[3.3]heptane-2- carbonyl]-7-oxa-2,5- diazaspiro[3.4]octan-6- one | A.2 and B.31 | 413.2 [M + H]⁺ |
| 52 | 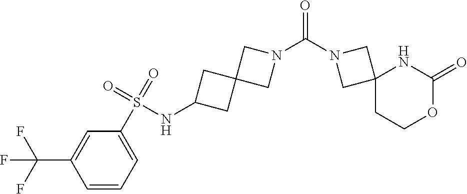 [SMILES: O=C1NC2(CCO1)CN(C(=O)N1CC3(CC(NS(=O)(=O)c4cccc(C(F)(F)F)c4)C3)C1)C2] | N-[2-(6-keto-7-oxa-2,5- diazaspiro[3.5]nonane- 2-carbonyl)-2- azaspiro[3.3]heptan-6- yl]-3- (trifluoromethyl)benzene- sulfonamide | A.4 and B.30 | 489.2 [M + H]⁺ |
| 53 | 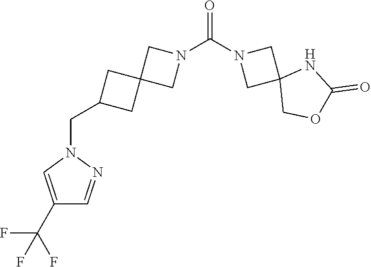 [SMILES: O=C1NC2(CO1)CN(C(=O)N1CC3(CC(Cn4cc(C(F)(F)F)cn4)C3)C1)C2] | 2-[6-[[4- (trifluoromethyl)pyrazol- 1-y1]methyl]-2- azaspiro[3.3]heptane-2- carbonyl]-7-oxa-2,5- diazaspiro[3.4]octan-6- one | A.2 and B.32 | 400.3 [M + H]⁺ |
| 54 | 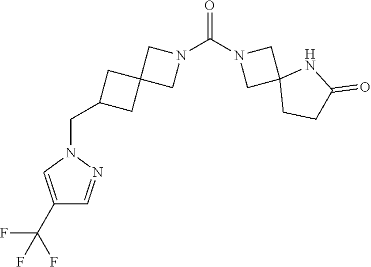 [SMILES: O=C1CCC2(CN(C(=O)N3CC4(CC(Cn5cc(C(F)(F)F)cn5)C4)C3)C2)N1] | 2-[6-[[4- (trifluoromethyl)pyrazol- 1-y1]methyl]-2- azaspiro[3.3]heptane-2- carbonyl]-2,5- diazaspiro[3.4]octan-6- one | A.1 and B.32 | 398.3 [M + H]⁺ |
| 56 | 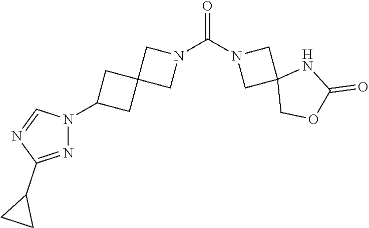 [SMILES: O=C1NC2(CO1)CN(C(=O)N1CC3(CC(n4cnc(C5CC5)n4)C3)C1)C2] | 2-[6-(3-cyclopropyl- 1,2,4-triazol-1-yl)-2- azaspiro[3.3]heptane-2- carbonyl]-7-oxa-2,5- diazaspiro[3.4]octan-6- one | A.2 and B.33 | 359.2 [M + H]⁺ |
| 57 | 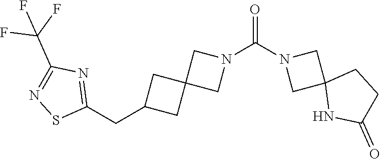 [SMILES: O=C1CCC2(CN(C(=O)N3CC4(CC(Cc5nc(C(F)(F)F)ns5)C4)C3)C2)N1] | 2-[6-[[3- (trifluoromethyl)-1,2,4- thiadiazol-5-y1]methyl]- 2-azaspiro[3.3]heptane- 2-carbonyl]-2,5- diazaspiro[3.4]octan-6- one | A.1 and B.34 | 416.3 [M + H]⁺ |
| 58 | 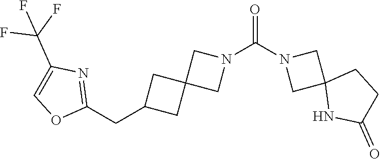 [SMILES: O=C1CCC2(CN(C(=O)N3CC4(CC(Cc5nc(C(F)(F)F)co5)C4)C3)C2)N1] | 2-[6-[[4- (trifluoromethyl)oxazol- 2-yl]methyl]-2- azaspiro[3.3]heptane-2- carbonyl]-2,5- diazaspiro[3.4]octan-6- one | A.1 and B.35 | 399.3 |
| 59 | 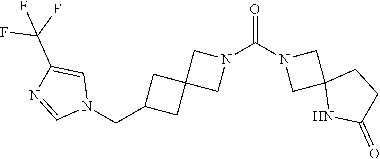 [SMILES: O=C1CCC2(CN(C(=O)N3CC4(CC(Cn5cnc(C(F)(F)F)c5)C4)C3)C2)N1] | 2-[6-[[4- (trifluoromethyl)imidazol- 1-yl]methyl]-2- azaspiro[3.3]heptane-2- carbonyl]-2,5- diazaspiro[3.4]octan-6- one | A.1 and B.36 | 398.2 [M + H]⁺ |
| 60 | 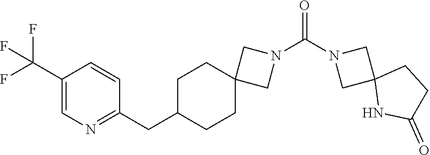 [SMILES: O=C1CCC2(CN(C(=O)N3CC4(CCC(Cc5ccc(C(F)(F)F)cn5)CC4)C3)C2)N1] | 2-[7-[[5- (trifluoromethyl)-2- pyridyl]methyl]-2- azaspiro[3.5]nonane-2- carbonyl]-2,5- diazaspiro[3.4]octan-6- one | A.1 and B.26 | 437.3 [M + H]⁺ |
| 61 | 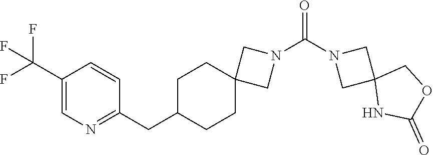 [SMILES: O=C1NC2(CO1)CN(C(=O)N1CC3(CCC(Cc4ccc(C(F)(F)F)cn4)CC3)C1)C2] | 2-[7-[[5- (trifluoromethyl)-2- pyridyl]methyl]-2- azaspiro[3.5]nonane-2- carbonyl]-7-oxa-2,5- diazaspiro[3.4]octan-6- one | A.2 and B.26 | 439.3 [M + H]⁺ |
| 62 | 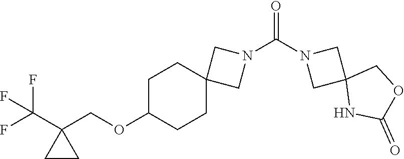 [SMILES: O=C1NC2(CO1)CN(C(=O)N1CC3(CCC(OCC4(C(F)(F)F)CC4)CC3)C1)C2] | 2-[7-[[1- (trifluoromethyl)cyclopro- pyl]methoxy]-2- azaspiro[3.5]nonane-2- carbonyl]-7-oxa-2,5- diazaspiro[3.4]octan-6- one | A.2 and B.37 | 418.3 [M + H]⁺ |
| 63 | 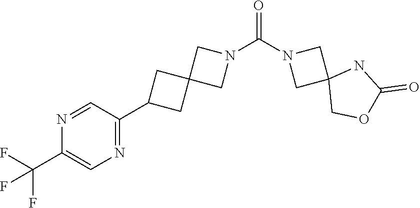 [SMILES: O=C1NC2(CO1)CN(C(=O)N1CC3(CC(c4cnc(C(F)(F)F)cn4)C3)C1)C2] | 2-[6-[5- (trifluoromethyl)pyrazin- 2-yl]-2- azaspiro[3.3]heptane-2- carbonyl]-7-oxa-2,5- diazaspiro[3.4]octan-6- one | A.2 and B.38 | 398.2 [M + H]⁺ |
| 64 | 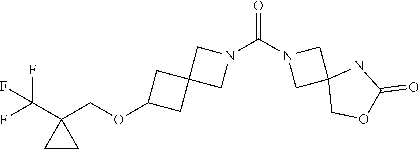 [SMILES: O=C1NC2(CO1)CN(C(=O)N1CC3(CC(OCC4(C(F)(F)F)CC4)C3)C1)C2] | 2-[6-[[1- (trifluoromethyl)cyclopro- pyl]methoxy]-2- azaspiro[3.3]heptane-2- carbonyl]-7-oxa-2,5- diazaspiro[3.4]octan-6- one | A.2 and B.39 | 390.2 [M + H]⁺ |
| 65 | 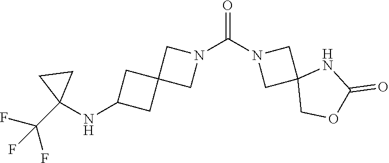 [SMILES: O=C1NC2(CO1)CN(C(=O)N1CC3(CC(NC4(C(F)(F)F)CC4)C3)C1)C2] | 2-[6-[[1- (trifluoromethyl)cyclopro- pyl]amino]-2- azaspiro[3.3]heptane-2- carbonyl]-7-oxa-2,5- diazaspiro[3.4]octan-6- one | A.2 and B.40 | 375.2 [M + H]⁺ |
| 66 | 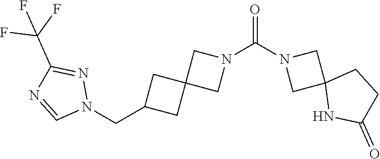 [SMILES: O=C1CCC2(CN(C(=O)N3CC4(CC(Cn5cnc(C(F)(F)F)n5)C4)C3)C2)N1] | 2-[6-[[3- (trifluoromethyl)-1,2,4- triazol-1-yl]methyl]-2- carbonyl]-2,5- diazaspiro[3.4]octan-6- one | A.1 and B.41 | 399.4 [M + H]⁺ |
| 67 | 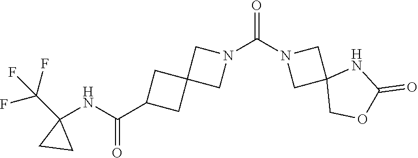 [SMILES: O=C1NC2(CO1)CN(C(=O)N1CC3(CC(C(=O)NC4(C(F)(F)F)CC4)C3)C1)C2] | 2-(6-keto-7-oxa-2,5- diazaspiro[3.4]octane- carbonyl)-N-[1- (trifluoromethyl)cyclopro- pyl]-2- azaspiro[3.3]heptane-6- carboxamide | A.2 and B.42 | 403.2 [M + H]⁺ |
| 68 | 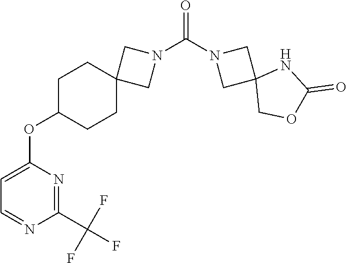 [SMILES: O=C1NC2(CO1)CN(C(=O)N1CC3(CCC(Oc4ccnc(C(F)(F)F)n4)CC3)C1)C2] | 2-[7-[2- (trifluoromethyl)pyrimi- din-4-yl]oxy-2- azaspiro[3.5]nonane-2- carbonyl]-7-oxa-2,5- diazaspiro[3.4]octan-6- one | A.2 and B.43 | 442.3 [M + H]⁺ |
| 69 | 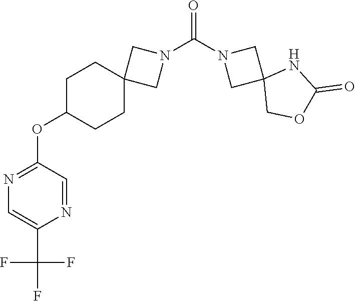 [SMILES: O=C1NC2(CO1)CN(C(=O)N1CC3(CCC(Oc4cnc(C(F)(F)F)cn4)CC3)C1)C2] | 2-[7-[5- (trifluoromethyl)pyrazin- 2-yl]oxy-2- azaspiro[3.5]nonane-2- carbonyl]-7-oxa-2,5- diazaspiro[3.4]octan-6- one | A.2 and B.44 | 442.3 [M + H]⁺ |
| 70 | 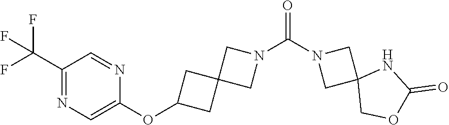 [SMILES: O=C1NC2(CO1)CN(C(=O)N1CC3(CC(Oc4cnc(C(F)(F)F)cn4)C3)C1)C2] | 2-[6-[5- (trifluoromethyl)pyrazin- 2-yl]oxy-2- azaspiro[3.3]heptane-2- carbonyl]-7-oxa-2,5- diazaspiro[3.4]octan-6- one | A.2 and B.45 | 414.2 [M + H]⁺ |
| 71 | 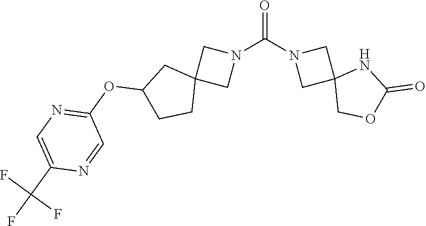 [SMILES: O=C1NC2(CO1)CN(C(=O)N1CC3(CCC(Oc4cnc(C(F)(F)F)cn4)C3)C1)C2] | 2-[6-[5- (trifluoromethyl)pyrazin- 2-yl]oxy-2- azaspiro[3.4]octane-2- carbonyl]-7-oxa-2,5- diazaspiro[3.4]octan-6- one | A.2 and B.46 | 428.2 [M + H]⁺ |
| 72 | 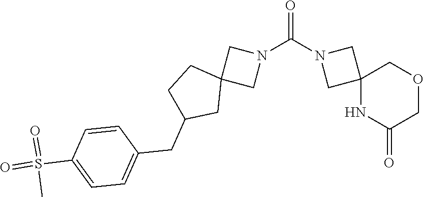 [SMILES: CS(=O)(=O)c1ccc(CC2CCC3(C2)CN(C(=O)N2CC4(COCC(=O)N4)C2)C3)cc1] | 2-[6-(4-mesylbenzyl)-2- azaspiro[3.4]octane-2- carbonyl]-8-oxa-2,5- diazaspiro[3.5]nonan-6- one | A5 and B.47 | 448.3 [M + H]⁺ |
| 73 | 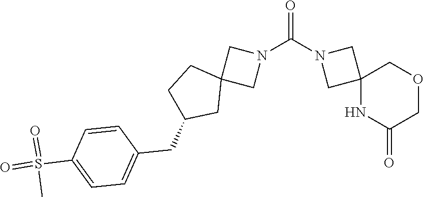 [SMILES: CS(=O)(=O)c1ccc(C[C@@H]2CCC3(C2)CN(C(=O)N2CC4(COCC(=O)N4)C2)C3)cc1] | 2-[(6S)-6-(4- mesylbenzyl)-2- azaspiro[3.4]octane-2- carbonyl]-8-oxa-2,5- diazaspiro[3.5]nonan-6- one | A.5 and B.47 | 448.3 [M + H]⁺ |
| | enantiomer separated from Ex. 72 | | | |
| | by chiral SFC, arbitrarily assigned | | | |
| 74 | 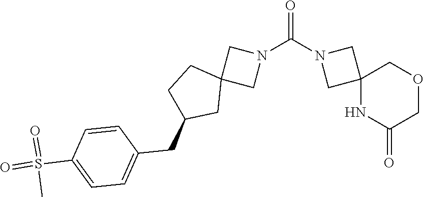 [SMILES: CS(=O)(=O)c1ccc(C[C@H]2CCC3(C2)CN(C(=O)N2CC4(COCC(=O)N4)C2)C3)cc1] | 2-[(6R)-6-(4- mesylbenzyl)-2- azaspiro[3.4]octane-2- carbonyl]-8-oxa-2,5- diazaspiro[3.5]nonan-6- one | A.5 and B.47 | 448.3 [M + H]⁺ |
| | enantiomer separated from Ex. 72 | | | |
| | by chiral SFC, arbitrarily assigned | | | |
| 76 | 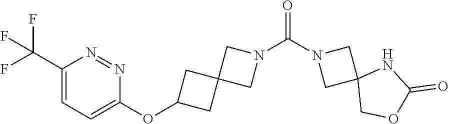 [SMILES: O=C1NC2(CO1)CN(C(=O)N1CC3(CC(Oc4ccc(C(F)(F)F)nn4)C3)C1)C2] | 2-[6-[6- (trifluoromethyl)pyrida- zin-3-yl]oxy-2- azaspiro[3.3]heptane-2- carbonyl]-7-oxa-2,5- diazaspiro[3.4]octan-6- one | A.2 and B.48 | 414.2 [M + H]⁺ |
| 77 | 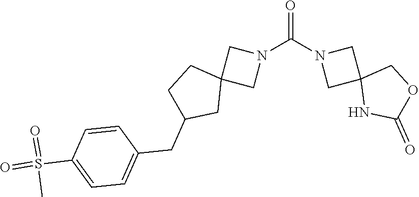 [SMILES: CS(=O)(=O)c1ccc(CC2CCC3(C2)CN(C(=O)N2CC4(COC(=O)N4)C2)C3)cc1] | 2-[6-(4-mesylbenzyl)-2- azaspiro[3.4]octane-2- carbonyl]-7-oxa-2,5- diazaspiro[3.4]octan-6- one | A.2 and B.47 | 434.4 [M + H]⁺ |
| 78 | 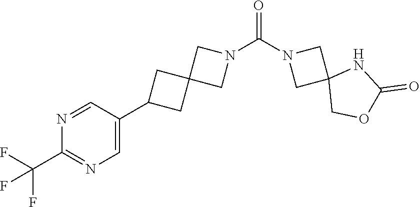 [SMILES: O=C1NC2(CO1)CN(C(=O)N1CC3(CC(c4cnc(C(F)(F)F)nc4)C3)C1)C2] | 2-[6-[2- (trifluoromethyl)pyrimi- din-5-yl]-2- azaspiro[3.3]heptane-2- carbonyl]-7-oxa-2,5- diazaspiro[3.4]octan-6- one | A.2 and B.49 | 398.1 [M + H]⁺ |
| 79 | 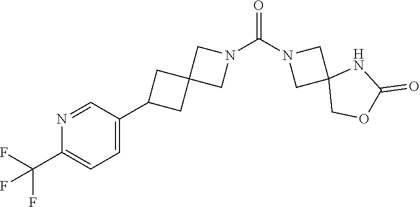 [SMILES: O=C1NC2(CO1)CN(C(=O)N1CC3(CC(c4ccc(C(F)(F)F)nc4)C3)C1)C2] | 2-[6-[6- (trifluoromethyl)-3- pyridyl]-2- azaspiro[3.3]heptane-2- carbonyl]-7-oxa-2,5- diazaspiro[3.4]octan-6- one | A.2 and B.50 | 397.1 [M + H]⁺ |
| 80 | 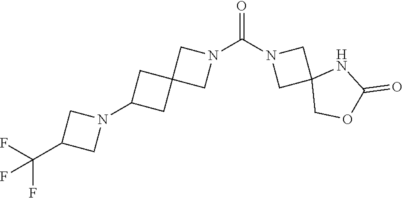 [SMILES: O=C1NC2(CO1)CN(C(=O)N1CC3(CC(N4CC(C(F)(F)F)C4)C3)C1)C2] | 2-[6-[3- (trifluoromethyl)azetidin- 1-y1]-2- azaspiro[3.3]heptane-2- carbonyl]-7-oxa-2,5- diazaspiro[3.4]octan-6- one | A.2 and B.51 | 375.2 [M + H]⁺ |
| 81 | 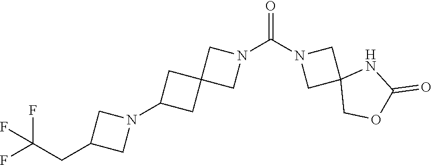 [SMILES: O=C1NC2(CO1)CN(C(=O)N1CC3(CC(N4CC(CC(F)(F)F)C4)C3)C1)C2] | 2-[6-[3-(2,2,2- trifluoroethyl)azetidin- 1-y1]-2- azaspiro[3.3]heptane-2- carbonyl]-7-oxa-2,5- diazaspiro[3.4]octan-6- one | A.2 and B.52 | 389.2 [M + H]⁺ |
| 82 | 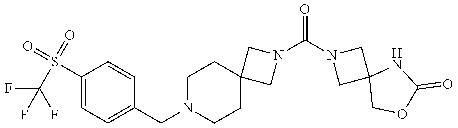 [SMILES: O=C1NC2(CO1)CN(C(=O)N1CC3(CCN(Cc4ccc(S(=O)(=O)C(F)(F)F)cc4)CC3)C1)C2] | 2-[7-(4-triflylbenzyl)- 2,7- diazaspiro[3.5]nonane- 2-carbonyl]-2,5- diazaspiro[3.4]octan-6- one | A.1 and B.53 | 501.2 [M + H]⁺ |
| 83 | 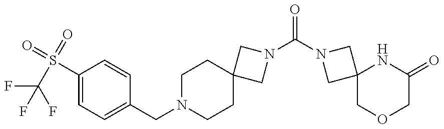 [SMILES: O=C1COCC2(CN(C(=O)N3CC4(CCN(Cc5ccc(S(=O)(=O)C(F)(F)F)cc5)CC4)C3)C2)N1] | 2-[7-(4-triflylbenzyl)- 2,7- diazaspiro[3.5]nonane- 2-carbonyl]-8-oxa-2,5- diazaspiro[3.5]nonan-6- one | A.5 and B.53 | 517.3 [M + H]⁺ |
| 84 | 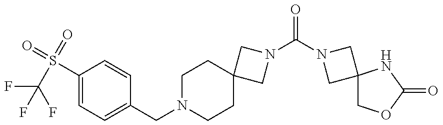 [SMILES: O=C1NC2(CO1)CN(C(=O)N1CC3(CCN(Cc4ccc(S(=O)(=O)C(F)(F)F)cc4)CC3)C1)C2] | 2-[7-(4-triflylbenzyl)- 2,7- diazaspiro[3.5]nonane- 2-carbonyl]-7-oxa-2,5- diazaspiro[3.4]octan-6- one | A.2 and B.53 | 503.2 [M + H]⁺ |
| 85 | 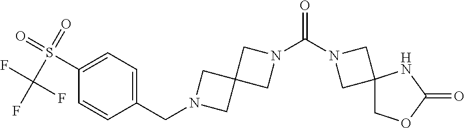 [SMILES: O=C1NC2(CO1)CN(C(=O)N1CC3(CN(Cc4ccc(S(=O)(=O)C(F)(F)F)cc4)C3)C1)C2] | 2-[6-(4-triflylbenzyl)- 2,6- diazaspiro[3.3]heptane- 2-carbonyl]-7-oxa-2,5- diazaspiro[3.4]octan-6- one | A.2 and B.54 | 475.2 [M + H]⁺ |
| 86 | 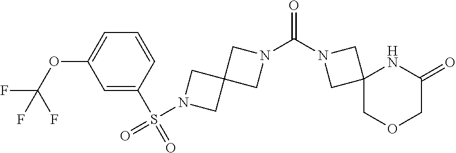 [SMILES: O=C1COCC2(CN(C(=O)N3CC4(C3)CN(S(=O)(=O)c3cccc(OC(F)(F)F)c3)C4)C2)N1] | 2-[2-[3- (trifluoromethoxy)phenyl] sulfony1-2,6- diazaspiro[3.3]heptane- 6-carbonyl]-8-oxa-2,5- diazaspiro[3.5]nonan-6- one | A.5 and B.55 | 491.2 [M + H]⁺ |
| 87 | 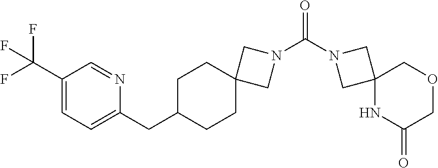 [SMILES: O=C1COCC2(CN(C(=O)N3CC4(CCC(Cc5ccc(C(F)(F)F)cn5)CC4)C3)C2)N1] | 2-[7-[[5- (trifluoromethyl)-2- pyridyl]methyl]-2- azaspiro[3.5]nonane-2- carbonyl]-8-oxa-2,5- diazaspiro[3.5]nonan-6- one | A.5 and B.25 | 453.4 [M + H]⁺ |
| 88 | 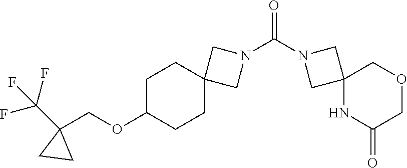 [SMILES: O=C1COCC2(CN(C(=O)N3CC4(CCC(OCC5(C(F)(F)F)CC5)CC4)C3)C2)N1] | 2-[7-[[1- (trifluoromethyl)cyclopro- pyl]methoxy]-2- azaspiro[3.5]nonane-2- carbonyl]-8-oxa-2,5- diazaspiro[3.5]nonan-6- one | A.5 and B.37 | 432.3 [M + H]⁺ |
| 89 | 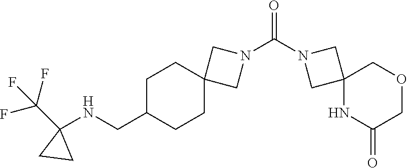 [SMILES: O=C1COCC2(CN(C(=O)N3CC4(CCC(CNC5(C(F)(F)F)CC5)CC4)C3)C2)N1] | 2-[6-[[[1- (trifluoromethyl)cyclopro- pyl]amino]methyl]-2- azaspiro[3.3]heptane-2- carbonyl]-8-oxa-2,5- diazaspiro[3.5]nonan-6- one | A.5 and B.29 | 403.3 [M + H]⁺ |
| 91 | 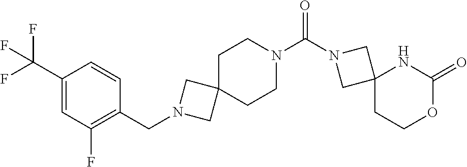 [SMILES: O=C1NC2(CCO1)CN(C(=O)N1CCC3(CC1)CN(Cc1ccc(C(F)(F)F)cc1F)C3)C2] | 2-[2-[2-fluoro-4- (trifluoromethyl)benzyl]- 2,7- diazaspiro[3.5]nonane- 7-carbonyl]-7-oxa-2,5- diazaspiro[3.5]nonan-6- one | A.4 and B.56 | 471.2 [M + H]⁺ |
| 92 | 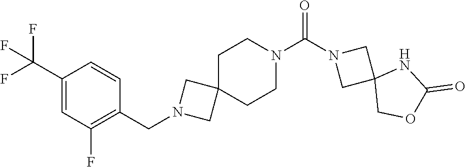 [SMILES: O=C1NC2(CO1)CN(C(=O)N1CCC3(CC1)CN(Cc1ccc(C(F)(F)F)cc1F)C3)C2] | 2-[2-[2-fluoro-4- (trifluoromethyl)benzyl]- 2,7- diazaspiro[3.5]nonane- 7-carbonyl]-7-oxa-2,5- diazaspiro[3.4]octan-6- one | A.2 and B.56 | 457.2 [M + H]⁺ |
| 93 | 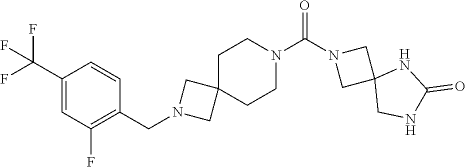 [SMILES: O=C1NCC2(CN(C(=O)N3CCC4(CC3)CN(Cc3ccc(C(F)(F)F)cc3F)C4)C2)N1] | 2-[2-[2-fluoro-4- (trifluoromethyl)benzyl]- 2,7- diazaspiro[3.5]nonane- 7-carbonyl]-2,5,7- triazaspiro[3.4]octan-6- one | A.3 and B.56 | 456.2 [M + H]⁺ |
| 94 | 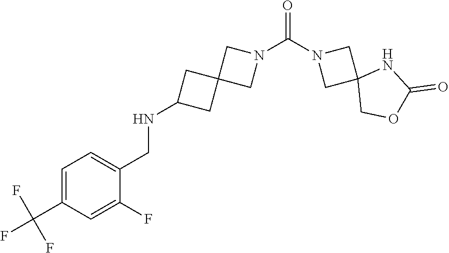 [SMILES: O=C1NC2(CO1)CN(C(=O)N1CC3(CC(NCc4ccc(C(F)(F)F)cc4F)C3)C1)C2] | 2-[6-[[2-fluoro-4- (trifluoromethyl)benzyl- amino]-2- azaspiro[3.3]heptane-2- carbonyl]-7-oxa-2,5- diazaspiro[3.4]octan-6- one | A.2 and B.125 | 443.2 [M + H]⁺ |
| 95 | 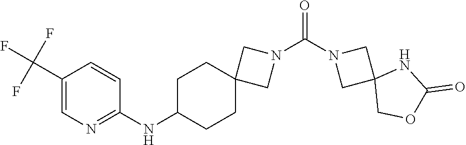 [SMILES: O=C1NC2(CO1)CN(C(=O)N1CC3(CCC(Nc4ccc(C(F)(F)F)cn4)CC3)C1)C2] | 2-[7-[[5- (trifluoromethyl)-2- pyridyl]amino]-2- azaspiro[3.5]nonane-2- carbonyl]-7-oxa-2,5- diazaspiro[3.4]octan-6- one | A.2 and B.369 | 440.3 [M + H]⁺ |
| 96 | 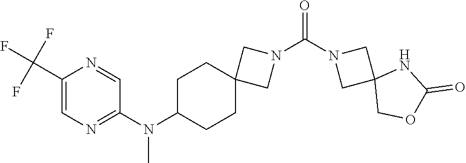 [SMILES: CN(c1cnc(C(F)(F)F)cn1)C1CCC2(CC1)CN(C(=O)N1CC3(COC(=O)N3)C1)C2] | 2-[7-[methyl-[5- (trifluoromethyl)pyrazin- 2-yl]amino]-2- azaspiro[3.5]nonane-2- carbonyl]-7-oxa-2,5- diazaspiro[3.4]octan-6- one | A.2 and B.368 | 455.3 [M + H]⁺ |
| 97 | 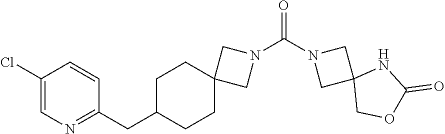 [SMILES: O=C1NC2(CO1)CN(C(=O)N1CC3(CCC(Cc4ccc(Cl)cn4)CC3)C1)C2] | 2-[7-[(5-chloro-2- pyridyl)methyl]-2- azaspiro[3.5]nonane-2- carbonyl]-7-oxa-2,5- diazaspiro[3.4]octan-6- one | A.2 and B.87 | 405.2 [M + H]⁺ |
| 98 | 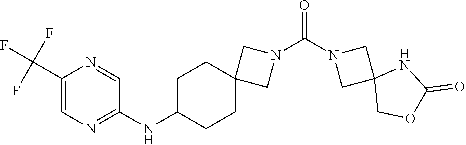 [SMILES: O=C1NC2(CO1)CN(C(=O)N1CC3(CCC(Nc4cnc(C(F)(F)F)cn4)CC3)C1)C2] | 2-[7-[[5- (trifluoromethyl)pyrazin- 2-yl]amino]-2- azaspiro[3.5]nonane-2- carbonyl]-7-oxa-2,5- diazaspiro[3.4]octan-6- one | A.2 and B.265 | 441.3 [M + H]⁺ |
| 99 | 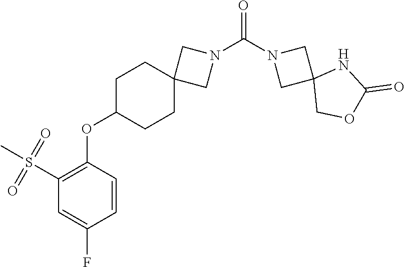 [SMILES: CS(=O)(=O)c1cc(F)ccc1OC1CCC2(CC1)CN(C(=O)N1CC3(COC(=O)N3)C1)C2] | 2-[7-(4-fluoro-2-mesyl- phenoxy)-2- azaspiro[3.5]nonane-2- carbonyl]-7-oxa-2,5- diazaspiro[3.4]octan-6- one | A.2 and B.269 | 468.3 [M + H]⁺ |
| 100 | 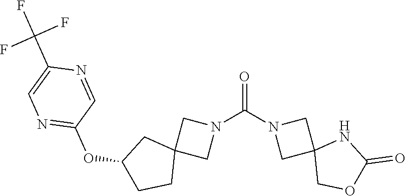 [SMILES: O=C1NC2(CO1)CN(C(=O)N1CC3(CC[C@H](Oc4cnc(C(F)(F)F)cn4)C3)C1)C2] | 2-[(6S)-6-[5- (trifluoromethyl)pyrazin- 2-yl]oxy-2- azaspiro[3.4]octane-2- carbonyl]-7-oxa-2,5- diazaspiro[3.4]octan-6- one | A.2 and B.319 | 428.2 [M + H]⁺ |
| | enantiomer separated by chiral | | | |
| | SFC, arbitrarily assigned | | | |
| 101 | 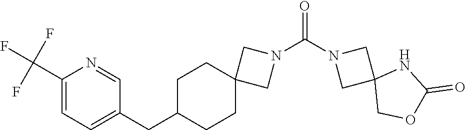 [SMILES: O=C1NC2(CO1)CN(C(=O)N1CC3(CCC(Cc4ccc(C(F)(F)F)nc4)CC3)C1)C2] | 2-[7-[[6- (trifluoromethyl)-3- pyridyl]methyl]-2- azaspiro[3.5]nonane-2- carbonyl]-7-oxa-2,5- diazaspiro[3.4]octan-6- one | A.2 and B.88 | 439.3 [M + H]⁺ |
| 102 | 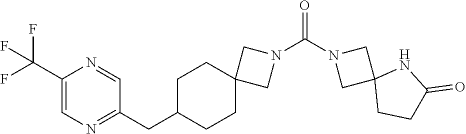 [SMILES: O=C1CCC2(CN(C(=O)N3CC4(CCC(Cc5cnc(C(F)(F)F)cn5)CC4)C3)C2)N1] | 2-[7-[[5- (trifluoromethyl)pyrazin- 2-yl]methyl]-2- azaspiro[3.5]nonane-2- carbonyl]-2,5- diazaspiro[3.4]octan-6- one | A.1 and B.86 | 438.3 [M + H]⁺ |
| 103 | 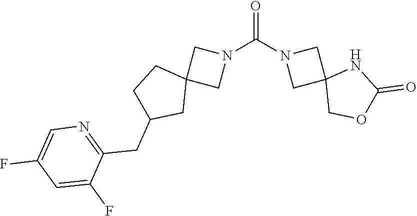 [SMILES: O=C1NC2(CO1)CN(C(=O)N1CC3(CCC(Cc4ncc(F)cc4F)C3)C1)C2] | 2-[6-[(3,5-difluoro-2- pyridyl)methyl]-2- azaspiro[3.4]octane-2- carbonyl]-7-oxa-2,5- diazaspiro[3.4]octan-6- one | A.2 and B.92 | 393.3 [M + H]⁺ |
| 104 | 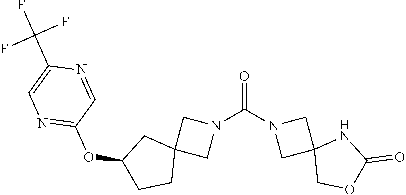 [SMILES: O=C1NC2(CO1)CN(C(=O)N1CC3(CC[C@@H](Oc4cnc(C(F)(F)F)cn4)C3)C1)C2] | 2-[(6R)-6-[5- (trifluoromethyl)pyrazin- 2-yl]oxy-2- azaspiro[3.4]octane-2- carbonyl]-7-oxa-2,5- diazaspiro[3.4]octan-6- one | A.2 and B.319 | 428.2 [M + H]⁺ |
| | enantiomer separated by chiral | | | |
| | SFC, arbitrarily assigned | | | |
| 105 | 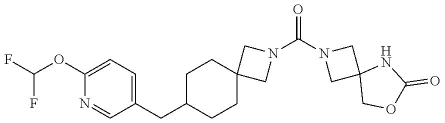 [SMILES: O=C1NC2(CO1)CN(C(=O)N1CC3(CCC(Cc4ccc(OC(F)F)nc4)CC3)C1)C2] | 2-[7-[[6- (difluoromethoxy)-3- pyridyl]methyl]-2- azaspiro[3.5]nonane-2- carbonyl]-7-oxa-2,5- diazaspiro[3.4]octan-6- one | A.2 and B.320 | 437.2 [M + H]⁺ |
| 106 | 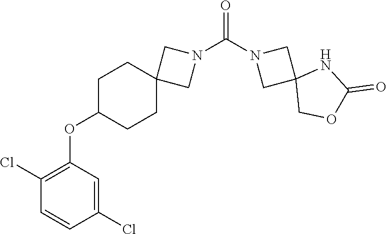 [SMILES: O=C1NC2(CO1)CN(C(=O)N1CC3(CCC(Oc4cc(Cl)ccc4Cl)CC3)C1)C2] | 2-[7-(2,5- dichlorophenoxy)-2- azaspiro[3.5]nonane-2- carbonyl]-7-oxa-2,5- diazaspiro[3.4]octan-6- one | A.2 and B.260 | 440.1 [M + H]⁺ |
| 107 | 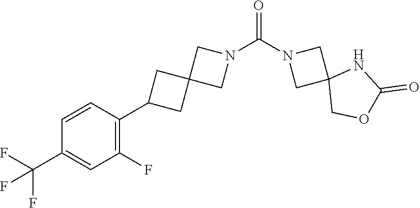 [SMILES: O=C1NC2(CO1)CN(C(=O)N1CC3(CC(c4ccc(C(F)(F)F)cc4F)C3)C1)C2] | 2-[6-[2-fluoro-4- (trifluoromethyl)phenyl- 2- azaspiro[3.3]heptane-2- carobnyl]-7-oxa-2,5- diazaspiro[3.4]octan-6- one | A.2 and B.328 | 414.2 [M + H]⁺ |
| 108 | 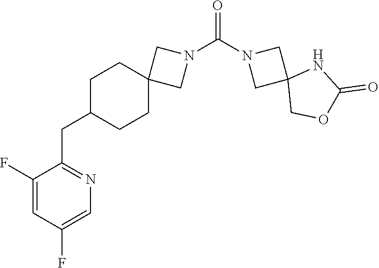 [SMILES: O=C1NC2(CO1)CN(C(=O)N1CC3(CCC(Cc4ncc(F)cc4F)CC3)C1)C2] | 2-[7-[(3,5-difluoro-2- pyridyl)methyl]-2- azaspiro[3.5]nonane-2- carbonyl]-7-oxa-2,5- diazaspiro[3.4]octan-6- one | A.2 and B.89 | 407.2 [M + H]⁺ |
| 109 | 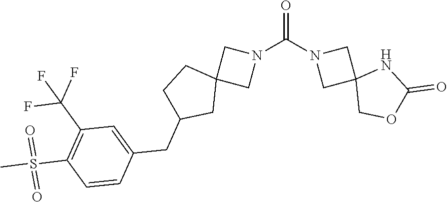 [SMILES: CS(=O)(=O)c1ccc(CC2CCC3(C2)CN(C(=O)N2CC4(COC(=O)N4)C2)C3)cc1C(F)(F)F] | 2-[6-[4-mesyl-3- (trifluoromethyl)benzyl]- 2-azaspiro[3.4]octane- 2-carbonyl]-7-oxa-2,5- diazaspiro[3.4]octan-6- one | A.2 and B.96 | 502.5 [M + H]⁺ |
| 110 | 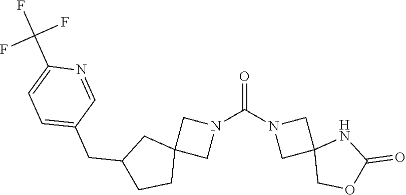 [SMILES: O=C1NC2(CO1)CN(C(=O)N1CC3(CCC(Cc4ccc(C(F)(F)F)nc4)C3)C1)C2] | 2-[6-[[6- (trifluoromethyl)-3- pyridyl]methyl]-2- azaspiro[3.4]octane-2- carbonyl]-7-oxa-2,5- diazaspiro[3.4]octan-6- one | A.2 and B.95 | 425.3 [M + H]⁺ |
| 111 | 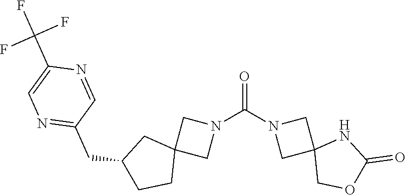 [SMILES: O=C1NC2(CO1)CN(C(=O)N1CC3(CC[C@H](Cc4cnc(C(F)(F)F)cn4)C3)C1)C2] | 2-[(6S)-6-[[5- (trifluoromethyl)pyrazin- 2-yl]methyl]-2- azaspiro[3.4]octane-2- carbonyl]-7-oxa-2,5- diazaspiro[3.4]octan-6- one | A.2 and P.92 | 426.3 [M + H]⁺ |
| 112 | 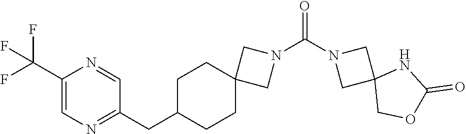 [SMILES: O=C1NC2(CO1)CN(C(=O)N1CC3(CCC(Cc4cnc(C(F)(F)F)cn4)CC3)C1)C2] | 2-[7-[[5- (trifluoromethyl)pyrazin- 2-yl]methyl]-2- azaspiro[3.5]nonane-2- carbonyl]-7-oxa-2,5- diazaspiro[3.4]octan-6- one | A.2 and B.86 | 440.3 [M + H]⁺ |
| 113 | 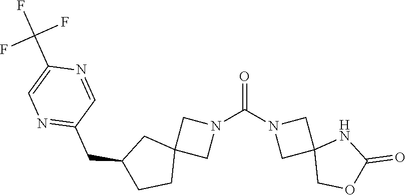 [SMILES: O=C1NC2(CO1)CN(C(=O)N1CC3(CC[C@@H](Cc4cnc(C(F)(F)F)cn4)C3)C1)C2] | 2-[(6R)-6-[[5- (trifluoromethyl)pyrazin- 2-yl]methyl]-2- azaspiro[3.4]octane-2- carbonyl]-7-oxa-2,5- diazaspiro[3.4]octan-6- one | A.2 and P.93 | 426.3 [M + H]⁺ |
| 114 | 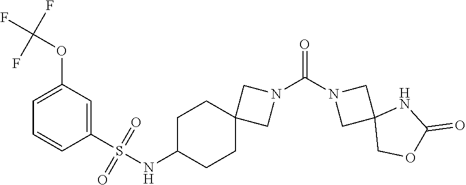 [SMILES: O=C1NC2(CO1)CN(C(=O)N1CC3(CCC(NS(=O)(=O)c4cccc(OC(F)(F)F)c4)CC3)C1)C2] | N-[2-(6-keto-7-oxa-2,5- diazaspiro[3.4]octane- 2-carbonyl)-2- azaspiro[3.5]nonan-7- yl]-3- (trifluoromethoxy)benzene- sulfonamide | A.2 and B.262 | 319.2 [M + H]⁺ |
| 115 | 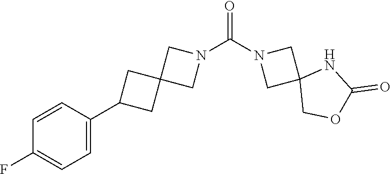 [SMILES: O=C1NC2(CO1)CN(C(=O)N1CC3(CC(c4ccc(F)cc4)C3)C1)C2] | 2-[6-(4-fluorophenyl)- 2-azaspiro[3.3]heptane- 2-carbonyl]-7-oxa-2,5- diazaspiro[3.4]octan-6- one | A.2 and B.370 | 374.2 [M + H]⁺ |
| 116 | 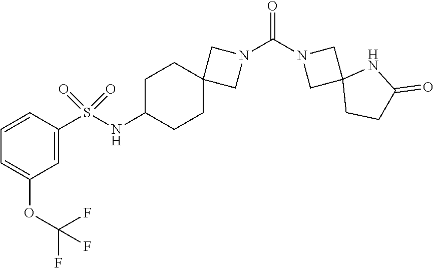 [SMILES: O=C1CCC2(CN(C(=O)N3CC4(CCC(NS(=O)(=O)c5cccc(OC(F)(F)F)c5)CC4)C3)C2)N1] | N-[2-(6-keto-2,5- diazaspiro[3.4]octane- 2-carbonyl)-2- azaspiro[3.5]nonan-7- yl]-3- (trifluoromethoxy)benzene- sulfonamide | A.1 and B.262 | 517.3 [M + H]⁺ |
| 117 | 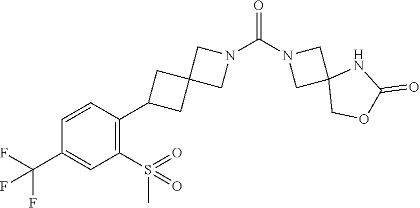 [SMILES: CS(=O)(=O)c1cc(C(F)(F)F)ccc1C1CC2(C1)CN(C(=O)N1CC3(COC(=O)N3)C1)C2] | 2-[6-[2-mesyl-4- (trifluoromethyl)phenyl- 2- azaspiro[3.3]heptane-2- carbonyl]-7-oxa-2,5- diazaspiro[3.4]octan-6- one | A.2 and B.371 | 474.2 [M + H]⁺ |
| 118 | 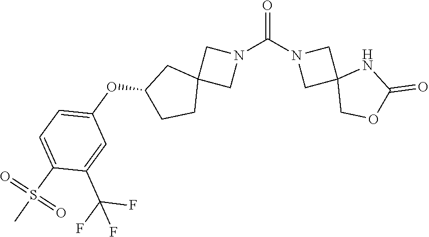 [SMILES: CS(=O)(=O)c1ccc(O[C@H]2CCC3(C2)CN(C(=O)N2CC4(COC(=O)N4)C2)C3)cc1C(F)(F)F] | 2-[(6S)-6-[4-mesy1-3- (trifluoromethyl)phenoxy]- 2- azaspiro[3.4]octane-2- carbonyl]-7-oxa-2,5- diazaspiro[3.4]octan-6- one | A.2 and B.98 | 504.4 [M + H]⁺ |
| 119 | 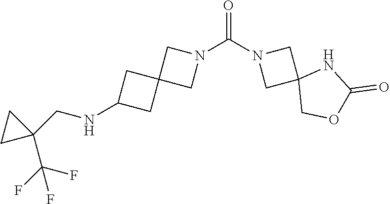 [SMILES: O=C1NC2(CO1)CN(C(=O)N1CC3(CC(NCC4(C(F)(F)F)CC4)C3)C1)C2] | 2-[6-[[1- (trifluoromethyl)cyclopro- pyl]methylamino]-2- azaspiro[3.3]heptane-2- carbonyl]-7-oxa-2,5- diazaspiro[3.4]octan-6- one | A.2 and B.156 | 389.2 [M + H]⁺ |
| 120 | 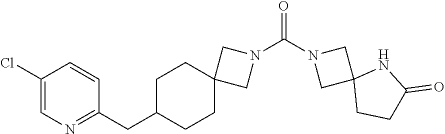 [SMILES: O=C1CCC2(CN(C(=O)N3CC4(CCC(Cc5ccc(Cl)cn5)CC4)C3)C2)N1] | 2-[7-[(5-chloro-2- pyridyl)methyl]-2- azaspiro[3.5]nonane-2- carbonyl]-2,5- diazaspiro[3.4]octan-6- one | A.1 and B.87 | 403.2 [M + H]⁺ |
| 121 | 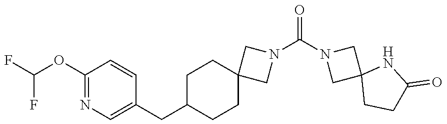 [SMILES: O=C1CCC2(CN(C(=O)N3CC4(CCC(Cc5ccc(OC(F)F)nc5)CC4)C3)C2)N1] | 2-[7-[[6- (difluoromethoxy)-3- pyridyl]methyl]-2- azaspiro[3.5]nonane-2- carbonyl]-2,5- diazaspiro[3.4]octan-6- one | A.1 and B.320 | 435.3 [M + H]⁺ |
| 122 | 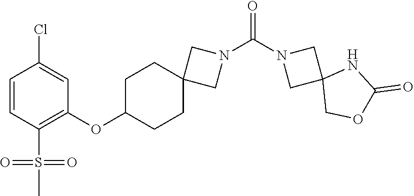 [SMILES: CS(=O)(=O)c1ccc(Cl)cc1OC1CCC2(CC1)CN(C(=O)N1CC3(COC(=O)N3)C1)C2] | 2-[7-(5-chloro-2-mesyl- phenoxy)-2- azaspiro[3.5]nonane-2- carobnyl]-7-oxa-2,5- diazaspiro[3.4]octan-6- one | A.2 and B.264 | 484.2 [M + H]⁺ |
| 123 | 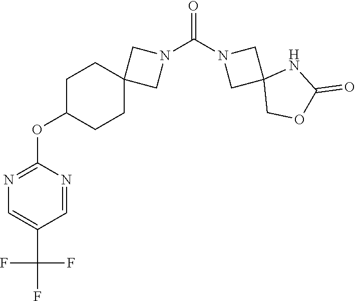 [SMILES: O=C1NC2(CO1)CN(C(=O)N1CC3(CCC(Oc4ncc(C(F)(F)F)cn4)CC3)C1)C2] | 2-[7-[5- (trifluoromethyl)pyrimi- din-2-yl]oxy-2- azaspiro[3.5]nonane-2- carbonyl]-7-oxa-2,5- diazaspiro[3.4]octan-6- one | A.2 and B.85 | 442.3 [M + H]⁺ |
| 124 | 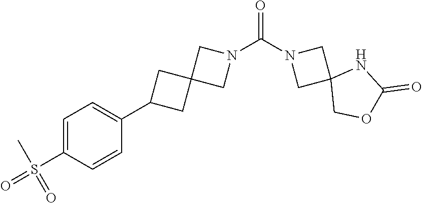 [SMILES: CS(=O)(=O)c1ccc(C2CC3(C2)CN(C(=O)N2CC4(COC(=O)N4)C2)C3)cc1] | 2-[6-(4-mesylphenyl)- 2-azaspiro[3.3]heptane- 2-carbonyl]-7-oxa-2,5- diazaspiro[3.4]octan-6- one | A.2 and B.124 | 406.2 [M + H]⁺ |
| 125 | 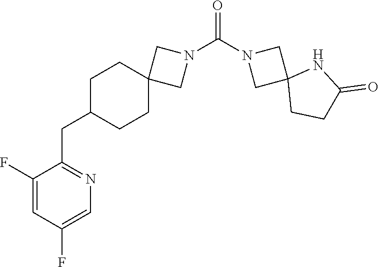 [SMILES: O=C1CCC2(CN(C(=O)N3CC4(CCC(Cc5ncc(F)cc5F)CC4)C3)C2)N1] | 2-[7-[(3,5-difluoro-2- pyridyl)methyl]-2- azaspiro[3.5]nonane-2- carbonyl]-2,5- diazaspiro[3.4]octan-6- one | A.1 and B.89 | 405.3 [M + H]⁺ |
| 126 | 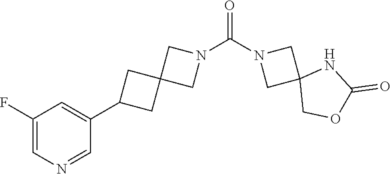 [SMILES: O=C1NC2(CO1)CN(C(=O)N1CC3(CC(c4cncc(F)c4)C3)C1)C2] | 2-[6-(5-fluoro-3- pyridyl)-2- azaspiro[3.3]heptane-2- carbonyl]-7-oxa-2,5- diazaspiro[3.4]octan-6- one | A.2 and B.325 | 347.1 [M + H]⁺ |
| 127 | 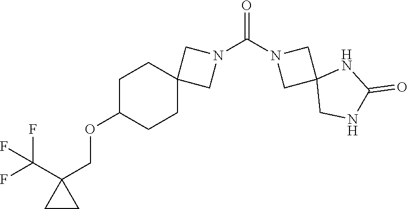 [SMILES: O=C1NCC2(CN(C(=O)N3CC4(CCC(OCC5(C(F)(F)F)CC5)CC4)C3)C2)N1] | 2-[7-[[1- (trifluoromethyl)cyclopro- pyl]methoxy]-2- azaspiro[3.5]nonane-2- carbonyl]-2,5,7- triazaspiro[3.4]octan-6- one | A.3 and B.37 | 417.3 [M + H]⁺ |
| 128 | 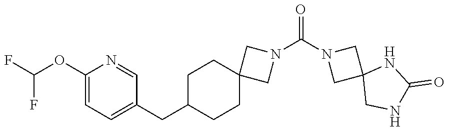 [SMILES: O=C1NCC2(CN(C(=O)N3CC4(CCC(Cc5ccc(OC(F)F)nc5)CC4)C3)C2)N1] | 2-[7-[[6- (difluoromethoxy)-3- pyridyl]methyl]-2- azaspiro[3.5]nonane-2- carbonyl]-2,5,7- triazaspiro[3.4]octan-6- one | A.3 and B.320 | 436.3 [M + H]⁺ |
| 130 | 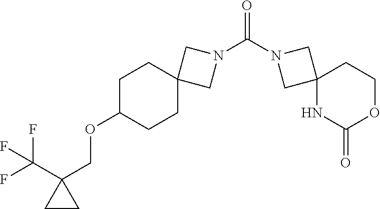 [SMILES: O=C1NC2(CCO1)CN(C(=O)N1CC3(CCC(OCC4(C(F)(F)F)CC4)CC3)C1)C2] | 2-[7-[[1- (trifluoromethyl)cyclopro- pyl]methoxy]-2- azaspiro[3.5]nonane-2- carbonyl]-7-oxa-2,5- diazaspiro[3.5]nonan-6- one | A.4 and B.37 | 432.3 [M + H]⁺ |
| 131 | 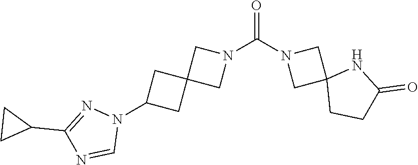 [SMILES: O=C1CCC2(CN(C(=O)N3CC4(CC(n5cnc(C6CC6)n5)C4)C3)C2)N1] | 2-[6-(3-cyclopropyl- 1,2,4-triazol-1-yl)-2- azaspiro[3.3]heptane-2- carbonyl]-2,5- diazaspiro[3.4]octan-6- one | A.1 and B.33 | 357.2 [M + H]⁺ |
| 132 | 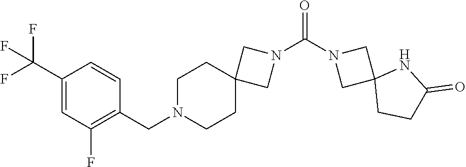 [SMILES: O=C1CCC2(CN(C(=O)N3CC4(CCN(Cc5ccc(C(F)(F)F)cc5F)CC4)C3)C2)N1] | 2-[7-[2-fluoro-4- (trifluoromethyl)benzyl- 2,7- diazaspiro[3.5]nonane- 2-carbonyl]-2,5- diazaspiro[3.4]octan-6- one | A.1 and B.117 | 455.3 [M + H]⁺ |
| 133 | 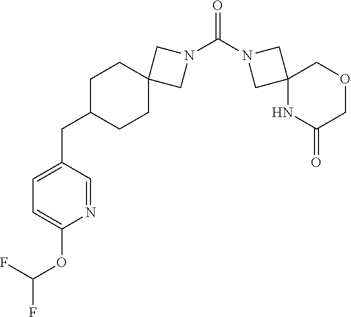 [SMILES: O=C1COCC2(CN(C(=O)N3CC4(CCC(Cc5ccc(OC(F)F)nc5)CC4)C3)C2)N1] | 2-[7-[[6- (difluoromethoxy)-3- pyridyl]methyl]-2- azaspiro[3.5]nonane-2- carbonyl]-8-oxa-2,5- diazaspiro[3.5]nonan-6- one | A.5 and B.320 | 451.4 [M + H]⁺ |
| 134 | 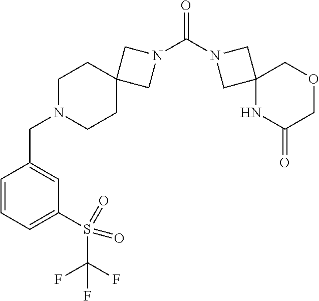 [SMILES: O=C1COCC2(CN(C(=O)N3CC4(CCN(Cc5cccc(S(=O)(=O)C(F)(F)F)c5)CC4)C3)C2)N1] | 2-[7-(3-triflylbenzyl)- 2,7- diazaspiro[3.5]nonane- 2-carbonyl]-8-oxa-2,5- diazaspiro[3.5]nonan-6- one | A.5 and B.120 | 517.4 [M + H]⁺ |
| 135 | 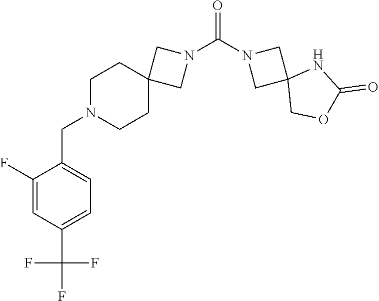 [SMILES: O=C1NC2(CO1)CN(C(=O)N1CC3(CCN(Cc4ccc(C(F)(F)F)cc4F)CC3)C1)C2] | 2-[7-[2-fluoro-4- (trifluoromethyl)benzyl- 2,7- diazaspiro[3.5]nonane- 2-carbonyl]-7-oxa-2,5- diazaspiro[3.4]octan-6- one | A.2 and B.117 | 457.2 [M + H]⁺ |
| 136 | 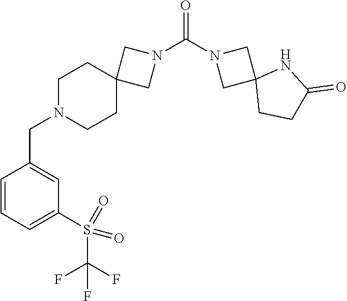 [SMILES: O=C1CCC2(CN(C(=O)N3CC4(CCN(Cc5cccc(S(=O)(=O)C(F)(F)F)c5)CC4)C3)C2)N1] | 2-[7-(3-triflylbenzyl)- 2,7- diazaspiro[3.5]nonane- 2-carbonyl]-2,5- diazaspiro[3.4]octan-6- one | A.1 and B.120 | 501.2 [M + H]⁺ |
| 137 | 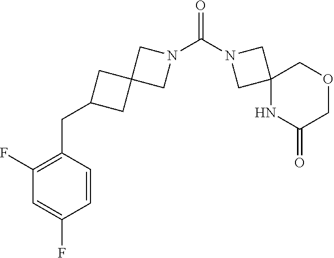 [SMILES: O=C1COCC2(CN(C(=O)N3CC4(CC(Cc5ccc(F)cc5F)C4)C3)C2)N1] | 2-[6-(2,4- difluorobenzyl)-2- azaspiro[3.3]heptane-2- carbonyl]-8-oxa-2,5- diazaspiro[3.5]nonan-6- one | A.5 and B.18 | 392.3 [M + H]⁺ |
| 138 | 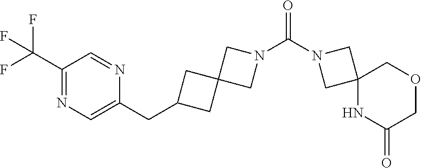 [SMILES: O=C1COCC2(CN(C(=O)N3CC4(CC(Cc5cnc(C(F)(F)F)cn5)C4)C3)C2)N1] | 2-[6-[[5- (trifluoromethyl)pyrazin- 2-yl]methyl]-2- azaspiro[3.3]heptane-2- carbonyl]-8-oxa-2,5- diazaspiro[3.5]nonan-6- one | A.5 and B.4 | 426.2 [M + H]⁺ |
| 139 | 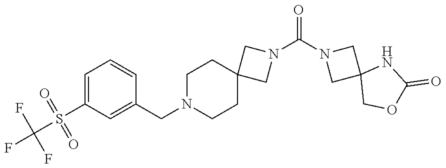 [SMILES: O=C1NC2(CO1)CN(C(=O)N1CC3(CCN(Cc4cccc(S(=O)(=O)C(F)(F)F)c4)CC3)C1)C2] | 2-[7-(3-triflylbenzyl)- 2,7- diazaspiro[3.5]nonane- 2-carbonyl]-7-oxa-2,5- diazaspiro[3.4]octan-6- one | A.2 and B.120 | 503.2 [M + H]⁺ |
| 140 | 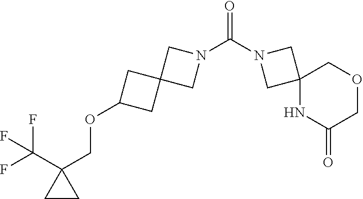 [SMILES: O=C1COCC2(CN(C(=O)N3CC4(CC(OCC5(C(F)(F)F)CC5)C4)C3)C2)N1] | 2-[6-[[1- (trifluoromethyl)cyclopro- pyl]methoxy]-2- azaspiro[3.3]heptane-2- carbonyl]-8-oxa-2,5- diazaspiro[3.5]nonan-6- one | A.5 and B.39 | 404.3 [M + H]⁺ |
| 141 | 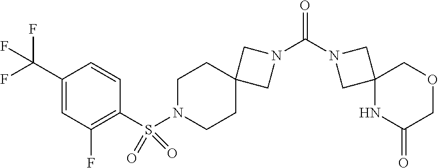 [SMILES: O=C1COCC2(CN(C(=O)N3CC4(CCN(S(=O)(=O)c5ccc(C(F)(F)F)cc5F)CC4)C3)C2)N1] | 2-[7-[2-fluoro-4- (trifluoromethyl)phenyl] sulfonyl-2,7- diazaspiro[3.5]nonane- 2-carbonyl]-8-oxa-2,5- diazaspiro[3.5]nonan-6- one | A.5 and B.168 | 521.2 [M + H]⁺ |
| 142 | 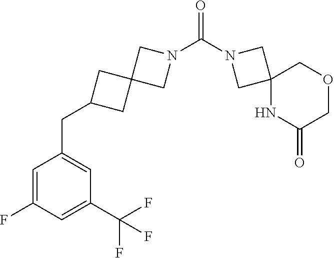 [SMILES: O=C1COCC2(CN(C(=O)N3CC4(CC(Cc5cc(F)cc(C(F)(F)F)c5)C4)C3)C2)N1] | 2-[6-[3-fluoro-5- (trifluoromethyl)benzyl- 2- azaspiro[3.3]heptane-2- carbonyl]-8-oxa-2,5- diazaspiro[3.5]nonan-6- one | A.5 and B.77 | 442.3 [M + H]⁺ |
| 143 | 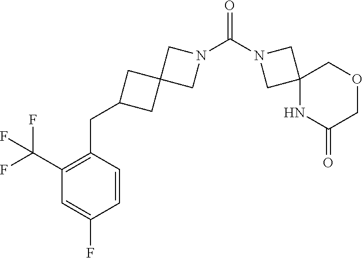 [SMILES: O=C1COCC2(CN(C(=O)N3CC4(CC(Cc5ccc(F)cc5C(F)(F)F)C4)C3)C2)N1] | 2-[6-[4-fluoro-2- (trifluoromethyl)benzyl- 2- azaspiro[3.3]heptane-2- carbonyl]-8-oxa-2,5- diazaspiro[3.5]nonan-6- one | A.5 and B.17 | 442.3 [M + H]⁺ |
| 144 | 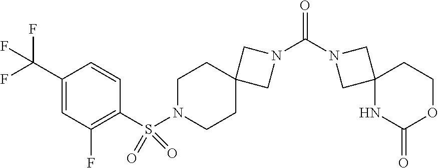 [SMILES: O=C1NC2(CCO1)CN(C(=O)N1CC3(CCN(S(=O)(=O)c4ccc(C(F)(F)F)cc4F)CC3)C1)C2] | 2-[7-[2-fluoro-4- (trifluoromethyl)phenyl] sulfonyl-2,7- diazaspiro[3.5]nonane- 2-carbonyl]-7-oxa-2,5- diazaspiro[3.5]nonan-6- one | A.4 and B.168 | 521.2 [M + H]⁺ |
| 145 | 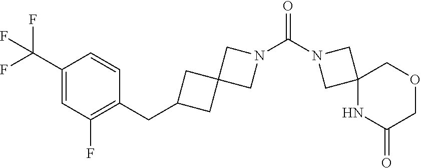 [SMILES: O=C1COCC2(CN(C(=O)N3CC4(CC(Cc5ccc(C(F)(F)F)cc5F)C4)C3)C2)N1] | 2-[6-[2-fluoro-4- (trifluoromethyl)benzyl- 2- azaspiro[3.3]heptane-2- carbonyl]-8-oxa-2,5- diazaspiro[3.5]nonan-6- one | A.5 and B.19 | 442.4 [M + H]⁺ |
| 146 | 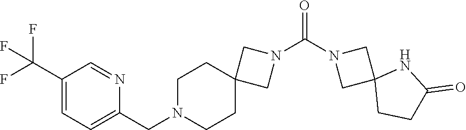 [SMILES: O=C1CCC2(CN(C(=O)N3CC4(CCN(Cc5ccc(C(F)(F)F)cn5)CC4)C3)C2)N1] | 2-[7-[[5- (trifluoromethyl)-2- pyridyl]methyl]-2,7- diazaspiro[3.5]nonane- 2-carbonyl]-2,5- diazaspiro[3.4]octan-6- one | A.1 and B.118 | 438.3 [M + H]⁺ |
| 147 | 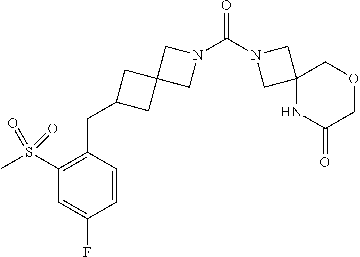 [SMILES: CS(=O)(=O)c1cc(F)ccc1CC1CC2(C1)CN(C(=O)N1CC3(COCC(=O)N3)C1)C2] | 2-[6-(4-fluoro-2-mesyl- benzyl)-2- azaspiro[3.3]heptane-2- carbonyl]-8-oxa-2,5- diazaspiro[3.5]nonan-6- one | A.5 and B.12 | 452.2 [M + H]⁺ |
| 148 | 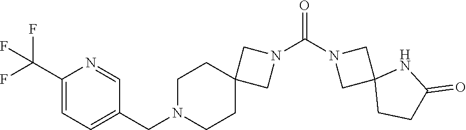 [SMILES: O=C1CCC2(CN(C(=O)N3CC4(CCN(Cc5ccc(C(F)(F)F)nc5)CC4)C3)C2)N1] | 2-[7-[[6- (trifluoromethyl)-3- pyridyl]methyl]-2,7- diazaspiro[3.5]nonane- 2-carbonyl]-2,5- diazaspiro[3.4]octan-6- one | A.1 and B.116 | 438.3 [M + H]⁺ |
| 149 | 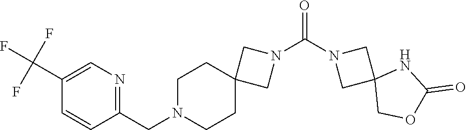 [SMILES: O=C1NC2(CO1)CN(C(=O)N1CC3(CCN(Cc4ccc(C(F)(F)F)cn4)CC3)C1)C2] | 2-[7-[[5- (trifluoromethyl)-2- pyridyl]methyl]-2,7- diazaspiro[3.5]nonane- 2-carbonyl]-7-oxa-2,5- diazaspiro[3.4]octan-6- one | A.2 and B.118 | 440.4 [M + H]⁺ |
| 150 | 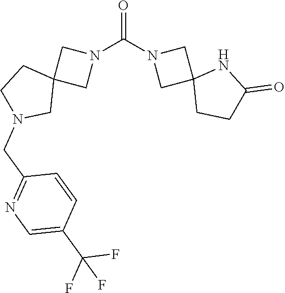 [SMILES: O=C1CCC2(CN(C(=O)N3CC4(CCN(Cc5ccc(C(F)(F)F)cn5)C4)C3)C2)N1] | 2-[7-[[5- (trifluoromethyl)-2- pyridyl]methyl]-2,7- diazaspiro[3.4]octane- 2-carbonyl]-2,5- diazaspiro[3.4]octan-6- one | A.1 and B.122 | 424.4 [M + H]⁺ |
| 151 | 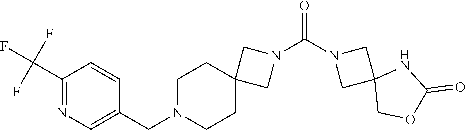 [SMILES: O=C1NC2(CO1)CN(C(=O)N1CC3(CCN(Cc4ccc(C(F)(F)F)nc4)CC3)C1)C2] | 2-[7-[[6- (trifluoromethyl)-3- pyridyl]methyl]-2,7- diazaspiro[3.5]nonane- 2-carbonyl]-7-oxa-2,5- diazaspiro[3.4]octan-6- one | A.2 and B.116 | 440.4 [M + H]⁺ |
| 152 | 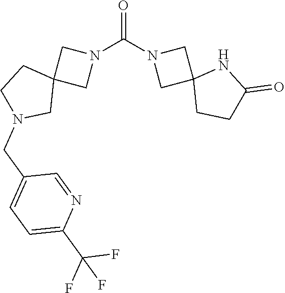 [SMILES: O=C1CCC2(CN(C(=O)N3CC4(CCN(Cc5ccc(C(F)(F)F)nc5)C4)C3)C2)N1] | 2-[7-[[6- (trifluoromethyl)-3- pyridyl]methyl]-2,7- diazaspiro[3.4]octane- 2-carbonyl]-2,5- diazaspiro[3.4]octan-6- one | A.1 and B.326 | 424.4 [M + H]⁺ |
| 153 | 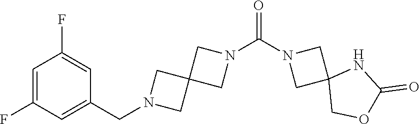 [SMILES: O=C1NC2(CO1)CN(C(=O)N1CC3(CN(Cc4cc(F)cc(F)c4)C3)C1)C2] | 2-[6-(3,5- difluorobenzyl)-2,6- diazaspiro[3.3]heptane- 2-carbonyl]-7-oxa-2,5- diazaspiro[3.4]octan-6- one | A.2 and B.100 | 379.2 [M + H]⁺ |
| 154 | 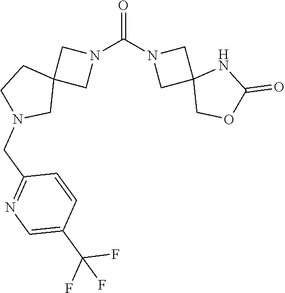 [SMILES: O=C1NC2(CO1)CN(C(=O)N1CC3(CCN(Cc4ccc(C(F)(F)F)cn4)C3)C1)C2] | 2-[7-[[5- (trifluoromethyl)-2- pyridyl]methyl]-2,7- diazaspiro[3.4]octane- 2-carbonyl]-7-oxa-2,5- diazaspiro[3.4]octan-6- one | A.2 and B.122 | 426.3 [M + H]⁺ |
| 155 | 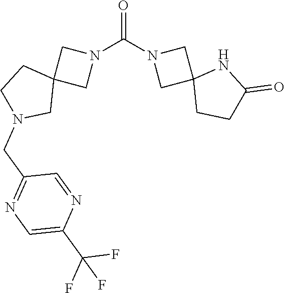 [SMILES: O=C1CCC2(CN(C(=O)N3CC4(CCN(Cc5cnc(C(F)(F)F)cn5)C4)C3)C2)N1] | 2-[7-[[5- (trifluoromethyl)pyrazin- 2-yl]methyl]-2,7- diazaspiro[3.4]octane- 2-carbonyl]-2,5- diazaspiro[3.4]octan-6- one | A.1 and B.124 | 425.4 [M + H]⁺ |
| 156 | 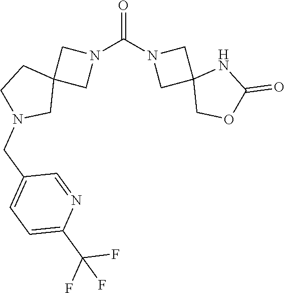 [SMILES: O=C1NC2(CO1)CN(C(=O)N1CC3(CCN(Cc4ccc(C(F)(F)F)nc4)C3)C1)C2] | 2-[7-[[6- (trifluoromethyl)-3- pyridyl]methyl]-2,7- diazaspiro[3.4]octane- 2-carbonyl]-7-oxa-2,5- diazaspiro[3.4]octan-6- one | A.2 and B.326 | 426.3 [M + H]⁺ |
| 157 | 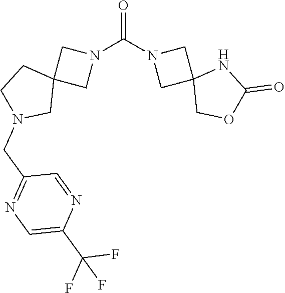 [SMILES: O=C1NC2(CO1)CN(C(=O)N1CC3(CCN(Cc4cnc(C(F)(F)F)cn4)C3)C1)C2] | 2-[7-[[5- (trifluoromethyl)pyrazin- 2-yl]methyl]-2,7- diazaspiro[3.4]octane- 2-carbonyl]-7-oxa-2,5- diazaspiro[3.4]octan-6- one | A.2 and B.124 | 427.3 [M + H]⁺ |
| 158 | 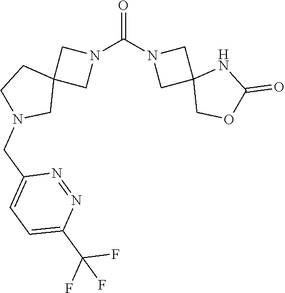 [SMILES: O=C1NC2(CO1)CN(C(=O)N1CC3(CCN(Cc4ccc(C(F)(F)F)nn4)C3)C1)C2] | 2-[7-[[6- (trifluoromethyl)pyrida- zin-3-yl]methyl]-2,7- diazaspiro[3.4]octane- 2-carbonyl]-7-oxa-2,5- diazaspiro[3.4]octan-6- one | A.2 and B.123 | 427.3 [M + H]⁺ |
| 159 | 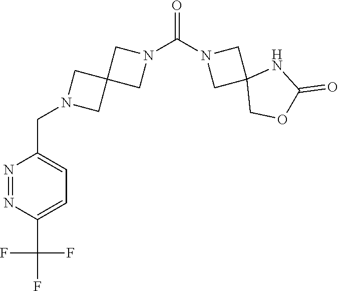 [SMILES: O=C1NC2(CO1)CN(C(=O)N1CC3(CN(Cc4ccc(C(F)(F)F)nn4)C3)C1)C2] | 2-[6-[[6- (trifluoromethyl)pyrida- zin-3-yl]methyl]-2,6- diazaspiro[3.3]heptane- 2-carbonyl]-7-oxa-2,5- diazaspiro[3.4]octan-6- one | A.2 and B.101 | 413.1 [M + H]⁺ |
| 160 | 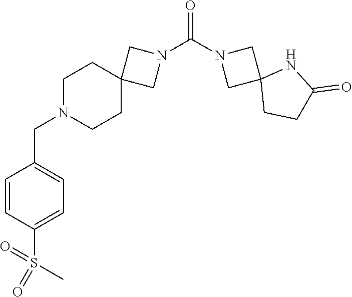 [SMILES: CS(=O)(=O)c1ccc(CN2CCC3(CC2)CN(C(=O)N2CC4(CCC(=O)N4)C2)C3)cc1] | 2-[7-(4-mesylbenzyl)- 2,7- diazaspiro[3.5]nonane- 2-carbonyl]-2,5- diazaspiro[3.4]octan-6- one | A.1 and B.119 | 447.3 [M + H]⁺ |
| 161 | 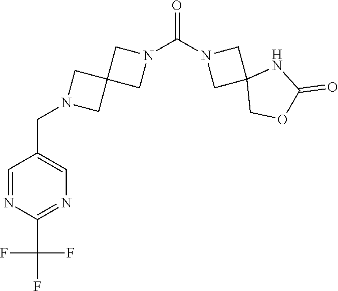 [SMILES: O=C1NC2(CO1)CN(C(=O)N1CC3(CN(Cc4cnc(C(F)(F)F)nc4)C3)C1)C2] | 2-[6-[[2- (trifluoromethyl)pyrimi- din-5-yl]methyl]-2,6- diazaspiro[3.3]heptane- 2-carbonyl]-7-oxa-2,5- diazaspiro[3.4]octan-6- one | A.2 and B.103 | 413.2 [M + H]⁺ |
| 162 | 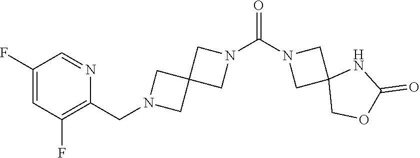 [SMILES: O=C1NC2(CO1)CN(C(=O)N1CC3(CN(Cc4ncc(F)cc4F)C3)C1)C2] | 2-[6-[(3,5-difluoro-2- pyridyl)methyl]-2,6- diazaspiro[3.3]heptane- 2-carbonyl]-7-oxa-2,5- diazaspiro[3.4]octan-6- one | A.2 and B.106 | 380.2 [M + H]⁺ |
| 163 | 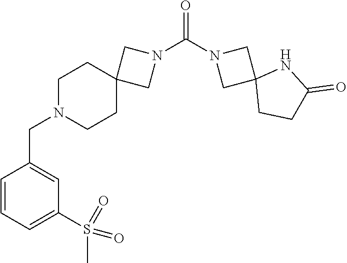 [SMILES: CS(=O)(=O)c1cccc(CN2CCC3(CC2)CN(C(=O)N2CC4(CCC(=O)N4)C2)C3)c1] | 2-[7-(3-mesylbenzyl)- 2,7- diazaspiro[3.5]nonane- 2-carbonyl]-2,5- diazaspiro[3.4]octan-6- one | A.1 and B.121 | 447.3 [M + H]⁺ |
| 164 | 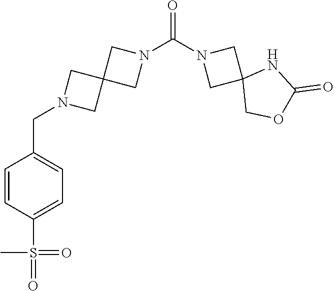 [SMILES: CS(=O)(=O)c1ccc(CN2CC3(C2)CN(C(=O)N2CC4(COC(=O)N4)C2)C3)cc1] | 2-[6-(4-mesylbenzyl)- 2,6- diazaspiro[3.3]heptane- 2-carbonyl]-7-oxa-2,5- diazaspiro[3.4]octan-6- one | A.2 and B.107 | 421.2 [M + H]⁺ |
| 165 | 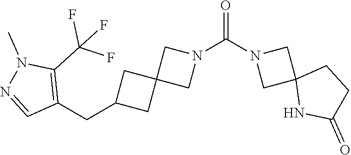 [SMILES: Cn1ncc(CC2CC3(C2)CN(C(=O)N2CC4(CCC(=O)N4)C2)C3)c1C(F)(F)F] | 2-[6-[1-methyl-5- (trifluoromethyl)pyrazol- 4-yl]methyl]-2- azaspiro[3.3]heptane-2- carbonyl]-2,5- diazaspiro[3.4]octan-6- one | A.1 and B.308 | 412.2 [M + H]⁺ |
| 166 | 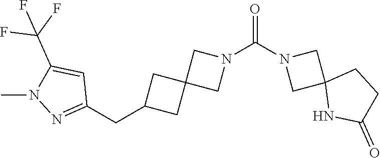 [SMILES: Cn1nc(CC2CC3(C2)CN(C(=O)N2CC4(CCC(=O)N4)C2)C3)cc1C(F)(F)F] | 2-[6-[[1-methyl-5- (trifluoromethyl)pyrazol- 3-yl]methyl]-2- azaspiro[3.3]heptane-2- carbonyl]-2,5- diazaspiro[3.4]octan-6- one | A.1 and B.309 | 412.3 [M + H]⁺ |
| 167 | 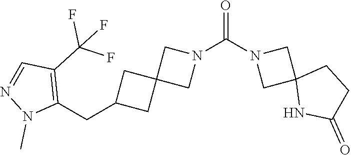 [SMILES: Cn1ncc(C(F)(F)F)c1CC1CC2(C1)CN(C(=O)N1CC3(CCC(=O)N3)C1)C2] | 2-[6-[[2-methyl-4- (trifluoromethyl)pyrazol- 3-yl]methyl]-2- azaspiro[3.3]heptane-2- carbonyl]-2,5- diazaspiro[3.4]octan-6- one | A.1 and B.310 | 412.2 [M + H]⁺ |
| 168 | 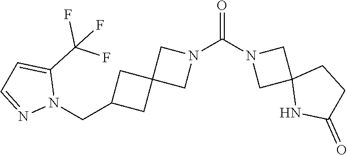 [SMILES: O=C1CCC2(CN(C(=O)N3CC4(CC(Cn5nccc5C(F)(F)F)C4)C3)C2)N1] | 2-[6-[[5- (trifluoromethyl)pyrazol- 1-yl]methyl]-2- azaspiro[3.3]heptane-2- carbonyl]-2,5- diazaspiro[3.4]octan-6- one | A.1 and B.311 | 398.2 [M + H]⁺ |
| 169 | 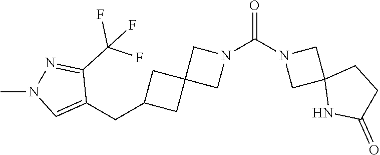 [SMILES: Cn1cc(CC2CC3(C2)CN(C(=O)N2CC4(CCC(=O)N4)C2)C3)c(C(F)(F)F)n1] | 2-[6-[[1-methyl-3- (trifluoromethyl)pyrazol- 4-yl]methyl]-2- azaspiro[3.3]heptane-2- carbonyl]-2,5- diazaspiro[3.4]octan-6- one | A.1 and B.312 | 412.2 [M + H]⁺ |
| 170 | 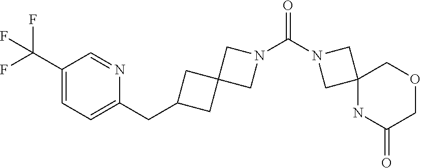 [SMILES: O=C1COCC2(CN(C(=O)N3CC4(CC(Cc5ccc(C(F)(F)F)cn5)C4)C3)C2)N1] | 2-[6-[[5- (trifluoromethyl)-2- pyridyl]methyl]-2- azaspiro[3.3]heptane-2- carbonyl]-8-oxa-2,5- diazaspiro[3.5]nonan-6- one | A.5 and B.69 | 425.3 [M + H]⁺ |
| 171 | 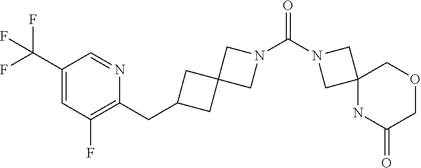 [SMILES: O=C1COCC2(CN(C(=O)N3CC4(CC(Cc5ncc(C(F)(F)F)cc5F)C4)C3)C2)N1] | 2-[6-[[3-fluoro-5- (trifluoromethyl)-2- pyridyl]methyl]-2- azaspiro[3.3]heptane-2- carbonyl]-8-oxa-2,5- diazaspiro[3.5]nonan-6- one | A.5 and B.6 | 443.3 [M + H]⁺ |
| 172 | 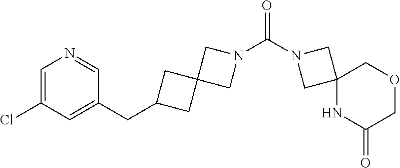 [SMILES: O=C1COCC2(CN(C(=O)N3CC4(CC(Cc5cncc(Cl)c5)C4)C3)C2)N1] | 2-[6-[(5-chloro-3- pyridyl)methyl]-2- azaspiro[3.3]heptane-2- carbonyl]-8-oxa-2,5- diazaspiro[3.5]nonan-6- one | A.5 and B.70 | 391.2 [M + H]⁺ |
| 173 | 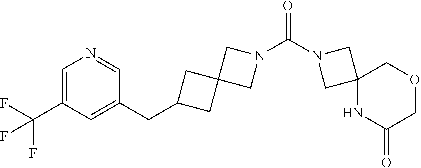 [SMILES: O=C1COCC2(CN(C(=O)N3CC4(CC(Cc5cncc(C(F)(F)F)c5)C4)C3)C2)N1] | 2-[6-[[5- (trifluoromethyl)-3- pyridyl]methyl]-2- azaspiro[3.3]heptane-2- carbonyl]-8-oxa-2,5- diazaspiro[3.5]nonan-6- one | A.5 and B.71 | 425.3 [M + H]⁺ |
| 174 | 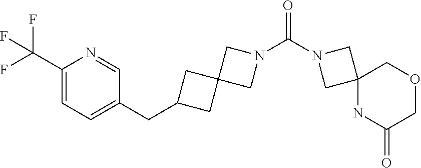 [SMILES: O=C1COCC2(CN(C(=O)N3CC4(CC(Cc5ccc(C(F)(F)F)nc5)C4)C3)C2)N1] | 2-[6-[[6- (trifluoromethyl)-3- pyridyl]methyl]-2- azaspiro[3.3]heptane-2- carbonyl]-8-oxa-2,5- diazaspiro[3.5]nonan-6- one | A.5 and B.3 | 425.3 [M + H]⁺ |
| 175 | 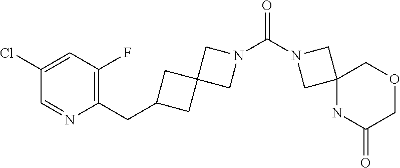 [SMILES: O=C1COCC2(CN(C(=O)N3CC4(CC(Cc5ncc(Cl)cc5F)C4)C3)C2)N1] | 2-[6-[(5-chloro-3- fluoro-2- pyridyl)methyl]-2- azaspiro[3.3]heptane-2- carbonyl]-2,5- diazaspiro[3.4]octan-6- one | A.1 and B.305 | 393.2 [M + H]⁺ |
| 176 | 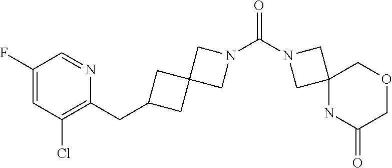 [SMILES: O=C1COCC2(CN(C(=O)N3CC4(CC(Cc5ncc(F)cc5Cl)C4)C3)C2)N1] | 2-[6-[(3-chloro-5- fluoro-2- pyridyl)methyl]-2- azaspiro[3.3]heptane-2- carbonyl]-8-oxa-2,5- diazaspiro[3.5]nonan-6- one | A.5 and B.73 | 409.2 [M + H]⁺ |
| 177 | 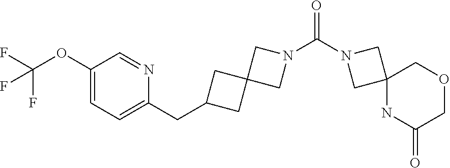 [SMILES: O=C1COCC2(CN(C(=O)N3CC4(CC(Cc5ccc(OC(F)(F)F)cn5)C4)C3)C2)N1] | 2-[6-[[5- (trifluoromethoxy)-2- pyridyl]methyl]-2- azaspiro[3.3]heptane-2- carbonyl]-8-oxa-2,5- diazaspiro[3.5]nonan-6- one | A.5 and B.5 | 441.3 [M + H]⁺ |
| 178 | 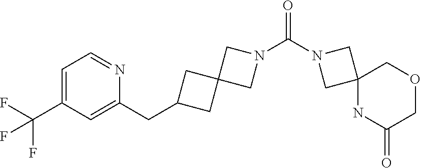 [SMILES: O=C1COCC2(CN(C(=O)N3CC4(CC(Cc5cc(C(F)(F)F)ccn5)C4)C3)C2)N1] | 2-[6-[[4- (trifluoromethyl)-2- pyridyl]methyl]-2- azaspiro[3.3]heptane-2- carbonyl]-8-oxa-2,5- diazaspiro[3.5]nonan-6- one | A.5 and B.74 | 425.3 [M + H]⁺ |
| 179 | 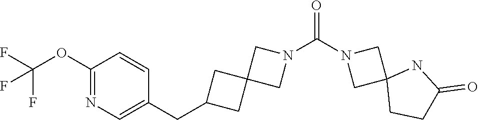 [SMILES: O=C1CCC2(CN(C(=O)N3CC4(CC(Cc5ccc(OC(F)(F)F)nc5)C4)C3)C2)N1] | 2-[6-[[6- (trifluoromethoxy)-3- pyridyl]methyl]-2- azaspiro[3.3]heptane-2- carbonyl]-2,5- diazaspiro[3.4]octan-6- one | A.1 and B.302 | 425.3 [M + H]⁺ |
| 180 | 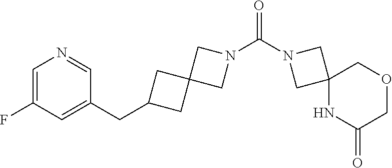 [SMILES: O=C1COCC2(CN(C(=O)N3CC4(CC(Cc5cncc(F)c5)C4)C3)C2)N1] | 2-[6-[(5-fluoro-3- pyridyl)methyl]-2- azaspiro[3.3]heptane-2- carbonyl]-8-oxa-2,5- diazaspiro[3.5]nonan-6- one | A.5 and B.23 | 375.2 [M + H]⁺ |
| 181 | 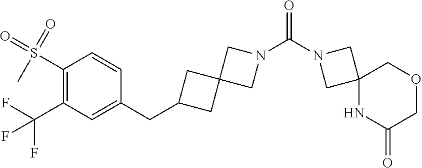 | 2-[6-[[4- methylsulfonyl-3- (trifluoromethyl)phenyl] methyl]-2- azaspiro[3.3]heptane-2- carbonyl]-8-oxa-2,5- diazaspiro[3.5]nonan-6- one | A.5 and B.24 | 488.2 [M + H]⁺ |
| 182 | 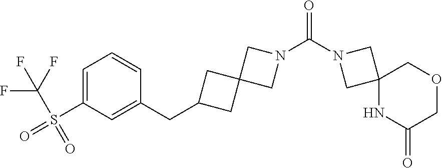 [SMILES: O=C1COCC2(CN(C(=O)N3CC4(CC(Cc5cccc(S(=O)(=O)C(F)(F)F)c5)C4)C3)C2)N1] | 2-[6-[[3- (trifluoromethylsulfonyl) phenyl]methyl]-2- azaspiro[3.3]heptane-2- carbonyl]-8-oxa-2,5- diazaspiro[3.5]nonan-6- one | A.5 and B.16 | 500.2 [M + H]⁺ |
| 183 | 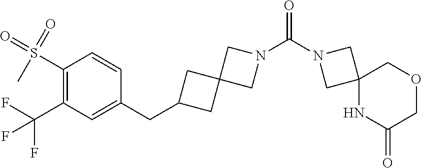 [SMILES: CS(=O)(=O)c1ccc(CC2CC3(C2)CN(C(=O)N2CC4(COCC(=O)N4)C2)C3)cc1C(F)(F)F] | 2-[6-[[4- methylsulfonyl-3- (trifluoromethyl)phenyl] methyl]-2- azaspiro[3.3]heptane-2- carbonyl]-2,5- diazaspiro[3.5]nonan-6- one | A.6 and B.24 | 502.3 [M + H]⁺ |
| 184 | 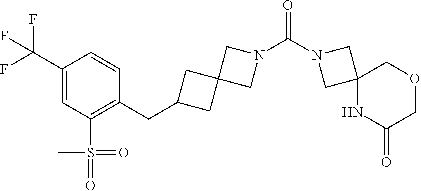 [SMILES: CS(=O)(=O)c1cc(C(F)(F)F)ccc1CC1CC2(C1)CN(C(=O)N1CC3(COCC(=O)N3)C1)C2] | 2-[6-[[2- methylsulfonyl-4- (trifluoromethyl)phenyl] methyl]-2- azaspiro[3.3]heptane-2- carbonyl]-8-oxa-2,5- diazaspiro[3.5]nonan-6- one | A.5 and B.19 | 500.2 [M + H]⁺ |
| 185 | 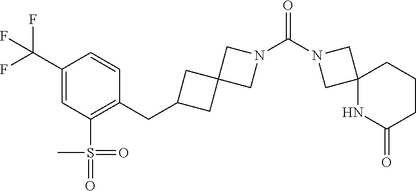 | 2-[6-[[2- methylsulfonyl-4- (trifluoromethyl)phenyl] methyl]-2- azaspiro[3.3]heptane-2- carbonyl]-2,5- diazaspiro[3.5]nonan-6- one | A.6 and B.19 | 486.2 [M + H]⁺ |
| 186 | 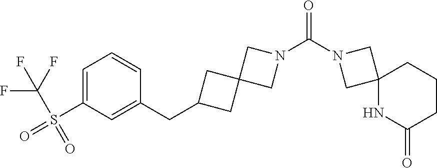 [SMILES: O=C1CCCC2(CN(C(=O)N3CC4(CC(Cc5cccc(S(=O)(=O)C(F)(F)F)c5)C4)C3)C2)N1] | 2-[6-[[3- (trifluoromethylsulfonyl) phenyl]methyl]-2- azaspiro[3.3]heptane-2- carbonyl]-2,5- diazaspiro[3.5]nonan-6- one | A.6 and B.16 | 421.2 [M + H]⁺ |
| 187 | 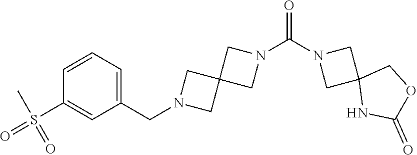 [SMILES: CS(=O)(=O)c1cccc(CN2CC3(C2)CN(C(=O)N2CC4(COC(=O)N4)C2)C3)c1] | 2-[6-[(3- methylsulfonylphenyl) methyl]-2,6- diazaspiro[3.3]heptane- 2-carbonyl]-7-oxa-2,5- diazaspiro[3.4]octan-6- one | A.2 and B.108 | 421.2 [M + H]⁺ |
| 188 | 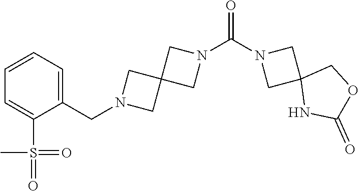 [SMILES: CS(=O)(=O)c1ccccc1CN1CC2(C1)CN(C(=O)N1CC3(COC(=O)N3)C1)C2] | 2-[6-[(2- methylsulfonylphenyl) methyl]-2,6- diazaspiro[3.3]heptane- 2-carbonyl]-7-oxa-2,5- diazaspiro[3.4]octan-6- one | A.2 and B.109 | 421.2 [M + H]⁺ |
| 189 | 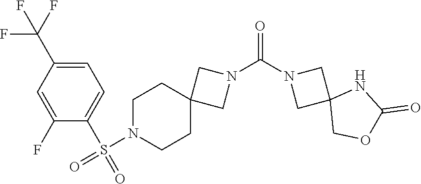 [SMILES: O=C1NC2(CO1)CN(C(=O)N1CC3(CCN(S(=O)(=O)c4ccc(C(F)(F)F)cc4F)CC3)C1)C2] | 2-[7-[2-fluoro-4- (trifluoromethyl)phenyl] sulfonyl-2,7- diazaspiro[3.5]nonane- 2-carbonyl]-7-oxa-2,5- diazaspiro[3.4]octan-6- one | A.2 and B.168 | 507.2 [M + H]⁺ |
| 190 | 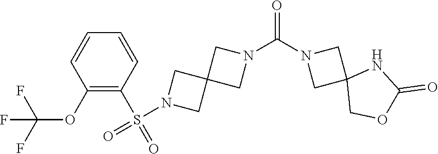 [SMILES: O=C1NC2(CO1)CN(C(=O)N1CC3(C1)CN(S(=O)(=O)c1ccccc1OC(F)(F)F)C3)C2] | 2-[2-[2- (trifluoromethoxy)phenyl] sulfonyl-2,6- diazaspiro[3.3]heptane- 6-carbonyl]-7-oxa-2,5- diazaspiro[3.4]octan-6- one | A.2 and B.57 | 477.2 [M + H]⁺ |
| 191 | 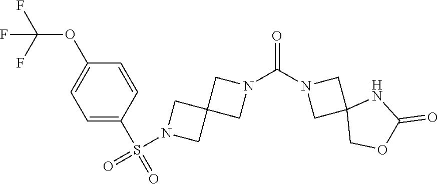 [SMILES: O=C1NC2(CO1)CN(C(=O)N1CC3(C1)CN(S(=O)(=O)c1ccc(OC(F)(F)F)cc1)C3)C2] | 2-[2-[4- (trifluoromethoxy)phenyl] sulfonyl-2,6- diazaspiro[3.3]heptane- 6-carbonyl]-7-oxa-2,5- diazaspiro[3.4]octan-6- one | A.2 and B.56 | 477.2 [M + H]⁺ |
| 192 | 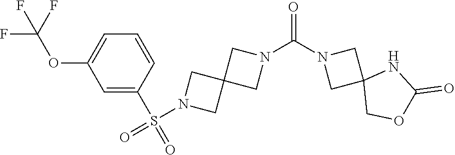 [SMILES: O=C1NC2(CO1)CN(C(=O)N1CC3(C1)CN(S(=O)(=O)c1cccc(OC(F)(F)F)c1)C3)C2] | 2-[2-[3- (trifluoromethoxy)phenyl] sulfonyl-2,6- diazaspiro[3.3]heptane- 6-carbonyl]-7-oxa-2,5- diazaspiro[3.4]octan-6- one | A.2 and B.55 | 477.2 [M + H]⁺ |
| 193 | 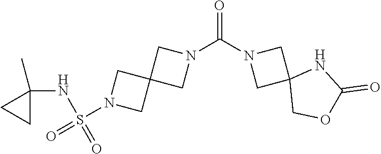 [SMILES: CC1(NS(=O)(=O)N2CC3(CN(C(=O)N4CC5(COC(=O)N5)C4)C3)C2)CC1] | N-(1- methylcyclopropyl)-6- (6-oxo-7-oxa-2,5- diazaspiro[3.4]octane- 2-carbonyl)-2,6- diazaspiro[3.3]heptane- 2-sulfonamide | A.2 and B.149 | 386.2 [M + H]⁺ |
| 195 | 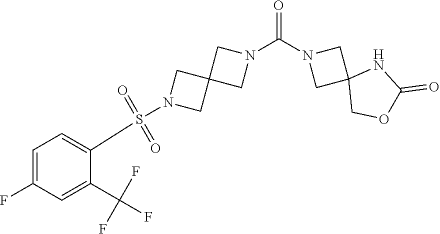 [SMILES: O=C1NC2(CO1)CN(C(=O)N1CC3(C1)CN(S(=O)(=O)c1ccc(F)cc1C(F)(F)F)C3)C2] | 2-[2-[4-fluoro-2- (trifluoromethyl)phenyl] sulfonyl-2,6- diazaspiro[3.3]heptane- 6-carbonyl]-7-oxa-2,5- diazaspiro[3.4]octan-6- one | A.2 and B.59 | 479.1 [M + H]⁺ |
| 196 | 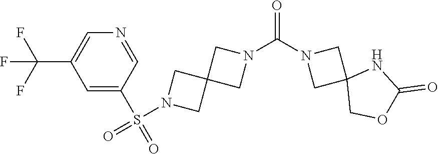 [SMILES: O=C1NC2(CO1)CN(C(=O)N1CC3(C1)CN(S(=O)(=O)c1cncc(C(F)(F)F)c1)C3)C2] | 2-[2-[[5- (trifluoromethyl)-3- pyridyl]sulfonyl]-2,6- diazaspiro[3.3]heptane- 6-carbonyl]-7-oxa-2,5- diazaspiro[3.4]octan-6- one | A.2 and B.68 | 462.2 [M + H]⁺ |
| 197 | 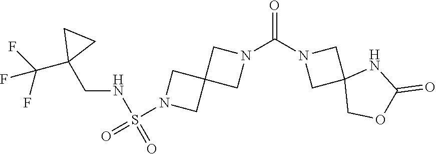 [SMILES: O=C1NC2(CO1)CN(C(=O)N1CC3(C1)CN(S(=O)(=O)NCC1(C(F)(F)F)CC1)C3)C2] | 6-(6-oxo-7-oxa-2,5- diazaspiro[3.4]octane- 2-carbonyl)-N-[[1- (trifluoromethyl)cyclopro- pyl]methyl]-2,6- diazaspiro[3.3]heptane- 2-sulfonamide | A.2 and B.152 | 454.2 [M + H]⁺ |
| 201 | 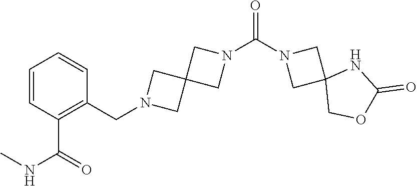 [SMILES: CNC(=O)c1ccccc1CN1CC2(C1)CN(C(=O)N1CC3(COC(=O)N3)C1)C2] | N-methyl-2-[[2-(6-oxo- 7-oxa-2,5- diazaspiro[3.4]octane- 2-carbonyl)-2,6- diazaspiro[3.3]heptan- 6-yl]methyl]benzamide | A.2 and B.113 | 400.2 [M + H]⁺ |
| 202 | 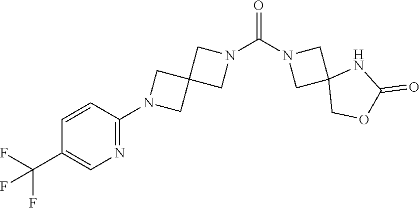 [SMILES: O=C1NC2(CO1)CN(C(=O)N1CC3(C1)CN(c1ccc(C(F)(F)F)cn1)C3)C2] | 2-[6-[5- (trifluoromethyl)-2- pyridyl]-2,6- diazaspiro[3.3]heptane- 2-carbonyl]-7-oxa-2,5- diazaspiro[3.4]octan-6- one | A.2 and B.366 | 398.2 [M + H]⁺ |
| 203 | 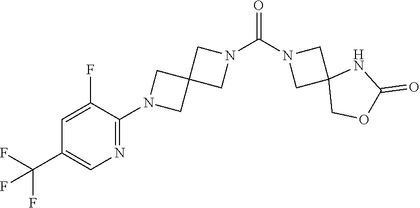 [SMILES: O=C1NC2(CO1)CN(C(=O)N1CC3(C1)CN(c1ncc(C(F)(F)F)cc1F)C3)C2] | 2-[6-[3-fluoro-5- (trifluoromethyl)-2- pyridyl]-2,6- diazaspiro[3.3]heptane- 2-carbonyl]-7-oxa-2,5- diazaspiro[3.4]octan-6- one | A.2 and B.367 | 416.2 [M + H]⁺ |
| 204 | 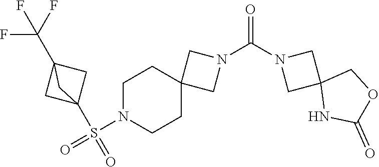 [SMILES: O=C1NC2(CO1)CN(C(=O)N1CC3(CCN(S(=O)(=O)C45CC(C(F)(F)F)(C4)C5)CC3)C1)C2] | 2-[7-[[3- (trifluoromethyl)-1- bicyclo[1.1.1]pentanyl] sulfonyl]-2,7- diazaspiro[3.5]nonane- 2-carbonyl]-7-oxa-2,5- diazaspiro[3.4]octan-6- one | A.2 and B.167 | 479.2 [M + H]⁺ |
| 206 | 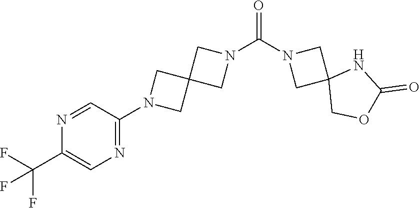 [SMILES: O=C1NC2(CO1)CN(C(=O)N1CC3(C1)CN(c1cnc(C(F)(F)F)cn1)C3)C2] | 2-[6-[5- (trifluoromethyl)pyrazin- 2-yl]-2,6- diazaspiro[3.3]heptane- 2-carbonyl]-7-oxa-2,5- diazaspiro[3.4]octan-6- one | A.2 and B.153 | 399.1 [M + H]⁺ |
| 207 | 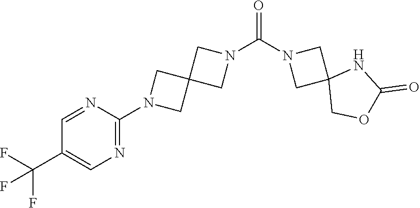 [SMILES: O=C1NC2(CO1)CN(C(=O)N1CC3(C1)CN(c1ncc(C(F)(F)F)cn1)C3)C2] | 2-[6-[5- (trifluoromethyl)pyrimi- din-2-yl]-2,6- diazaspiro[3.3]heptane- 2-carbonyl]-7-oxa-2,5- diazaspiro[3.4]octan-6- one | A.2 and B.372 | 399.1 [M + H]⁺ |
| 208 | 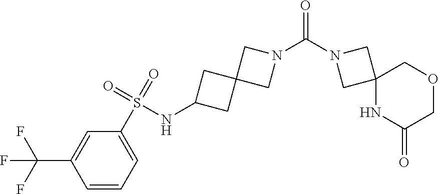 [SMILES: O=C1COCC2(CN(C(=O)N3CC4(CC(NS(=O)(=O)c5cccc(C(F)(F)F)c5)C4)C3)C2)N1] | N-[2-(6-oxo-8-oxa-2,5- diazaspiro[3.5]nonane- 2-carbonyl)-2- azaspiro[3.3]heptan-6- yl]-3- (trifluoromethyl)benzene- sulfonamide | A.5 and B.30 | 487.2 [M − H]⁻ |
| 209 | 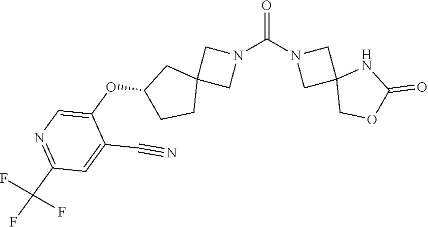 [SMILES: N#Cc1cc(C(F)(F)F)ncc1O[C@H]1CCC2(C1)CN(C(=O)N1CC3(COC(=O)N3)C1)C2] | 5-[[(6S)-2-(6-oxo-7- oxa-2,5- diazaspiro[3.4]octane- 2-carbonyl)-2- azaspiro[3.4]octan-6- yl]oxy]-2- (trifluoromethyl)pyridine- 4-carbonitrile | A.2 and B.97 | 452.3 [M + H]⁺ |
| 210 | 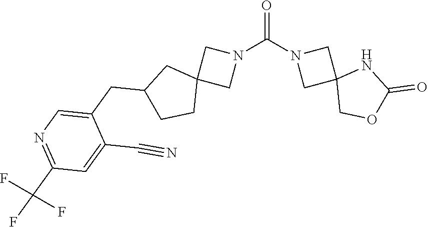 [SMILES: N#Cc1cc(C(F)(F)F)ncc1CC1CCC2(C1)CN(C(=O)N1CC3(COC(=O)N3)C1)C2] | 5-[[2-(6-oxo-7-oxa-2,5- diazaspiro[3.4]octane- 2-carbonyl)-2- azaspiro[3.4]octan-6- yl]methyl]-2- (trifluoromethyl)pyridine- 4-carbonitrile | A.2 and B.373 | 450.3 [M + H]⁺ |
| 211 | 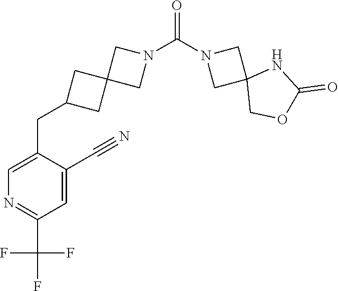 [SMILES: N#Cc1cc(C(F)(F)F)ncc1CC1CC2(C1)CN(C(=O)N1CC3(COC(=O)N3)C1)C2] | 5-[[2-(6-oxo-7-oxa-2,5- diazaspiro[3.4]octane- 2-carbonyl)-2- azaspiro[3.3]heptan-6- yl]methyl]-2- (trifluoromethyl)pyridine- 4-carbonitrile | A.2 and B.374 | 436.4 [M + H]⁺ |
| 212 | 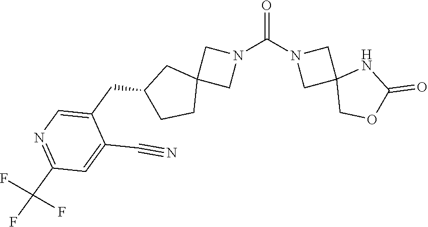 [SMILES: N#Cc1cc(C(F)(F)F)ncc1C[C@H]1CCC2(C1)CN(C(=O)N1CC3(COC(=O)N3)C1)C2] | 5-[[(6S)-2-(6-oxo-7- oxa-2,5- diazaspiro[3.4]octane- 2-carbonyl)-2- azaspiro[3.4]octan-6- yl]methyl]-2- (trifluoromethyl)pyridine- 4-carbonitrile | A.2 and P.90 | 450.4 [M + H]⁺ |
| 213 | 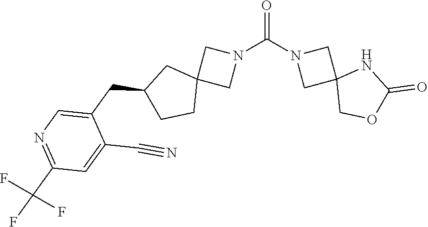 [SMILES: N#Cc1cc(C(F)(F)F)ncc1C[C@@H]1CCC2(C1)CN(C(=O)N1CC3(COC(=O)N3)C1)C2] | 5-[[(6R)-2-(6-oxo-7- oxa-2,5- diazaspiro[3.4]octane- 2-carbonyl)-2- azaspiro[3.4]octan-6- yl]methyl]-2- (trifluoromethyl)pyridine- 4-carbonitrile | A.2 and P.91 | 450.4 [M + H]⁺ |
| 214 | 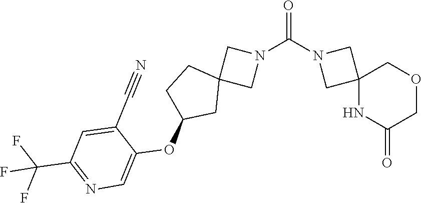 [SMILES: N#Cc1cc(C(F)(F)F)ncc1O[C@H]1CCC2(C1)CN(C(=O)N1CC3(COCC(=O)N3)C1)C2] | 5-[[(6S)-2-(6-oxo-8- oxa-2,5- diazaspiro[3.5]nonane- 2-carbonyl)-2- azaspiro[3.4]octan-6- yl]oxy]-2- (trifluoromethyl)pyridine- 4-carbonitrile | A.5 and B.97 | 466.3 [M + H]⁺ |
| 215 | 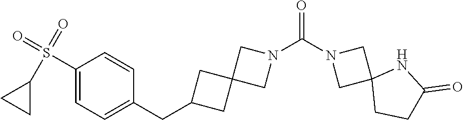 [SMILES: O=C1CCC2(CN(C(=O)N3CC4(CC(Cc5ccc(S(=O)(=O)C6CC6)cc5)C4)C3)C2)N1] | 2-[6-[(4- cyclopropylsulfonylphe- nyl)methyl]-2- azaspiro[3.3]heptane-2- carbonyl]-2,5- diazaspiro[3.4]octan-6- one | A.2 and B.331 | 444.3 [M + H]⁺ |
| 216 | 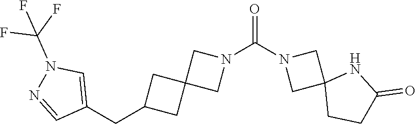 [SMILES: O=C1CCC2(CN(C(=O)N3CC4(CC(Cc5cnn(C(F)(F)F)c5)C4)C3)C2)N1] | 2-[6-[[1- (trifluoromethyl)pyrazol- 4-yl]methyl]-2- azaspiro[3.3]heptane-2- carbonyl]-2,5- diazaspiro[3.4]octan-6- one | A.1 and B.335 | 398.3 [M + H]⁺ |
| 217 | 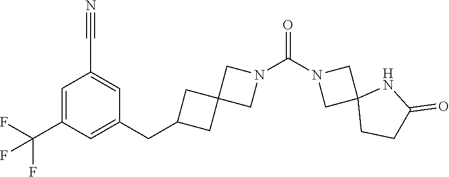 [SMILES: N#Cc1cc(CC2CC3(C2)CN(C(=O)N2CC4(CCC(=O)N4)C2)C3)cc(C(F)(F)F)c1] | 3-[[2-(6-oxo-2,5- diazaspiro[3.4]octane- 2-carbonyl)-2- azaspiro[3.3]heptan-6- yl]methyl]-5- (trifluoromethyl)benzoni- trile | A.1 and B.306 | 433.3 [M + H]⁺ |
| 218 | 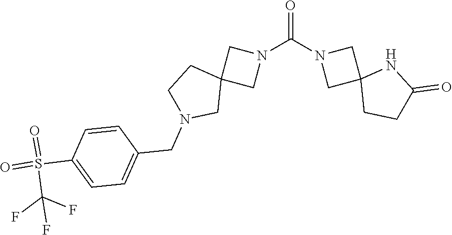 [SMILES: O=C1CCC2(CN(C(=O)N3CC4(CCN(Cc5ccc(S(=O)(=O)C(F)(F)F)cc5)C4)C3)C2)N1] | 2-[7-[[4- (trifluoromethylsulfonyl) phenyl]methyl]-2,7- diazaspiro[3.4]octane- 2-carbonyl]-2,5- diazaspiro[3.4]octan-6- one | A.2 and B.327 | 487.3 [M + H]⁺ |
| 219 | 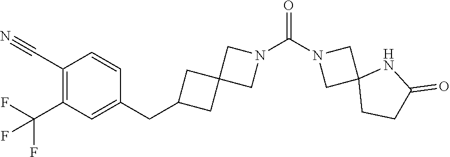 [SMILES: N#Cc1ccc(CC2CC3(C2)CN(C(=O)N2CC4(CCC(=O)N4)C2)C3)cc1C(F)(F)F] | 4-[[2-(6-oxo-2,5- diazaspiro[3.4]octane- 2-carbonyl)-2- azaspiro[3.3]heptan-6- yl]methyl]-2- (trifluoromethyl)benzoni- trile | A.1 and B.307 | 433.3 [M + H]⁺ |
| 220 | 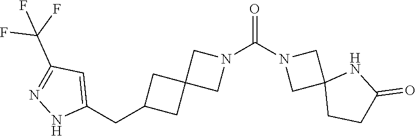 [SMILES: O=C1CCC2(CN(C(=O)N3CC4(CC(Cc5cc(C(F)(F)F)n[nH]5)C4)C3)C2)N1] | 2-[6-[[3- (trifluoromethyl)-1H- pyrazol-5-yl]methyl]-2- azaspiro[3.3]heptane-2- carbonyl]-2,5- diazaspiro[3.4]octan-6- one | A.1 and P.7 | 398.3 [M + H]⁺ |
| 221 | 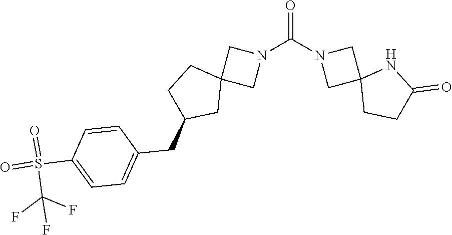 [SMILES: O=C1CCC2(CN(C(=O)N3CC4(CC[C@H](Cc5ccc(S(=O)(=O)C(F)(F)F)cc5)C4)C3)C2)N1] | 2-[(6R)-6-[[4- (trifluoromethylsulfonyl) phenyl]methyl]-2- azaspiro[3.4]octane-2- carbonyl]-2,5- diazaspiro[3.4]octan-6- one | A.1 and P.95 | 486.3 [M + H]⁺ |
| 222 | 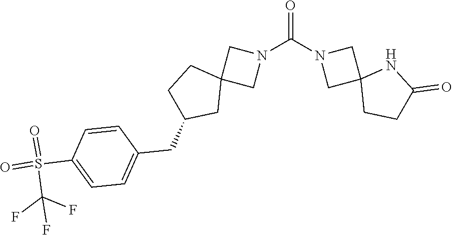 [SMILES: O=C1CCC2(CN(C(=O)N3CC4(CC[C@@H](Cc5ccc(S(=O)(=O)C(F)(F)F)cc5)C4)C3)C2)N1] | 2-[(6S)-6-[[4- (trifluoromethylsulfonyl) phenyl]methyl]-2- azaspiro[3.4]octane-2- carbonyl]-2,5- diazaspiro[3.4]octan-6- one | A.1 and B.316 | 486.3 [M + H]⁺ |
| 223 | 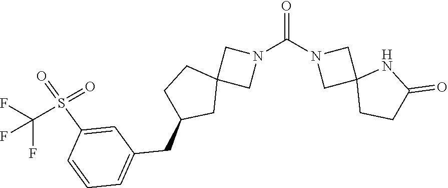 [SMILES: O=C1CCC2(CN(C(=O)N3CC4(CC[C@H](Cc5cccc(S(=O)(=O)C(F)(F)F)c5)C4)C3)C2)N1] | 2-[(6R)-6-[[3- (trifluoromethylsulfonyl) phenyl]methyl]-2- azaspiro[3.4]octane-2- carbonyl]-2,5- diazaspiro[3.4]octan-6- one | A.1 and P.96 | 486.3 [M + H]⁺ |
| 224 | 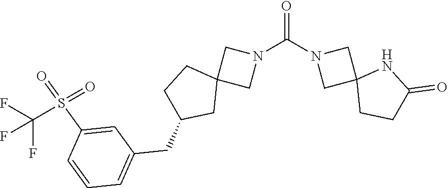 [SMILES: O=C1CCC2(CN(C(=O)N3CC4(CC[C@@H](Cc5cccc(S(=O)(=O)C(F)(F)F)c5)C4)C3)C2)N1] | 2-[(6S)-6-(3- triflylbenzyl)-2- azaspiro[3.4]octane-2- carbonyl]-2,5- diazaspiro[3.4]octan-6- one | A.1 and B.316 | 486.3 [M + H]⁺ |
| 225 | 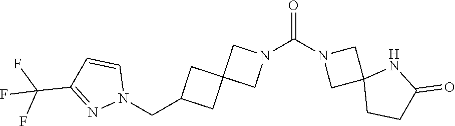 [SMILES: O=C1CCC2(CN(C(=O)N3CC4(CC(Cn5ccc(C(F)(F)F)n5)C4)C3)C2)N1] | 2-[6-[[3- (trifluoromethyl)pyrazol- 1-yl]methyl]-2- azaspiro[3.3]heptane-2- carbonyl]-2,5- diazaspiro[3.4]octan-6- one | A.1 and P.51 | 398.2 [M + H]⁺ |
| 226 | 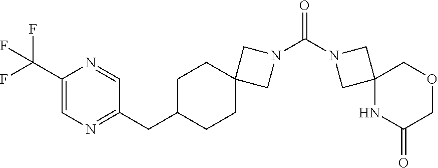 [SMILES: O=C1COCC2(CN(C(=O)N3CC4(CCC(Cc5cnc(C(F)(F)F)cn5)CC4)C3)C2)N1] | 2-[7-[[5- (trifluoromethyl)pyrazin- 2-yl]methyl]-2- azaspiro[3.5]nonane-2- carbonyl]-8-oxa-2,5- diazaspiro[3.5]nonan-6- one | A.5 and B.86 | 454.3 [M + H]⁺ |
| 227 | 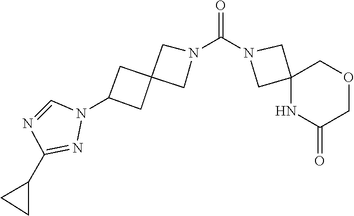 [SMILES: O=C1COCC2(CN(C(=O)N3CC4(CC(n5cnc(C6CC6)n5)C4)C3)C2)N1] | 2-[6-(3-cyclopropyl- 1,2,4-triazol-1-yl)-2- azaspiro[3.3]heptane-2- carbonyl]-8-oxa-2,5- diazaspiro[3.5]nonan-6- one | A.5 and B.33 | 373.2 [M + H]⁺ |
| 228 | 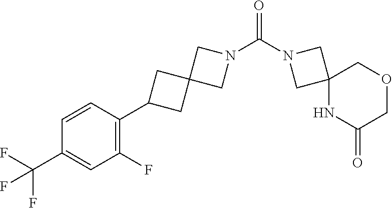 [SMILES: O=C1COCC2(CN(C(=O)N3CC4(CC(c5ccc(C(F)(F)F)cc5F)C4)C3)C2)N1] | 2-[6-[2-fluoro-4- (trifluoromethyl)phenyl- 2- azaspiro[3.3]heptane-2- carbonyl]-8-oxa-2,5- diazaspiro[3.5]nonan-6- one | A.5 and B.328 | 428.2 [M + H]⁺ |
| 229 | 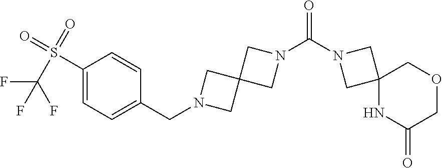 [SMILES: O=C1COCC2(CN(C(=O)N3CC4(CN(Cc5ccc(S(=O)(=O)C(F)(F)F)cc5)C4)C3)C2)N1] | 2-[6-[[4- (trifluoromethylsulfonyl) phenyl]methyl]-2,6- diazaspiro[3.3]heptane- 2-carbonyl]-8-oxa-2,5- diazaspiro[3.5]nonan-6- one | A.5 and B.54 | 489.2 [M + H]⁺ |
| 230 | 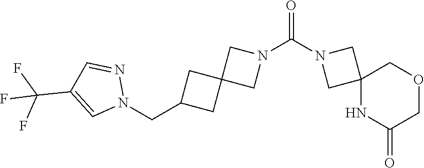 [SMILES: O=C1COCC2(CN(C(=O)N3CC4(CC(Cn5cc(C(F)(F)F)cn5)C4)C3)C2)N1] | 2-[6-[[4- (trifluoromethyl)pyrazol- 1-yl]methyl]-2- azaspiro[3.3]heptane-2- carbonyl]-8-oxa-2,5- diazaspiro[3.5]nonan-6- one | A.5 and B.32 | 414.2 [M + H]⁺ |
| 231 | 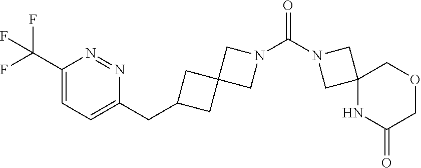 [SMILES: O=C1COCC2(CN(C(=O)N3CC4(CC(Cc5ccc(C(F)(F)F)nn5)C4)C3)C2)N1] | 2-[6-[[6- (trifluoromethyl)pyrida- zin-3-yl]methyl]-2- azaspiro[3.3]heptane-2- carbonyl]-8-oxa-2,5- diazaspiro[3.5]nonan-6- one | A.5 and B.11 | 426.3 [M + H]⁺ |
| 232 | 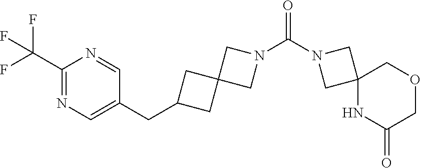 [SMILES: O=C1COCC2(CN(C(=O)N3CC4(CC(Cc5cnc(C(F)(F)F)nc5)C4)C3)C2)N1] | 2-[6-[[2- (trifluoromethyl)pyrimi- din-5-yl]methyl]-2- azaspiro[3.3]heptane-2- carbonyl]-8-oxa-2,5- diazaspiro[3.5]nonan-6- one | A.5 and B.10 | 426.2 [M + H]⁺ |
| 233 | 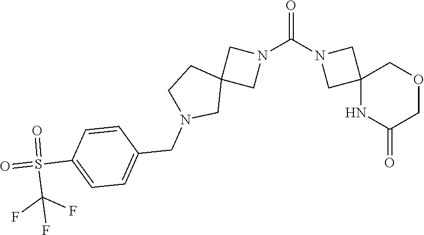 [SMILES: O=C1COCC2(CN(C(=O)N3CC4(CCN(Cc5ccc(S(=O)(=O)C(F)(F)F)cc5)C4)C3)C2)N1] | 2-[7-[[4- (trifluoromethylsulfonyl) phenyl]methyl]-2,7- diazaspiro[3.4]octane- 2-carbonyl]-8-oxa-2,5- diazaspiro[3.5]nonan-6- one | A.5 and B.327 | 503.2 [M + H]⁺ |
| 234 | 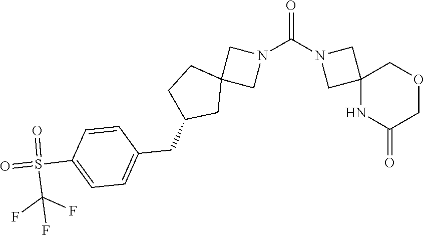 [SMILES: O=C1COCC2(CN(C(=O)N3CC4(CC[C@@H](Cc5ccc(S(=O)(=O)C(F)(F)F)cc5)C4)C3)C2)N1] | 2-[(6S)-6-[[4- (trifluoromethylsulfonyl) phenyl]methyl]-2- azaspiro[3.4]octane-2- carbonyl]-8-oxa-2,5- diazaspiro[3.5]nonan-6- one | A.5 and B.316 | 502.3 [M + H]⁺ |
| 235 | 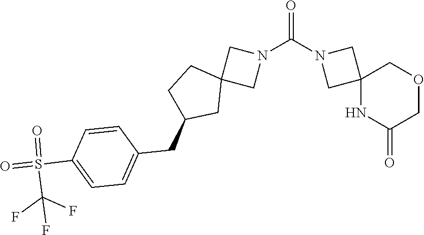 [SMILES: O=C1COCC2(CN(C(=O)N3CC4(CC[C@H](Cc5ccc(S(=O)(=O)C(F)(F)F)cc5)C4)C3)C2)N1] | 2-[(6R)-6-[[4- (trifluoromethylsulfonyl) phenyl]methyl]-2- azaspiro[3.4]octane-2- carbonyl]-8-oxa-2,5- diazaspiro[3.5]nonan-6- one | A.5 and P.95 | 502.3 [M + H]⁺ |
| 236 | 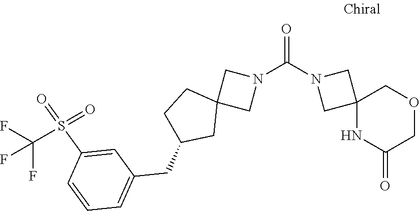 [SMILES: O=C1COCC2(CN(C(=O)N3CC4(CC[C@@H](Cc5cccc(S(=O)(=O)C(F)(F)F)c5)C4)C3)C2)N1] | 2-[(6S)-6-[[3- (trifluoromethylsulfonyl) phenyl]methyl]-2- azaspiro[3.4]octane-2- carobnyl]-8-oxa-2,5- diazaspiro[3.5]nonan-6- one | A.5 and B.316 | 502.3 [M + H]⁺ |
| 237 | 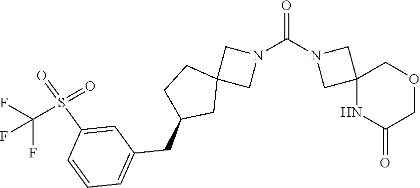 [SMILES: O=C1COCC2(CN(C(=O)N3CC4(CC[C@H](Cc5cccc(S(=O)(=O)C(F)(F)F)c5)C4)C3)C2)N1] | 2-[(6R)-6-[[3- (trifluoromethylsulfonyl) phenyl]methyl]-2- azaspiro[3.4]octane-2- carbonyl]-8-oxa-2,5- diazaspiro[3.5]nonan-6- one | A.5 and P.96 | 502.3 [M + H]⁺ |
| 238 | 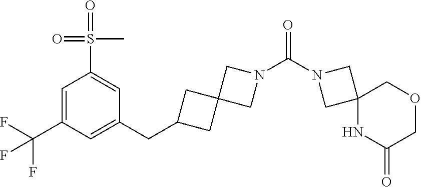 [SMILES: CS(=O)(=O)c1cc(CC2CC3(C2)CN(C(=O)N2CC4(COCC(=O)N4)C2)C3)cc(C(F)(F)F)c1] | 2-[6-[[3- methylsulfonyl-5- (trifluoromethyl)phenyl] methyl]-2- azaspiro[3.3]heptane-2- carbonyl]-8-oxa-2,5- diazaspiro[3.5]nonan-6- one | A.5 and B.330 | 502.3 [M + H]⁺ |
| 239 | 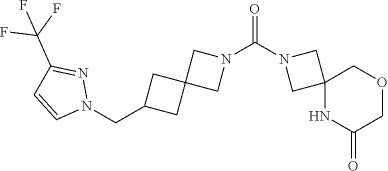 [SMILES: O=C1COCC2(CN(C(=O)N3CC4(CC(Cn5ccc(C(F)(F)F)n5)C4)C3)C2)N1] | 2-[6-[[3- (trifluoromethyl)pyrazol- 1-yl]methyl]-2- azaspiro[3.3]heptane-2- carbonyl]-8-oxa-2,5- diazaspiro[3.5]nonan-6- one | A.5 and P.51 | 414.2 [M + H]⁺ |
| 240 | 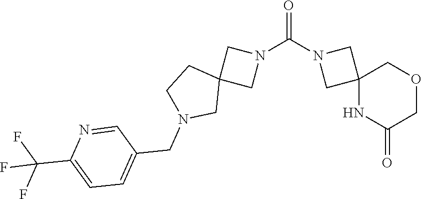 [SMILES: O=C1COCC2(CN(C(=O)N3CC4(CCN(Cc5ccc(C(F)(F)F)nc5)C4)C3)C2)N1] | 2-[7-[[6- (trifluoromethyl)-3- pyridyl]methyl]-2,7- diazaspiro[3.4]octane- 2-carbonyl]-8-oxa-2,5- diazaspiro[3.5]nonan-6- one | A.5 and B.326 | 440.3 [M + H]⁺ |
| 241 | 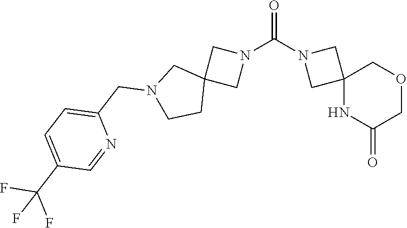 [SMILES: O=C1COCC2(CN(C(=O)N3CC4(CCN(Cc5ccc(C(F)(F)F)cn5)C4)C3)C2)N1] | 2-[7-[[5- (trifluoromethyl)-2- pyridyl]methyl]-2,7- diazaspiro[3.4]octane- 2-carbonyl]-8-oxa-2,5- diazaspiro[3.5]nonan-6- one | A.5 and B.122 | 440.3 [M + H]⁺ |
| 242 | 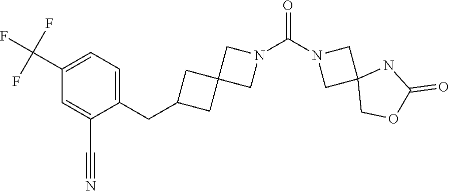 [SMILES: N#Cc1cc(C(F)(F)F)ccc1CC1CC2(C1)CN(C(=O)N1CC3(COC(=O)N3)C1)C2] | 2-[[2-(6-oxo-7-oxa-2,5- diazaspiro[3.4]octane- 2-carbonyl)-2- azaspiro[3.3]heptan-6- y]methyl]-5- (trifluoroemthyl)benzoni- trile | A.2 and B.321 | 435.2 [M + H]⁺ |
| 243 | 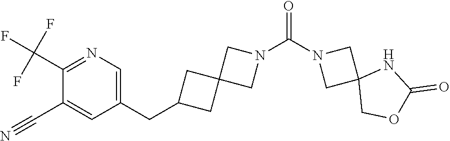 [SMILES: N#Cc1cc(CC2CC3(C2)CN(C(=O)N2CC4(COC(=O)N4)C2)C3)cnc1C(F)(F)F] | 5-[[2-(6-oxo-7-oxa-2,5- diazaspiro[3.4]octane- 2-carbonyl)-2- azaspiro[3.3]heptan-6- yl]methyl]-2- (trifluoromethyl)pyridine- 3-carbonitrile | A.2 and P.11 | 436.2 [M + H]⁺ |
| 244 | 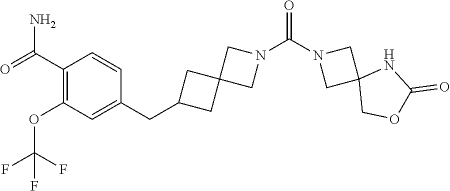 [SMILES: NC(=O)c1ccc(CC2CC3(C2)CN(C(=O)N2CC4(COC(=O)N4)C2)C3)cc1OC(F)(F)F] | 4-[[2-(6-oxo-7-oxa-2,5- diazaspiro[3.4]octane- 2-carbonyl)-2- azaspiro[3.3]heptan-6- yl]methyl]-2- (trifluoromethoxy)benz- amide | A.2 and B.358 | 469.2 [M + H]⁺ |
| 245 | 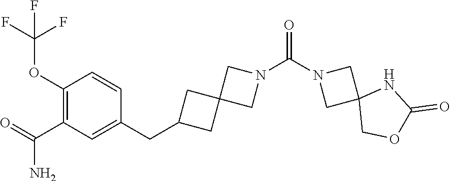 [SMILES: NC(=O)c1cc(CC2CC3(C2)CN(C(=O)N2CC4(COC(=O)N4)C2)C3)ccc1OC(F)(F)F] | 5-[[2-(6-oxo-7-oxa-2,5- diazaspiro[3.4]octane- 2-carbonyl)-2- azaspiro[3.3]heptan-6- yl]methyl]-2- (trifluoromethoxy)benz- amide | A.2 and B.359 | 469.2 [M + H]⁺ |
| 246 | 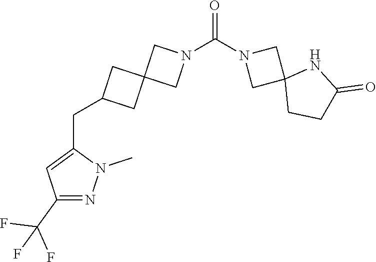 [SMILES: Cn1nc(C(F)(F)F)cc1CC1CC2(C1)CN(C(=O)N1CC3(CCC(=O)N3)C1)C2] | 2-[6-[[2-methyl-5- (trifluoromethyl)pyrazol- 3-yl]methyl]-2- azaspiro[3.3]heptane-2- carbonyl]-2,5- diazaspiro[3.4]octan-6- one | A.1 and P.31 | 412.2 [M + H]⁺ |
| 247 | 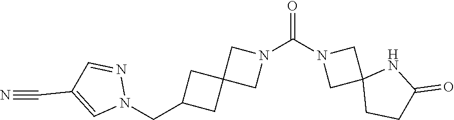 [SMILES: N#Cc1cnn(CC2CC3(C2)CN(C(=O)N2CC4(CCC(=O)N4)C2)C3)c1] | 1-[[2-(6-oxo-2,5- diazaspiro[3.4]octane- 2-carbonyl)-2- azaspiro[3.3]heptan-6- yl]methyl]pyrazole-4- carbonitrile | A.1 and B.354 | 355.2 [M + H]⁺ |
| 248 | 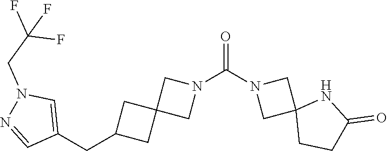 [SMILES: O=C1CCC2(CN(C(=O)N3CC4(CC(Cc5cnn(CC(F)(F)F)c5)C4)C3)C2)N1] | 2-[6-[[1-(2,2,2- trifluoroethyl)pyrazol- 4-yl]methyl]-2- azaspiro[3.3]heptane-2- carbonyl]-2,5- diazaspiro[3.4]octan-6- one | A.1 and P.37 | 412.2 [M + H]⁺ |
| 249 | 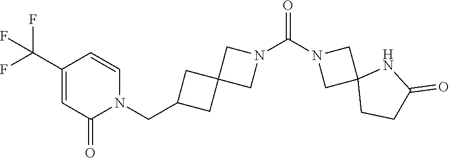 [SMILES: O=C1CCC2(CN(C(=O)N3CC4(CC(Cn5ccc(C(F)(F)F)cc5=O)C4)C3)C2)N1] | 2-[6-[[2-oxo-4- (trifluoromethyl)-1- pyridyl]methyl]-2- azaspiro[3.3]heptane-2- carbonyl]-2,5- diazaspiro[3.4]octan-6- one | A.1 and P.23 | 425.2 [M + H]⁺ |
| 251 | 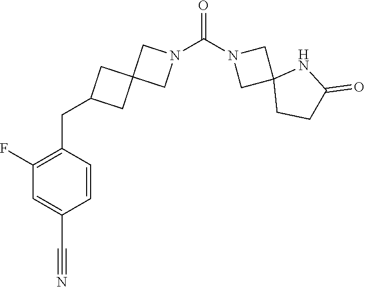 [SMILES: N#Cc1ccc(CC2CC3(C2)CN(C(=O)N2CC4(CCC(=O)N4)C2)C3)c(F)c1] | 3-fluoro-4-[[2-(6-oxo- 2,5- diazaspiro[3.4]octane- 2-carbonyl)-2- azaspiro[3.3]heptan-6- yl]methyl]benzonitrile | A.1 and B.322 | 383.2 [M + H]⁺ |
| 252 | 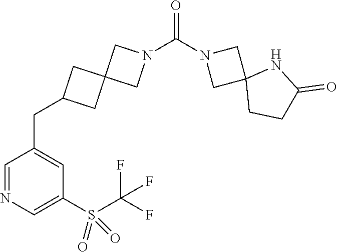 [SMILES: O=C1CCC2(CN(C(=O)N3CC4(CC(Cc5cncc(S(=O)(=O)C(F)(F)F)c5)C4)C3)C2)N1] | 2-[6-[[5- (trifluoromethylsulfonyl)- 3-pyridyl]methyl]-2- azaspiro[3.3]heptane-2- carbonyl]-2,5- diazaspiro[3.4]octan-6- one | A.1 and P.13 | 473.2 [M + H]⁺ |
| 253 | 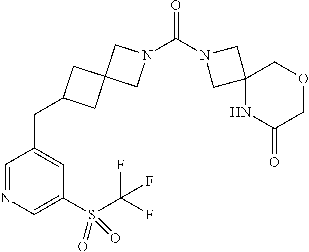 [SMILES: O=C1COCC2(CN(C(=O)N3CC4(CC(Cc5cncc(S(=O)(=O)C(F)(F)F)c5)C4)C3)C2)N1] | 2-[6-[[5- (trifluoromethylsulfonyl)- 3-pyridyl]methyl]-2- azaspiro[3.3]heptane-2- carbonyl]-8-oxa-2,5- diazaspiro[3.5]nonan-6- one | A.5 and P.13 | 489.2 [M + H]⁺ |
| 254 | 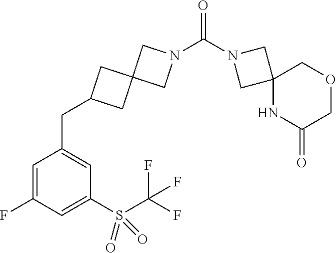 [SMILES: O=C1COCC2(CN(C(=O)N3CC4(CC(Cc5cc(F)cc(S(=O)(=O)C(F)(F)F)c5)C4)C3)C2)N1] | 2-[6-[[3-fluoro-5- (trifluoromethylsulfonyl) phenyl]methyl]-2- azaspiro[3.3]heptane-2- carbonyl]-8-oxa-2,5- diazaspiro[3.5]nonan-6- one | A.5 and P.66 | 506.2 [M + H]⁺ |
| 255 | 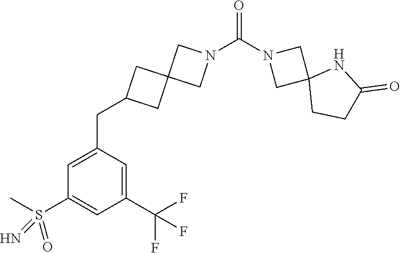 [SMILES: CS(=N)(=O)c1cc(CC2CC3(C2)CN(C(=O)N2CC4(CCC(=O)N4)C2)C3)cc(C(F)(F)F)c1] | 2-[6-[[3- (methylsulfonimidoyl)- 5- (trifluoromethyl)phenyl] methyl]-2- azaspiro[3.3]heptane-2- carbonyl]-2,5- diazaspiro[3.4]octan-6- one | A.1 and P.71 | 485.2 [M + H]⁺ |
| 256 | 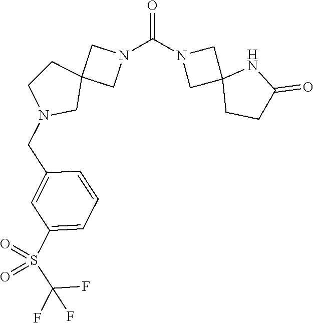 [SMILES: O=C1CCC2(CN(C(=O)N3CC4(CCN(Cc5cccc(S(=O)(=O)C(F)(F)F)c5)C4)C3)C2)N1] | 2-[7-[[3- (trifluoromethylsulfonyl) phenyl]methyl]-2,7- diazaspiro[3.4]octane- 2-carbonyl]-2,5- diazaspiro[3.4]octan-6- one | A.1 and B.323 | 487.2 [M + H]⁺ |
| 257 | 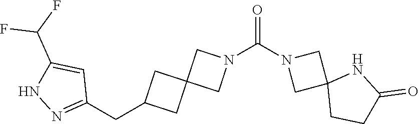 [SMILES: O=C1CCC2(CN(C(=O)N3CC4(CC(Cc5cc(C(F)F)[nH]n5)C4)C3)C2)N1] | 2-[6-[[5- (difluoromethyl)-1H- pyrazol-3-yl]methyl]-2- azaspiro[3.4]heptane-2- carbonyl]-2,5- diazaspiro[3.4]octan-6- one | A.1 and P.62 | 380.2 [M + H]⁺ |
| 258 | 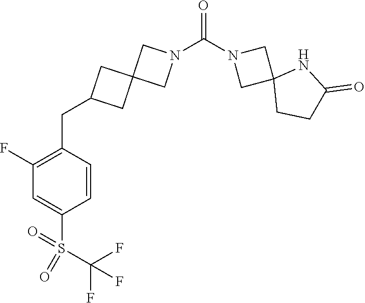 [SMILES: O=C1CCC2(CN(C(=O)N3CC4(CC(Cc5ccc(S(=O)(=O)C(F)(F)F)cc5F)C4)C3)C2)N1] | 2-[6-[[2-fluoro-4- (trifluoromethylsulfonyl) phenyl]methyl]-2,6- diazaspiro[3.4]heptane- 2-carbonyl]-2,5- diazaspiro[3.4]octan-6- one | A.1 and B.396 | 491.2 [M + H]⁺ |
| 259 | 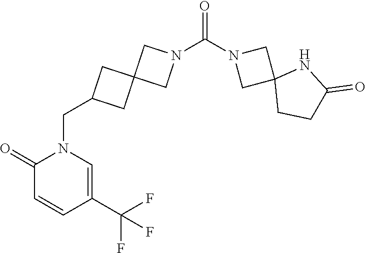 [SMILES: O=C1CCC2(CN(C(=O)N3CC4(CC(Cn5cc(C(F)(F)F)ccc5=O)C4)C3)C2)N1] | 2-[6-[[2-oxo-5- (trifluoromethyl)-1- pyridyl]methyl]-2- azaspiro[3.3]heptane-2- carbonyl]-2,5- diazaspiro[3.4]octan-6- one | A.1 and P.24 | 425.2 [M + H]⁺ |
| 260 | 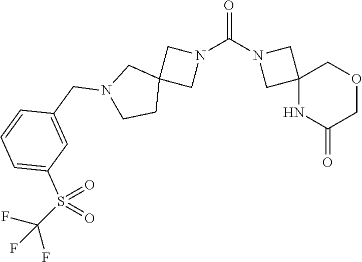 [SMILES: O=C1COCC2(CN(C(=O)N3CC4(CCN(Cc5cccc(S(=O)(=O)C(F)(F)F)c5)C4)C3)C2)N1] | 2-[7-[[3- (trifluoromethylsulfonyl) phenyl]methyl]-2,7- diazaspiro[3.4]octane- 2-carbonyl]-8-oxa-2,5- diazaspiro[3.5]nonan-6- one | A.5 and B.323 | 503.2 [M + H]⁺ |
| 261 | 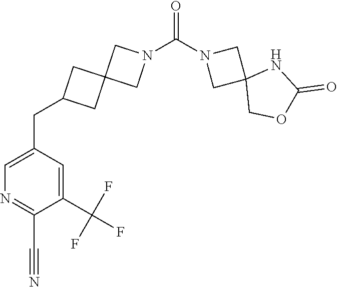 [SMILES: N#Cc1ncc(CC2CC3(C2)CN(C(=O)N2CC4(COC(=O)N4)C2)C3)cc1C(F)(F)F] | 5-[[2-(6-oxo-7-oxa-2,5- diazaspiro[3.4]octane- 2-carbonyl)-2- azaspiro[3.3]heptan-6- yl]methyl]-3- (trifluoromethyl)pyridine- 2-carbonitrile | A.2 and P.10 | 436.2 [M + H]⁺ |
| 262 | 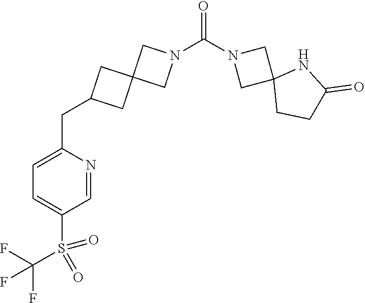 [SMILES: O=C1CCC2(CN(C(=O)N3CC4(CC(Cc5ccc(S(=O)(=O)C(F)(F)F)cn5)C4)C3)C2)N1] | 2-[6-[[5- (trifluoromethylsulfonyl)- 2-pyridyl]methyl]-2- azaspiro[3.3]heptane-2- carbonyl]-2,5- diazaspiro[3.4]octan-6- one | A.1 and P.12 | 473.2 [M + H]⁺ |
| 263 | 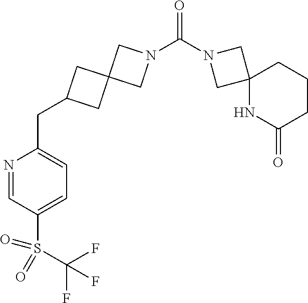 [SMILES: O=C1CCCC2(CN(C(=O)N3CC4(CC(Cc5ccc(S(=O)(=O)C(F)(F)F)cn5)C4)C3)C2)N1] | 2-[6-[[5- (trifluoromethylsulfonyl)- 2-pyridyl]methyl]-2- azaspiro[3.3]heptane-2- carbonyl]-2,5- diazaspiro[3.5]nonan-6- one | A.6 and P.12 | 487.2 [M + H]⁺ |
| 264 | 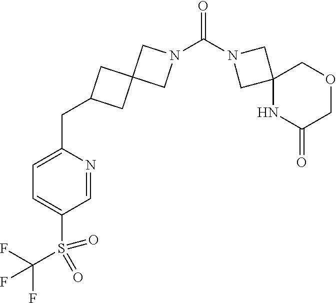 [SMILES: O=C1COCC2(CN(C(=O)N3CC4(CC(Cc5ccc(S(=O)(=O)C(F)(F)F)cn5)C4)C3)C2)N1] | 2-[6-[[5- (trifluoromethylsulfonyl)- 2-pyridyl]methyl]-2- azaspiro[3.3]heptane-2- carbonyl]-8-oxa-2,5- diazaspiro[3.5]nonan-6- one | A.5 and P.12 | 489.2 [M + H]⁺ |
| 265 | 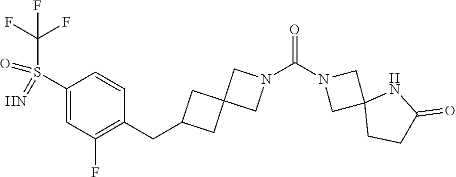 [SMILES: N=S(=O)(c1ccc(CC2CC3(C2)CN(C(=O)N2CC4(CCC(=O)N4)C2)C3)c(F)c1)C(F)(F)F] | 2-[6-[[2-fluoro-4- (trifluoromethylsulfoni- midoyl)phenyl]methyl]- 2-azaspiro[3.3]heptane- 2-carbonyl]-2,5- diazaspiro[3.5]octan-6- one | A.1 and B.329 | 489.2 [M + H]⁺ |
| 266 | 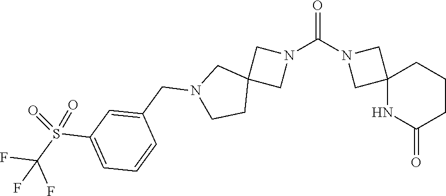 [SMILES: O=C1CCCC2(CN(C(=O)N3CC4(CCN(Cc5cccc(S(=O)(=O)C(F)(F)F)c5)C4)C3)C2)N1] | 2-[7-[[3- (trifluoromethylsulfonyl) phenyl]methyl]-2,7- diazaspiro[3.4]octane- 2-carbonyl]-2,5- diazaspiro[3.5]nonan-6- one | A.6 and B.323 | 501.2 [M + H]⁺ |
| 267 | 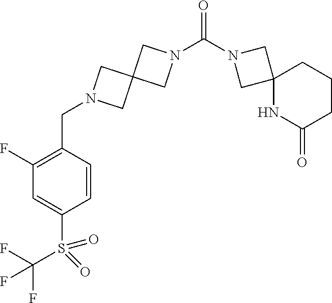 [SMILES: O=C1CCCC2(CN(C(=O)N3CC4(CN(Cc5ccc(S(=O)(=O)C(F)(F)F)cc5F)C4)C3)C2)N1] | 2-[6-[[2-fluoro-4- (trifluoromethylsulfonyl) phenyl]methyl]-2,6- diazaspiro[3.3]heptane- 2-carbonyl]-2,5- diazaspiro[3.5]nonan-6- one | A.6 and B.396 | 505.1 [M + H]⁺ |
| 268 | 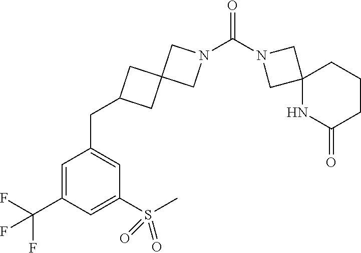 [SMILES: CS(=O)(=O)c1cc(CC2CC3(C2)CN(C(=O)N2CC4(CCCC(=O)N4)C2)C3)cc(C(F)(F)F)c1] | 2-[6-[[3- methylsulfonyl-5- (trifluoromethyl)phenyl] methyl]-2- azaspiro[3.3]heptane-2- carbonyl]-2,5- diazaspiro[3.5]nonan-6- one | A.6 and B.330 | 500.2 [M + H]⁺ |
| 269 | 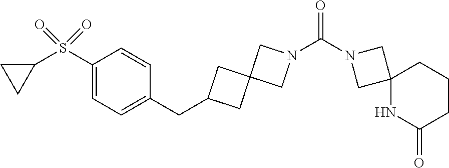 [SMILES: O=C1CCCC2(CN(C(=O)N3CC4(CC(Cc5ccc(S(=O)(=O)C6CC6)cc5)C4)C3)C2)N1] | 2-[6-[(4- cyclopropylsulfonylphe- nyl)methyl]-2- azaspiro[3.3]heptane-2- carbonyl]-2,5- diazaspiro[3.5]nonan-6- one | A.6 and B.331 | 458.2 [M + H]⁺ |
| 270 | 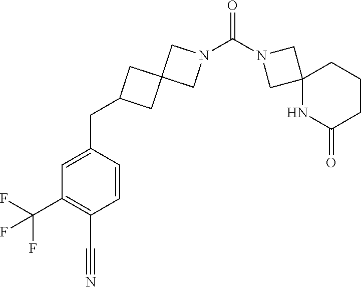 [SMILES: N#Cc1ccc(CC2CC3(C2)CN(C(=O)N2CC4(CCCC(=O)N4)C2)C3)cc1C(F)(F)F] | 4-[[2-(6-oxo-2,5- diazaspiro[3.5]nonane- 2-carbonyl)-2- azaspiro[3.3]heptan-6- yl[methyl]-2- (trifluoromethyl)benzoni- trile | A.6 and B.307 | 447.2 [M + H]⁺ |
| 271 | 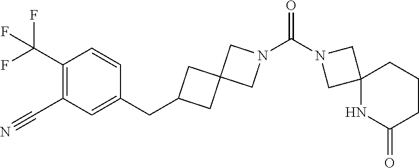 [SMILES: N#Cc1cc(CC2CC3(C2)CN(C(=O)N2CC4(CCCC(=O)N4)C2)C3)ccc1C(F)(F)F] | 5-[[2-(6-oxo-2,5- diazaspiro[3.5]nonane- 2-carbonyl)-2- azaspiro[3.3]heptan-6- yl]methyl]-2- (trifluoromethyl)benzoni- trile | A.6 and B.13 | 447.2 [M + H]⁺ |
| 272 | 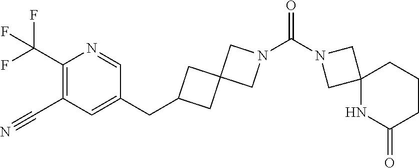 [SMILES: N#Cc1cc(CC2CC3(C2)CN(C(=O)N2CC4(CCCC(=O)N4)C2)C3)cnc1C(F)(F)F] | 5-[[2-(6-oxo-2,5- diazaspiro[3.5]nonane- 2-carbonyl)-2- azaspiro[3.3]heptan-6- yl]methyl]-2- (trifluoromethyl)pyridine- 3-carbonitrile | A.6 and P.11 | 448.2 [M + H]⁺ |
| 273 | 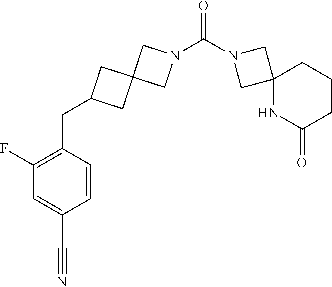 [SMILES: N#Cc1ccc(CC2CC3(C2)CN(C(=O)N2CC4(CCCC(=O)N4)C2)C3)c(F)c1] | 3-fluoro-4-[[2-(6-oxo- 2,5- diazaspiro[3.5]nonane- 2-carbonyl)-2- azaspiro[3.3]heptan-6- yl]methyl]benzonitrile | A.6 and B.322 | 397.2 [M + H]⁺ |
| 274 | 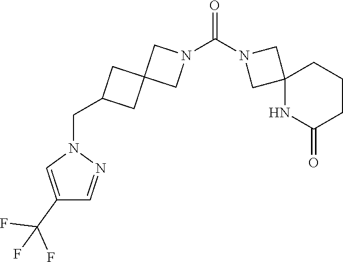 [SMILES: O=C1CCCC2(CN(C(=O)N3CC4(CC(Cn5cc(C(F)(F)F)cn5)C4)C3)C2)N1] | 2-[6-[[4- (trifluoromethyl)pyrazol- 1-yl]methyl]-2- azaspiro[3.3]heptane-2- carbonyl]-2,5- diazaspiro[3.5]nonan-6- one | A.6 and B.32 | 412.2 [M + H]⁺ |
| 275 | 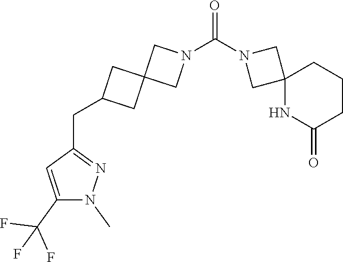 [SMILES: Cn1nc(CC2CC3(C2)CN(C(=O)N2CC4(CCCC(=O)N4)C2)C3)cc1C(F)(F)F] | 2-[6-[[1-methyl-5- (trifluoromethyl)pyrazol- 3-yl]methyl]-2- azaspiro[3.3]heptane-2- carbonyl]-2,5- diazaspiro[3.5]nonan-6- one | A.6 and B.309 | 426.2 [M + H]⁺ |
| 276 | 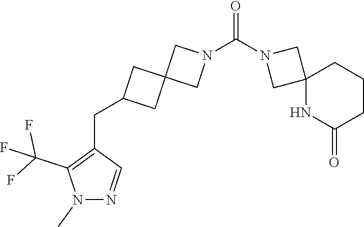 [SMILES: Cn1ncc(CC2CC3(C2)CN(C(=O)N2CC4(CCCC(=O)N4)C2)C3)c1C(F)(F)F] | 2-[6-[[1-methyl-5- (trifluoromethyl)pyrazol- 4-yl]methyl]-2- azaspiro[3.3]heptane-2- carbonyl]-2,5- diazaspiro[3.5[nonan-6- one | A.6 and B.308 | 426.2 [M + H]⁺ |
| 277 | 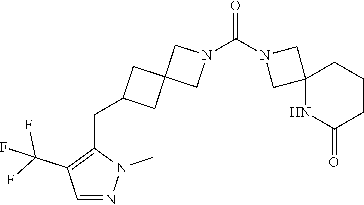 [SMILES: Cn1ncc(C(F)(F)F)c1CC1CC2(C1)CN(C(=O)N1CC3(CCCC(=O)N3)C1)C2] | 2-[6-[[2-methyl-4- (trifluoromethyl)pyrazol- 3-yl]methyl]-2- azaspiro[3.3]heptane-2- carbonyl]-2,5- diazaspiro[3.5]nonan-6- one | A.6 and B.310 | 426.2 [M + H]⁺ |
| 278 | 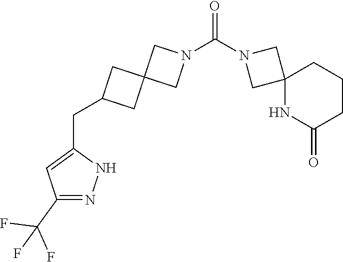 [SMILES: O=C1CCCC2(CN(C(=O)N3CC4(CC(Cc5cc(C(F)(F)F)n[nH]5)C4)C3)C2)N1] | 2-[6-[[3- (trifluoromethyl)-1H- pyrazol-5-y1]methyl]-2- azaspiro[3.3]heptane-2- carbonyl]-2,5- diazaspiro[3.5]nonan-6- one | A.6 and P.7 | 412.2 [M + H]⁺ |
| 279 | 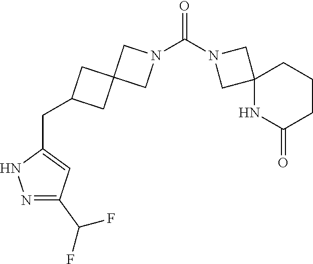 [SMILES: O=C1CCCC2(CN(C(=O)N3CC4(CC(Cc5cc(C(F)F)n[nH]5)C4)C3)C2)N1] | 2-[6-[[3- (difluoromethyl)-1H- pyrazol-5-yl]methyl]-2- azaspiro[3.3]heptane-2- carbonyl]-2,5- diazaspiro[3.5]nonan-6- one | A.6 and P.62 | 394.2 [M + H]⁺ |
| 280 | 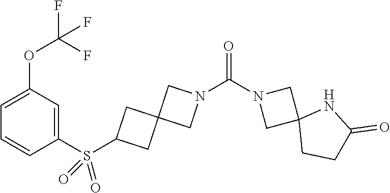 [SMILES: O=C1CCC2(CN(C(=O)N3CC4(CC(S(=O)(=O)c5cccc(OC(F)(F)F)c5)C4)C3)C2)N1] | 2-[6-[3- (trifluoromethoxy)phenyl] sulfonyl-2- azaspiro[3.3]heptane-2- carbonyl]2,5- diazaspiro[3.4]octan-6- one | A.1 and B.336 | 474.2 [M + H]⁺ |
| 281 | 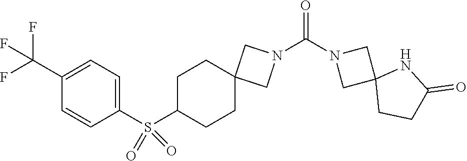 [SMILES: O=C1CCC2(CN(C(=O)N3CC4(CCC(S(=O)(=O)c5ccc(C(F)(F)F)cc5)CC4)C3)C2)N1] | 2-[7-[4- (trifluoromethyl)phenyl] sulfonyl-2- azaspiro[3.5]nonane-2- carbonyl]-2,5- diazaspiro[3.4]octan-6- one | A.1 and B.337 | 486.2 [M + H]⁺ |
| 282 | 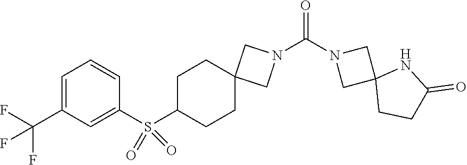 [SMILES: O=C1CCC2(CN(C(=O)N3CC4(CCC(S(=O)(=O)c5cccc(C(F)(F)F)c5)CC4)C3)C2)N1] | 2-[7-[3- (trifluoromethyl)phenyl] sulfonyl-2- azaspiro[3.5]nonane-2- carbonyl]-2,5- diazaspiro[3.4]octan-6- one | A.1 and B.338 | 486.2 [M + H]⁺ |
| 283 | 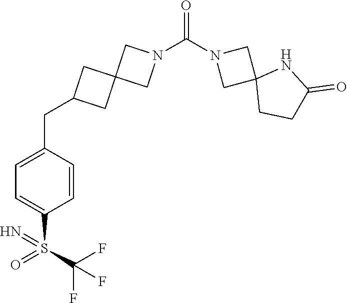 [SMILES: N=[S@](=O)(c1ccc(CC2CC3(C2)CN(C(=O)N2CC4(CCC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-2,5- diazaspiro[3.4]octan-6- one | A.1 and B.314 | 471.2 [M + H]⁺ |
| 284 | 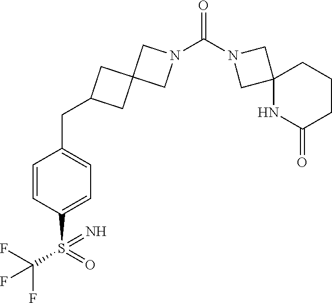 [SMILES: N=[S@@](=O)(c1ccc(CC2CC3(C2)CN(C(=O)N2CC4(CCCC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-2,5- diazaspiro[3.5]nonan-6- one | A.6 and B.314 | 485.2 [M + H]⁺ |
| 285 | 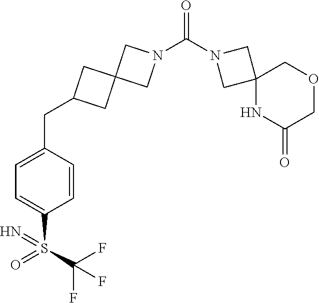 [SMILES: N=[S@](=O)(c1ccc(CC2CC3(C2)CN(C(=O)N2CC4(COCC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-8-oxa-2,5- diazaspiro[3.5]nonan-6- one | A.5 and B.314 | 487.2 [M + H]⁺ |
| 286 | 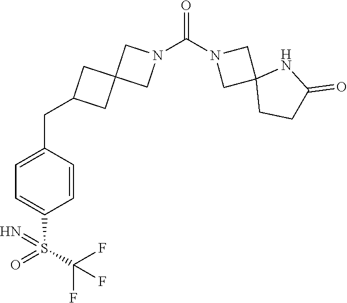 [SMILES: N=[S@@](=O)(c1ccc(CC2CC3(C2)CN(C(=O)N2CC4(CCC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-2,5- diazaspiro[3.4]octan-6- one | A.1 and B.313 | 471.2 [M + H]⁺ |
| 287 | 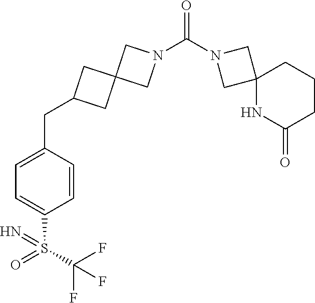 [SMILES: N=[S@@](=O)(c1ccc(CC2CC3(C2)CN(C(=O)N2CC4(CCCC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-2,5- diazaspiro[3.5]nonan-6- one | A.6 and B.313 | 485.2 [M + H]⁺ |
| 288 | 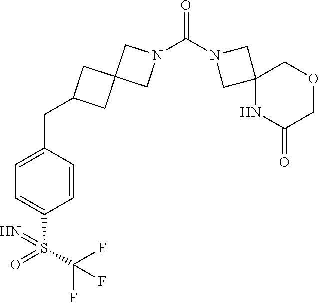 [SMILES: N=[S@@](=O)(c1ccc(CC2CC3(C2)CN(C(=O)N2CC4(COCC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-8-oxa-2,5- diazaspiro[3.5]nonan-6- one | A.5 and B.313 | 487.2 [M + H]⁺ |
| 289 | 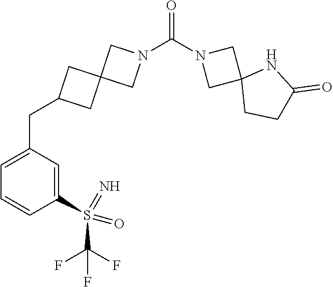 [SMILES: N=[S@@](=O)(c1cccc(CC2CC3(C2)CN(C(=O)N2CC4(CCC(=O)N4)C2)C3)c1)C(F)(F)F] | 2-[6-[[3- (trifluoromethylsulfoni- midoyl)phenyl]methyl]- 2-azaspiro[3.3]heptane- 2-carbonyl]-2,5- diazaspiro[3.4]octan-6- one | A.1 and P.78 | 471.2 [M + H]⁺ |
| 290 | 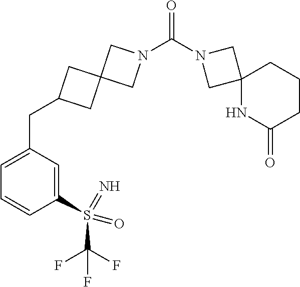 [SMILES: N=[S@@](=O)(c1cccc(CC2CC3(C2)CN(C(=O)N2CC4(CCCC(=O)N4)C2)C3)c1)C(F)(F)F] | 2-[6-[[3- (trifluoromethylsulfoni- midoyl)phenyl]methyl]- 2-azaspiro[3.3]heptane- 2-carbonyl]-2,5- diazaspiro[3.5]nonan-6- one | A.6 and P.78 | 485.2 [M + H]⁺ |
| 291 | 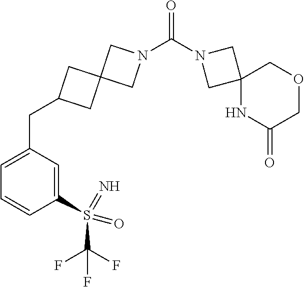 [SMILES: N=[S@@](=O)(c1cccc(CC2CC3(C2)CN(C(=O)N2CC4(COCC(=O)N4)C2)C3)c1)C(F)(F)F] | 2-[6-[[3- (trifluoromethylsulfoni- midoyl)phenyl]methyl]- 2-azaspiro[3.3]heptane- 2-carbonyl]-8-oxa-2,5- diazaspiro[3.5]nonan-6- one | A.5 and P.78 | 487.2 [M + H]⁺ |
| 292 | 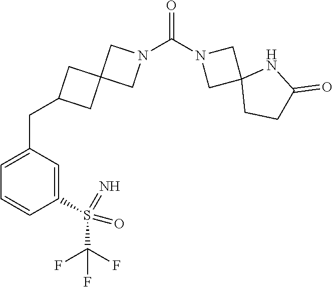 [SMILES: N=[S@](=O)(c1cccc(CC2CC3(C2)CN(C(=O)N2CC4(CCC(=O)N4)C2)C3)c1)C(F)(F)F] | 2-[6-[[3- (trifluoromethylsulfoni- midoyl)phenyl]methyl]- 2-azaspiro[3.3]heptane- 2-carbonyl]-2,5- diazaspiro[3.4]octan-6- one | A.1 and P.77 | 471.2 [M + H]⁺ |
| 293 | 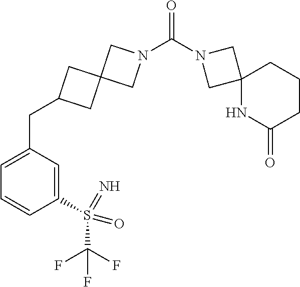 [SMILES: N=[S@](=O)(c1cccc(CC2CC3(C2)CN(C(=O)N2CC4(CCCC(=O)N4)C2)C3)c1)C(F)(F)F] | 2-[6-[[3- (trifluoromethylsulfoni- midoyl)phenyl]methyl]- 2-azaspiro[3.3]heptane- 2-carbonyl]-2,5- diazaspiro[3.5]nonan-6- one | A.6 and P.77 | 485.2 [M + H]⁺ |
| 294 | 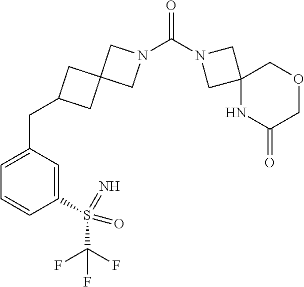 [SMILES: N=[S@](=O)(c1cccc(CC2CC3(C2)CN(C(=O)N2CC4(COCC(=O)N4)C2)C3)c1)C(F)(F)F] | 2-[6-[[3- (trifluoromethylsulfoni- midoyl)phenyl]methyl]- 2-azaspiro[3.3]heptane- 2-carbonyl]-8-oxa-2,5- diazaspiro[3.5]nonan-6- one | A.5 and P.77 | 487.2 [M + H]⁺ |
| 295 | 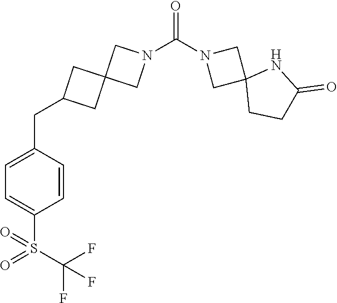 [SMILES: O=C1CCC2(CN(C(=O)N3CC4(CC(Cc5ccc(S(=O)(=O)C(F)(F)F)cc5)C4)C3)C2)N1] | 2-[6-[[4- (trifluoromethylsulfonyl) phenyl]methy]-2- azaspiro[3.3]heptane-2- carbonyl]-2,5- diazaspiro[3.4]octan-6- one | A.1 and P.74 | 472.2 [M + H]⁺ |
| 296 | 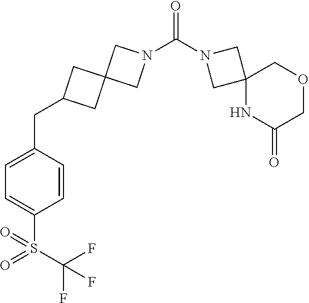 [SMILES: O=C1COCC2(CN(C(=O)N3CC4(CC(Cc5ccc(S(=O)(=O)C(F)(F)F)cc5)C4)C3)C2)N1] | 2-[6-[[4- (trifluoromethylsulfonyl) phenyl]methyl]-2- azaspiro[3.3]heptane-2- carbonyl]-8-oxa-2,5- diazaspiro[3.5]nonan-6- one | A.5 and P.74 | 488.2 [M + H]⁺ |
| 297 | 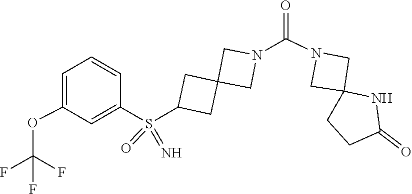 [SMILES: N=S(=O)(c1cccc(OC(F)(F)F)c1)C1CC2(C1)CN(C(=O)N1CC3(CCC(=O)N3)C1)C2] | 2-[6-[[3- (trifluoromethoxy)phenyl] sulfonimidoyl]-2- azaspiro[3.3]heptane-2- carbonyl]-2,5- diazaspiro[3.4]octan-6- one | A.1 and B.339 | 473.2 [M + H]⁺ |
| 298 | 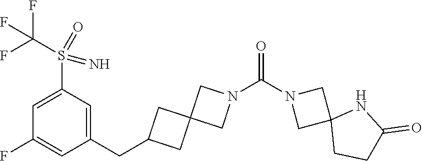 [SMILES: N=S(=O)(c1cc(F)cc(CC2CC3(C2)CN(C(=O)N2CC4(CCC(=O)N4)C2)C3)c1)C(F)(F)F] | 2-[6-[[3-fluoro-5- (trifluoromethylsulfoni- midoyl)phenyl]methyl]- 2-azaspiro[3.3]heptane- 2-carbonyl]-2,5- diazaspiro[3.4]octan-6- one | A.1 and P.67 | 489.2 [M + H]⁺ |
| 299 | 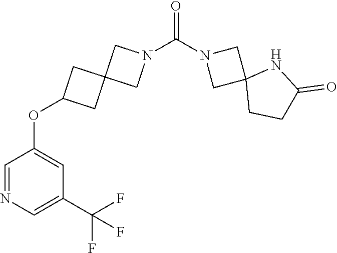 [SMILES: O=C1CCC2(CN(C(=O)N3CC4(CC(Oc5cncc(C(F)(F)F)c5)C4)C3)C2)N1] | 2-[6-[[5- (trifluoromethyl)-3- pyridyl]oxy]-2- azaspiro[3.3]heptane-2- carbonyl]-2,5- diazaspiro[3.4]octan-6- one | A.1 and B.332 | 411.2 [M + H]⁺ |
| 300 | 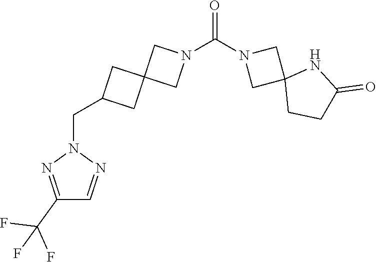 [SMILES: O=C1CCC2(CN(C(=O)N3CC4(CC(Cn5ncc(C(F)(F)F)n5)C4)C3)C2)N1] | 2-[6-[[4- (trifluoromethyl)triazol- 2-yl]methyl]-2- azaspiro[3.3]heptane-2- carbonyl]-2,5- diazaspiro[3.4]octan-6- one | A.1 and P.50 | 399.2 [M + H]⁺ |
| 301 | 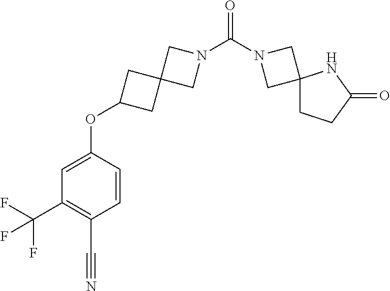 [SMILES: N#Cc1ccc(OC2CC3(C2)CN(C(=O)N2CC4(CCC(=O)N4)C2)C3)cc1C(F)(F)F] | 4-[[2-(6-oxo-2,5- diazaspiro[3.4]octane- 2-carbonyl)-2- azaspiro[3.3]heptan-6- yl]oxy]-2- (trifluoromethyl)benzoni- trile | A.1 and B.340 | 435.2 [M + H]⁺ |
| 302 | 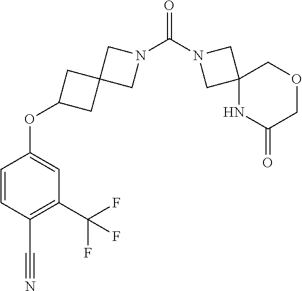 [SMILES: N#Cc1ccc(OC2CC3(C2)CN(C(=O)N2CC4(COCC(=O)N4)C2)C3)cc1C(F)(F)F] | 4-[[2-(6-oxo-8-oxa-2,5- diazaspiro[3.5]nonane- 2-carbonyl)-2- azaspiro[3.3]heptan-6- yl]oxy]-2- (trifluoromethyl)benzoni- trile | A.5 and B.340 | 451.2 [M + H]⁺ |
| 303 | 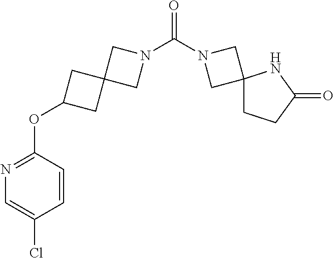 [SMILES: O=C1CCC2(CN(C(=O)N3CC4(CC(Oc5ccc(Cl)cn5)C4)C3)C2)N1] | 2-[6-[(5-chloro-2- pyridyl)oxy]-2- azaspiro[3.3]heptane-2- carbonyl]-2,5- diazaspiro[3.4]octan-6- one | A.1 and B.341 | 377.2 [M + H]⁺ |
| 304 | 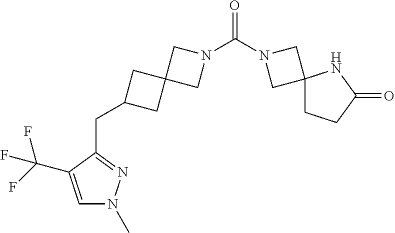 [SMILES: Cn1cc(C(F)(F)F)c(CC2CC3(C2)CN(C(=O)N2CC4(CCC(=O)N4)C2)C3)n1] | 2-[6-[[1-methyl-4- (trifluoromethyl)pyrazol- 3-yl]methyl]-2- azaspiro[3.3]heptane-2- carbonyl]-2,5- diazaspiro[3.4]octan-6- one | A.1 and P.34 | 412.2 [M + H]⁺ |
| 305 | 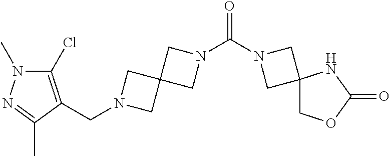 [SMILES: Cc1nn(C)c(Cl)c1CN1CC2(C1)CN(C(=O)N1CC3(COC(=O)N3)C1)C2] | 2-[6-[(5-chloro-1,3- dimethyl-pyrazol-4- yl)methyl]-2,6- diazaspiro[3.3]heptane- 2-carbonyl]-7-oxa-2,5- diazaspiro[3.4]octan-6- one | A.2 and B.375 | 395.1 [M + H]⁺ |
| 306 | 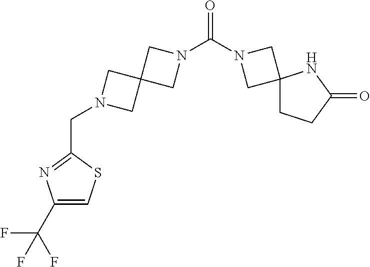 [SMILES: O=C1CCC2(CN(C(=O)N3CC4(CN(Cc5nc(C(F)(F)F)cs5)C4)C3)C2)N1] | 2-[6-[[4- (trifluoromethyl)thiazol- 2-yl]methyl]-2,6- diazaspiro[3.3]heptane- 2-carbonyl]-2,5- diazaspiro[3.4]octan-6- one | A.1 and B.363 | 416.2 [M + H]⁺ |
| 307 | 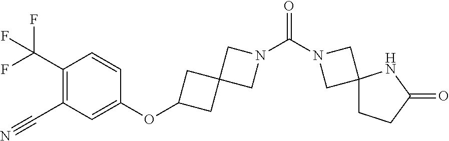 [SMILES: N#Cc1cc(OC2CC3(C2)CN(C(=O)N2CC4(CCC(=O)N4)C2)C3)ccc1C(F)(F)F] | 5-[[2-(6-oxo-2,5- diazaspiro[3.4]octane- 2-carbonyl)-2- azaspiro[3.3]heptan-6- yl]oxy]-2- (trifluoromethyl)benzoni- trile | A.1 and B.334 | 435.2 [M + H]⁺ |
| 308 | 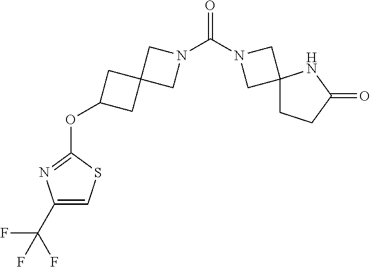 [SMILES: O=C1CCC2(CN(C(=O)N3CC4(CC(Oc5nc(C(F)(F)F)cs5)C4)C3)C2)N1] | 2-[6-[4- (trifluoromethyl)thiazol- 2-yl]oxy-2- azaspiro[3.3]heptane-2- carbonyl]-2,5- diazaspiro[3.4]octan-6- one | A.1 and B.333 | 417.2 [M + H]⁺ |
| 309 | 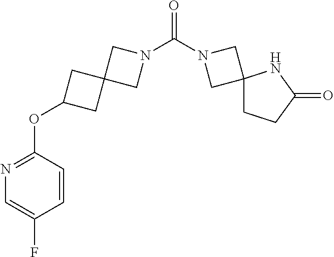 [SMILES: O=C1CCC2(CN(C(=O)N3CC4(CC(Oc5ccc(F)cn5)C4)C3)C2)N1] | 2-[6-[(5-fluoro-2- pyridyl)oxy]-2- azaspiro[3.3]heptane-2- carbonyl]-2,5- diazaspiro[3.4]octan-6- one | A.1 and B.342 | 361.2 [M + H]⁺ |
| 310 | 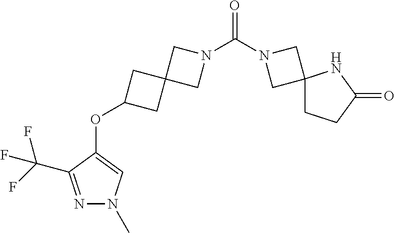 [SMILES: Cn1cc(OC2CC3(C2)CN(C(=O)N2CC4(CCC(=O)N4)C2)C3)c(C(F)(F)F)n1] | 2-[6-[1-methyl-3- (trifluoromethyl)pyrazol- 4-yl]oxy-2- azaspiro[3.3]heptane-2- carbonyl]-2,5- diazaspiro[3.4]octan-6- one | A.1 and B.376 | 414.2 [M + H]⁺ |
| 311 | 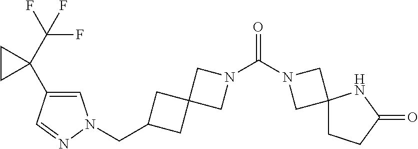 [SMILES: O=C1CCC2(CN(C(=O)N3CC4(CC(Cn5cc(C6(C(F)(F)F)CC6)cn5)C4)C3)C2)N1] | 2-[6-[[4-[1- (trifluoromethyl)cyclopro- pyl]pyrazol-1- yl]methyl]-2- azaspiro[3.3]heptane-2- carbonyl]-2,5- diazaspiro[3.4]octan-6- one | A.1 and B.377 | 438.3 [M + H]⁺ |
| 312 | 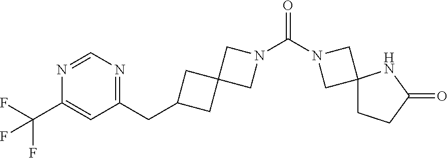 [SMILES: O=C1CCC2(CN(C(=O)N3CC4(CC(Cc5cc(C(F)(F)F)ncn5)C4)C3)C2)N1] | 2-[6-[[6- (trifluoromethyl)pyrimi- din-4-yl]methyl]-2- azaspiro[3.3]heptane-2- carbonyl]-2,5- diazaspiro[3.4]octan-6- one | A.1 and P.41 | 410.2 [M + H]⁺ |
| 313 | 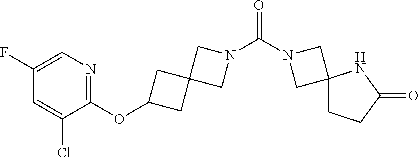 [SMILES: O=C1CCC2(CN(C(=O)N3CC4(CC(Oc5ncc(F)cc5Cl)C4)C3)C2)N1] | 2-[6-[(3-chloro-5- fluoro-2-pyridyl)oxy]- 2-azaspiro[3.3]heptane- 2-carbonyl]-2,5- diazaspiro[3.4]octan-6- one | A.1 and B.343 | 395.2 [M + H]⁺ |
| 314 | 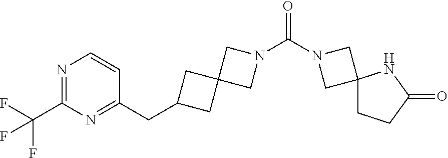 [SMILES: O=C1CCC2(CN(C(=O)N3CC4(CC(Cc5ccnc(C(F)(F)F)n5)C4)C3)C2)N1] | 2-[6-[[2- (trifluoromethyl)pyrimi- din-4-yl]methyl]-2- azaspiro[3.3]heptane-2- carbonyl]-2,5- diazaspiro[3.4]octan-6- one | A.1 and P.42 | 410.2 [M + H]⁺ |
| 315 | 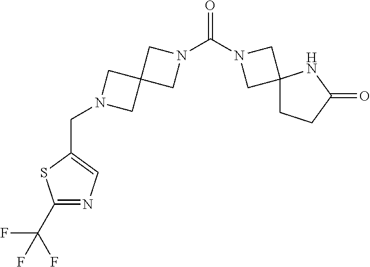 [SMILES: O=C1CCC2(CN(C(=O)N3CC4(CN(Cc5cnc(C(F)(F)F)s5)C4)C3)C2)N1] | 2-[6-[[2- (trifluoromethyl)thiazol- 5-yl]methyl]-2,6- diazaspiro[3.3]heptane- 2-carbonyl]-2,5- diazaspiro[3.4]octan-6- one | A.1 and B.347 | 416.2 [M + H]⁺ |
| 316 | 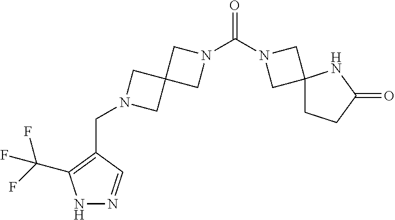 [SMILES: O=C1CCC2(CN(C(=O)N3CC4(CN(Cc5cn[nH]c5C(F)(F)F)C4)C3)C2)N1] | 2-[6-[[5- (trifluoromethyl)-1H- pyrazol-4-yl]methyl]- 2,6- diazaspiro[3.3]heptane- 2-carbonyl]-2,5- diazaspiro[3.4]octan-6- one | A.1 and B.362 | 399.2 [M + H]⁺ |
| 317 | 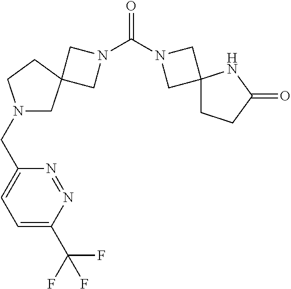 [SMILES: O=C1CCC2(CN(C(=O)N3CC4(CCN(Cc5ccc(C(F)(F)F)nn5)C4)C3)C2)N1] | 2-[7-[[6- (trifluoromethyl)pyrida- zin-3-yl]methyl]-2,7- diazaspiro[3.4]octane- 2-carbonyl]-2,5- diazaspiro[3.4]octan-6- one | A.1 and B.123 | 425.4 [M + H]⁺ |
| 318 | 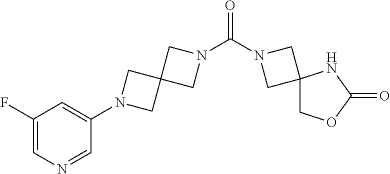 [SMILES: O=C1NC2(CO1)CN(C(=O)N1CC3(C1)CN(c1cncc(F)c1)C3)C2] | 2-[6-(5-fluoro-3- pyridyl)-2,6- diazaspiro[3.4]heptane- 2-carbonyl]-7-oxa-2,5- diazaspiro[3.4]octan-6- one | A.2 and B.378 | 348.2 [M + H]⁺ |
| 319 | 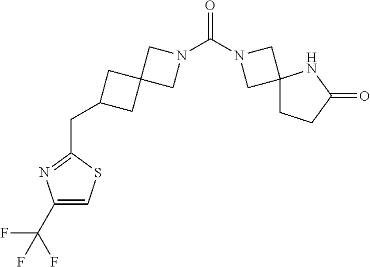 [SMILES: O=C1CCC2(CN(C(=O)N3CC4(CC(Cc5nc(C(F)(F)F)cs5)C4)C3)C2)N1] | 2-[6-[[4- (trifluoromethyl)thiazol- 2-yl]methyl]-2- azaspiro[3.3]heptane-2- carbonyl]-2,5- diazaspiro[3.4]octan-6- one | A.1 and B.344 | 415.3 [M + H]⁺ |
| 320 | 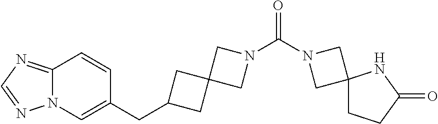 [SMILES: O=C1CCC2(CN(C(=O)N3CC4(CC(Cc5ccc6ncnn6c5)C4)C3)C2)N1] | 2-[6- ([1,2,4]triazolo[1,5- a]pyridin-6-ylmethyl)- 2-azaspiro[3.3]heptane- 2-carbonyl]-2,5- diazaspiro[3.4]octan-6- one | A.1 and B.345 | 381.3 [M + H]⁺ |
| 321 | 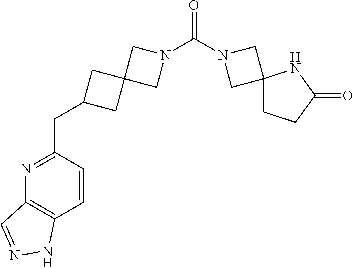 [SMILES: O=C1CCC2(CN(C(=O)N3CC4(CC(Cc5ccc6[nH]ncc6n5)C4)C3)C2)N1] | 2-[6-(1H-pyrazolo[4,3- b]pyridin-5-ylmethyl)- 2-azaspiro[3.3]heptane- 2-carbonyl]-2,5- diazaspiro[3.4]octan-6- one | A.1 and B.346 | 381.3 [M + H]⁺ |
| 322 | 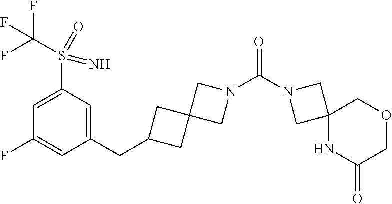 [SMILES: N=S(=O)(c1cc(F)cc(CC2CC3(C2)CN(C(=O)N2CC4(COCC(=O)N4)C2)C3)c1)C(F)(F)F] | 2-[6-[[3-fluoro-5- (trifluoromethylsulfoni- midoyl)phenyl]methyl]- 2-azaspiro[3.3]heptane- 2-carbonyl]-8-oxa-2,5- diazaspiro[3.5]nonan-6- one | A.5 and P.67 | 505.2 [M + H]⁺ |
| 323 | 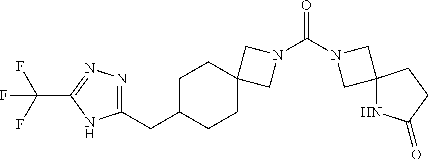 [SMILES: O=C1CCC2(CN(C(=O)N3CC4(CCC(Cc5nnc(C(F)(F)F)[nH]5)CC4)C3)C2)N1] | 2-[7-[[5- (trifluoromethyl)-4H- 1,2,4-triazol-3- yl]methyl]-2- azaspiro[3.5]nonane-2- carbonyl]-2,5- diazaspiro[3.4]octan-6- one | A.1 and B.353 | 427.4 [M + H]⁺ |
| 324 | 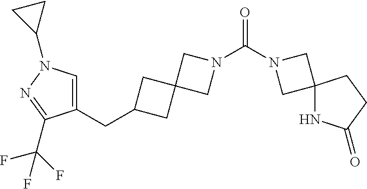 [SMILES: O=C1CCC2(CN(C(=O)N3CC4(CC(Cc5cn(C6CC6)nc5C(F)(F)F)C4)C3)C2)N1] | 2-[6-[[1-cyclopropyl-3- (trifluoromethyl)pyrazol- 4-yl]methyl]-2- azaspiro[3.4]heptane-2- carbonyl]-2,5- diazaspiro[3.4]octan-6- one | A.1 and B.379 | 438.4 [M + H]⁺ |
| 325 | 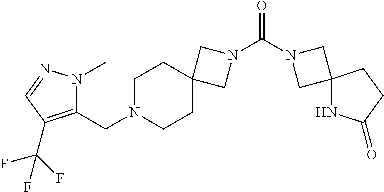 [SMILES: Cn1ncc(C(F)(F)F)c1CN1CCC2(CC1)CN(C(=O)N1CC3(CCC(=O)N3)C1)C2] | 2-[7-[[2-methyl-4- (trifluoromethyl)pyrazol- 3-yl]methyl]-2,7- diazaspiro[3.5]nonane- 2-carbonyl]-2,5- diazaspiro[3.4]octan-6- one | A.1 and B.380 | 441.4 [M + H]⁺ |
| 326 | 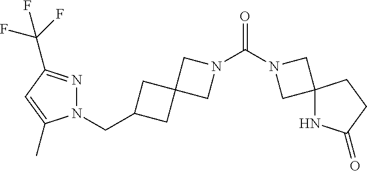 [SMILES: Cc1cc(C(F)(F)F)nn1CC1CC2(C1)CN(C(=O)N1CC3(CCC(=O)N3)C1)C2] | 2-[6-[[5-methyl-3- (trifluoromethyl)pyrazol- 1-yl]methyl]-2- azaspiro[3.3]heptane-2- carbonyl]-2,5- diazaspiro[3.4]octan-6- one | A.1 and B.381 | 412.3 [M + H]⁺ |
| 327 | 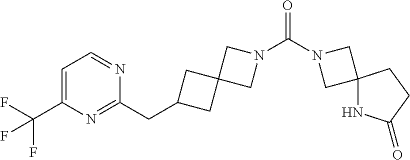 [SMILES: O=C1CCC2(CN(C(=O)N3CC4(CC(Cc5nccc(C(F)(F)F)n5)C4)C3)C2)N1] | 2-[6-[[4- (trifluoromethyl)pyrimi- din-2-yl]methyl]-2- azaspiro[3.3]heptane-2- carbonyl]-2,5- diazaspiro[3.4]octan-6- one | A.1 and B.355 | 410.3 [M + H]⁺ |
| 328 | 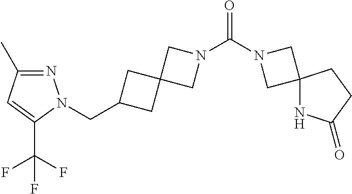 [SMILES: Cc1cc(C(F)(F)F)n(CC2CC3(C2)CN(C(=O)N2CC4(CCC(=O)N4)C2)C3)n1] | 2-[6-[[3-methyl-5- (trifluoromethyl)pyrazol- 1-yl]methyl]-2- azaspiro[3.3]heptane-2- carbonyl]-2,5- diazaspiro[3.4]octan-6- one | A.1 and B.382 | 412.3 [M + H]⁺ |
| 329 | 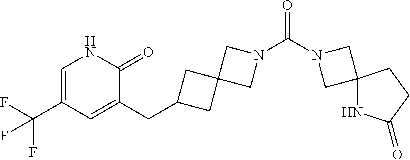 [SMILES: O=C1CCC2(CN(C(=O)N3CC4(CC(Cc5cc(C(F)(F)F)c[nH]c5=O)C4)C3)C2)N1] | 2-[6-[[2-keto-5- (trifluoromethyl)-1H- pyridin-3-yl]methyl]-2- azaspiro[3.3]heptane-2- carbonyl]-2,5- diazaspiro[3.4]octan-6- one | A.1 and B.383 | 425.3 [M + H]⁺ |
| 330 | 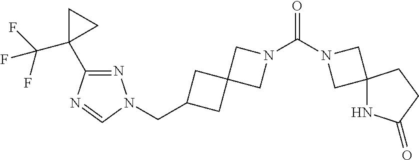 [SMILES: O=C1CCC2(CN(C(=O)N3CC4(CC(Cn5cnc(C6(C(F)(F)F)CC6)n5)C4)C3)C2)N1] | 2-[6-[[3-[1- (trifluoromethyl)cyclopro- pyl]-1,2,4-triazol-1- yl]methyl]-2- azaspiro[3.3]heptane-2- carbonyl]-2,5- diazaspiro[3.4]octan-6- one | A.1 and B.384 | 439.3 [M + H]⁺ |
| 331 | 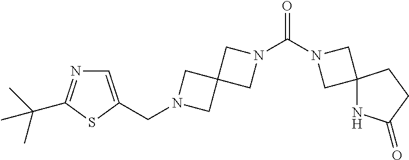 [SMILES: CC(C)(C)c1ncc(CN2CC3(C2)CN(C(=O)N2CC4(CCC(=O)N4)C2)C3)s1] | 2-[6-[(2-tert- butylthiazol-5- yl)methyl]-2,6- diazaspiro[3.3]heptane- 2-carbonyl]-2,5- diazaspiro[3.4]octan-6- one | A.1 and B.365 | 404.4 [M + H]⁺ |
| 332 | 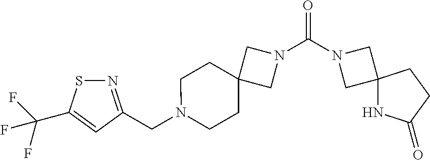 [SMILES: O=C1CCC2(CN(C(=O)N3CC4(CCN(Cc5cc(C(F)(F)F)sn5)CC4)C3)C2)N1] | 2-[7-[[5- (trifluoromethyl)isothia- zol-3-yl]methyl]-2,7- diazaspiro[3.5]nonane- 2-carbonyl]-2,5- diazaspiro[3.4]octan-6- one | A.1 and B.361 | 444.3 [M + H]⁺ |
| 333 | 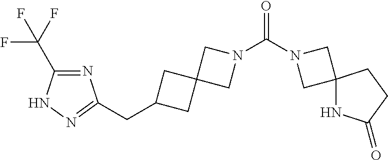 [SMILES: O=C1CCC2(CN(C(=O)N3CC4(CC(Cc5n[nH]c(C(F)(F)F)n5)C4)C3)C2)N1] | 2-[6-[[5- (trifluoromethyl)-1H- 1,2,4-triazol-3- yl]methyl]-2- azaspiro[3.3]heptane-2- carbonyl]-2,5- diazaspiro[3.4]octan-6- one | A.1 and B.318 | 399.3 [M + H]⁺ |
| 334 | 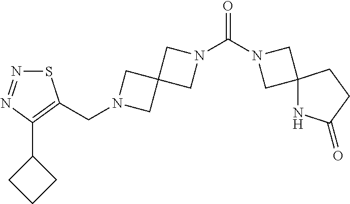 [SMILES: O=C1CCC2(CN(C(=O)N3CC4(CN(Cc5snnc5C5CCC5)C4)C3)C2)N1] | 2-[6-[(4- cyclobutylthiadiazol-5- yl)methyl]-2,6- diazaspiro[3.3]heptane- 2-carbonyl]-2,5- diazaspiro[3.4]octan-6- one | A.1 and B.385 | 403.3 [M + H]⁺ |
| 335 | 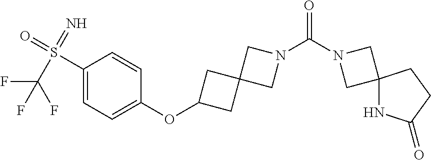 [SMILES: N=S(=O)(c1ccc(OC2CC3(C2)CN(C(=O)N2CC4(CCC(=O)N4)C2)C3)cc1)C(F)(F)F] | 2-[6-[4- (trifluoromethyl)sulfoni- midoyl)phenoxy]-2- azaspiro[3.3]heptane-2- carbonyl]-2,5- diazaspiro[3.4]octan-6- one | A.1 and B.386 | 473.3 [M + H]⁺ |
| 336 | 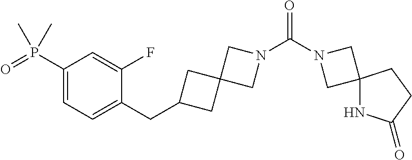 [SMILES: CP(C)(=O)c1ccc(CC2CC3(C2)CN(C(=O)N2CC4(CCC(=O)N4)C2)C3)c(F)c1] | 2-[6-(4- dimethylphosphoryl-2- fluoro-benzyl)-2- azaspiro[3.3]heptane-2- carbonyl]-2,5- diazaspiro[3.4]octan-6- one | A.1 and B.357 | 434.3 [M + H]⁺ |
| 337 | 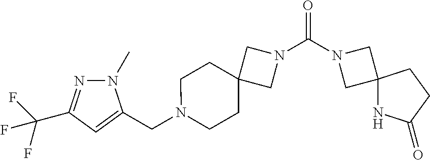 [SMILES: Cn1nc(C(F)(F)F)cc1CN1CCC2(CC1)CN(C(=O)N1CC3(CCC(=O)N3)C1)C2] | 2-[7-[[2-methyl-5- (trifluoromethyl)pyrazol- 3-yl]methyl]-2,7- diazaspiro[3.5]nonane- 2-carbonyl]-2,5- diazaspiro[3.4]octan-6- one | A.1 and B.350 | 441.4 [M + H]⁺ |
| 338 | 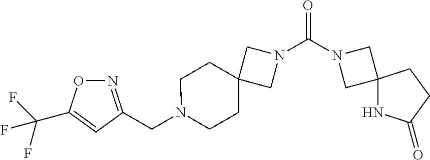 [SMILES: O=C1CCC2(CN(C(=O)N3CC4(CCN(Cc5cc(C(F)(F)F)on5)CC4)C3)C2)N1] | 2-[7-[[5- (trifluoromethyl)isoxazol- 3-yl]methyl]-2,7- diazaspiro[3.5]nonane- 2-carbonyl]-2,5- diazaspiro[3.4]octan-6- one | A.1 and B.387 | 428.3 [M + H]⁺ |
| 339 | 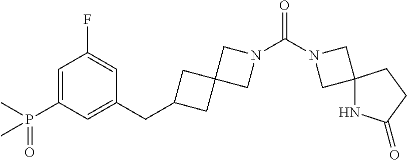 [SMILES: CP(C)(=O)c1cc(F)cc(CC2CC3(C2)CN(C(=O)N2CC4(CCC(=O)N4)C2)C3)c1] | 2-[6-(3- dimethylphosphoryl-5- fluoro-benzyl)-2- azaspiro[3.3]heptane-2- carbonyl]-2,5- diazaspiro[3.4]octan-6- one | A.1 and B.356 | 434.3 [M + H]⁺ |
| 340 | 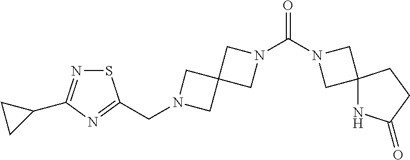 [SMILES: O=C1CCC2(CN(C(=O)N3CC4(CN(Cc5nc(C6CC6)ns5)C4)C3)C2)N1] | 2-[6-[(3-cyclopropyl- 1,2,4-thiadiazol-5- yl)methyl]-2,6- diazaspiro[3.4]heptane- 2-carbonyl]-2,5- diazaspiro[3.4]octan-6- one | A.1 and B.360 | 389.3 [M + H]⁺ |
| 341 | 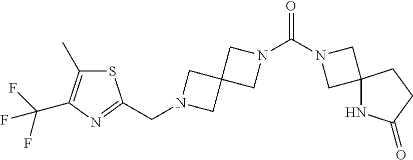 [SMILES: Cc1sc(CN2CC3(C2)CN(C(=O)N2CC4(CCC(=O)N4)C2)C3)nc1C(F)(F)F] | 2-[6-[[5-methyl-4- (trifluoromethyl)thiazol- 2-yl]methyl]-2,6- diazaspiro[3.3]heptane- 2-carbonyl]-2,5- diazaspiro[3.4]octan-6- one | A.1 and B.364 | 430.3 [M + H]⁺ |
| 342 | 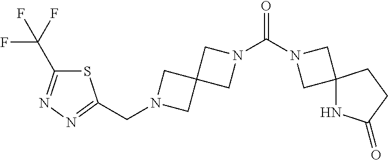 [SMILES: O=C1CCC2(CN(C(=O)N3CC4(CN(Cc5nnc(C(F)(F)F)s5)C4)C3)C2)N1] | 2-[6-[[5- (trifluoromethyl)-1,3,4- thiadiazol-2-yl]methyl]- 2,6- diazaspiro[3.3]heptane- 2-carbonyl]-2,5- diazaspiro[3.4]octan-6- one | A.1 and B.388 | 417.3 [M + H]⁺ |
| 343 | 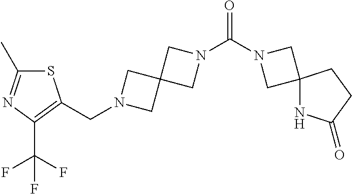 [SMILES: Cc1nc(C(F)(F)F)c(CN2CC3(C2)CN(C(=O)N2CC4(CCC(=O)N4)C2)C3)s1] | 2-[6-[[2-methyl-4- (trifluoromethyl)thiazol- 5-yl]methyl]-2,6- diazaspiro[3.4]heptane- 2-carbonyl]-2,5- diazaspiro[3.4]octan-6- one | A.1 and B.389 | 430.3 [M + H]⁺ |
| 344 | 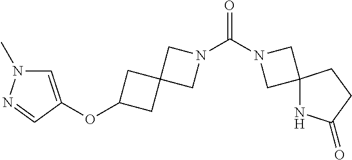 [SMILES: Cn1cc(OC2CC3(C2)CN(C(=O)N2CC4(CCC(=O)N4)C2)C3)cn1] | 2-[6-(1-methylpyrazol- 4-yl)oxy-2- azaspiro[3.3]heptane-2- carbonyl]-2,5- diazaspiro[3.4]octan-6- one | A.1 and B.390 | 346.2 [M + H]⁺ |
| 345 | 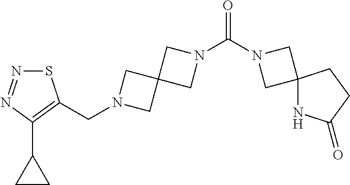 [SMILES: O=C1CCC2(CN(C(=O)N3CC4(CN(Cc5snnc5C5CC5)C4)C3)C2)N1] | 2-[6-[(4- cyclopropylthiadiazol- 5-yl)methyl]-2,6- diazaspiro[3.3]heptane- 2-carbonyl]-2,5- diazaspiro[3.4]octan-6- one | A.1 and B.391 | 389.3 [M + H]⁺ |
| 346 | 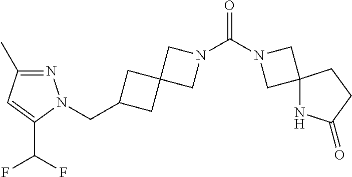 [SMILES: Cc1cc(C(F)F)n(CC2CC3(C2)CN(C(=O)N2CC4(CCC(=O)N4)C2)C3)n1] | 2-[6-[[5- (difluoromethyl)-3- methyl-pyrazol-1- yl]methyl]-2- azaspiro[3.3]heptane-2- carbonyl]-2,5- diazaspiro[3.4]octan-6- one | A.1 and B.392 | 394.4 [M + H]⁺ |
| 347 | 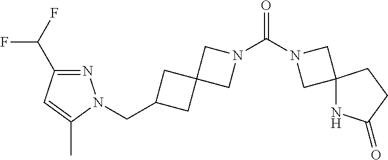 [SMILES: Cc1cc(C(F)F)nn1CC1CC2(C1)CN(C(=O)N1CC3(CCC(=O)N3)C1)C2] | 2-[6-[[3- (difluoromethyl)-5- methyl-pyrazol-1- yl]methyl]-2- azaspiro[3.3]heptane-2- carbonyl]-2,5- diazaspiro[3.4]octan-6- one | A.1 and B.393 | 394.3 [M + H]⁺ |
| 348 | 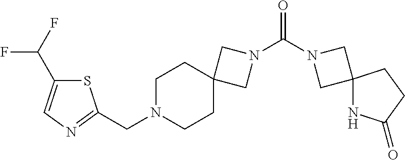 [SMILES: O=C1CCC2(CN(C(=O)N3CC4(CCN(Cc5ncc(C(F)F)s5)CC4)C3)C2)N1] | 2-[7-[[5- (difluoromethyl)thiazol- 2-yl]methyl]-2,7- diazaspiro[3.5]nonane- 2-carbonyl]-2,5- diazaspiro[3.4]octan-6- one | A.1 and B.352 | 426.4 [M + H]⁺ |
| 349 | 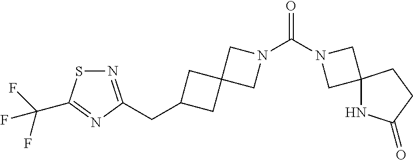 [SMILES: O=C1CCC2(CN(C(=O)N3CC4(CC(Cc5nsc(C(F)(F)F)n5)C4)C3)C2)N1] | 2-[6-[[5- (trifluoromethyl)-1,2,4- thiadiazol-3-yl]methyl]- 2-azaspiro[3.3]heptane- 2-carbonyl]-2,5- diazaspiro[3.4]octan-6- one | A.1 and B.348 | 416.3 [M + H]⁺ |
| 350 | 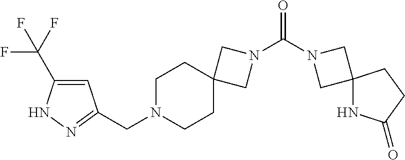 [SMILES: O=C1CCC2(CN(C(=O)N3CC4(CCN(Cc5cc(C(F)(F)F)[nH]n5)CC4)C3)C2)N1] | 2-[7-[[5- (trifluoromethyl)-1H- pyrazol-3-yl]methyl]- 2,7- diazaspiro[3.5]nonane- 2-carbonyl]-2,5- diazaspiro[3.4]octan-6- one | A.1 and B.351 | 427.4 [M + H]⁺ |
| 351 | 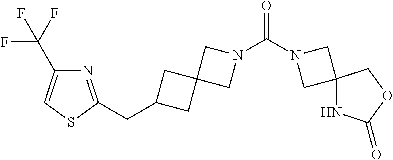 [SMILES: O=C1NC2(CO1)CN(C(=O)N1CC3(CC(Cc4nc(C(F)(F)F)cs4)C3)C1)C2] | 2-[6-[[4- (trifluoromethyl)thiazol- 2-yl]methyl]-2- azaspiro[3.3]heptane-2- carbonyl]-7-oxa-2,5- diazaspiro[3.4]octan-6- one | A.2 and B.344 | 417.3 [M + H]⁺ |
| 352 | 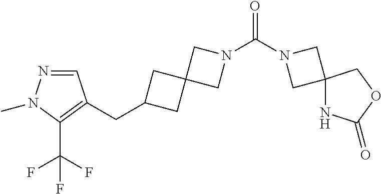 [SMILES: Cn1ncc(CC2CC3(C2)CN(C(=O)N2CC4(COC(=O)N4)C2)C3)c1C(F)(F)F] | 2-[6-[[1-methyl-5- (trifluoromethyl)pyrazol- 4-yl]methyl]-2- azaspiro[3.3]heptane-2- carbonyl]-7-oxa-2,5- diazaspiro[3.4]octan-6- one | A.2 and B.308 | 414.3 [M + H]⁺ |
| 353 | 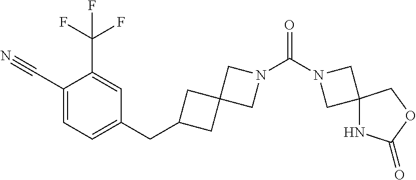 [SMILES: N#Cc1ccc(CC2CC3(C2)CN(C(=O)N2CC4(COC(=O)N4)C2)C3)cc1C(F)(F)F] | 4-[[2-(6-keto-7-oxa- 2,5- diazaspiro[3.4]octane- 2-carbonyl)-2- azaspiro[3.3]heptan-6- yl[methyl]-2- (trifluoromethyl)benzoni- trile | A.2 and B.307 | 435.3 [M + H]⁺ |
| 354 | 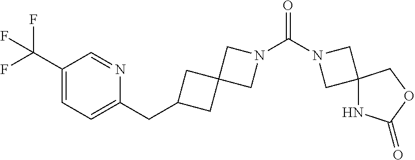 [SMILES: O=C1NC2(CO1)CN(C(=O)N1CC3(CC(Cc4ccc(C(F)(F)F)cn4)C3)C1)C2] | 2-[6-[[5- (trifluoromethyl)-2- pyridyl]methyl]-2- azaspiro[3.3]heptane-2- carbonyl]-7-oxa-2,5- diazaspiro[3.4]octan-6- one | A.2 and B.69 | 411.4 [M + H]⁺ |
| 355 | 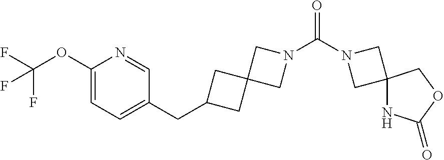 [SMILES: O=C1NC2(CO1)CN(C(=O)N1CC3(CC(Cc4ccc(OC(F)(F)F)nc4)C3)C1)C2] | 2-[6-[[6- (trifluoromethoxy)-3- pyridyl]methyl]-2- azaspiro[3.3]heptane-2- carbonyl]-7-oxa-2,5- diazaspiro[3.4]octan-6- one | A.2 and B.302 | 427.3 [M + H]⁺ |
| 356 | 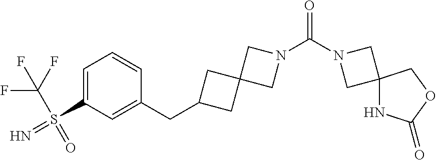 [SMILES: N=[S@](=O)(c1cccc(CC2CC3(C2)CN(C(=O)N2CC4(COC(=O)N4)C2)C3)c1)C(F)(F)F] | 2-[6-[3- (trifluoromethylsulfoni- midoyl)benzyl]-2- azaspiro[3.3]heptane-2- carbonyl]-7-oxa-2,5- diazaspiro[3.4]octan-6- one | A.2 and P.77 | 473.3 [M + H]⁺ |
| 357 | 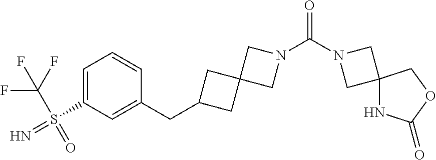 [SMILES: N=[S@@](=O)(c1cccc(CC2CC3(C2)CN(C(=O)N2CC4(COC(=O)N4)C2)C3)c1)C(F)(F)F] | 2-[6-[3- (trifluoromethylsulfoni- midoyl)benzyl]-2- azaspiro[3.3]heptane-2- carbonyl]-7-oxa-2,5- diazaspiro[3.4]octan-6- one | A.2 and P.78 | 473.3 [M + H]⁺ |
| 358 | 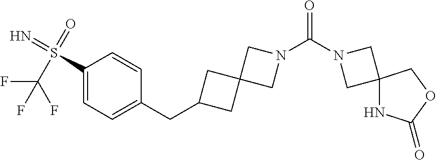 [SMILES: N=[S@@](=O)(c1ccc(CC2CC3(C2)CN(C(=O)N2CC4(COC(=O)N4)C2)C3)cc1)C(F)(F)F] | 2-[6-[4- (trifluoromethylsulfoni- midoyl)benzyl]-2- azaspiro[3.3]heptane-2- carbonyl]-7-oxa-2,5- diazaspiro[3.4]octan-6- one | A.2 and B.313 | 473.3 [M + H]⁺ |
| 359 | 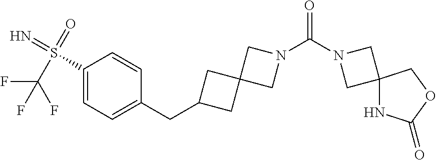 [SMILES: N=[S@](=O)(c1ccc(CC2CC3(C2)CN(C(=O)N2CC4(COC(=O)N4)C2)C3)cc1)C(F)(F)F] | 2-[6-[[4- (trifluoromethylsulfoni- midoyl)phenyl]methyl]- 2-azaspiro[3.3]heptane- 2-carbonyl]-7-oxa-2,5- diazaspiro[3.4[octan-6- one | A.2 and B.314 | 473.3 [M + H]⁺ |
| 360 | 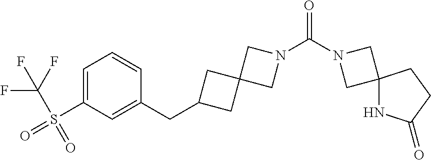 [SMILES: O=C1CCC2(CN(C(=O)N3CC4(CC(Cc5cccc(S(=O)(=O)C(F)(F)F)c5)C4)C3)C2)N1] | 2-[6-(3-triflylbenzyl)-2- azaspiro[3.3]heptane-2- carbonyl]-2,5- diazaspiro[3.4]octan-6- one | A.2 and B.16 | 472.3 [M + H]⁺ |
| 361 | 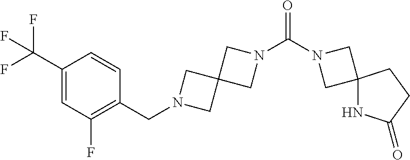 [SMILES: O=C1CCC2(CN(C(=O)N3CC4(CN(Cc5ccc(C(F)(F)F)cc5F)C4)C3)C2)N1] | 2-[6-[2-fluoro-4- (trifluoromethyl)benzyl]- 2,6- diazaspiro[3.3]heptane- 2-carbonyl]-2,5- diazaspiro[3.4]octan-6- one | A.1 and B.303 | 427.3 [M + H]⁺ |
| 362 | 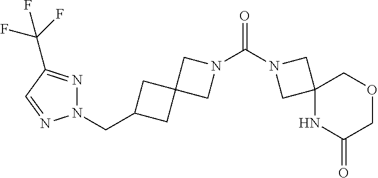 [SMILES: O=C1COCC2(CN(C(=O)N3CC4(CC(Cn5ncc(C(F)(F)F)n5)C4)C3)C2)N1] | 2-[6-[[4- (trifluoromethyl)triazol- 2-yl]methyl]-2- azaspiro[3.3]heptane-2- carbonyl]-8-oxa-2,5- diazaspiro[3.5]nonan-6- one | A.5 and P.50 | 415.3 [M + H]⁺ |
| 363 | 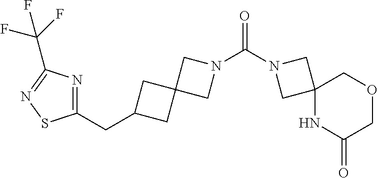 [SMILES: O=C1COCC2(CN(C(=O)N3CC4(CC(Cc5nc(C(F)(F)F)ns5)C4)C3)C2)N1] | 2-[6-[[3- (trifluoromethyl)-1,2,4- thiadiazol-5-yl]methyl]- 2-azaspiro[3.3]heptane- 2-carbonyl]-8-oxa-2,5- diazaspiro[3.5]nonan-6- one | A.5 and B.34 | 432.3 [M + H]⁺ |
| 364 | 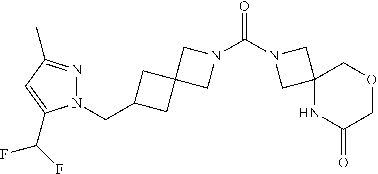 [SMILES: Cc1cc(C(F)F)n(CC2CC3(C2)CN(C(=O)N2CC4(COCC(=O)N4)C2)C3)n1] | 2-[6-[[5- (difluoromethyl)-3- methyl-pyrazol-1- yl]methyl]-2- azaspiro[3.3]heptane-2- carbonyl]-8-oxa-2,5- diazaspiro[3.5]nonan-6- one | A.5 and B.392 | 410.4 [M + H]⁺ |
| 365 | 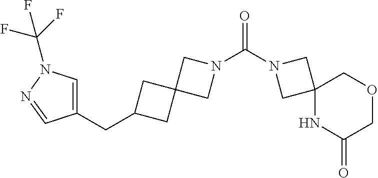 [SMILES: O=C1COCC2(CN(C(=O)N3CC4(CC(Cc5cnn(C(F)(F)F)c5)C4)C3)C2)N1] | 2-[6-[[1- (trifluoromethyl)pyrazol- 4-yl]methyl]-2- azaspiro[3.3]heptane-2- carbonyl]-8-oxa-2,5- diazaspiro[3.5]nonan-6- one | A.5 and B.335 | 414.3 [M + H]⁺ |
| 366 | 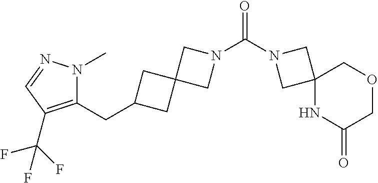 [SMILES: Cn1ncc(C(F)(F)F)c1CC1CC2(C1)CN(C(=O)N1CC3(COCC(=O)N3)C1)C2] | 2-[6-[[2-methyl-4- (trifluoromethyl)pyrazol- 3-yl]methyl]-2- azaspiro[3.3]heptane-2- carbonyl]-8-oxa-2,5- diazaspiro[3.5]nonan-6- one | A.5 and B.310 | 428.4 [M + H]⁺ |
| 367 | 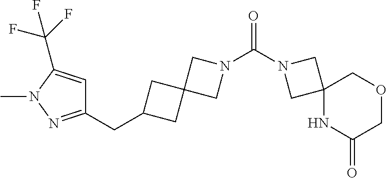 [SMILES: Cn1nc(CC2CC3(C2)CN(C(=O)N2CC4(COCC(=O)N4)C2)C3)cc1C(F)(F)F] | 2-[6-[[1-methyl-5- (trifluoromethyl)pyrazol- 3-yl]methyl]-2- azaspiro[3.3]heptane-2- carbonyl]-8-oxa-2,5- diazaspiro[3.5]nonan-6- one | A.5 and B.309 | 428.4 [M + H]⁺ |
| 368 | 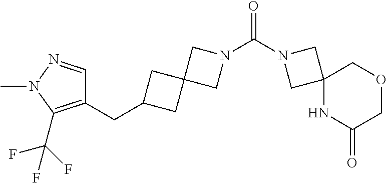 [SMILES: Cn1ncc(CC2CC3(C2)CN(C(=O)N2CC4(COCC(=O)N4)C2)C3)c1C(F)(F)F] | 2-[6-[[1-methyl-5- (trifluoromethyl)pyrazol- 4-yl]methyl]-2- azaspiro[3.3]heptane-2- carbonyl]-8-oxa-2,5- diazaspiro[3.5]nonan-6- one | A.5 and B.308 | 428.4 [M + H]⁺ |
| 369 | 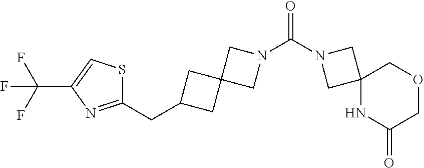 [SMILES: O=C1COCC2(CN(C(=O)N3CC4(CC(Cc5nc(C(F)(F)F)cs5)C4)C3)C2)N1] | 2-[6-[[4- (trifluoromethyl)thiazol- 2-yl]methyl]-2- azaspiro[3.3[heptane-2- carbonyl]-8-oxa-2,5- diazaspiro[3.5]nonan-6- one | A.5 and B.344 | 431.3 [M + H]⁺ |
| 370 | 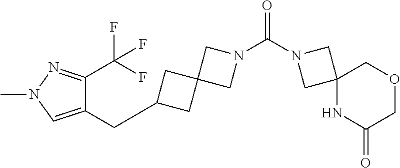 [SMILES: Cn1cc(CC2CC3(C2)CN(C(=O)N2CC4(COCC(=O)N4)C2)C3)c(C(F)(F)F)n1] | 2-[6-[[1-methyl-3- (trifluoromethyl)pyrazol- 4-yl]methyl]-2- azaspiro[3.3]heptane-2- carbonyl]-8-oxa-2,5- diazaspiro[3.5]nonan-6- one | A.5 and B.312 | 428.4 [M + H]⁺ |
| 371 | 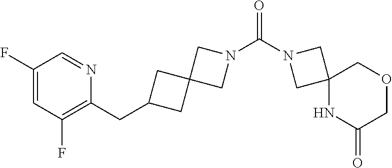 [SMILES: O=C1COCC2(CN(C(=O)N3CC4(CC(Cc5ncc(F)cc5F)C4)C3)C2)N1] | 2-[6-[(3,5-difluoro-2- pyridyl)methyl]-2- azaspiro[3.3]heptane-2- carbonyl]-8-oxa-2,5- diazaspiro[3.5]nonan-6- one | A.5 and B.72 | 393.3 [M + H]⁺ |
| 372 | 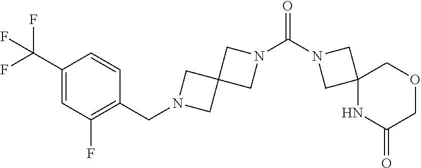 [SMILES: O=C1COCC2(CN(C(=O)N3CC4(CN(Cc5ccc(C(F)(F)F)cc5F)C4)C3)C2)N1] | 2-[6-[2-fluoro-4- (trifluoromethyl)benzyl]- 2,6- diazaspiro[3.3]heptane- 2-carbonyl]-8-oxa-2,5- diazaspiro[3.5]nonan-6- one | A.5 and B.303 | 443.4 [M + H]⁺ |
| 373 | 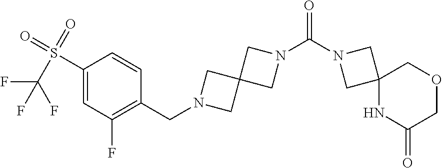 [SMILES: O=C1COCC2(CN(C(=O)N3CC4(CN(Cc5ccc(S(=O)(=O)C(F)(F)F)cc5F)C4)C3)C2)N1] | 2-[6-(2-fluoro-4-triflyl- benzyl)-2,6- diazaspiro[3.3]heptane- 2-carbonyl]-8-oxa-2,5- diazaspiro[3.5]nonan-6- one | A.5 and B.396 | 507.3 [M + H]⁺ |
| 374 | 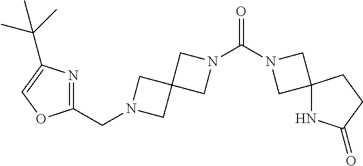 [SMILES: CC(C)(C)c1coc(CN2CC3(C2)CN(C(=O)N2CC4(CCC(=O)N4)C2)C3)n1] | 2-[6-[(4-tert- butyloxazol-2- yl)methyl]-2,6- diazaspiro[3.3]heptane- 2-carbonyl]-2,5- diazaspiro[3.4]octan-6- one | A.1 and B.394 | 388.4 [M + H]⁺ |
| 375 | 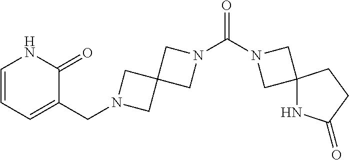 [SMILES: O=C1CCC2(CN(C(=O)N3CC4(CN(Cc5ccc[nH]c5=O)C4)C3)C2)N1] | 2-[6-[(2-keto-1H- pyridin-3-yl)methyl]- 2,6- diazaspiro[3.3]heptane- 2-carbonyl]-2,5- diazaspiro[3.4]octna-6- one | A.1 and B.395 | 358.3 [M + H]⁺ |

### Example 3

### 2-[6-[[6-(trifluoromethyl)-3-pyridyl]methyl]-2-azaspiro[3.3]heptane-2-carbonyl]-2,5-diazaspiro[3.4]octan-6-one

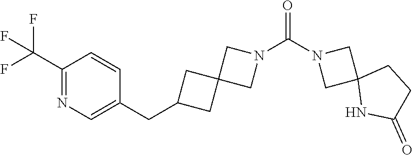 [SMILES: O=C1CCC2(CN(C(=O)N3CC4(CC(Cc5ccc(C(F)(F)F)nc5)C4)C3)C2)N1]

To a solution of 6-[[6-(trifluoromethyl)-3-pyridyl]methyl]-2-azaspiro[3.3]heptane; 4-methylbenzenesulfonic acid (B.3) (79.4 mg, 0.132 mmol) in N,N-dimethylformamide (0.781 mL) was added DIPEA (109 mg, 147 μL, 0.841 mmol). 4-nitrophenyl 6-oxo-2,5-diazaspiro[3.4]octane-2-carboxylate (35 mg, 0.120 mmol) was added to the reaction mixture which was then stirred at 50° C. for 18 h. The crude reaction mixture was directly submitted for reversed-phase HPLC purification to yield the title compound (28.7 mg). MS (ESI): m/z=409.3 [M+H]⁺

### Step a) 4-nitrophenyl 6-oxo-2,5-diazaspiro[3.4]octane-2-carboxylate

To a solution of 2,5-diazaspiro[3.4]octan-6-one hydrochloride (A.1) (205 mg, 1.26 mmol) and TEA (383 mg, 527 μL, 3.78 mmol) in DCM (3 ml) cooled to 0° C. with an ice bath, was added 4-nitrophenyl carbonochloridate (280 mg, 1.39 mmol) in three portions and the reaction mixture was stirred at room temperature for 3.5 h. The reaction was filtered, the solid was the title compound (126 mg, 34% yield). MS (ESI): m/z=292.2 [M+H]⁺

In analogy to Example 3, Examples in the following table were generated, using the respective building blocks A.X and B.X.

| | | | | MS, |
|---|---|---|---|---|
| | | | Building | ESI: |
| Ex. | Structure | Systematic Name | Blocks | m/z |
| 4 | 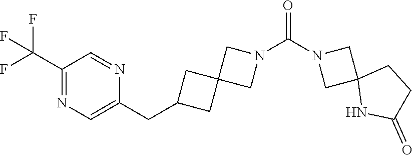 [SMILES: O=C1CCC2(CN(C(=O)N3CC4(CC(Cc5cnc(C(F)(F)F)cn5)C4)C3)C2)N1] | 2-[6-[[5- (trifluoromethyl)pyrazin- 2-yl]methyl]-2- azaspiro[3.3]heptane-2- carbonyl]-2,5- diazaspiro[3.4]octan-6- one | A.1 and B.4 | 410.3 [M + H]⁺ |
| 8 | 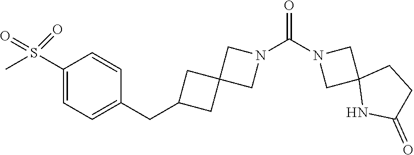 [SMILES: CS(=O)(=O)c1ccc(CC2CC3(C2)CN(C(=O)N2CC4(CCC(=O)N4)C2)C3)cc1] | 2-[6-(4-mesylbenzyl)-2- azaspiro[3.3]heptane-2- carbonyl]-2,5- diazaspiro[3.4]octan-6- one | A.1 and B.7 | 418.3 [M + H]⁺ |
| 9 | 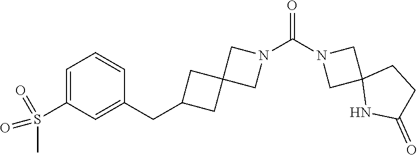 [SMILES: CS(=O)(=O)c1cccc(CC2CC3(C2)CN(C(=O)N2CC4(CCC(=O)N4)C2)C3)c1] | 2-[6-(3-mesylbenzyl)-2- azaspiro[3.3]heptane-2- carbonyl]-2,5- diazaspiro[3.4]octan-6- one | A.1 and B.8 | 418.2 [M + H]⁺ |
| 41 | 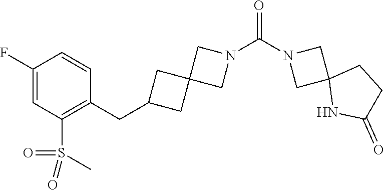 [SMILES: CS(=O)(=O)c1cc(F)ccc1CC1CC2(C1)CN(C(=O)N1CC3(CCC(=O)N3)C1)C2] | 2-[6-(4-fluoro-2-mesyl- benzyl)-2- azaspiro[3.3]heptane-2- carbonyl]-2,5- diazaspiro[3.4]octan-6- one | A.1 and B.12 | 436.2 [M + H]⁺ |
| 55 | 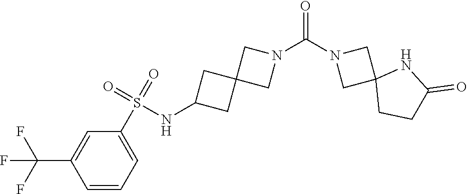 [SMILES: O=C1CCC2(CN(C(=O)N3CC4(CC(NS(=O)(=O)c5cccc(C(F)(F)F)c5)C4)C3)C2)N1] | N-[2-(6-keto-2,5- diazaspiro[3.4]octane- 2-carbonyl)-2- azaspiro[3.3]heptan-6- yl]-3- (trifluoromethyl)benzen esulfonamide | A.1 and B.30 | 473.2 [M + H]⁺ |
| 75 | 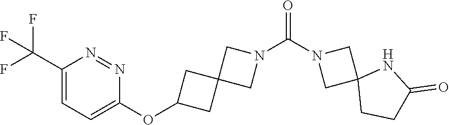 [SMILES: O=C1CCC2(CN(C(=O)N3CC4(CC(Oc5ccc(C(F)(F)F)nn5)C4)C3)C2)N1] | 2-[6-[6- (trifluoromethyl) pyridazin-3- yl]oxy-2- azaspiro[3.3]heptane-2- carbonyl]-2,5- diazaspiro[3.4]octan-6- one | A.1 and B.48 | 412.2 [M + H]⁺ |
| 90 | 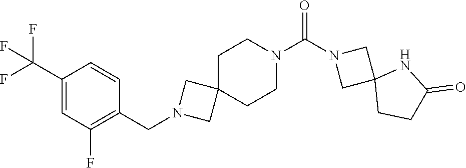 [SMILES: O=C1CCC2(CN(C(=O)N3CCC4(CC3)CN(Cc3ccc(C(F)(F)F)cc3F)C4)C2)N1] | 2-[2-[2-fluoro-4- (trifluoromethyl) benzyl]-2,7- diazaspiro[3.5]nonane- 7-carbonyl]-2,5- diazaspiro[3.4]octan-6- one | A.1 and B.56 | 455.3 [M + H]⁺ |
| 129 | 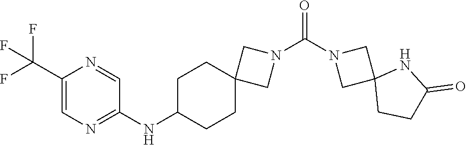 [SMILES: O=C1CCC2(CN(C(=O)N3CC4(CCC(Nc5cnc(C(F)(F)F)cn5)CC4)C3)C2)N1] | 2-[7-[[5- (trifluoromethyl) pyrazin- 2-yl]amino]-2- azaspiro[3.5]nonane-2- carbonyl]-2,5- diazaspiro[3.4]octan-6- one | A.1 and B.265 | 439.3 [M + H]⁺ |
| 198 | 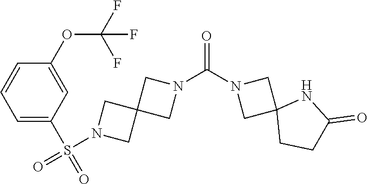 [SMILES: O=C1CCC2(CN(C(=O)N3CC4(C3)CN(S(=O)(=O)c3cccc(OC(F)(F)F)c3)C4)C2)N1] | 2-[2-[3- (trifluoromethoxy) phenyl]sulfonyl-2,6- diazaspiro[3.3]heptane- 6-carbonyl]-2,5- diazaspiro[3.4]octan-6- one | A.1 and B.55 | 475.2 [M + H]⁺ |
| 199 | 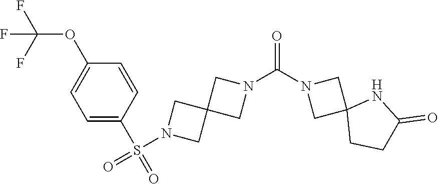 [SMILES: O=C1CCC2(CN(C(=O)N3CC4(C3)CN(S(=O)(=O)c3ccc(OC(F)(F)F)cc3)C4)C2)N1] | 2-[2-[4- (trifluoromethoxy) phenyl]sulfonyl-2,6- diazaspiro[3.3]heptane- 6-carbonyl]-2,5- diazaspiro[3.4]octan-6- one | A.1 and B.56 | 475.2 [M + H]⁺ |
| 194 | 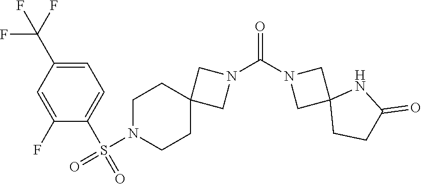 [SMILES: O=C1CCC2(CN(C(=O)N3CC4(CCN(S(=O)(=O)c5ccc(C(F)(F)F)cc5F)CC4)C3)C2)N1] | 2-[7-[2-fluoro-4- (trifluoromethyl)phenyl] sulfonyl-2,7- diazaspiro[3.5]nonane- 2-carbonyl]-2,5- diazaspiro[3.4]octan-6- one | A.1 and B.168 | 505.2 [M + H]⁺ |
| 200 | 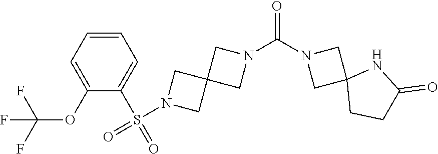 [SMILES: O=C1CCC2(CN(C(=O)N3CC4(C3)CN(S(=O)(=O)c3ccccc3OC(F)(F)F)C4)C2)N1] | 2-[2-[2- (trifluoromethoxy) phenyl]sulfonyl-2,6- diazaspiro[3.3]heptane- 6-carbonyl]-2,5- diazaspiro[3.4]octan-6- one | A.1 and B.57 | 475.2 [M + H]⁺ |
| 205 | 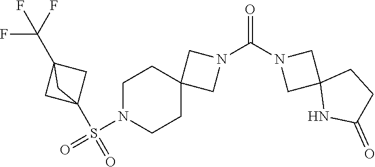 [SMILES: O=C1CCC2(CN(C(=O)N3CC4(CCN(S(=O)(=O)C56CC(C(F)(F)F)(C5)C6)CC4)C3)C2)N1] | 2-[7-[[3- (trifluoromethyl)-1- bicyclo[1.1.1]pentanyl] sulfonyl]-2,7- diazaspiro[3.5]nonane- 2-carbonyl]-2,5- diazaspiro[3.4]octan-6- one | A.1 and B.167 | 477.2 [M + H]⁺ |

### Example 376

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 377

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (I) 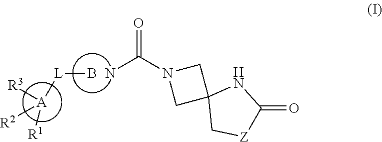
or a pharmaceutically acceptable salt thereof, wherein:
X¹, X², X³, and X⁴ are each independently selected from CH and N;
W is selected from O and NH;
Z is selected from O, CH₂, CH₂CH₂, NH, CH₂O, and OCH₂;
L is selected from a covalent bond, CH₂, NR⁴, NHCH₂, CH₂NH, NHCO, CONH, CH₂O, OCH₂, O, NHSO₂, SO₂, SO₂NH, 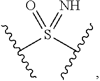 [SMILES: *S(*)(=N)=O]
and CH₂NHSO₂;
A is selected from pyridyl, phenyl, azetidinyl, cyclopropyl, triazolyl, thiazolyl, isothiazolyl, thiadiazolyl, imidazolyl, pyrimidinyl, pyrazinyl, pyridazinyl, pyrazolyl, oxazolyl, isoxazolyl, [1,2,4]triazolo[1,5-a]pyridyl, 1H-pyrazolo[4,3-b]pyridyl, 1,2-dihydropyridyl, and bicyclo[1.1.1]pentanyl;
B is selected from: 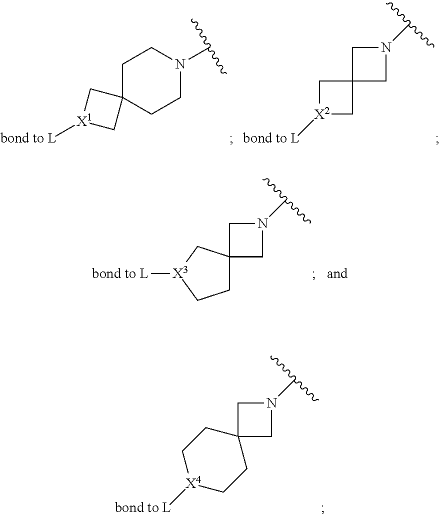
R¹ is selected from hydrogen, halogen, cyano, C₁-C₆-alkyl, C₁-C₆-alkyl-NHC(O)—, carbamoyl, C₁-C₆-alkoxy, halo-C₁-C₆-alkyl, halo-C₁-C₆-alkoxy, C₃-C₁₀-cycloalkyl, (C₁-C₆-alkyl)₂PO—, and a group 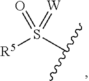
wherein said C₃-C₁₀-cycloalkyl is optionally substituted with one halo-C₁-C₆-alkyl substituent;
R² and R³ are independently selected from hydrogen, halogen, cyano, oxo, C₁-C₆-alkyl, halo-C₁-C₆-alkyl, and halo-C₁-C₆-alkoxy;
R⁴ is selected from hydrogen and C₁-C₆-alkyl; and
R⁵ is selected from C₃-C₁₀-cycloalkyl, C₁-C₆-alkyl and halo-C₁-C₆-alkyl.

2. The compound of formula (I) according to claim 1, or a pharmaceutically acceptable salt thereof, wherein:
B is selected from: 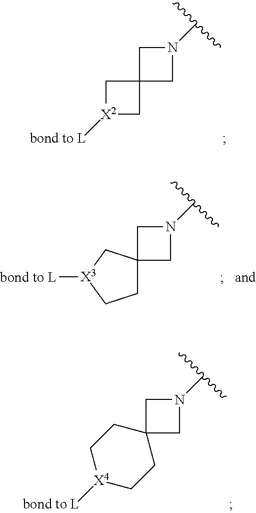
wherein
X² and X³ are both CH; and
X⁴ is selected from CH and N.

3. The compound of formula (I) according to claim 1, or a pharmaceutically acceptable salt thereof, wherein Z is selected from O, CH₂, CH₂CH₂, CH₂O, and NH.

4. The compound of formula (I) according to claim 1, or a pharmaceutically acceptable salt thereof, wherein:
L is selected from a covalent bond, CH₂, NR₄, NHCH₂, CH₂NH, NHCO, CH₂O, O, SO₂NH, NHSO₂, SO₂, 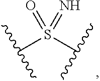 [SMILES: *S(*)(=N)=O]
and CH₂NHSO₂; and
R⁴ is selected from hydrogen and C₁-C₆-alkyl.

5. The compound of formula (I) according to claim 4, or a pharmaceutically acceptable salt thereof, wherein:
L is selected from CH₂, SO₂, NR₄, and O; and
R⁴ is hydrogen.

6. The compound of formula (I) according to claim 1, or a pharmaceutically acceptable salt thereof, wherein:
W is selected from O and NH;
A is selected from pyridyl, phenyl, azetidinyl, cyclopropyl, triazolyl, thiazolyl, thiadiazolyl, imidazolyl, pyrimidinyl, pyrazinyl, pyridazinyl, pyrazolyl, oxazolyl, [1,2,4]triazolo[1,5-a]pyridyl, 1H-pyrazolo[4,3-b]pyridyl, 1,2-dihydropyridyl, and bicyclo[1.1.1]pentanyl;
R¹ is selected from hydrogen, cyano, C₁-C₆-alkyl, C₁-C₆-alkyl-NHC(O)—, carbamoyl, halogen, halo-C₁-C₆-alkyl, halo-C₁-C₆-alkoxy, C₃-C₁₀-cycloalkyl, and a group 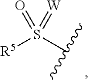
wherein said C₃-C₁₀-cycloalkyl is optionally substituted with one halo-C₁-C₆-alkyl substituent;
R² is selected from hydrogen, halogen, cyano, oxo, C₁-C₆-alkyl, halo-C₁-C₆-alkyl, and halo-C₁-C₆-alkoxy;
R³ is selected from hydrogen and C₁-C₆-alkyl; and
R⁵ is selected from C₃-C₁₀-cycloalkyl, C₁-C₆-alkyl and halo-C₁-C₆-alkyl.

7. The compound of formula (I) according to claim 6, or a pharmaceutically acceptable salt thereof, wherein:
W is selected from O and NH;
A is selected from pyridyl, phenyl, triazolyl, pyrazolyl, pyrimidinyl, and pyridazinyl;
R¹ is selected from halo-C₁-C₆-alkyl, halo-C₁-C₆-alkoxy, and a group 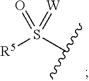
R² is selected from hydrogen, halogen, C₁-C₆-alkyl, and cyano;
R³ is hydrogen; and
R⁵ is selected from C₁-C₆-alkyl and halo-C₁-C₆-alkyl.

8. The compound of formula (I) according to claim 7, or a pharmaceutically acceptable salt thereof, wherein:
W is selected from O and NH;
A is selected from pyridyl, phenyl, pyrimidinyl, triazolyl, pyrazolyl, and pyridazinyl;
R¹ is selected from CF₃, CF₃O and a group 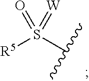
R² is selected from hydrogen, fluoro, methyl, and cyano;
R³ is hydrogen; and
R⁵ is selected from methyl and CF₃.

9. The compound of formula (I) according to claim 1, or a pharmaceutically acceptable salt thereof, wherein:
X¹, X², X³, and X⁴ are each independently selected from CH and N;
W is selected from O and NH;
Z is selected from O, CH₂, CH₂CH₂, NH, CH₂O, and OCH₂;
L is selected from a covalent bond, CH₂, NR₄, NHCH₂, CH₂NH, NHCO, CH₂O, O, SO₂NH, NHSO₂, SO₂, 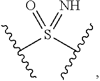 [SMILES: *S(*)(=N)=O]
and CH₂NHSO₂;
A is selected from pyridyl, phenyl, azetidinyl, cyclopropyl, triazolyl, thiazolyl, isothiazolyl, thiadiazolyl, imidazolyl, pyrimidinyl, pyrazinyl, pyridazinyl, pyrazolyl, oxazolyl, isoxazolyl, [1,2,4]triazolo[1,5-a]pyridyl, 1H-pyrazolo[4,3-b]pyridyl, 1,2-dihydropyridyl, and bicyclo[1.1.1]pentanyl;
B is selected from: 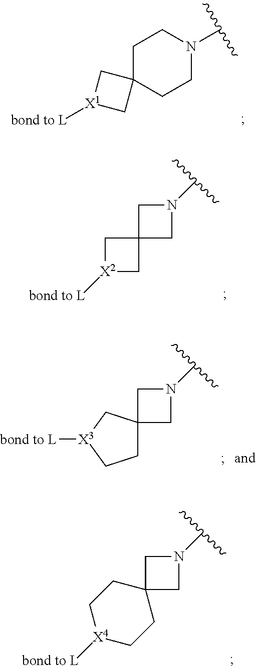
R¹ is selected from hydrogen, halogen, cyano, C₁-C₆-alkyl, C₁-C₆-alkyl-NHC(O)—, carbamoyl, halogen, halo-C₁-C₆-alkyl, halo-C₁-C₆-alkoxy, C₃-C₁₀-cycloalkyl, (C₁-C₆-alkyl)₂PO—, and a group 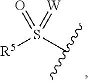
wherein said C₃-C₁₀-cycloalkyl is optionally substituted with one halo-C₁-C₆-alkyl substituent;
R² is selected from hydrogen, halogen, cyano, oxo, C₁-C₆-alkyl, halo-C₁-C₆-alkyl, and halo-C₁-C₆-alkoxy;
R³ is selected from hydrogen and C₁-C₆-alkyl;
R⁴ is selected from hydrogen and C₁-C₆-alkyl; and
R⁵ is selected from C₃-C₁₀-cycloalkyl, C₁-C₆-alkyl and halo-C₁-C₆-alkyl.

10. The compound of formula (I) according to claim 9, or a pharmaceutically acceptable salt thereof, wherein:
X² and X³ are both CH;
X⁴ is selected from CH and N;
W is selected from O and NH;
Z is selected from O, CH₂CH₂, CH₂O, CH₂, and NH;
L is selected from CH₂, SO₂, NR₄, and O;
A is selected from pyridyl, phenyl, triazolyl, pyrazolyl, pyrimidinyl, and pyridazinyl;
B is selected from: 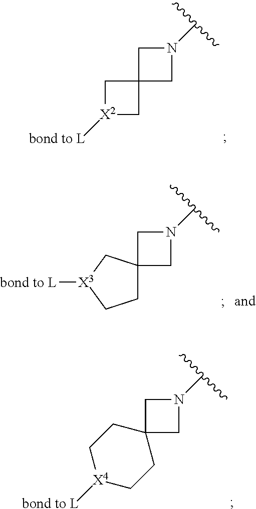
R¹ is selected from halo-C₁-C₆-alkyl, halo-C₁-C₆-alkoxy, and a group 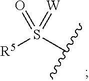
R² is selected from hydrogen, halogen, C₁-C₆-alkyl, and cyano;
R³ is hydrogen;
R⁴ is hydrogen; and
R⁵ is selected from C₁-C₆-alkyl and halo-C₁-C₆-alkyl.

11. The compound of formula (I) according to claim 10, or a pharmaceutically acceptable salt thereof, wherein:
X² and X³ are both CH;
X⁴ is selected from CH and N;
W is selected from O, CH₂CH₂, CH₂O, and NH;
Z is selected from O, CH₂, and NH;
L is selected from CH₂, SO₂, NR₄, and O;
A is selected from pyridyl, phenyl, triazolyl, pyrazolyl, pyrimidinyl, and pyridazinyl;
B is selected from: 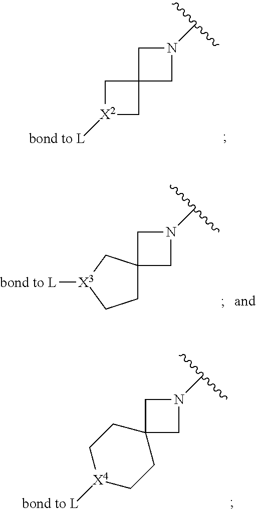
R¹ is selected from CF₃, CF₃O, and a group 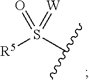
R² is selected from hydrogen, fluoro, methyl, and cyano;
R³ is hydrogen;
R⁴ is hydrogen; and
R⁵ is selected from methyl and CF₃.

12. The compound of formula (I) according to claim 1, wherein the compound is:
2-[6-(4-mesylbenzyl)-2-azaspiro[3.3]heptane-2-carbonyl]-7-oxa-2,5-diazaspiro[3.4]octan-6-one;
2-[7-[[5-(trifluoromethyl)-2-pyridyl]amino]-2-azaspiro[3.5]nonane-2-carbonyl]-7-oxa-2,5-diazaspiro[3.4]octan-6-one;
2-[6-[[4-(trifluoromethylsulfonimidoyl)benzyl]-2-azaspiro[3.3]heptane-2-carbonyl]-2,5-diazaspiro[3.4]octan-6-one;
2-[6-[[3-(trifluoromethylsulfonimidoyl)benzyl]-2-azaspiro[3.3]heptane-2-carbonyl]-2,5-diazaspiro[3.4]octan-6-one;
2-[6-[6-(trifluoromethyl)-3-pyridyl]methyl]-2-azaspiro[3.3]heptane-2-carbonyl]-7-oxa-2,5-diazaspiro[3.4]octan-6-one;
2-[6-(4-fluoro-2-mesyl-benzyl)-2-azaspiro[3.3]heptane-2-carbonyl]-7-oxa-2,5-diazaspiro[3.4]octan-6-one;
2-[7-[[6-(trifluoromethyl)pyridazin-3-yl]oxy-2-azaspiro[3.5]nonane-2-carbonyl]-7-oxa-2,5-diazaspiro[3.4]octan-6-one;
5-[[2-(6-keto-2,5-diazaspiro[3.4]octane-2-carbonyl)-2-azaspiro[3.3]heptan-6-yl]methyl]-2-(trifluoromethyl)benzonitrile;
2-[7-[methyl-[5-(trifluoromethyl) pyrazin-2-yl]amino]-2-azaspiro[3.5]nonane-2-carbonyl]-7-oxa-2,5-diazaspiro[3.4]octan-6-one;
5-[[2-(6-keto-2,5,7-triazaspiro[3.4]octane-2-carbonyl)-2-azaspiro[3.3]heptan-6-yl]methyl]-2-(trifluoromethyl)benzonitrile;
2-[7-[[5-(trifluoromethyl)-2-pyridyl]methyl]-2-azaspiro[3.5]nonane-2-carbonyl]-2,5,7-triazaspiro[3.4]octan-6-one;
2-[7-[[2-(trifluoromethyl)pyrimidin-4-yl]oxy-2-azaspiro[3.5]nonane-2-carbonyl]-7-oxa-2,5-diazaspiro[3.4]octan-6-one;
5-[[2-(6-keto-7-oxa-2,5-diazaspiro[3.4]octane-2-carbonyl)-2-azaspiro[3.3]heptan-6-yl]methyl]-2-(trifluoromethyl)benzonitrile;
2-[7-[(5-chloro-2-pyridyl)methyl]-2-azaspiro[3.5]nonane-2-carbonyl]-7-oxa-2,5-diazaspiro[3.4]octan-6-one;
2-[7-[[5-(trifluoromethyl) pyrazin-2-yl]amino]-2-azaspiro[3.5]nonane-2-carbonyl]-7-oxa-2,5-diazaspiro[3.4]octan-6-one;
2-[7-[[5-(trifluoromethyl) pyrazin-2-yl]oxy-2-azaspiro[3.5]nonane-2-carbonyl]-7-oxa-2,5-diazaspiro[3.4]octan-6-one;
2-[6-[[5-(trifluoromethyl)pyrimidin-2-yl]methyl]-2-azaspiro[3.3]heptane-2-carbonyl]-2,5-diazaspiro[3.4]octan-6-one;
2-[7-[[5-(trifluoromethyl)-2-pyridyl]methyl]-2-azaspiro[3.5]nonane-2-carbonyl]-2,5-diazaspiro[3.4]octan-6-one;
2-[6-[[6-(trifluoromethyl)-3-pyridyl]methyl]-2-azaspiro[3.3]heptane-2-carbonyl]-2,5-diazaspiro[3.4]octan-6-one;
2-[7-[[5-(trifluoromethyl)-2-pyridyl]methyl]-2-azaspiro[3.5]nonane-2-carbonyl]-7-oxa-2,5-diazaspiro[3.4]octan-6-one;
2-[6-[[4-(trifluoromethyl) pyrazol-1-yl]methyl]-2-azaspiro[3.3]heptane-2-carbonyl]-7-oxa-2,5-diazaspiro[3.4]octan-6-one;
2-[6-[[5-(trifluoromethyl) pyrazin-2-yl]methyl]-2-azaspiro[3.3]heptane-2-carbonyl]-2,5-diazaspiro[3.4]octan-6-one;
2-[7-(4-fluoro-2-mesyl-phenoxy)-2-azaspiro[3.5]nonane-2-carbonyl]-7-oxa-2,5-diazaspiro[3.4]octan-6-one;
2-[(6S)-6-[5-(trifluoromethyl) pyrazin-2-yl]oxy-2-azaspiro[3.4]octane-2-carbonyl]-7-oxa-2,5-diazaspiro[3.4]octan-6-one;
2-[6-[(5-chloro-2-pyridyl)methyl]-2-azaspiro[3.3]heptane-2-carbonyl]-7-oxa-2,5-diazaspiro[3.4]octan-6-one;
2-[6-(3-triflylbenzyl)-2-azaspiro[3.3]heptane-2-carbonyl]-7-oxa-2,5-diazaspiro[3.4]octan-6-one;
2-[6-[[5-(trifluoromethyl) pyrazin-2-yl]methyl]-2-azaspiro[3.3]heptane-2-carbonyl]-7-oxa-2,5-diazaspiro[3.4]octan-6-one;
2-[7-[[6-(trifluoromethyl)-3-pyridyl]methyl]-2-azaspiro[3.5]nonane-2-carbonyl]-7-oxa-2,5-diazaspiro[3.4]octan-6-one;
2-[7-[[5-(trifluoromethyl) pyrazin-2-yl]methyl]-2-azaspiro[3.5]nonane-2-carbonyl]-2,5-diazaspiro[3.4]octan-6-one;
2-[6-[(3,5-difluoro-2-pyridyl)methyl]-2-azaspiro[3.4]octane-2-carbonyl]-7-oxa-2,5-diazaspiro[3.4]octan-6-one;
2-[6-(4-triflylphenyl)-2-azaspiro[3.3]heptane-2-carbonyl]-7-oxa-2,5-diazaspiro[3.4]octan-6-one;
2-[(6R)-6-[5-(trifluoromethyl) pyrazin-2-yl]oxy-2-azaspiro[3.4]octane-2-carbonyl]-7-oxa-2,5-diazaspiro[3.4]octan-6-one;
2-[7-[[6-(difluoromethoxy)-3-pyridyl]methyl]-2-azaspiro[3.5]nonane-2-carbonyl]-7-oxa-2,5-diazaspiro[3.4]octan-6-one;
2-[6-[[5-(trifluoromethoxy)-2-pyridyl]methyl]-2-azaspiro[3.3]heptane-2-carbonyl]-2,5-diazaspiro[3.4]octan-6-one;
2-[6-[[3-fluoro-5-(trifluoromethyl)-2-pyridyl]methyl]-2-azaspiro[3.3]heptane-2-carbonyl]-2,5-diazaspiro[3.4]octan-6-one;
2-[7-[[1-(trifluoromethyl)cyclopropyl]methoxy]-2-azaspiro[3.5]nonane-2-carbonyl]-7-oxa-2,5-diazaspiro[3.4]octan-6-one;
2-[7-(2,5-dichlorophenoxy)-2-azaspiro[3.5]nonane-2-carbonyl]-7-oxa-2,5-diazaspiro[3.4]octan-6-one;
2-[6-[[4-fluoro-2-(trifluoromethyl)benzyl]-2-azaspiro[3.3]heptane-2-carbonyl]-7-oxa-2,5-diazaspiro[3.4]octan-6-one;
2-[6-(2,4-difluorobenzyl)-2-azaspiro[3.3]heptane-2-carbonyl]-7-oxa-2,5-diazaspiro[3.4]octan-6-one;
2-[6-[[2-fluoro-4-(trifluoromethyl)benzyl]-2-azaspiro[3.3]heptane-2-carbonyl]-7-oxa-2,5-diazaspiro[3.4]octan-6-one;
2-[6-[[2-fluoro-4-(trifluoromethyl)benzyl]-2-azaspiro[3.3]heptane-2-carbonyl]-2,5,7-triazaspiro[3.4]octan-6-one;
2-[6-[[2-fluoro-4-(trifluoromethyl)phenyl]-2-azaspiro[3.3]heptane-2-carbonyl]-7-oxa-2,5-diazaspiro[3.4]octan-6-one;
2-[6-[[5-(trifluoromethoxy)-2-pyridyl]methyl]-2-azaspiro[3.3]heptane-2-carbonyl]-7-oxa-2,5-diazaspiro[3.4]octan-6-one;
2-[6-[[2-mesyl-4-(trifluoromethyl)benzyl]-2-azaspiro[3.3]heptane-2-carbonyl]-7-oxa-2,5-diazaspiro[3.4]octan-6-one;
2-[6-[[5-(trifluoromethyl)-2-pyridyl]methyl]-2-azaspiro[3.4]octane-2-carbonyl]-7-oxa-2,5-diazaspiro[3.4]octan-6-one;
2-[7-[(3,5-difluoro-2-pyridyl)methyl]-2-azaspiro[3.5]nonane-2-carbonyl]-7-oxa-2,5-diazaspiro[3.4]octan-6-one;
2-[6-[[4-mesyl-3-(trifluoromethyl)benzyl]-2-azaspiro[3.4]octane-2-carbonyl]-7-oxa-2,5-diazaspiro[3.4]octan-6-one;
2-[6-[[3-fluoro-5-(trifluoromethyl)-2-pyridyl]methyl]-2-azaspiro[3.3]heptane-2-carbonyl]-7-oxa-2,5-diazaspiro[3.4]octan-6-one;
2-[6-[6-(trifluoromethyl)-3-pyridyl]methyl]-2-azaspiro[3.4]octane-2-carbonyl]-7-oxa-2,5-diazaspiro[3.4]octan-6-one;
2-[(6S)-6-[[5-(trifluoromethyl) pyrazin-2-yl]methyl]-2-azaspiro[3.4]octane-2-carbonyl]-7-oxa-2,5-diazaspiro[3.4]octan-6-one;
2-[6-[[4-mesyl-3-(trifluoromethyl)benzyl]-2-azaspiro[3.3]heptane-2-carbonyl]-7-oxa-2,5-diazaspiro[3.4]octan-6-one;
2-[6-[[3-mesyl-4-(trifluoromethyl)benzyl]-2-azaspiro[3.3]heptane-2-carbonyl]-7-oxa-2,5-diazaspiro[3.4]octan-6-one;
2-[6-(3-fluoro-5-mesyl-benzyl)-2-azaspiro[3.3]heptane-2-carbonyl]-7-oxa-2,5-diazaspiro[3.4]octan-6-one;
2-[7-[[5-(trifluoromethyl) pyrazin-2-yl]methyl]-2-azaspiro[3.5]nonane-2-carbonyl]-7-oxa-2,5-diazaspiro[3.4]octan-6-one;
2-[6-[(5-fluoro-2-pyridyl)methyl]-2-azaspiro[3.3]heptane-2-carbonyl]-7-oxa-2,5-diazaspiro[3.4]octan-6-one;
2-[(6R)-6-[[5-(trifluoromethyl) pyrazin-2-yl]methyl]-2-azaspiro[3.4]octane-2-carbonyl]-7-oxa-2,5-diazaspiro[3.4]octan-6-one;
N-[2-(6-keto-7-oxa-2,5-diazaspiro[3.4]octane-2-carbonyl)-2-azaspiro[3.5]nonan-7-yl]-3-(trifluoromethoxy)benzenesulfonamide;
2-[6-[[2-(trifluoromethyl)pyrimidin-5-yl]methyl]-2-azaspiro[3.3]heptane-2-carbonyl]-7-oxa-2,5-diazaspiro[3.4]octan-6-one;
2-[6-[[[1-(trifluoromethyl)cyclopropyl]amino]methyl]-2-azaspiro[3.3]heptane-2-carbonyl]-7-oxa-2,5-diazaspiro[3.4]octan-6-one;
N-[2-(6-keto-7-oxa-2,5-diazaspiro[3.4]octane-2-carbonyl)-2-azaspiro[3.3]heptan-6-yl]-3-(trifluoromethyl)benzenesulfonamide;
2-[6-(3-mesylbenzyl)-2-azaspiro[3.3]heptane-2-carbonyl]-7-oxa-2,5-diazaspiro[3.4]octan-6-one;
2-[6-(4-mesylbenzyl)-2-azaspiro[3.4]octane-2-carbonyl]-7-oxa-2,5-diazaspiro[3.4]octan-6-one;
2-[6-(4-fluorophenyl)-2-azaspiro[3.3]heptane-2-carbonyl]-7-oxa-2,5-diazaspiro[3.4]octan-6-one;
2-[6-[[4-fluoro-2-(trifluoromethyl)benzyl]-2-azaspiro[3.3]heptane-2-carbonyl]-2,5,7-triazaspiro[3.4]octan-6-one;
2-[6-[[5-(trifluoromethyl) pyrazin-2-yl]-2-azaspiro[3.3]heptane-2-carbonyl]-7-oxa-2,5-diazaspiro[3.4]octan-6-one;
2-[6-[[1-(trifluoromethyl)cyclopropyl]methoxy]-2-azaspiro[3.3]heptane-2-carbonyl]-7-oxa-2,5-diazaspiro[3.4]octan-6-one;
2-[6-[[2-mesyl-4-(trifluoromethyl)benzyl]-2-azaspiro[3.3]heptane-2-carbonyl]-2,5-diazaspiro[3.4]octan-6-one;
2-[6-[6-(trifluoromethyl)pyridazin-3-yl]methyl]-2-azaspiro[3.3]heptane-2-carbonyl]-7-oxa-2,5-diazaspiro[3.4]octan-6-one;
N-[2-(6-keto-2,5-diazaspiro[3.4]octane-2-carbonyl)-2-azaspiro[3.5]nonan-7-yl]-3-(trifluoromethoxy)benzenesulfonamide;
2-[6-[[1-(trifluoromethyl)cyclopropyl]amino]-2-azaspiro[3.3]heptane-2-carbonyl]-7-oxa-2,5-diazaspiro[3.4]octan-6-one;
2-[6-[(5-fluoro-3-pyridyl)methyl]-2-azaspiro[3.3]heptane-2-carbonyl]-7-oxa-2,5-diazaspiro[3.4]octan-6-one;
2-[6-[[2-mesyl-4-(trifluoromethyl)phenyl]-2-azaspiro[3.3]heptane-2-carbonyl]-7-oxa-2,5-diazaspiro[3.4]octan-6-one;
2-[6-[[2-(trifluoromethyl)pyrimidin-5-yl]-2-azaspiro[3.3]heptane-2-carbonyl]-7-oxa-2,5-diazaspiro[3.4]octan-6-one;
2-[6-(2,4-difluorobenzyl)-2-azaspiro[3.3]heptane-2-carbonyl]-2,5,7-triazaspiro[3.4]octan-6-one;
2-[6-[[4-mesyl-3-(trifluoromethyl)benzyl]-2-azaspiro[3.3]heptane-2-carbonyl]-2,5-diazaspiro[3.4]octan-6-one;
2-[(6S)-6-[[4-mesyl-3-(trifluoromethyl) phenoxy]-2-azaspiro[3.4]octane-2-carbonyl]-7-oxa-2,5-diazaspiro[3.4]octan-6-one;
2-[6-[[1-(trifluoromethyl)cyclopropyl]methylamino]-2-azaspiro[3.3]heptane-2-carbonyl]-7-oxa-2,5-diazaspiro[3.4]octan-6-one;
2-[7-[(5-chloro-2-pyridyl)methyl]-2-azaspiro[3.5]nonane-2-carbonyl]-2,5-diazaspiro[3.4]octan-6-one;
2-[6-[[3-mesyl-4-(trifluoromethyl)benzyl]-2-azaspiro[3.3]heptane-2-carbonyl]-2,5-diazaspiro[3.4]octan-6-one;
2-[6-[[5-(trifluoromethyl) pyrazin-2-yl]oxy-2-azaspiro[3.4]octane-2-carbonyl]-7-oxa-2,5-diazaspiro[3.4]octan-6-one;
2-[6-[[5-(trifluoromethyl) pyrazin-2-yl]oxy-2-azaspiro[3.3]heptane-2-carbonyl]-7-oxa-2,5-diazaspiro[3.4]octan-6-one;
2-[6-[6-(trifluoromethyl)-3-pyridyl]-2-azaspiro[3.3]heptane-2-carbonyl]-7-oxa-2,5-diazaspiro[3.4]octan-6-one;
2-[7-[[6-(difluoromethoxy)-3-pyridyl]methyl]-2-azaspiro[3.5]nonane-2-carbonyl]-2,5-diazaspiro[3.4]octan-6-one;
2-[7-(5-chloro-2-mesyl-phenoxy)-2-azaspiro[3.5]nonane-2-carbonyl]-7-oxa-2,5-diazaspiro[3.4]octan-6-one;
2-[7-[[5-(trifluoromethyl)pyrimidin-2-yl]oxy-2-azaspiro[3.5]nonane-2-carbonyl]-7-oxa-2,5-diazaspiro[3.4]octan-6-one;
2-[6-(4-mesylphenyl)-2-azaspiro[3.3]heptane-2-carbonyl]-7-oxa-2,5-diazaspiro[3.4]octan-6-one;
2-[6-(4-fluoro-2-mesyl-benzyl)-2-azaspiro[3.3]heptane-2-carbonyl]-2,5-diazaspiro[3.4]octan-6-one;
2-[7-[(3,5-difluoro-2-pyridyl)methyl]-2-azaspiro[3.5]nonane-2-carbonyl]-2,5-diazaspiro[3.4]octan-6-one;
2-[6-[[3-(trifluoromethyl) azetidin-1-yl]-2-azaspiro[3.3]heptane-2-carbonyl]-7-oxa-2,5-diazaspiro[3.4]octan-6-one;
2-[6-[[3-(2,2,2-trifluoroethyl) azetidin-1-yl]-2-azaspiro[3.3]heptane-2-carbonyl]-7-oxa-2,5-diazaspiro[3.4]octan-6-one;
2-[6-[6-(trifluoromethyl)pyridazin-3-yl]oxy-2-azaspiro[3.3]heptane-2-carbonyl]-7-oxa-2,5-diazaspiro[3.4]octan-6-one;
2-[6-[[4-(trifluoromethyl) pyrazol-1-yl]methyl]-2-azaspiro[3.3]heptane-2-carbonyl]-2,5-diazaspiro[3.4]octan-6-one;
N-[2-(6-keto-2,5-diazaspiro[3.4]octane-2-carbonyl)-2-azaspiro[3.3]heptan-6-yl]-3-(trifluoromethyl)benzenesulfonamide;
2-[6-(3-cyclopropyl-1,2,4-triazol-1-yl)-2-azaspiro[3.3]heptane-2-carbonyl]-7-oxa-2,5-diazaspiro[3.4]octan-6-one;
2-[6-[[3-(trifluoromethyl)-1,2,4-thiadiazol-5-yl]methyl]-2-azaspiro[3.3]heptane-2-carbonyl]-2,5-diazaspiro[3.4]octan-6-one;
2-[6-(4-mesylbenzyl)-2-azaspiro[3.3]heptane-2-carbonyl]-2,5-diazaspiro[3.4]octan-6-one;
2-[6-[[4-(trifluoromethyl) oxazol-2-yl]methyl]-2-azaspiro[3.3]heptane-2-carbonyl]-2,5-diazaspiro[3.4]octan-6-one;
2-[6-(3-mesylbenzyl)-2-azaspiro[3.3]heptane-2-carbonyl]-2,5-diazaspiro[3.4]octan-6-one;
2-[6-(5-fluoro-3-pyridyl)-2-azaspiro[3.3]heptane-2-carbonyl]-7-oxa-2,5-diazaspiro[3.4]octan-6-one;
2-[6-[[6-(trifluoromethyl)pyridazin-3-yl]amino]-2-azaspiro[3.3]heptane-2-carbonyl]-7-oxa-2,5-diazaspiro[3.4]octan-6-one;
2-[6-(3-fluoro-5-mesyl-benzyl)-2-azaspiro[3.3]heptane-2-carbonyl]-2,5-diazaspiro[3.4]octan-6-one;
2-[6-[[3-(trifluoromethyl)-1,2,4-triazol-1-yl]methyl]-2-azaspiro[3.3]heptane-2-carbonyl]-2,5-diazaspiro[3.4]octan-6-one;
2-[7-[[1-(trifluoromethyl)cyclopropyl]methoxy]-2-azaspiro[3.5]nonane-2-carbonyl]-2,5,7-triazaspiro[3.4]octan-6-one;
2-[6-[[2-(trifluoromethyl)pyrimidin-5-yl]methyl]-2-azaspiro[3.3]heptane-2-carbonyl]-2,5-diazaspiro[3.4]octan-6-one;
2-[6-[[6-(trifluoromethyl)pyridazin-3-yl]methyl]-2-azaspiro[3.3]heptane-2-carbonyl]-2,5-diazaspiro[3.4]octan-6-one;
2-[6-[[4-(trifluoromethyl) imidazol-1-yl]methyl]-2-azaspiro[3.3]heptane-2-carbonyl]-2,5-diazaspiro[3.4]octan-6-one;
2-[7-[[6-(difluoromethoxy)-3-pyridyl]methyl]-2-azaspiro[3.5]nonane-2-carbonyl]-2,5,7-triazaspiro[3.4]octan-6-one;
2-[6-[[6-(trifluoromethyl)-3-pyridyl]methyl]-2-azaspiro[3.3]heptane-2-carbonyl]-2,5,7-triazaspiro[3.4]octan-6-one;
2-[7-[[5-(trifluoromethyl) pyrazin-2-yl]amino]-2-azaspiro[3.5]nonane-2-carbonyl]-2,5-diazaspiro[3.4]octan-6-one;
2-[(6 #R!)-6-[(4-methylsulfonylphenyl)methyl]-2-azaspiro[3.4]octane-2-carbonyl]-8-oxa-2,5-diazaspiro[3.5]nonan-6-one;
2-[(6 #S!)-6-[(4-methylsulfonylphenyl)methyl]-2-azaspiro[3.4]octane-2-carbonyl]-8-oxa-2,5-diazaspiro[3.5]nonan-6-one;
2-(6-keto-7-oxa-2,5-diazaspiro[3.4]octane-2-carbonyl)-N-[1-(trifluoromethyl)cyclopropyl]-2-azaspiro[3.3]heptane-6-carboxamide;
2-[6-(4-mesylbenzyl)-2-azaspiro[3.4]octane-2-carbonyl]-8-oxa-2,5-diazaspiro[3.5]nonan-6-one;
2-[7-[[1-(trifluoromethyl)cyclopropyl]methoxy]-2-azaspiro[3.5]nonane-2-carbonyl]-7-oxa-2,5-diazaspiro[3.5]nonan-6-one;
2-[6-(3-cyclopropyl-1,2,4-triazol-1-yl)-2-azaspiro[3.3]heptane-2-carbonyl]-2,5-diazaspiro[3.4]octan-6-one;
2-[6-[6-(trifluoromethyl)pyridazin-3-yl]oxy-2-azaspiro[3.3]heptane-2-carbonyl]-2,5-diazaspiro[3.4]octan-6-one;
2-[6-[[5-(trifluoromethyl) pyrazin-2-yl]methyl]-2-azaspiro[3.3]heptane-2-carbonyl]-7-oxa-2,5-diazaspiro[3.5]nonan-6-one;
N-[2-(6-keto-7-oxa-2,5-diazaspiro[3.5]nonane-2-carbonyl)-2-azaspiro[3.3]heptan-6-yl]-3-(trifluoromethyl)benzenesulfonamide;
2-[7-[[5-(trifluoromethyl)-2-pyridyl]methyl]-2-azaspiro[3.5]nonane-2-carbonyl]-8-oxa-2,5-diazaspiro[3.5]nonan-6-one;
2-[7-[[2-fluoro-4-(trifluoromethyl)benzyl]-2,7-diazaspiro[3.5]nonane-2-carbonyl]-2,5-diazaspiro[3.4]octan-6-one;
2-[7-[[6-(difluoromethoxy)-3-pyridyl]methyl]-2-azaspiro[3.5]nonane-2-carbonyl]-8-oxa-2,5-diazaspiro[3.5]nonan-6-one;
2-[2-[2-fluoro-4-(trifluoromethyl)benzyl]-2,7-diazaspiro[3.5]nonane-7-carbonyl]-2,5-diazaspiro[3.4]octan-6-one;
2-[2-[2-fluoro-4-(trifluoromethyl)benzyl]-2,7-diazaspiro[3.5]nonane-7-carbonyl]-7-oxa-2,5-diazaspiro[3.5]nonan-6-one;
2-[7-(3-triflylbenzyl)-2,7-diazaspiro[3.5]nonane-2-carbonyl]-8-oxa-2,5-diazaspiro[3.5]nonan-6-one;
2-[7-[[2-fluoro-4-(trifluoromethyl)benzyl]-2,7-diazaspiro[3.5]nonane-2-carbonyl]-7-oxa-2,5-diazaspiro[3.4]octan-6-one;
2-[7-[[1-(trifluoromethyl)cyclopropyl]methoxy]-2-azaspiro[3.5]nonane-2-carbonyl]-8-oxa-2,5-diazaspiro[3.5]nonan-6-one;
2-[2-[2-fluoro-4-(trifluoromethyl)benzyl]-2,7-diazaspiro[3.5]nonane-7-carbonyl]-7-oxa-2,5-diazaspiro[3.4]octan-6-one;
2-[2-[2-fluoro-4-(trifluoromethyl)benzyl]-2,7-diazaspiro[3.5]nonane-7-carbonyl]-7-oxa-2,5-diazaspiro[3.4]octan-6-one”;
2-[7-(4-triflylbenzyl)-2,7-diazaspiro[3.5]nonane-2-carbonyl]-8-oxa-2,5-diazaspiro[3.5]nonan-6-one;
2-[7-(3-triflylbenzyl)-2,7-diazaspiro[3.5]nonane-2-carbonyl]-2,5-diazaspiro[3.4]octan-6-one;
2-[7-(4-triflylbenzyl)-2,7-diazaspiro[3.5]nonane-2-carbonyl]-2,5-diazaspiro[3.4]octan-6-one;
2-[6-[[[1-(trifluoromethyl)cyclopropyl]amino]methyl]-2-azaspiro[3.3]heptane-2-carbonyl]-8-oxa-2,5-diazaspiro[3.5]nonan-6-one;
2-[6-(2,4-difluorobenzyl)-2-azaspiro[3.3]heptane-2-carbonyl]-8-oxa-2,5-diazaspiro[3.5]nonan-6-one;
2-[2-[2-fluoro-4-(trifluoromethyl)benzyl]-2,7-diazaspiro[3.5]nonane-7-carbonyl]-2,5,7-triazaspiro[3.4]octan-6-one;
2-[6-[[5-(trifluoromethyl) pyrazin-2-yl]methyl]-2-azaspiro[3.3]heptane-2-carbonyl]-8-oxa-2,5-diazaspiro[3.5]nonan-6-one;
2-[7-(3-triflylbenzyl)-2,7-diazaspiro[3.5]nonane-2-carbonyl]-7-oxa-2,5-diazaspiro[3.4]octan-6-one;
2-[6-[[1-(trifluoromethyl)cyclopropyl]methoxy]-2-azaspiro[3.3]heptane-2-carbonyl]-8-oxa-2,5-diazaspiro[3.5]nonan-6-one;
2-[7-[[2-fluoro-4-(trifluoromethyl)phenyl]sulfonyl-2,7-diazaspiro[3.5]nonane-2-carbonyl]-8-oxa-2,5-diazaspiro[3.5]nonan-6-one;
2-[6-[[3-fluoro-5-(trifluoromethyl)benzyl]-2-azaspiro[3.3]heptane-2-carbonyl]-8-oxa-2,5-diazaspiro[3.5]nonan-6-one;
2-[6-[[4-fluoro-2-(trifluoromethyl)benzyl]-2-azaspiro[3.3]heptane-2-carbonyl]-8-oxa-2,5-diazaspiro[3.5]nonan-6-one;
2-[7-(4-triflylbenzyl)-2,7-diazaspiro[3.5]nonane-2-carbonyl]-7-oxa-2,5-diazaspiro[3.4]octan-6-one;
2-[7-[[2-fluoro-4-(trifluoromethyl)phenyl]sulfonyl-2,7-diazaspiro[3.5]nonane-2-carbonyl]-7-oxa-2,5-diazaspiro[3.5]nonan-6-one;
2-[6-[[2-fluoro-4-(trifluoromethyl)benzyl]-2-azaspiro[3.3]heptane-2-carbonyl]-8-oxa-2,5-diazaspiro[3.5]nonan-6-one;
2-[2-[3-(trifluoromethoxy)phenyl]sulfonyl-2,6-diazaspiro[3.3]heptane-6-carbonyl]-8-oxa-2,5-diazaspiro[3.5]nonan-6-one;
2-[7-[[5-(trifluoromethyl)-2-pyridyl]methyl]-2,7-diazaspiro[3.5]nonane-2-carbonyl]-2,5-diazaspiro[3.4]octan-6-one;
2-[6-(4-fluoro-2-mesyl-benzyl)-2-azaspiro[3.3]heptane-2-carbonyl]-8-oxa-2,5-diazaspiro[3.5]nonan-6-one;
2-[7-[[6-(trifluoromethyl)-3-pyridyl]methyl]-2,7-diazaspiro[3.5]nonane-2-carbonyl]-2,5-diazaspiro[3.4]octan-6-one;
2-[7-[[5-(trifluoromethyl)-2-pyridyl]methyl]-2,7-diazaspiro[3.5]nonane-2-carbonyl]-7-oxa-2,5-diazaspiro[3.4]octan-6-one;
2-[6-(4-triflylbenzyl)-2,6-diazaspiro[3.3]heptane-2-carbonyl]-7-oxa-2,5-diazaspiro[3.4]octan-6-one;
2-[7-[[5-(trifluoromethyl)-2-pyridyl]methyl]-2,7-diazaspiro[3.4]octane-2-carbonyl]-2,5-diazaspiro[3.4]octan-6-one;
2-[7-[[6-(trifluoromethyl)-3-pyridyl]methyl]-2,7-diazaspiro[3.4]octane-2-carbonyl]-2,5-diazaspiro[3.4]octan-6-one;
2-[7-[[6-(trifluoromethyl)-3-pyridyl]methyl]-2,7-diazaspiro[3.5]nonane-2-carbonyl]-7-oxa-2,5-diazaspiro[3.4]octan-6-one;
2-[6-(3,5-difluorobenzyl)-2,6-diazaspiro[3.3]heptane-2-carbonyl]-7-oxa-2,5-diazaspiro[3.4]octan-6-one;
2-[7-[[5-(trifluoromethyl)-2-pyridyl]methyl]-2,7-diazaspiro[3.4]octane-2-carbonyl]-7-oxa-2,5-diazaspiro[3.4]octan-6-one;
2-[7-[[5-(trifluoromethyl) pyrazin-2-yl]methyl]-2,7-diazaspiro[3.4]octane-2-carbonyl]-2,5-diazaspiro[3.4]octan-6-one;
2-[7-[[6-(trifluoromethyl)-3-pyridyl]methyl]-2,7-diazaspiro[3.4]octane-2-carbonyl]-7-oxa-2,5-diazaspiro[3.4]octan-6-one;
2-[7-[[5-(trifluoromethyl) pyrazin-2-yl]methyl]-2,7-diazaspiro[3.4]octane-2-carbonyl]-7-oxa-2,5-diazaspiro[3.4]octan-6-one;
2-[7-[[6-(trifluoromethyl)pyridazin-3-yl]methyl]-2,7-diazaspiro[3.4]octane-2-carbonyl]-7-oxa-2,5-diazaspiro[3.4]octan-6-one;
2-[6-[[6-(trifluoromethyl)pyridazin-3-yl]methyl]-2,6-diazaspiro[3.3]heptane-2-carbonyl]-7-oxa-2,5-diazaspiro[3.4]octan-6-one;
2-[7-(4-mesylbenzyl)-2,7-diazaspiro[3.5]nonane-2-carbonyl]-2,5-diazaspiro[3.4]octan-6-one;
2-[6-[[2-(trifluoromethyl)pyrimidin-5-yl]methyl]-2,6-diazaspiro[3.3]heptane-2-carbonyl]-7-oxa-2,5-diazaspiro[3.4]octan-6-one;
2-[6-[(3,5-difluoro-2-pyridyl)methyl]-2,6-diazaspiro[3.3]heptane-2-carbonyl]-7-oxa-2,5-diazaspiro[3.4]octan-6-one;
2-[7-(3-mesylbenzyl)-2,7-diazaspiro[3.5]nonane-2-carbonyl]-2,5-diazaspiro[3.4]octan-6-one;
2-[6-(4-mesylbenzyl)-2,6-diazaspiro[3.3]heptane-2-carbonyl]-7-oxa-2,5-diazaspiro[3.4]octan-6-one;
2-[6-[[1-methyl-5-(trifluoromethyl) pyrazol-4-yl]methyl]-2-azaspiro[3.3]heptane-2-carbonyl]-2,5-diazaspiro[3.4]octan-6-one;
2-[6-[[1-methyl-5-(trifluoromethyl) pyrazol-3-yl]methyl]-2-azaspiro[3.3]heptane-2-carbonyl]-2,5-diazaspiro[3.4]octan-6-one;
2-[6-[[2-methyl-4-(trifluoromethyl) pyrazol-3-yl]methyl]-2-azaspiro[3.3]heptane-2-carbonyl]-2,5-diazaspiro[3.4]octan-6-one;
2-[6-[[5-(trifluoromethyl) pyrazol-1-yl]methyl]-2-azaspiro[3.3]heptane-2-carbonyl]-2,5-diazaspiro[3.4]octan-6-one;
2-[6-[[1-methyl-3-(trifluoromethyl) pyrazol-4-yl]methyl]-2-azaspiro[3.3]heptane-2-carbonyl]-2,5-diazaspiro[3.4]octan-6-one;
2-[6-[[5-(trifluoromethyl)-2-pyridyl]methyl]-2-azaspiro[3.3]heptane-2-carbonyl]-8-oxa-2,5-diazaspiro[3.5]nonan-6-one;
2-[6-[[3-fluoro-5-(trifluoromethyl)-2-pyridyl]methyl]-2-azaspiro[3.3]heptane-2-carbonyl]-8-oxa-2,5-diazaspiro[3.5]nonan-6-one;
2-[6-[(5-chloro-3-pyridyl)methyl]-2-azaspiro[3.3]heptane-2-carbonyl]-8-oxa-2,5-diazaspiro[3.5]nonan-6-one;
2-[6-[[5-(trifluoromethyl)-3-pyridyl]methyl]-2-azaspiro[3.3]heptane-2-carbonyl]-8-oxa-2,5-diazaspiro[3.5]nonan-6-one;
2-[6-[[6-(trifluoromethyl)-3-pyridyl]methyl]-2-azaspiro[3.3]heptane-2-carbonyl]-8-oxa-2,5-diazaspiro[3.5]nonan-6-one;
2-[6-[(5-chloro-3-fluoro-2-pyridyl)methyl]-2-azaspiro[3.3]heptane-2-carbonyl]-2,5-diazaspiro[3.4]octan-6-one;
2-[6-[(3-chloro-5-fluoro-2-pyridyl)methyl]-2-azaspiro[3.3]heptane-2-carbonyl]-8-oxa-2,5-diazaspiro[3.5]nonan-6-one;
2-[6-[[5-(trifluoromethoxy)-2-pyridyl]methyl]-2-azaspiro[3.3]heptane-2-carbonyl]-8-oxa-2,5-diazaspiro[3.5]nonan-6-one;
2-[6-[[4-(trifluoromethyl)-2-pyridyl]methyl]-2-azaspiro[3.3]heptane-2-carbonyl]-8-oxa-2,5-diazaspiro[3.5]nonan-6-one;
2-[6-[[6-(trifluoromethoxy)-3-pyridyl]methyl]-2-azaspiro[3.3]heptane-2-carbonyl]-2,5-diazaspiro[3.4]octan-6-one;
2-[6-[(5-fluoro-3-pyridyl)methyl]-2-azaspiro[3.3]heptane-2-carbonyl]-8-oxa-2,5-diazaspiro[3.5]nonan-6-one;
2-[6-[[4-methylsulfonyl-3-(trifluoromethyl)phenyl]methyl]-2-azaspiro[3.3]heptane-2-carbonyl]-8-oxa-2,5-diazaspiro[3.5]nonan-6-one;
2-[6-[[3-(trifluoromethylsulfonyl)phenyl]methyl]-2-azaspiro[3.3]heptane-2-carbonyl]-8-oxa-2,5-diazaspiro[3.5]nonan-6-one;
2-[6-[[4-methylsulfonyl-3-(trifluoromethyl)phenyl]methyl]-2-azaspiro[3.3]heptane-2-carbonyl]-2,5-diazaspiro[3.5]nonan-6-one;
2-[6-[[2-methylsulfonyl-4-(trifluoromethyl)phenyl]methyl]-2-azaspiro[3.3]heptane-2-carbonyl]-8-oxa-2,5-diazaspiro[3.5]nonan-6-one;
2-[6-[[2-methylsulfonyl-4-(trifluoromethyl)phenyl]methyl]-2-azaspiro[3.3]heptane-2-carbonyl]-2,5-diazaspiro[3.5]nonan-6-one;
2-[6-[[3-(trifluoromethylsulfonyl)phenyl]methyl]-2-azaspiro[3.3]heptane-2-carbonyl]-2,5-diazaspiro[3.5]nonan-6-one;
2-[6-[(2-methylsulfonylphenyl)methyl]-2,6-diazaspiro[3.3]heptane-2-carbonyl]-7-oxa-2,5-diazaspiro[3.4]octan-6-one;
2-[7-[[2-fluoro-4-(trifluoromethyl)phenyl]sulfonyl-2,7-diazaspiro[3.5]nonane-2-carbonyl]-7-oxa-2,5-diazaspiro[3.4]octan-6-one;
2-[2-[2-(trifluoromethoxy)phenyl]sulfonyl-2,6-diazaspiro[3.3]heptane-6-carbonyl]-7-oxa-2,5-diazaspiro[3.4]octan-6-one;
2-[2-[4-(trifluoromethoxy)phenyl]sulfonyl-2,6-diazaspiro[3.3]heptane-6-carbonyl]-7-oxa-2,5-diazaspiro[3.4]octan-6-one;
2-[2-[3-(trifluoromethoxy)phenyl]sulfonyl-2,6-diazaspiro[3.3]heptane-6-carbonyl]-7-oxa-2,5-diazaspiro[3.4]octan-6-one;
N-(1-methylcyclopropyl)-6-(6-oxo-7-oxa-2,5-diazaspiro[3.4]octane-2-carbonyl)-2,6-diazaspiro[3.3]heptane-2-sulfonamide;
2-[7-[[2-fluoro-4-(trifluoromethyl)phenyl]sulfonyl-2,7-diazaspiro[3.5]nonane-2-carbonyl]-2,5-diazaspiro[3.4]octan-6-one;
2-[2-[[4-fluoro-2-(trifluoromethyl)phenyl]sulfonyl-2,6-diazaspiro[3.3]heptane-6-carbonyl]-7-oxa-2,5-diazaspiro[3.4]octan-6-one;
2-[2-[[5-(trifluoromethyl)-3-pyridyl]sulfonyl]-2,6-diazaspiro[3.3]heptane-6-carbonyl]-7-oxa-2,5-diazaspiro[3.4]octan-6-one;
6-(6-oxo-7-oxa-2,5-diazaspiro[3.4]octane-2-carbonyl)-N-[1-(trifluoromethyl)cyclopropyl]methyl]-2,6-diazaspiro[3.3]heptane-2-sulfonamide;
2-[2-[3-(trifluoromethoxy)phenyl]sulfonyl-2,6-diazaspiro[3.3]heptane-6-carbonyl]-2,5-diazaspiro[3.4]octan-6-one;
2-[2-[4-(trifluoromethoxy)phenyl]sulfonyl-2,6-diazaspiro[3.3]heptane-6-carbonyl]-2,5-diazaspiro[3.4]octan-6-one;
2-[2-[2-(trifluoromethoxy)phenyl]sulfonyl-2,6-diazaspiro[3.3]heptane-6-carbonyl]-2,5-diazaspiro[3.4]octan-6-one;
N-methyl-2-[[2-(6-oxo-7-oxa-2,5-diazaspiro[3.4]octane-2-carbonyl)-2,6-diazaspiro[3.3]heptan-6-yl]methyl]benzamide;
2-[6-[[5-(trifluoromethyl)-2-pyridyl]-2,6-diazaspiro[3.3]heptane-2-carbonyl]-7-oxa-2,5-diazaspiro[3.4]octan-6-one;
2-[6-[[3-fluoro-5-(trifluoromethyl)-2-pyridyl]-2,6-diazaspiro[3.3]heptane-2-carbonyl]-7-oxa-2,5-diazaspiro[3.4]octan-6-one;
2-[7-[3-(trifluoromethyl)-1-bicyclo[1.1.1]pentanyl]sulfonyl]-2,7-diazaspiro[3.5]nonane-2-carbonyl]-7-oxa-2,5-diazaspiro[3.4]octan-6-one;
2-[7-[3-(trifluoromethyl)-1-bicyclo[1.1.1]pentanyl]sulfonyl]-2,7-diazaspiro[3.5]nonane-2-carbonyl]-2,5-diazaspiro[3.4]octan-6-one;
2-[6-[[5-(trifluoromethyl) pyrazin-2-yl]-2,6-diazaspiro[3.3]heptane-2-carbonyl]-7-oxa-2,5-diazaspiro[3.4]octan-6-one;
2-[6-[[5-(trifluoromethyl)pyrimidin-2-yl]-2,6-diazaspiro[3.3]heptane-2-carbonyl]-7-oxa-2,5-diazaspiro[3.4]octan-6-one;
N-[2-(6-oxo-8-oxa-2,5-diazaspiro[3.5]nonane-2-carbonyl)-2-azaspiro[3.3]heptan-6-yl]-3-(trifluoromethyl)benzenesulfonamide;
5-[[(6S)-2-(6-oxo-7-oxa-2,5-diazaspiro[3.4]octane-2-carbonyl)-2-azaspiro[3.4]octan-6-yl]oxy]-2-(trifluoromethyl)pyridine-4-carbonitrile;
5-[[2-(6-oxo-7-oxa-2,5-diazaspiro[3.4]octane-2-carbonyl)-2-azaspiro[3.4]octan-6-yl]methyl]-2-(trifluoromethyl)pyridine-4-carbonitrile;
5-[[2-(6-oxo-7-oxa-2,5-diazaspiro[3.4]octane-2-carbonyl)-2-azaspiro[3.3]heptan-6-yl]methyl]-2-(trifluoromethyl)pyridine-4-carbonitrile;
5-[[(6S)-2-(6-oxo-7-oxa-2,5-diazaspiro[3.4]octane-2-carbonyl)-2-azaspiro[3.4]octan-6-yl]methyl]-2-(trifluoromethyl)pyridine-4-carbonitrile;
5-[[(6R)-2-(6-oxo-7-oxa-2,5-diazaspiro[3.4]octane-2-carbonyl)-2-azaspiro[3.4]octan-6-yl]methyl]-2-(trifluoromethyl)pyridine-4-carbonitrile;
5-[[(6S)-2-(6-oxo-8-oxa-2,5-diazaspiro[3.5]nonane-2-carbonyl)-2-azaspiro[3.4]octan-6-yl]oxy]-2-(trifluoromethyl)pyridine-4-carbonitrile;
2-[6-[(4-cyclopropylsulfonylphenyl)methyl]-2-azaspiro[3.3]heptane-2-carbonyl]-2,5-diazaspiro[3.4]octan-6-one;
2-[6-[[1-(trifluoromethyl) pyrazol-4-yl]methyl]-2-azaspiro[3.3]heptane-2-carbonyl]-2,5-diazaspiro[3.4]octan-6-one;
3-[[2-(6-oxo-2,5-diazaspiro[3.4]octane-2-carbonyl)-2-azaspiro[3.3]heptan-6-yl]methyl]-5-(trifluoromethyl)benzonitrile;
2-[7-[[4-(trifluoromethylsulfonyl)phenyl]methyl]-2,7-diazaspiro[3.4]octane-2-carbonyl]-2,5-diazaspiro[3.4]octan-6-one;
4-[[2-(6-oxo-2,5-diazaspiro[3.4]octane-2-carbonyl)-2-azaspiro[3.3]heptan-6-yl]methyl]-2-(trifluoromethyl)benzonitrile;
2-[6-[[3-(trifluoromethyl)-1H-pyrazol-5-yl]methyl]-2-azaspiro[3.3]heptane-2-carbonyl]-2,5-diazaspiro[3.4]octan-6-one;
2-[(6R)-6-[[4-(trifluoromethylsulfonyl)phenyl]methyl]-2-azaspiro[3.4]octane-2-carbonyl]-2,5-diazaspiro[3.4]octan-6-one;
2-[(6S)-6-[[4-(trifluoromethylsulfonyl)phenyl]methyl]-2-azaspiro[3.4]octane-2-carbonyl]-2,5-diazaspiro[3.4]octan-6-one;
2-[(6R)-6-[[3-(trifluoromethylsulfonyl)phenyl]methyl]-2-azaspiro[3.4]octane-2-carbonyl]-2,5-diazaspiro[3.4]octan-6-one;
2-[(6S)-6-(3-triflylbenzyl)-2-azaspiro[3.4]octane-2-carbonyl]-2,5-diazaspiro[3.4]octan-6-one;
2-[6-[[3-(trifluoromethyl) pyrazol-1-yl]methyl]-2-azaspiro[3.3]heptane-2-carbonyl]-2,5-diazaspiro[3.4]octan-6-one;
2-[7-[[5-(trifluoromethyl) pyrazin-2-yl]methyl]-2-azaspiro[3.5]nonane-2-carbonyl]-8-oxa-2,5-diazaspiro[3.5]nonan-6-one;
2-[6-(3-cyclopropyl-1,2,4-triazol-1-yl)-2-azaspiro[3.3]heptane-2-carbonyl]-8-oxa-2,5-diazaspiro[3.5]nonan-6-one;
2-[6-[[2-fluoro-4-(trifluoromethyl)phenyl]-2-azaspiro[3.3]heptane-2-carbonyl]-8-oxa-2,5-diazaspiro[3.5]nonan-6-one;
2-[6-[[4-(trifluoromethylsulfonyl)phenyl]methyl]-2,6-diazaspiro[3.3]heptane-2-carbonyl]-8-oxa-2,5-diazaspiro[3.5]nonan-6-one;
2-[6-[[4-(trifluoromethyl) pyrazol-1-yl]methyl]-2-azaspiro[3.3]heptane-2-carbonyl]-8-oxa-2,5-diazaspiro[3.5]nonan-6-one;
2-[6-[6-(trifluoromethyl)pyridazin-3-yl]methyl]-2-azaspiro[3.3]heptane-2-carbonyl]-8-oxa-2,5-diazaspiro[3.5]nonan-6-one;
2-[6-[[2-(trifluoromethyl)pyrimidin-5-yl]methyl]-2-azaspiro[3.3]heptane-2-carbonyl]-8-oxa-2,5-diazaspiro[3.5]nonan-6-one;
2-[7-[[4-(trifluoromethylsulfonyl)phenyl]methyl]-2,7-diazaspiro[3.4]octane-2-carbonyl]-8-oxa-2,5-diazaspiro[3.5]nonan-6-one;
2-[(6S)-6-[[4-(trifluoromethylsulfonyl)phenyl]methyl]-2-azaspiro[3.4]octane-2-carbonyl]-8-oxa-2,5-diazaspiro[3.5]nonan-6-one;
2-[(6R)-6-[[4-(trifluoromethylsulfonyl)phenyl]methyl]-2-azaspiro[3.4]octane-2-carbonyl]-8-oxa-2,5-diazaspiro[3.5]nonan-6-one;
2-[(6S)-6-[[3-(trifluoromethylsulfonyl)phenyl]methyl]-2-azaspiro[3.4]octane-2-carbonyl]-8-oxa-2,5-diazaspiro[3.5]nonan-6-one;
2-[(6R)-6-[[3-(trifluoromethylsulfonyl)phenyl]methyl]-2-azaspiro[3.4]octane-2-carbonyl]-8-oxa-2,5-diazaspiro[3.5]nonan-6-one;
2-[6-[[3-methylsulfonyl-5-(trifluoromethyl)phenyl]methyl]-2-azaspiro[3.3]heptane-2-carbonyl]-8-oxa-2,5-diazaspiro[3.5]nonan-6-one;
2-[6-[[3-(trifluoromethyl) pyrazol-1-yl]methyl]-2-azaspiro[3.3]heptane-2-carbonyl]-8-oxa-2,5-diazaspiro[3.5]nonan-6-one;
2-[7-[[6-(trifluoromethyl)-3-pyridyl]methyl]-2,7-diazaspiro[3.4]octane-2-carbonyl]-8-oxa-2,5-diazaspiro[3.5]nonan-6-one;
2-[7-[[5-(trifluoromethyl)-2-pyridyl]methyl]-2,7-diazaspiro[3.4]octane-2-carbonyl]-8-oxa-2,5-diazaspiro[3.5]nonan-6-one;
2-[[2-(6-oxo-7-oxa-2,5-diazaspiro[3.4]octane-2-carbonyl)-2-azaspiro[3.3]heptan-6-yl]methyl]-5-(trifluoromethyl)benzonitrile;
5-[[2-(6-oxo-7-oxa-2,5-diazaspiro[3.4]octane-2-carbonyl)-2-azaspiro[3.3]heptan-6-yl]methyl]-2-(trifluoromethyl)pyridine-3-carbonitrile;
4-[[2-(6-oxo-7-oxa-2,5-diazaspiro[3.4]octane-2-carbonyl)-2-azaspiro[3.3]heptan-6-yl]methyl]-2-(trifluoromethoxy)benzamide;
5-[[2-(6-oxo-7-oxa-2,5-diazaspiro[3.4]octane-2-carbonyl)-2-azaspiro[3.3]heptan-6-yl]methyl]-2-(trifluoromethoxy)benzamide;
2-[6-[[2-methyl-5-(trifluoromethyl) pyrazol-3-yl]methyl]-2-azaspiro[3.3]heptane-2-carbonyl]-2,5-diazaspiro[3.4]octan-6-one;
1-[[2-(6-oxo-2,5-diazaspiro[3.4]octane-2-carbonyl)-2-azaspiro[3.3]heptan-6-yl]methyl]pyrazole-4-carbonitrile;
2-[6-[[1-(2,2,2-trifluoroethyl) pyrazol-4-yl]methyl]-2-azaspiro[3.3]heptane-2-carbonyl]-2,5-diazaspiro[3.4]octan-6-one;
2-[6-[[2-oxo-4-(trifluoromethyl)-1-pyridyl]methyl]-2-azaspiro[3.3]heptane-2-carbonyl]-2,5-diazaspiro[3.4]octan-6-one;
6-oxo-N-[[6-(trifluoromethyl)-3,4-diazatricyclo[7.1.1.03,7]undeca-4,6-dien-1-yl]methyl]-2,5-diazaspiro[3.4]octane-2-carboxamide;
3-fluoro-4-[[2-(6-oxo-2,5-diazaspiro[3.4]octane-2-carbonyl)-2-azaspiro[3.3]heptan-6-yl]methyl]benzonitrile;
2-[6-[[5-(trifluoromethylsulfonyl)-3-pyridyl]methyl]-2-azaspiro[3.3]heptane-2-carbonyl]-2,5-diazaspiro[3.4]octan-6-one;
2-[6-[[5-(trifluoromethylsulfonyl)-3-pyridyl]methyl]-2-azaspiro[3.3]heptane-2-carbonyl]-8-oxa-2,5-diazaspiro[3.5]nonan-6-one;
2-[6-[[3-fluoro-5-(trifluoromethylsulfonyl)phenyl]methyl]-2-azaspiro[3.3]heptane-2-carbonyl]-8-oxa-2,5-diazaspiro[3.5]nonan-6-one;
2-[6-[[3-(methylsulfonimidoyl)-5-(trifluoromethyl)phenyl]methyl]-2-azaspiro[3.3]heptane-2-carbonyl]-2,5-diazaspiro[3.4]octan-6-one;
2-[7-[[3-(trifluoromethylsulfonyl)phenyl]methyl]-2,7-diazaspiro[3.4]octane-2-carbonyl]-2,5-diazaspiro[3.4]octan-6-one;
2-[6-[[5-(difluoromethyl)-1H-pyrazol-3-yl]methyl]-2-azaspiro[3.3]heptane-2-carbonyl]-2,5-diazaspiro[3.4]octan-6-one;
2-[6-[[2-fluoro-4-(trifluoromethylsulfonyl)phenyl]methyl]-2,6-diazaspiro[3.3]heptane-2-carbonyl]-2,5-diazaspiro[3.4]octan-6-one;
2-[6-[[2-oxo-5-(trifluoromethyl)-1-pyridyl]methyl]-2-azaspiro[3.3]heptane-2-carbonyl]-2,5-diazaspiro[3.4]octan-6-one;
2-[7-[[3-(trifluoromethylsulfonyl)phenyl]methyl]-2,7-diazaspiro[3.4]octane-2-carbonyl]-8-oxa-2,5-diazaspiro[3.5]nonan-6-one;
5-[[2-(6-oxo-7-oxa-2,5-diazaspiro[3.4]octane-2-carbonyl)-2-azaspiro[3.3]heptan-6-yl]methyl]-3-(trifluoromethyl)pyridine-2-carbonitrile;
2-[6-[[5-(trifluoromethylsulfonyl)-2-pyridyl]methyl]-2-azaspiro[3.3]heptane-2-carbonyl]-2,5-diazaspiro[3.4]octan-6-one;
2-[6-[[5-(trifluoromethylsulfonyl)-2-pyridyl]methyl]-2-azaspiro[3.3]heptane-2-carbonyl]-2,5-diazaspiro[3.5]nonan-6-one;
2-[6-[[5-(trifluoromethylsulfonyl)-2-pyridyl]methyl]-2-azaspiro[3.3]heptane-2-carbonyl]-8-oxa-2,5-diazaspiro[3.5]nonan-6-one;
2-[6-[[2-fluoro-4-(trifluoromethylsulfonimidoyl)phenyl]methyl]-2-azaspiro[3.3]heptane-2-carbonyl]-2,5-diazaspiro[3.4]octan-6-one;
2-[7-[[3-(trifluoromethylsulfonyl)phenyl]methyl]-2,7-diazaspiro[3.4]octane-2-carbonyl]-2,5-diazaspiro[3.5]nonan-6-one;
2-[6-[[2-fluoro-4-(trifluoromethylsulfonyl)phenyl]methyl]-2,6-diazaspiro[3.3]heptane-2-carbonyl]-2,5-diazaspiro[3.5]nonan-6-one;
2-[6-[[3-methylsulfonyl-5-(trifluoromethyl)phenyl]methyl]-2-azaspiro[3.3]heptane-2-carbonyl]-2,5-diazaspiro[3.5]nonan-6-one;
2-[6-[(4-cyclopropylsulfonylphenyl)methyl]-2-azaspiro[3.3]heptane-2-carbonyl]-2,5-diazaspiro[3.5]nonan-6-one;
4-[[2-(6-oxo-2,5-diazaspiro[3.5]nonane-2-carbonyl)-2-azaspiro[3.3]heptan-6-yl]methyl]-2-(trifluoromethyl)benzonitrile;
5-[[2-(6-oxo-2,5-diazaspiro[3.5]nonane-2-carbonyl)-2-azaspiro[3.3]heptan-6-yl]methyl]-2-(trifluoromethyl)benzonitrile;
5-[[2-(6-oxo-2,5-diazaspiro[3.5]nonane-2-carbonyl)-2-azaspiro[3.3]heptan-6-yl]methyl]-2-(trifluoromethyl)pyridine-3-carbonitrile;
3-fluoro-4-[[2-(6-oxo-2,5-diazaspiro[3.5]nonane-2-carbonyl)-2-azaspiro[3.3]heptan-6-yl]methyl]benzonitrile;
2-[6-[[4-(trifluoromethyl) pyrazol-1-yl]methyl]-2-azaspiro[3.3]heptane-2-carbonyl]-2,5-diazaspiro[3.5]nonan-6-one;
2-[6-[[1-methyl-5-(trifluoromethyl) pyrazol-3-yl]methyl]-2-azaspiro[3.3]heptane-2-carbonyl]-2,5-diazaspiro[3.5]nonan-6-one;
2-[6-[[1-methyl-5-(trifluoromethyl) pyrazol-4-yl]methyl]-2-azaspiro[3.3]heptane-2-carbonyl]-2,5-diazaspiro[3.5]nonan-6-one;
2-[6-[[2-methyl-4-(trifluoromethyl) pyrazol-3-yl]methyl]-2-azaspiro[3.3]heptane-2-carbonyl]-2,5-diazaspiro[3.5]nonan-6-one;
2-[6-[[3-(trifluoromethyl)-1H-pyrazol-5-yl]methyl]-2-azaspiro[3.3]heptane-2-carbonyl]-2,5-diazaspiro[3.5]nonan-6-one;
2-[6-[[3-(difluoromethyl)-1H-pyrazol-5-yl]methyl]-2-azaspiro[3.3]heptane-2-carbonyl]-2,5-diazaspiro[3.5]nonan-6-one;
2-[6-[[3-(trifluoromethoxy)phenyl]sulfonyl-2-azaspiro[3.3]heptane-2-carbonyl]-2,5-diazaspiro[3.4]octan-6-one;
2-[7-[[4-(trifluoromethyl)phenyl]sulfonyl-2-azaspiro[3.5]nonane-2-carbonyl]-2,5-diazaspiro[3.4]octan-6-one;
2-[7-[3-(trifluoromethyl)phenyl]sulfonyl-2-azaspiro[3.5]nonane-2-carbonyl]-2,5-diazaspiro[3.4]octan-6-one;
2-[6-[[4-(trifluoromethylsulfonimidoyl)phenyl]methyl]-2-azaspiro[3.3]heptane-2-carbonyl]-2,5-diazaspiro[3.4]octan-6-one;
2-[6-[[4-(trifluoromethylsulfonimidoyl)phenyl]methyl]-2-azaspiro[3.3]heptane-2-carbonyl]-2,5-diazaspiro[3.5]nonan-6-one;
2-[6-[[4-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[4-(trifluoromethylsulfonimidoyl)phenyl]methyl]-2-azaspiro[3.3]heptane-2-carbonyl]-2,5-diazaspiro[3.4]octan-6-one;
2-[6-[[4-(trifluoromethylsulfonimidoyl)phenyl]methyl]-2-azaspiro[3.3]heptane-2-carbonyl]-2,5-diazaspiro[3.5]nonan-6-one;
2-[6-[[4-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[3-(trifluoromethylsulfonimidoyl)phenyl]methyl]-2-azaspiro[3.3]heptane-2-carbonyl]-2,5-diazaspiro[3.4]octan-6-one;
2-[6-[[3-(trifluoromethylsulfonimidoyl)phenyl]methyl]-2-azaspiro[3.3]heptane-2-carbonyl]-2,5-diazaspiro[3.5]nonan-6-one;
2-[6-[[3-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[3-(trifluoromethylsulfonimidoyl)phenyl]methyl]-2-azaspiro[3.3]heptane-2-carbonyl]-2,5-diazaspiro[3.4]octan-6-one;
2-[6-[[3-(trifluoromethylsulfonimidoyl)phenyl]methyl]-2-azaspiro[3.3]heptane-2-carbonyl]-2,5-diazaspiro[3.5]nonan-6-one;
2-[6-[[3-(trifluoromethylsulfonimidoyl)phenyl]methyl]-2-azaspiro[3.3]heptane-2-carbonyl]-8-oxa-2,5-diazaspiro[3.5]nonan-6-one;
2-[6-[[4-(trifluoromethylsulfonyl)phenyl]methyl]-2-azaspiro[3.3]heptane-2-carbonyl]-2,5-diazaspiro[3.4]octan-6-one;
2-[6-[[4-(trifluoromethylsulfonyl)phenyl]methyl]-2-azaspiro[3.3]heptane-2-carbonyl]-8-oxa-2,5-diazaspiro[3.5]nonan-6-one;
2-[6-[[3-(trifluoromethoxy)phenyl]sulfonimidoyl]-2-azaspiro[3.3]heptane-2-carbonyl]-2,5-diazaspiro[3.4]octan-6-one;
2-[6-[[3-fluoro-5-(trifluoromethylsulfonimidoyl)phenyl]methyl]-2-azaspiro[3.3]heptane-2-carbonyl]-2,5-diazaspiro[3.4]octan-6-one;
2-[6-[[5-(trifluoromethyl)-3-pyridyl]oxy]-2-azaspiro[3.3]heptane-2-carbonyl]-2,5-diazaspiro[3.4]octan-6-one;
2-[6-[[4-(trifluoromethyl)triazol-2-yl]methyl]-2-azaspiro[3.3]heptane-2-carbonyl]-2,5-diazaspiro[3.4]octan-6-one;
4-[[2-(6-oxo-2,5-diazaspiro[3.4]octane-2-carbonyl)-2-azaspiro[3.3]heptan-6-yl]oxy]-2-(trifluoromethyl)benzonitrile;
4-[[2-(6-oxo-8-oxa-2,5-diazaspiro[3.5]nonane-2-carbonyl)-2-azaspiro[3.3]heptan-6-yl]oxy]-2-(trifluoromethyl)benzonitrile;
2-[6-[(5-chloro-2-pyridyl)oxy]-2-azaspiro[3.3]heptane-2-carbonyl]-2,5-diazaspiro[3.4]octan-6-one;
2-[6-[[1-methyl-4-(trifluoromethyl) pyrazol-3-yl]methyl]-2-azaspiro[3.3]heptane-2-carbonyl]-2,5-diazaspiro[3.4]octan-6-one;
2-[6-[(5-chloro-1,3-dimethyl-pyrazol-4-yl)methyl]-2,6-diazaspiro[3.3]heptane-2-carbonyl]-7-oxa-2,5-diazaspiro[3.4]octan-6-one;
2-[6-[[4-(trifluoromethyl) thiazol-2-yl]methyl]-2,6-diazaspiro[3.3]heptane-2-carbonyl]-2,5-diazaspiro[3.4]octan-6-one;
5-[[2-(6-oxo-2,5-diazaspiro[3.4]octane-2-carbonyl)-2-azaspiro[3.3]heptan-6-yl]oxy]-2-(trifluoromethyl)benzonitrile;
2-[6-[[4-(trifluoromethyl) thiazol-2-yl]oxy-2-azaspiro[3.3]heptane-2-carbonyl]-2,5-diazaspiro[3.4]octan-6-one;
2-[6-[(5-fluoro-2-pyridyl)oxy]-2-azaspiro[3.3]heptane-2-carbonyl]-2,5-diazaspiro[3.4]octan-6-one;
2-[6-[[1-methyl-3-(trifluoromethyl) pyrazol-4-yl]oxy-2-azaspiro[3.3]heptane-2-carbonyl]-2,5-diazaspiro[3.4]octan-6-one;
2-[6-[[4-[1-(trifluoromethyl)cyclopropyl]pyrazol-1-yl]methyl]-2-azaspiro[3.3]heptane-2-carbonyl]-2,5-diazaspiro[3.4]octan-6-one;
2-[6-[6-(trifluoromethyl)pyrimidin-4-yl]methyl]-2-azaspiro[3.3]heptane-2-carbonyl]-2,5-diazaspiro[3.4]octan-6-one;
2-[6-[(3-chloro-5-fluoro-2-pyridyl)oxy]-2-azaspiro[3.3]heptane-2-carbonyl]-2,5-diazaspiro[3.4]octan-6-one;
2-[6-[[2-(trifluoromethyl)pyrimidin-4-yl]methyl]-2-azaspiro[3.3]heptane-2-carbonyl]-2,5-diazaspiro[3.4]octan-6-one;
2-[6-[[2-(trifluoromethyl) thiazol-5-yl]methyl]-2,6-diazaspiro[3.3]heptane-2-carbonyl]-2,5-diazaspiro[3.4]octan-6-one;
2-[6-[[5-(trifluoromethyl)-1H-pyrazol-4-yl]methyl]-2,6-diazaspiro[3.3]heptane-2-carbonyl]-2,5-diazaspiro[3.4]octan-6-one;
2-[7-[[6-(trifluoromethyl)pyridazin-3-yl]methyl]-2,7-diazaspiro[3.4]octane-2-carbonyl]-2,5-diazaspiro[3.4]octan-6-one;
2-[6-(5-fluoro-3-pyridyl)-2,6-diazaspiro[3.3]heptane-2-carbonyl]-7-oxa-2,5-diazaspiro[3.4]octan-6-one;
2-[6-[[4-(trifluoromethyl) thiazol-2-yl]methyl]-2-azaspiro[3.3]heptane-2-carbonyl]-2,5-diazaspiro[3.4]octan-6-one;
2-[6-([1,2,4]triazolo[1,5-a]pyridin-6-ylmethyl)-2-azaspiro[3.3]heptane-2-carbonyl]-2,5-diazaspiro[3.4]octan-6-one;
2-[6-(1H-pyrazolo[4,3-b]pyridin-5-ylmethyl)-2-azaspiro[3.3]heptane-2-carbonyl]-2,5-diazaspiro[3.4]octan-6-one;
2-[6-[[3-fluoro-5-(trifluoromethylsulfonimidoyl)phenyl]methyl]-2-azaspiro[3.3]heptane-2-carbonyl]-8-oxa-2,5-diazaspiro[3.5]nonan-6-one;
2-[7-[[5-(trifluoromethyl)-4H-1,2,4-triazol-3-yl]methyl]-2-azaspiro[3.5]nonane-2-carbonyl]-2,5-diazaspiro[3.4]octan-6-one;
2-[6-[[1-cyclopropyl-3-(trifluoromethyl) pyrazol-4-yl]methyl]-2-azaspiro[3.3]heptane-2-carbonyl]-2,5-diazaspiro[3.4]octan-6-one;
2-[7-[[2-methyl-4-(trifluoromethyl) pyrazol-3-yl]methyl]-2,7-diazaspiro[3.5]nonane-2-carbonyl]-2,5-diazaspiro[3.4]octan-6-one;
2-[6-[[5-methyl-3-(trifluoromethyl) pyrazol-1-yl]methyl]-2-azaspiro[3.3]heptane-2-carbonyl]-2,5-diazaspiro[3.4]octan-6-one;
2-[6-[[4-(trifluoromethyl)pyrimidin-2-yl]methyl]-2-azaspiro[3.3]heptane-2-carbonyl]-2,5-diazaspiro[3.4]octan-6-one;
2-[6-[[3-methyl-5-(trifluoromethyl) pyrazol-1-yl]methyl]-2-azaspiro[3.3]heptane-2-carbonyl]-2,5-diazaspiro[3.4]octan-6-one;
2-[6-[[2-keto-5-(trifluoromethyl)-1H-pyridin-3-yl]methyl]-2-azaspiro[3.3]heptane-2-carbonyl]-2,5-diazaspiro[3.4]octan-6-one;
2-[6-[[3-[1-(trifluoromethyl)cyclopropyl]-1,2,4-triazol-1-yl]methyl]-2-azaspiro[3.3]heptane-2-carbonyl]-2,5-diazaspiro[3.4]octan-6-one;
2-[6-[(2-tert-butylthiazol-5-yl)methyl]-2,6-diazaspiro[3.3]heptane-2-carbonyl]-2,5-diazaspiro[3.4]octan-6-one;
2-[7-[[5-(trifluoromethyl) isothiazol-3-yl]methyl]-2,7-diazaspiro[3.5]nonane-2-carbonyl]-2,5-diazaspiro[3.4]octan-6-one;
2-[6-[[5-(trifluoromethyl)-1H-1,2,4-triazol-3-yl]methyl]-2-azaspiro[3.3]heptane-2-carbonyl]-2,5-diazaspiro[3.4]octan-6-one;
2-[6-[(4-cyclobutylthiadiazol-5-yl)methyl]-2,6-diazaspiro[3.3]heptane-2-carbonyl]-2,5-diazaspiro[3.4]octan-6-one;
2-[6-[[4-(trifluoromethylsulfonimidoyl) phenoxy]-2-azaspiro[3.3]heptane-2-carbonyl]-2,5-diazaspiro[3.4]octan-6-one;
2-[6-(4-dimethylphosphoryl-2-fluoro-benzyl)-2-azaspiro[3.3]heptane-2-carbonyl]-2,5-diazaspiro[3.4]octan-6-one;
2-[7-[[2-methyl-5-(trifluoromethyl) pyrazol-3-yl]methyl]-2,7-diazaspiro[3.5]nonane-2-carbonyl]-2,5-diazaspiro[3.4]octan-6-one;
2-[7-[[5-(trifluoromethyl) isoxazol-3-yl]methyl]-2,7-diazaspiro[3.5]nonane-2-carbonyl]-2,5-diazaspiro[3.4]octan-6-one;
2-[6-(3-dimethylphosphoryl-5-fluoro-benzyl)-2-azaspiro[3.3]heptane-2-carbonyl]-2,5-diazaspiro[3.4]octan-6-one;
2-[6-[(3-cyclopropyl-1,2,4-thiadiazol-5-yl)methyl]-2,6-diazaspiro[3.3]heptane-2-carbonyl]-2,5-diazaspiro[3.4]octan-6-one;
2-[6-[[5-methyl-4-(trifluoromethyl) thiazol-2-yl]methyl]-2,6-diazaspiro[3.3]heptane-2-carbonyl]-2,5-diazaspiro[3.4]octan-6-one;
2-[6-[[5-(trifluoromethyl)-1,3,4-thiadiazol-2-yl]methyl]-2,6-diazaspiro[3.3]heptane-2-carbonyl]-2,5-diazaspiro[3.4]octan-6-one;
2-[6-[[2-methyl-4-(trifluoromethyl) thiazol-5-yl]methyl]-2,6-diazaspiro[3.3]heptane-2-carbonyl]-2,5-diazaspiro[3.4]octan-6-one;
2-[6-(1-methylpyrazol-4-yl)oxy-2-azaspiro[3.3]heptane-2-carbonyl]-2,5-diazaspiro[3.4]octan-6-one;
2-[6-[(4-cyclopropylthiadiazol-5-yl)methyl]-2,6-diazaspiro[3.3]heptane-2-carbonyl]-2,5-diazaspiro[3.4]octan-6-one;
2-[6-[[5-(difluoromethyl)-3-methyl-pyrazol-1-yl]methyl]-2-azaspiro[3.3]heptane-2-carbonyl]-2,5-diazaspiro[3.4]octan-6-one;
2-[6-[[3-(difluoromethyl)-5-methyl-pyrazol-1-yl]methyl]-2-azaspiro[3.3]heptane-2-carbonyl]-2,5-diazaspiro[3.4]octan-6-one;
2-[7-[[5-(difluoromethyl) thiazol-2-yl]methyl]-2,7-diazaspiro[3.5]nonane-2-carbonyl]-2,5-diazaspiro[3.4]octan-6-one;
2-[6-[[5-(trifluoromethyl)-1,2,4-thiadiazol-3-yl]methyl]-2-azaspiro[3.3]heptane-2-carbonyl]-2,5-diazaspiro[3.4]octan-6-one;
2-[7-[[5-(trifluoromethyl)-1H-pyrazol-3-yl]methyl]-2,7-diazaspiro[3.5]nonane-2-carbonyl]-2,5-diazaspiro[3.4]octan-6-one;
2-[6-[[4-(trifluoromethyl) thiazol-2-yl]methyl]-2-azaspiro[3.3]heptane-2-carbonyl]-7-oxa-2,5-diazaspiro[3.4]octan-6-one;
2-[6-[[1-methyl-5-(trifluoromethyl) pyrazol-4-yl]methyl]-2-azaspiro[3.3]heptane-2-carbonyl]-7-oxa-2,5-diazaspiro[3.4]octan-6-one;
4-[[2-(6-keto-7-oxa-2,5-diazaspiro[3.4]octane-2-carbonyl)-2-azaspiro[3.3]heptan-6-yl]methyl]-2-(trifluoromethyl)benzonitrile;
2-[6-[[5-(trifluoromethyl)-2-pyridyl]methyl]-2-azaspiro[3.3]heptane-2-carbonyl]-7-oxa-2,5-diazaspiro[3.4]octan-6-one;
2-[6-[[6-(trifluoromethoxy)-3-pyridyl]methyl]-2-azaspiro[3.3]heptane-2-carbonyl]-7-oxa-2,5-diazaspiro[3.4]octan-6-one;
2-[6-[[3-(trifluoromethylsulfonimidoyl)benzyl]-2-azaspiro[3.3]heptane-2-carbonyl]-7-oxa-2,5-diazaspiro[3.4]octan-6-one;
2-[6-[[3-(trifluoromethylsulfonimidoyl)benzyl]-2-azaspiro[3.3]heptane-2-carbonyl]-7-oxa-2,5-diazaspiro[3.4]octan-6-one;
2-[6-[[4-(trifluoromethylsulfonimidoyl)benzyl]-2-azaspiro[3.3]heptane-2-carbonyl]-7-oxa-2,5-diazaspiro[3.4]octan-6-one;
2-[6-[[4-(trifluoromethylsulfonimidoyl)phenyl]methyl]-2-azaspiro[3.3]heptane-2-carbonyl]-7-oxa-2,5-diazaspiro[3.4]octan-6-one;
2-[6-(3-triflylbenzyl)-2-azaspiro[3.3]heptane-2-carbonyl]-2,5-diazaspiro[3.4]octan-6-one;
2-[6-[[2-fluoro-4-(trifluoromethyl)benzyl]-2,6-diazaspiro[3.3]heptane-2-carbonyl]-2,5-diazaspiro[3.4]octan-6-one;
2-[6-[[4-(trifluoromethyl)triazol-2-yl]methyl]-2-azaspiro[3.3]heptane-2-carbonyl]-8-oxa-2,5-diazaspiro[3.5]nonan-6-one;
2-[6-[[3-(trifluoromethyl)-1,2,4-thiadiazol-5-yl]methyl]-2-azaspiro[3.3]heptane-2-carbonyl]-8-oxa-2,5-diazaspiro[3.5]nonan-6-one;
2-[6-[[5-(difluoromethyl)-3-methyl-pyrazol-1-yl]methyl]-2-azaspiro[3.3]heptane-2-carbonyl]-8-oxa-2,5-diazaspiro[3.5]nonan-6-one;
2-[6-[[1-(trifluoromethyl) pyrazol-4-yl]methyl]-2-azaspiro[3.3]heptane-2-carbonyl]-8-oxa-2,5-diazaspiro[3.5]nonan-6-one;
2-[6-[[2-methyl-4-(trifluoromethyl) pyrazol-3-yl]methyl]-2-azaspiro[3.3]heptane-2-carbonyl]-8-oxa-2,5-diazaspiro[3.5]nonan-6-one;
2-[6-[[1-methyl-5-(trifluoromethyl) pyrazol-3-yl]methyl]-2-azaspiro[3.3]heptane-2-carbonyl]-8-oxa-2,5-diazaspiro[3.5]nonan-6-one;
2-[6-[[1-methyl-5-(trifluoromethyl) pyrazol-4-yl]methyl]-2-azaspiro[3.3]heptane-2-carbonyl]-8-oxa-2,5-diazaspiro[3.5]nonan-6-one;
2-[6-[[4-(trifluoromethyl) thiazol-2-yl]methyl]-2-azaspiro[3.3]heptane-2-carbonyl]-8-oxa-2,5-diazaspiro[3.5]nonan-6-one;
2-[6-[[1-methyl-3-(trifluoromethyl) pyrazol-4-yl]methyl]-2-azaspiro[3.3]heptane-2-carbonyl]-8-oxa-2,5-diazaspiro[3.5]nonan-6-one;
2-[6-[(3,5-difluoro-2-pyridyl)methyl]-2-azaspiro[3.3]heptane-2-carbonyl]-8-oxa-2,5-diazaspiro[3.5]nonan-6-one;
2-[6-[[2-fluoro-4-(trifluoromethyl)benzyl]-2,6-diazaspiro[3.3]heptane-2-carbonyl]-8-oxa-2,5-diazaspiro[3.5]nonan-6-one;
2-[6-(2-fluoro-4-triflyl-benzyl)-2,6-diazaspiro[3.3]heptane-2-carbonyl]-8-oxa-2,5-diazaspiro[3.5]nonan-6-one;
2-[6-[(4-tert-butyloxazol-2-yl)methyl]-2,6-diazaspiro[3.3]heptane-2-carbonyl]-2,5-diazaspiro[3.4]octan-6-one; or
2-[6-[(2-keto-1H-pyridin-3-yl)methyl]-2,6-diazaspiro[3.3]heptane-2-carbonyl]-2,5-diazaspiro[3.4]octan-6-one;
or a pharmaceutically acceptable salt thereof.

13. The compound of formula (I) according to claim 11, wherein the compound is:
2-[6-(4-mesylbenzyl)-2-azaspiro[3.3]heptane-2-carbonyl]-7-oxa-2,5-diazaspiro[3.4]octan-6-one;
2-[7-[[5-(trifluoromethyl)-2-pyridyl]amino]-2-azaspiro[3.5]nonane-2-carbonyl]-7-oxa-2,5-diazaspiro[3.4]octan-6-one;
2-[6-[[4-(trifluoromethylsulfonimidoyl)benzyl]-2-azaspiro[3.3]heptane-2-carbonyl]-2,5-diazaspiro[3.4]octan-6-one;
2-[6-[[3-(trifluoromethylsulfonimidoyl)benzyl]-2-azaspiro[3.3]heptane-2-carbonyl]-2,5-diazaspiro[3.4]octan-6-one;
2-[6-[[6-(trifluoromethyl)-3-pyridyl]methyl]-2-azaspiro[3.3]heptane-2-carbonyl]-7-oxa-2,5-diazaspiro[3.4]octan-6-one;
2-[7-[[6-(trifluoromethyl)pyridazin-3-yl]oxy-2-azaspiro[3.5]nonane-2-carbonyl]-7-oxa-2,5-diazaspiro[3.4]octan-6-one;
5-[[2-(6-keto-2,5-diazaspiro[3.4]octane-2-carbonyl)-2-azaspiro[3.3]heptan-6-yl]methyl]-2-(trifluoromethyl)benzonitrile;
5-[[2-(6-keto-2,5,7-triazaspiro[3.4]octane-2-carbonyl)-2-azaspiro[3.3]heptan-6-yl]methyl]-2-(trifluoromethyl)benzonitrile;
5-[[2-(6-keto-7-oxa-2,5-diazaspiro[3.4]octane-2-carbonyl)-2-azaspiro[3.3]heptan-6-yl]methyl]-2-(trifluoromethyl)benzonitrile; or
2-[6-[[5-(trifluoromethyl)pyrimidin-2-yl]methyl]-2-azaspiro[3.3]heptane-2-carbonyl]-2,5-diazaspiro[3.4]octan-6-one;
or is a pharmaceutically acceptable salt thereof.

14. A process of manufacturing a compound of formula (I) according to claim 1, or a pharmaceutically acceptable salt thereof, comprising:
(a) reacting nucleophilic spirocyclic amine 1 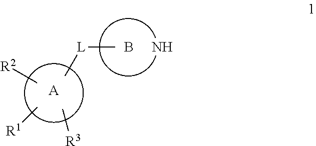
wherein R¹ to R³, L, A, and B are as defined in claim 1;
with an activated intermediate of formula 2 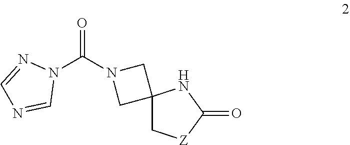
wherein Z is as defined in claim 1;
in a solvent and in the presence of a base to form said compound of formula (I); or
(b) reacting nucleophilic spirocyclic amine 1 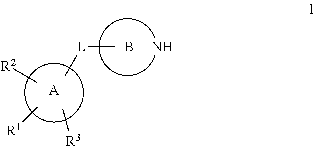
wherein R¹ to R³, L, A, and B are as defined in claim 1;
with an activated intermediate of formula 2a 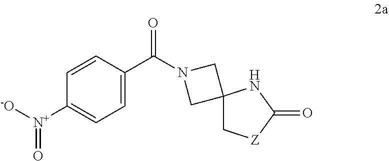
wherein Z is as defined in claim 1;
in a solvent and in the presence of a base to form said compound of formula (I); and
optionally
(c) contacting said compound of formula (I) with an acid to form a pharmaceutically acceptable salt thereof.

15-22. (canceled)

23. A pharmaceutical composition comprising a compound of formula (I) according to claim 1, or a pharmaceutically acceptable salt thereof, and a therapeutically inert carrier.

24. A pharmaceutical composition comprising a compound of formula (I) according to claim 12, or a pharmaceutically acceptable salt thereof, and a therapeutically inert carrier.

25. A pharmaceutical composition comprising a compound of formula (I) according to claim 13, or a pharmaceutically acceptable salt thereof, and a therapeutically inert carrier.

26. A method for the treatment or prophylaxis of a disorder in a human in need thereof, comprising administering a therapeutically effective amount of a compound of formula (I) according to claim 1, or of a pharmaceutically acceptable salt thereof, to the human, wherein the disorder is neuroinflammation, neurodegenerative diseases, pain, cancer, mental disorder, or inflammatory bowel disease.

27. A method for the treatment or prophylaxis of a disorder in a human in need thereof, comprising administering a therapeutically effective amount of a compound of formula (I) according to claim 12, or of a pharmaceutically acceptable salt thereof, to the human, wherein the disorder is neuroinflammation, neurodegenerative diseases, pain, cancer, mental disorder, or inflammatory bowel disease.

28. A method for the treatment or prophylaxis of a disorder in a human in need thereof, comprising administering a therapeutically effective amount of a compound of formula (I) according to claim 13, or of a pharmaceutically acceptable salt thereof, to the human, wherein the disorder is neuroinflammation, neurodegenerative diseases, pain, cancer, mental disorder, or inflammatory bowel disease.
